(19) United States
(12) Patent Application Publication
(10) Pub. No.: US 2017/0183386 A1
(43) Pub. Date: Jun. 29, 2017

(54) PEPTIDES, AND USES THEREOF

(71) Applicant: NURITAS LIMITED, Dublin 2 (IE)

(72) Inventors: Nora KHALDI, Shankill, Co. Dublin (IE); Cyril LOPEZ, Dublin 2 (IE); Alessandro ADELFIO, Rathmines, Dublin 6 (IE)

(73) Assignee: NURITAS LIMITED, Dublin 2 (IE)

(21) Appl. No.: 15/213,276

(22) Filed: Jul. 18, 2016

(30) Foreign Application Priority Data
Jul. 16, 2015 (EP) 15177013.8
Jul. 16, 2015 (EP) 15177017.9
Jul. 16, 2015 (EP) 15177018.7
Jul. 16, 2015 (EP) 15177175.5

(51) Int. Cl. C07K 14/415; A23L 33/18; A61Q 19/08; A01N 37/18; A61K 38/16; A61K 8/64

(52) U.S. Cl. CPC C07K 14/415; A61K 38/168; A61K 8/645; A61Q 19/08; A01N 37/18; A61K 47/48215; A23L 33/18; A23V 2002/00

(57) ABSTRACT

A composition comprising a plurality of bioactive peptides including SEQUENCE ID NO's 555 and 701 is described. The composition may be a powder that is enriched in peptides having a molecular weight less than 10 KD. The bioactive peptides included in the composition have been found to have anti-inflammatory, glucose-transport promoting, and cellular growth promoting activities.

### CROSS-REFERENCE TO RELATED APPLICATIONS

This application claims benefit under 35 U.S.C. §119(a) of European Application Nos. 15177103.8 filed Jul. 16, 2015; 15177017.9 filed Jul. 16, 2015; 15177018.7 filed Jul. 16, 2015; and 15177175.5 filed Jul. 16, 2015, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Mar. 6, 2017, is named 048262-087760-US_SL.TXT and is 363,792 bytes in size.

### STATEMENTS OF INVENTION

In a first aspect, the invention provides a peptide, typically having 4 to 50 amino acids, and comprising an amino acid sequence selected from SEQUENCE ID NO's 1 to 1312, or a variant or thereof (hereafter “peptide of the invention”).

In one embodiment, the peptide comprises (or consists of) an amino acid sequence selected from SEQUENCE ID NO'S 1 to 151 and 707, or a variant or fragment thereof, wherein the peptide typically has anti-inflammatory activity.

In one embodiment, the peptide comprises (or consists of) an amino acid sequence selected from SEQUENCE ID NO'S 152 to 554 and 655 to 701, or a variant or fragment thereof, wherein the peptide typically has cellular growth promoting activity.

In one embodiment, the peptide comprises (or consists of) an amino acid sequence selected from SEQUENCE ID NO'S 555 to 614 and 702 to 706, or a variant or fragment thereof, wherein the peptide typically has glucose transport promoting activity.

In one embodiment, the peptide comprises (or consists of) an amino acid sequence selected from SEQUENCE ID NO'S 615 to 643 and 644 to 654, or a variant or fragment thereof, wherein the peptide typically has anti-bacterial activity.

In one embodiment, the peptide of the invention comprises a sequence selected from SEQUENCE ID NO's: 1 to 1312.

In one embodiment, the peptide of the invention consists of a sequence selected from SEQUENCE ID NO's: 1 to 1312.

In one embodiment, the variant of the peptide has at least 70%, 75%, 80%, 85%, 90% or 95% sequence homology with the reference peptide of the invention.

In one embodiment, the peptide of the invention is a modified peptide.

In one embodiment, the invention provides a composition comprising a peptide of the invention, or a variant or fragment thereof (hereafter “composition of the invention”).

In one embodiment, the composition comprises a peptide comprising the amino acid sequence of SEQUENCE ID NO: 41 or a variant thereof selected from SEQUENCE ID NO 706.

In one embodiment, the composition comprises a peptide comprising the amino acid sequence of SEQUENCE ID NO: 555 or a variant or fragment thereof selected from SEQUENCE ID NO'S 3, 170, 204, 213, 556, 558, 563.

In one embodiment, the composition comprises a peptide comprising the amino acid sequence of SEQUENCE ID NO: 701 or a variant or fragment thereof.

In one embodiment, the composition of the invention comprises a plurality of peptides of the invention selected from SEQUENCE ID NO'S 1 to 39, 152 to 410, 555 to 594, and 615 to 638.

In one embodiment, the composition of the invention comprises a plurality of peptides of the invention selected from SEQUENCE ID NO'S 5, 23, 22, 38, 39, 21, 258, 242, 261, 211, 222, 249, 235, 295, 283, 284, 216, 555 and 701. In one embodiment, the composition comprises at least 2, 3, 4, 5, 6, 7, 8, 9 or 10 of the above-referenced peptides. In one embodiment, the composition comprises all of the above-referenced peptides.

In one embodiment, the composition comprises SEQUENCE ID NO's: 555 and 701, and one or more peptides (for example 1, 2, 3, 4, 5, 6, 7, 8, 9 or 10 peptides) selected from SEQUENCE ID NO'S 5, 23, 22, 38, 39, 21, 258, 242, 261, 211, 222, 249, 235, 295, 283, 284, 216.

In one embodiment, the composition of the invention comprises substantially all of the peptides of SEQUENCE ID NO'S 1 to 39, 152 to 410, 555 to 594, and 615 to 638.

In one embodiment, the composition of the invention comprises a plurality of peptides of the invention selected from SEQUENCE ID NO'S 40 to 151, 411 to 549, 595 to 614 and 639 to 643.

In one embodiment, the composition of the invention comprises a plurality of peptides of the invention selected from SEQUENCE ID NO'S 74, 40, 41, 502, 496, 417, 467, 448, 452, 451, 443, 447, 480, 444, 245 and 246.

In one embodiment, the composition comprises at least 2, 3, 4, 5, 6, 7, 8, 9 or 10 of the above-referenced peptides. In one embodiment, the composition comprises all of the above-referenced peptides.

In one embodiment, the composition comprises SEQUENCE ID NO: 41, and one or more peptides (for example 1, 2, 3, 4, 5, 6, 7, 8, 9 or 10 peptides) selected from SEQUENCE ID NO'S 74, 40, 502, 496, 417, 467, 448, 452, 451, 443, 447, 480, 444, 245 and 246.

In one embodiment, the composition of the invention comprises substantially all of the peptides of SEQUENCE ID NO'S 40 to 151, 411 to 549, 595 to 614 and 639 to 643.

In one embodiment, the composition is a powder. In one embodiment, the composition is a liquid. In one embodiment, the liquid has a pH between 5 and 9, preferably between 6 and 8, and ideally about 7. In one embodiment, the composition is a cream. In one embodiment, the cream has a pH between 5 and 9, preferably between 6 and 8, and ideally about 7.

In one embodiment, the composition is enriched in peptides having a molecular weight of less than 10 KD. This means that the weight % of peptides in the powder having a MW of less than 10KD is greater than the weight % of peptides in the powder having a weight of greater than 10 KD. Such a composition does not exist in nature. In one embodiment, the composition is depleted in cellular debris.

In one embodiment, the invention relates to a food product comprising a composition of the invention, in which the composition is optionally in powder form.

In one embodiment, the invention relates to a personal care product comprising a composition of the invention, in which the composition is optionally in powder form.

In one embodiment, the invention relates to a pharmaceutical product comprising a composition of the invention, in which the composition is optionally in powder form.

In one embodiment, the invention relates to a nutritional or dietary supplement comprising a composition of the invention, in which the composition is optionally in powder form.

In one embodiment, the invention relates to a topical composition comprising a composition of the invention, in which the composition of the invention is optionally in powder form.

The invention also relates to a comestible product comprising a peptide of the invention. Preferably the comestible product is man-made.

Preferably, the comestible product is a food product for human or animal (mammalian) consumption.

In one embodiment the man-made comestible product is a beverage. In one embodiment the man-made comestible product is a bakery product. In one embodiment the man-made comestible product is a dairy product. In one embodiment the man-made comestible product is a snack product. In one embodiment the man-made comestible product is a baked extruded food product. In one embodiment the man-made comestible product is powdered milk. In one embodiment the man-made comestible product is an infant formula product. In one embodiment the man-made comestible product is a confectionary product. In one embodiment the man-made comestible product is a yoghurt. In one embodiment the man-made comestible product is a yoghurt drink. In one embodiment the man-made comestible product is an ice cream product. In one embodiment the man-made comestible product is a frozen food product. In one embodiment the man-made comestible product is a breakfast cereal. In one embodiment the man-made comestible product is a bread. In one embodiment the man-made comestible product is a flavoured milk drink. In one embodiment the man-made comestible product is a confectionary bar. In one embodiment the man-made comestible product is a tea or tea product. In one embodiment the man-made comestible product is a based extruded snack product. In one embodiment the man-made comestible product is a fried snack product. In one embodiment the man-made comestible product is a nutritional supplement. In one embodiment the man-made comestible product is a sports nutritional product. In one embodiment the man-made comestible product is a baby food product. In one embodiment the man-made comestible product is a speciality food product for immunocompromised individuals. In one embodiment the man-made comestible product is a food for geriatric patients.

The invention also relates to a man-made personal care product comprising a peptide of the invention.

The invention also relates to a man-made personal care product comprising a composition of peptides of the invention.

In one embodiment, the personal care product is formulated for topical delivery to the skin of a human.

In one embodiment the personal care product is a skincare product. In one embodiment the personal care product is a haircare product. In one embodiment the personal care product is a dentrifice product. In one embodiment the personal care product is a perfumery product. In one embodiment the personal care product is a deodorant product. In one embodiment the personal care product is an anti-perspirant product. In one embodiment the personal care product is a soap. In one embodiment the personal care product is a liquid soap. In one embodiment the personal care product is a cream. In one embodiment the personal care product is a lotion. In one embodiment the personal care product is a gel. In one embodiment the personal care product is a powder.

The invention also relates to a peptide or composition of the invention for use in treatment or prevention of inflammation, or an inflammatory disorder, in a mammal. In one embodiment, the peptide is selected from, or the composition comprises one or more peptides selected from, SEQUENCE ID NO'S 1 to 151 and 707.

In one embodiment the inflammation is symptomatic inflammation.

In one embodiment the inflammatory disorder is an inflammatory disorder of the joints. In one embodiment the inflammatory disorder is an inflammatory disorder of the cardiovascular system. In one embodiment the inflammatory disorder is an autoimmune disease. In one embodiment the inflammatory disorder is a lung and airway inflammatory disorder. In one embodiment the inflammatory disorder is an intestinal inflammatory disorder. In one embodiment the inflammatory disorder is dermatitis. In one embodiment the inflammatory disorder is acne vulgaris. In one embodiment the inflammatory disorder is psoriasis. In one embodiment the inflammatory disorder is rheumatoid arthritis. In one embodiment the inflammatory disorder is cardiovascular disease. In one embodiment the inflammatory disorder is atherosclerosis. In one embodiment the inflammatory disorder is Type I diabetes.

In one embodiment the inflammatory disorder is Graves disease. In one embodiment the inflammatory disorder is Guillain-Barre disease. In one embodiment the inflammatory disorder is Lupus. In one embodiment the inflammatory disorder is Psoriatic arthritis. In one embodiment the inflammatory disorder is Ulcerative colitis. In one embodiment the inflammatory disorder is asthma. In one embodiment the inflammatory disorder is cystic fibrosis. In one embodiment the inflammatory disorder is COPD. In one embodiment the inflammatory disorder is emphysema. In one embodiment the inflammatory disorder is acute respiratory distress syndrome. In one embodiment the inflammatory disorder is colitis. In one embodiment the inflammatory disorder is inflammatory bowel disease.

The invention also relates to a peptide of the invention for use in treatment or prevention of pain in a mammal. In one embodiment, the peptide is selected from SEQUENCE ID NO'S 1 to 151 and 707.

The invention also relates to a composition of peptides of the invention for use in treatment or prevention of pain in a mammal. In one embodiment, the composition comprises one or more peptides selected from, SEQUENCE ID NO'S 1 to 151 and 707.

The invention also relates to a peptide of the invention for use in treatment or prevention of a metabolic disorder in a mammal. In one embodiment, the peptide is selected from SEQUENCE ID NO'S 1 to 151 and 707.

The invention also relates to a composition of peptides of the invention for use in treatment or prevention of a metabolic disorder in a mammal. In one embodiment, the composition comprises one or more peptides selected from, SEQUENCE ID NO'S 1 to 151 and 707.

In one embodiment, the metabolic disorder is pre-diabetes. In one embodiment, the metabolic disorder is diabetes. In one embodiment, the metabolic disorder is Type-1 diabetes. In one embodiment, the metabolic disorder is Type-2 diabetes. In one embodiment, the metabolic disorder is metabolic syndrome. In one embodiment, the metabolic disorder is obesity. In one embodiment, the metabolic disorder is diabetic dyslipidemia. In one embodiment, the metabolic disorder is hyperlipidemia. In one embodiment, the metabolic disorder is hypertension. In one embodiment, the metabolic disorder is hypertriglyceridemia. In one embodiment, the metabolic disorder is hyperfattyacidemia. In one embodiment, the metabolic disorder is hypercholerterolemia. In one embodiment, the metabolic disorder is hyperinsulinemia. In one embodiment, the metabolic disorder is MODY.

The invention also relates to a peptide of the invention for use in maintaining or restoring gut health in a mammal. In one embodiment, the peptide is selected from SEQUENCE ID NO'S 1 to 151 and 707.

The invention also relates to a composition of peptides of the invention for use in maintaining or restoring gut health in a mammal. In one embodiment, the composition comprises one or more peptides selected from, SEQUENCE ID NO'S 1 to 151 and 707.

Such peptides can be used in personal care, supplement, food and pharmaceutical products to treat and maintain healthy levels of inflammation throughout the body. The present invention is concerned with the huge need for food-derived specific peptides and peptide compositions that reduces inflammation in a way that is able to be processed by the body without completely blocking the immune response and causing autoimmune issues and other undesirable side effects. The invention may ultimately help the 2 billion people suffering from inflammation. The invention also relates to a man-made wound treatment composition comprising a peptide of the invention. In one embodiment, the peptide is selected from SEQUENCE ID NO'S 152 to 554 and 655 to 701. The invention also relates to a man-made wound treatment composition comprising a composition of the invention. In one embodiment, the composition comprises one or more peptides selected from, SEQUENCE ID NO'S 152 to 554 and 655 to 701. Typically, the wound treatment composition is formulated for topical application to a wound. In one embodiment, the composition comprises a cream, gel, lotion, powder.

The invention also relates to a plaster, bandage or dressing suitable for application to a wound and comprising a peptide or composition of the invention. In one embodiment, the peptide is selected from SEQUENCE ID NO'S 152 to 554 and 655 to 701.

The invention also relates to a man-made cell culture media comprising a peptide of the invention. In one embodiment, the peptide is selected from SEQUENCE ID NO'S 152 to 554 and 655 to 701. The invention also relates to a man-made cell culture media comprising a composition of the invention. In one embodiment, the composition comprises one or more peptides selected from SEQUENCE ID NO'S 152 to 554 and 655 to 701. In one embodiment, the cell culture media is formulated for culture of eukaryotic cells. In one embodiment, the cell culture media is formulated for culture of prokaryotic cells.

The invention also relates to a plaster, bandage or dressing suitable for application to a wound and comprising a peptide or composition of the invention.

The invention also relates to a peptide of the invention for use in promoting growth of a cell. In one embodiment, the peptide is selected from SEQUENCE ID NO'S 152 to 554 and 655 to 701.

The invention also relates to a peptide of the invention for use in promoting growth of a cell culture. In one embodiment, the peptide is selected from SEQUENCE ID NO'S 152 to 554 and 655 to 701.

The invention also relates to a peptide of the invention for use in promoting growth of a tissue. In one embodiment, the peptide is selected from SEQUENCE ID NO'S 152 to 554 and 655 to 701.

The invention also relates to a peptide of the invention for use in promoting growth of dermal or epithelial tissue. In one embodiment, the peptide is selected from SEQUENCE ID NO's 152 to 554 and 655 to 701.

The invention also relates to a peptide of the invention for use in promoting growth of skin. In one embodiment, the peptide is selected from SEQUENCE ID NO'S 152 to 554 and 655 to 701.

The invention also relates to a peptide of the invention for use in promoting growth of an organ. In one embodiment, the peptide is selected from SEQUENCE ID NO'S 152 to 554 and 655 to 701.

The invention also relates to a peptide of the invention for use in promoting growth of an organism. In one embodiment, the peptide is selected from SEQUENCE ID NO'S 152 to 554 and 655 to 701.

The invention also relates to a composition of the invention for use in promoting growth of a cell. In one embodiment, the composition comprises one or more peptides selected from SEQUENCE ID NO'S 152 to 554 and 655 to 701.

The invention also relates to a composition of the invention for use in promoting growth of a cell culture. In one embodiment, the composition comprises one or more peptides selected from SEQUENCE ID NO'S 152 to 554 and 655 to 701.

The invention also relates to a composition of the invention for use in promoting growth of a tissue. In one embodiment, the composition comprises one or more peptides selected from SEQUENCE ID NO'S 152 to 554 and 655 to 701.

The invention also relates to a composition of the invention for use in promoting growth of epithelial tissue. In one embodiment, the composition comprises one or more peptides selected from SEQUENCE ID NO'S 152 to 554 and 655 to 701.

The invention also relates to a composition of the invention for use in promoting growth of skin. In one embodiment, the composition comprises one or more peptides selected from SEQUENCE ID NO'S 152 to 554 and 655 to 701.

The invention also relates to a composition of the invention for use in promoting growth of an organ. In one embodiment, the composition comprises one or more peptides selected from SEQUENCE ID NO'S 152 to 554 and 655 to 701.

The invention also relates to a composition of the invention for use in promoting growth of an organism. In one embodiment, the composition comprises one or more peptides selected from SEQUENCE ID NO'S 152 to 554 and 655 to 701.

In one embodiment, the cell, tissue or organism has a normal pathology (for example ageing skin). In one embodiment of the invention, the cell, tissue or skin has abnormal pathology (for example tissue damaged due to trauma, drug use, or epithelial tissue in the GI tract damaged due to an inflammatory disorder).

The growth promoting uses may be in-vivo or in-vitro uses. The growth promoting uses may involve administration to mammal externally (i.e. to the skin) or internally (i.e. to the GI tract).

The invention also relates to a peptide of the invention for use in slowing or inhibiting ageing of human skin. In one embodiment, the peptide is selected from SEQUENCE ID NO'S 152 to 554 and 655 to 701.

The invention also relates to a method of slowing or inhibiting ageing of human skin comprising a step of administering a peptide of the invention to the human skin. Typically, the peptide of the invention is administered topically to the skin. Administration may be by means of a plaster or patch or a formulation suitable for topical application. In one embodiment, the peptide is selected from SEQUENCE ID NO'S 152 to 554 and 655 to 701.

The invention also relates to a composition of the invention for use in slowing or inhibiting ageing of human skin. The invention also relates to a peptide of the invention for use in preventing or slowing ageing of the human skin. In one embodiment, the peptide is selected from SEQUENCE ID NO'S 152 to 554 and 655 to 701.

The invention also relates to a method of slowing or inhibiting ageing of human skin comprising a step of administering a composition of the invention to the human skin. Typically, the composition of the invention is administered topically to the skin. In one embodiment, the composition comprises one or more peptides selected from SEQUENCE ID NO'S 152 to 554 and 655 to 701.

The invention also relates to a peptide of the invention for use in treatment of a wound in a mammal. In one embodiment, the peptide is selected from SEQUENCE ID NO'S 152 to 554 and 655 to 701.

The invention also relates to a composition of peptides of the invention for use in treatment of a wound in a mammal. In one embodiment, the composition comprises one or more peptides selected from SEQUENCE ID NO'S 152 to 554 and 655 to 701.

The invention also relates to a wound treatment composition or product of the invention for use in treatment of a wound in a mammal. In one embodiment, the composition comprises one or more peptides selected from SEQUENCE ID NO'S 152 to 554 and 655 to 701.

The invention also relates to a peptide of the invention for use in treatment or prevention of a disease or condition characterised by damaged epithelial cells or tissue. In one embodiment, the peptide is selected from SEQUENCE ID NO'S 152 to 554 and 655 to 701.

The invention also relates to a composition of peptides of the invention for use in treatment or prevention of a disease or condition characterised by damaged dermal or epithelial cells or tissue. In one embodiment, the composition comprises one or more peptides selected from SEQUENCE ID NO'S 152 to 554 and 655 to 701.

In one embodiment, the disease or condition characterised by damaged dermal or epithelial cells or tissue is selected from cancer, trauma

The invention also relates to a peptide of the invention for use in improving muscle status in a mammal. In one embodiment, the peptide is selected from SEQUENCE ID NO'S 555 to 614 and 702 to 706.

The invention also relates to a composition of the invention for use in improving muscle status in a mammal. In one embodiment, the composition comprises one or more peptides selected from SEQUENCE ID NO'S 555 to 614 and 702 to 706.

The invention also relates to a peptide of the invention for use in promoting recovery of muscle, typically following exercise. In one embodiment, the peptide is selected from SEQUENCE ID NO'S555 to 614 and 702 to 706.

The invention also relates to a composition of the invention for use in promoting recovery of muscle, typically following exercise. In one embodiment, the composition comprises one or more peptides selected from SEQUENCE ID NO'S 555 to 614 and 702 to 706.

The invention also relates to a peptide of the invention for use in maintaining or restoring muscle health (for example lean tissue mass) in a mammal. In one embodiment, the peptide is selected from SEQUENCE ID NO'S 555 to 614 and 702 to 706.

The invention also relates to a composition of peptides of the invention for use in maintaining or restoring muscle health (for example lean tissue mass) in a mammal. In one embodiment, the composition comprises one or more peptides selected from SEQUENCE ID NO'S 555 to 614 and 702 to 706.

The invention also relates to a peptide of the invention for use in enhancing physical performance. In one embodiment, the peptide is selected from SEQUENCE ID NO'S 555 to 614 and 702 to 706.

The invention also relates to a composition of the invention for use in enhancing physical performance. In one embodiment, the composition comprises one or more peptides selected from SEQUENCE ID NO'S 555 to 614 and 702 to 706.

The invention also relates to a peptide of the invention for use in treatment or prevention of a disease or condition characterised by lethargy or low energy levels. In one embodiment, the peptide is selected from SEQUENCE ID NO'S 555 to 614 and 702 to 706.

The invention also relates to a composition of peptides of the invention for use in treatment or prevention of a disease or condition characterised by lethargy or low energy levels. In one embodiment, the composition comprises one or more peptides selected from SEQUENCE ID NO'S 555 to 614 and 702 to 706.

The invention also relates to a peptide or composition of the invention for use in treating or preventing a bacterial infection in a mammal. In one embodiment, the peptide is selected from SEQUENCE ID NO'S 615 to 643 and 644 to 654. In one embodiment, the composition comprises one or more peptides selected from SEQUENCE ID NO'S 615 to 643 and 644 to 654.

The invention also relates to a peptide or composition of the invention for use as an antimicrobial or antibacterial agent. In one embodiment, the peptide is selected from SEQUENCE ID NO'S 615 to 643 and 644 to 654. In one embodiment, the composition comprises one or more peptides selected from SEQUENCE ID NO'S 615 to 643 and 644 to 654.

The invention also relates to a peptide or composition of the invention for use as a preservative. In one embodiment, the peptide is selected from SEQUENCE ID NO'S 615 to 643 and 644 to 654 . In one embodiment, the composition comprises one or more peptides selected from SEQUENCE ID NO'S 615 to 643 and 644 to 654.

The invention also relates to a peptide or composition of the invention for use as a preservative in a perishable product, such as a food product or a personal care composition. In one embodiment, the peptide is selected from SEQUENCE ID NO'S 615 to 643 and 644 to 654. In one embodiment, the composition comprises one or more peptides selected from SEQUENCE ID NO'S 615 to 643 and 644 to 654.

The invention also relates to a peptide or composition of the invention for use as an anti-bacterial agent in a personal care composition. In one embodiment, the peptide is selected from SEQUENCE ID NO'S 615 to 643 and 644 to 654. In one embodiment, the composition comprises one or more peptides selected from SEQUENCE ID NO'S 615 to 643 and 644 to 654.

The invention also relates to a peptide or composition of the invention for use as an anti-bacterial agent in a household cleaning product. In one embodiment, the peptide is selected from SEQUENCE ID NO'S 615 to 643 and 644 to 654. In one embodiment, the composition comprises one or more peptides selected from SEQUENCE ID NO'S 615 to 643 and 644 to 654.

The invention also relates to a peptide or composition of the invention for use as a plant biocidal agent. In one embodiment, the peptide is selected from SEQUENCE ID NO'S 615 to 643 and 644 to 654. In one embodiment, the composition comprises one or more peptides selected from SEQUENCE ID NO'S 615 to 643 and 644 to 654.

The invention also relates to a peptide of the invention for use in treatment or prevention of a disease or condition characterised by a bacterial infection. The invention also relates to a composition of peptides of the invention for use in treatment or prevention of a disease or condition characterised by a bacterial infection. In one embodiment, the bacterial infection is a MRSA infection. In one embodiment, the peptide is selected from SEQUENCE ID NO'S 615 to 643 and 644 to 654. In one embodiment, the composition comprises one or more peptides selected from SEQUENCE ID NO'S 615 to 643 and 644 to 654.

The invention also relates to a pharmaceutical composition comprising a peptide of the invention in combination with a pharmaceutically acceptable carrier.

The invention also relates to a pharmaceutical composition comprising a composition of peptides of the invention in combination with a pharmaceutically acceptable carrier.

The invention also relates to a comestible product, for example a food product comprising a composition of the invention, for example a dairy or non-dairy product, a solid food or a beverage, a food additive or supplement. The dairy product may be a milk, a cheese, or yoghurt. In one embodiment, the food product is a snack bar. The food product may comprise any amount of the composition of the invention, for example from 0.1% to 30% (w/w).

The food product may be a Food for Specific Medicinal Purposes (FSMP) which is defined as foods that are specifically formulated, processed and intended for the dietary management of diseases, disorders or medical conditions of individuals who are being treated under medical supervision. These foods are intended for the exclusive or partial feeding of people whose nutritional requirements cannot be met by normal foods.

The invention also relates to a conjugate comprising a peptide of the invention conjugated to a binding partner. The binding partner may be selected from a drug, an agent to increase the lipophilicity of the conjugate, or an agent to prolong the plasma half-life of the peptide of the invention. In one embodiment, the peptide is modified to facilitate covalent bonding between the peptide and the binding partner.

The peptides of the invention are used in the topical cosmetic or pharmaceutical composition of this invention at cosmetically or pharmaceutically effective concentrations to achieve the desired effect; in a preferred form with regards to the total weight of the composition, between 0.00000001% (in weight) and 20% (in weight); preferably between 0.000001% (in weight) and 15% (in weight), more preferably between 0.0001% (in weight) and 10% (in weight) and even more preferably between 0.0001% (in weight) and 5% (in weight). Ideally, the peptides of the present invention are preferably used from about 0.00001% w/w to about 0.5% w/w [0.1 to 5000 ppm], and more preferably from 0.00005 w/w to about 0.05 w/w [0.5 to 500 ppm], and most preferably from about 0.0001 w/w to about 0.01 w/w of the composition [1 to 100 ppm]. Ideally, the peptides of the present invention are preferably used from about 0.0001% w/w to about 0.004% w/w of the composition.

For compositions of peptides of the invention, a typical daily dosage may be 0.2g to 100g. However, when administered as a food for special medicinal purpose, or medical food, the daily dosage may be 50-500g per day.

The dosage of compositions of the invention for use in food products and food supplements (i.e. comestible compositions) will be broadly in the 0.2-100 g/day range. In one embodiment, the daily dosage is 1-10 g/day, ideally about 3-8 g/day. In one embodiment, the daily dosage is 10-20 g/day. In one embodiment, the daily dosage is 20-30 g/day. In one embodiment, the daily dosage is 30-40 g/day. In one embodiment, the daily dosage is 10-100 g/day. In one embodiment, the daily dosage is about 5 g/day, ideally about 3-8 g/day. In one embodiment, the dosage is 2-1000 mg/day/kg body weight. In one embodiment, the dosage is 10-500 mg/day/kg body weight. In one embodiment, the dosage is 10-100 mg/day/kg body weight. In one embodiment, the dosage is 30-70 mg/day/kg body weight.

The invention also provides topical composition comprising a peptide of the invention. It will be appreciated that the topical composition may comprise a plurality of peptides, fragments and/or variants. In one embodiment, the topical composition comprises substantially all the peptides. In one embodiment, the topical composition comprises substantially all the variants.

The topical composition of the invention may be presented in a formulation selected from the group comprising creams, multiple emulsions, anhydrous compositions, aqueous dispersions, oils, milks, balsams, foams, lotions, gels, cream gels, hydro-alcoholic solutions, hydro-glycolic solutions, cosmetic, personal care product, hydrogels, liniments, sera, soaps, dusting powder, paste, semi solid formulations, liniments, serums, shampoo, conditioner, ointments, any rinse off formulation, talc, mousses, powders, sprays, aerosols, solutions, suspensions, emulsions, syrups, elixirs, polysaccharide films, patches, gel patches, bandages, an adhesive system, water-in-oil emulsions, oil-in-water emulsions, and silicone emulsions.

In an embodiment of the current invention, the emulsion contains a lipid or oil. The emulsion may be, but is not limited to, oil-in-water, water-in-oil, water-in-oil-in-water and oil-in-water-in-silcone emulsions. The emulsion may contain a humectant. The emulsion may contain an anti-foaming agent, such as silicone. The emulsion may have any suitable viscosity. Emulsions may further contain an emulsifier and/or an anti-foaming agent. Methods of preparing an emulsion are known to a person skilled in the art.

The topical composition of the invention may be incorporated into a medical device for administration. Such a device can include but is not limited to a fabric, patch, bandage, gauge, sock, tight, underwear, dressing, glove, mask, adhesive patches, non-adhesive patches, occlusive patches and microelectric patches or suitable adhesive system. In such an embodiment, the device is in direct contact with the keratinous layer such as the skin, thus releasing the peptides of the invention. It will be understood that the topical composition may be incorporated in any suitable form as detailed herein. For example, the topical composition or peptides of the invention can be incorporated into the device or be present on the surface of the device or can be in a cream, gel or wax formulation or any suitable formulation defined herein and incorporated into the device or on the surface of the device.

The device may be adapted for adhesion or attachment to the skin.

In one embodiment the device is adapted to release a constant quantity of the composition or the peptides of the invention. It will be understood that the amount of the composition contained in the sustained release system will depend, for example, on where the composition is to be administered, the kinetics and duration of the release of the composition of the invention, as well as the nature of the condition, disorder and/or disease to be treated and/or cared for. The device may be such that the composition is released by biodegradation of the device, or by friction between the device and the body, due to bodily moisture, the skin's pH or body temperature.

In an embodiment of the invention the topical composition may further comprise at least one cosmetically or pharmaceutically acceptable excipient. Excipient may be used interchangeably with functional ingredient or additive. It will be understood that although the topical compositions of the current invention can be administered alone, they will generally be administered in admixture with a cosmetic or pharmaceutical excipient. Cosmetically or pharmaceutically acceptable excipient are well known in the art and any known excipient, may be used provided that it is suitable for topical administration and is dermatologically acceptable without undue toxicity, incompatibility and/or allergic reaction.

Preferably any excipient included is present in trace amounts. The amount of excipient included will depend on numerous factors, including the type of excipient used, the nature of the excipient, the component(s) of the topical composition, the amount of active or peptide in the topical composition and/or the intended use of the topical composition. The nature and amount of any excipient should not unacceptably alter the benefits of the peptides of this invention.

In an embodiment of the invention the excipient may be a suitable diluent, carrier, binder, lubricant, suspending agent, coating agent, preservative, stabilisers, dyes, vehicle, solubilising agent, base, emollient, emulsifying agent, fragrance, humectant, and/or surfactants.

Examples of suitable diluents include, but are not limited to, any diluent disclosed in disclosed in US2014120131 or US2004132667. Examples include ethanol, glycerol and water. Examples of suitable carriers include, but are not limited to, lactose, starch, glucose, methyl cellulose, magnesium stearate, mannitol, sorbitol and any suitable carrier disclosed in US2014120131 or US2004132667.

Examples of suitable binders include, but are not limited to, starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol and any suitable binder disclosed in US2014120131 or US2004132667.

Examples of suitable lubricants include, but are not limited to, sodium oleate, sodium stearate, magnesium stearate, sodium benzoate, sodium acetate, and sodium chloride and any suitable lubricant disclosed in US2014120131 or US2004132667.

The carrier may be any suitable carried known in the art or disclosed in US2014120131 or US2004132667. In some embodiments, the carrier may include, but is not limited to, a liquid, such as water, oils or surfactants, including those of petroleum, animal, plant or synthetic origin, polymer, oil, such as peanut oil, mineral oil, castor oil, soybean oil, alcohol, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, or digitonin. It will be understood that the carrier will be dermatologically acceptable. Preferred carriers contain an emulsion such as oil-in-water, water-in-oil, water-in-oil-in-water and oil-in-water-in-silicone emulsions. Emulsions may further contain an emulsifier and/or an anti-foaming agent.

In an embodiment of the invention, the topical composition may further comprise one or more additional ingredients. The topical composition of the invention may be administered consecutively, simultaneously or sequentially with the one or more other additional agents. Such additional ingredients may be those of benefit to include in a topical composition, or of benefit depending on the intended use of the topical composition. The additional ingredient may be active or functional or both.

Examples of such additional ingredients include, but are not limited to, one or more of cleaning agents, conditioning agents, sunscreen, pigment, moisturiser, thickening agents, gelling agents, essential oil, astringents, pigments, anti-caking agent, anti-foaming agent, binders, additives, buffers, chelating agents, external analgesics, film formers or materials, bulking agents, polymers, opacifying agents, pH adjusters, propellants, reducing agents, sequestrants, skin bleaching and lightening agents, skin conditioning agents, aloe vera, healing agents, soothing agents, smoothing agents, pantothenic acid, treating agents, thickeners, vitamins. colourants, pharmaceuticals, antiseptic agents, antifoaming agents, buffering agents, astringents, polymers, pH adjuster, deodorant or any other dermatologically acceptable carrier or surfactant.

It is to be understood that additional ingredients listed may provide more than one benefit. The classification given herein is for clarity and convenience only and not intended to limit the additional ingredient to that particular application or category listed.

Any additional ingredients should be suitable for application to the skin without undue toxicity, incompatibility and/or allergic reaction.

In some embodiments, the additional ingredient has glucose transport activity or aids glucose transport activity. In some embodiments, the additional ingredient has anti-inflammatory activity or aids anti-inflammatory activity. In some embodiments, the additional ingredient has anti-aging activity or aids anti-aging activity. In some embodiments, the additional ingredient is for keratinous layer health and/or development, skin health and/or development, and/or muscle health, recovery and/or development. The active agent may be a pharmacological enhancer. Such active agents are known and available on the market. In such cases, the topical composition of the invention may be administered consecutively, simultaneously or sequentially with the one or more other active agents.

In some embodiments, the additional ingredient may be farnesol ([2E, 6E], -3,7,11,-trimethyl-2, 6, 10,dodecatrien-1-ol), phytantriol (3, 7, 11, 15, tetramethylhexadecane-1,2,3,-triol), desquamation actives, enzymes, enzyme inhibitors, enzyme activators, botanical extracts and marine extracts, anti-acne actives, anti-wrinkle or anti atrophy actives, anti-oxidant/radical scavengers, chelators, flavonoids, anti-inflammatory agents, anti-cellulite agents, topical anaesthetics, tanning actives, skin lightening agents, skin healing agents, bisabolol, antimicrobial or antifungal active, sunscreen actives, particulate material, conditioning agents, structuring agents, thickening agent,

The desquamation active may be any suitable agent that enhances the skin appearance or texture of the skin and is as disclosed in US2014120131 or US2004132667.

Examples of anti-acne actives are as disclosed in US2014120131 or US2004132667 and include, resorcinol, salicylic acid, erythromycin, zine, sulfur, benzoyl peroxides.

Examples of thickening agents are as disclosed in US2014120131 or US2004132667 and include carboxylic acid polymers, crosslinked polyacrylate polymers, polyacrylamide polymers, polysaccharides.

Examples of conditioning agents are as disclosed in US2014120131 or US2004132667 and include humectants, moisturiser or skin conditioner.

Examples of structuring agents are as disclosed in US2014120131 or US2004132667 and include any agent that provide rheological characteristics to the composition and contributes to the stability of the composition.

Any suitable antimicrobial or antifungal active may be used and examples are as disclosed in US2014120131 or US2004132667. Such actives are capable of destroying microbes, preventing growth or action of microbes. Examples include but are not limited to β-lactam drugs, quinolone drugs, tetracycline, erythromycin, streptomycin sulfate, salicylic acid, benzoyl peroxide.

Examples of a particulate material include metallic oxide. Examples of anti-cellulite agents include xanthine agents. Examples of tanning actives includes 1,3-dihydroxy-2-propanone and those disclosed in US2014120131 or US2004132667. Examples of topical anaesthetics include benzocaine, lidocaine and bupivacaine and those disclosed in US2014120131 or US2004132667. Examples of skin lightening agents include any agent known in the art such as kojic acid, ascorbic acid and those disclosed in US2014120131 or US2004132667.

Examples of sunscreen actives include any suitable organic or inorganic sunscreen active. Examples include metallic oxides, 2-ethylhexyl-p-methoxycinnamate and those disclosed in US2014120131 or US2004132667.

Examples of skin healing agents includes panthenoic acid as disclosed in US2014120131 or US2004132667.

Examples of anti-inflammatory agents include any agent that enhances the skin appearance, tone or colour and include but are not limited to corticosteroids, hydrocortisone, non-steroidal agents such as ibuprofen and aspirin and those disclosed in US2014120131 or US2004132667.

Examples of flavonoids includes flavanones, methoxy flavonones, unsubstituted chalcone and mixtures thereof and those disclosed in US2014120131 or US2004132667.

Examples of enzymes include lipases, proteases, catalase, super oxide-dismutase, amylase, peroxidase, glucuronidase, ceramidases, hyaluronidases. Examples of enzyme inhibitors include trypsine inhibitors, Bowmann Birk inhibitors, chymotrypsin inhibitors, botanical extracts, flavonoids, quercetin chalcone and those disclosed in US2014120131 or US2004132667 and mixtures thereof. Examples of enzyme activators include coenzyme A, Q10 (ubiquinone), glycyrrhizin, berberine, chrysin and those disclosed in US2014120131 or US2004132667 and mixtures thereof.

Examples of anti-wrinkle or anti atrophy actives include sulfur containing D and L amino acids, particular, N-acyl derivatives such as N-acetyl-L-cysteine, hydroxyl acids, phytic acid, lipoic acid, lysophosphatidic acid, skin peel agents, vitamin B3, retinoids and those disclosed in US2014120131 or US2004132667 and mixtures thereof.

The anti-oxidant/radical scavenger agent may be any agent that is useful for providing protection against UV radiation or other environmental agents which may cause skin damage such as those disclosed in US2014120131 or US2004132667. Examples of anti-oxidant/radical scavengers include ascorbic acid, its salts and derivatives (vitamin C), tocopherol its salts and derivatives (vitamin E), butylated hydroxyl benzoic acids and their salts, peroxides, gallic acids and alkyl esters, sorbic acid, lipoic acid, amines, lycine pidolate, arginine pilolate, nordihydroguaiaretic acid, bioflavonoids, curcumin, lysine, methionine, proline, superoxide dismutase, silymarin, tea extracts and mixtures thereof.

Examples of chelators include EDTA, NTA, hydoxamic acids, phytic acid, lactoferrin and those disclosed in US2014120131 or US2004132667 and mixtures thereof. A chelator means an agent capable of removing a metal ion by forming a complex so that the metal ion cannot participate in or catalyse chemical reactions. A chelator is useful for protection against UV radiation or other environmental agents that can cause skin damage.

It will be appreciated that a plurality of additional ingredients may be added. The amount of the additional ingredient may be from about 0.001% to about 50% weight of the composition, preferably, about 0.01% to about 20%, preferably about 0.1% to about 10%, about 0.5% to about 10%, about 1% to about 5%, preferably 2% weight of the composition. The amount of additional ingredient included will depend on numerous factors, including the type of additional ingredient used, the nature of the additional ingredient, the component(s) of the topical composition, the amount of active or peptide in the topical composition and/or the intended use of the topical composition. The nature and amount of any additional ingredient should not unacceptably alter the benefits of the peptides of this invention.

The topical composition may be alcohol free.

In some embodiments of the invention, the composition further comprises one or more additional active agents, in addition to the peptide of the invention (also known as the active of the composition). In addition, or alternatively, the composition may be administered with one or more other additional active agents. Typical said additional active agent is present in trace amounts only. In some embodiments, there may be no additional active agent present in the composition. The amount of additional active agent included will depend on numerous factors, including the type of additional active agent used, the nature of the additional active agent, the component(s) of the topical composition, the amount of active or peptide in the topical composition and/or the intended use of the topical composition. The nature and amount of any additional active agent should not unacceptably alter the benefits of the peptides of this invention.

It is to be understood that an ingredient that is considered to be an “active” ingredient in one product may be a “functional” or “excipient” ingredient in another and vice versa. It will also be appreciated that some ingredients play a dual role as both an active ingredient and as a functional or excipient ingredient.

Examples of the additional active agents include glucose transport promoting drugs, skin supplement, agent for treatment and/or care of the skin, anti-inflammatory agent, an anti-aging agent, a cellular growth promoting agent and pharmacological enhancers. Such agents are well known in the art and it will be appreciated that any suitable additional active agent may be used. Additional active agents for treatment and/or care of the skin may include collagen synthesis agents, retinoids, exfoliating agents, anti-cellulite agents, elastase inhibiting agents, melanin synthesis stimulating or inhibiting agents, self-tanning agents, antiaging agents, antimicrobial agents, antifungal agents, fungistatic agents, bactericidal agents, and healing agents. Active agents also include anti-inflammatory agents.

Any additional active agent should be suitable for application to the skin without undue toxicity, incompatibility and/or allergic reaction.

It will be understood that the classification given herein is for clarity and convenience only and not intended to limit the additional ingredient, excipient, or active to that particular application or category listed.

In a particularly preferred embodiment, the methods and uses of the invention involve administration of a peptide or composition of the invention in combination with one or more other active agents, for example, existing growth promoting drugs or pharmacological enhancers available on the market. In such cases, the compounds of the invention may be administered consecutively, simultaneously or sequentially with the one or more other active agents.

The effect of the current invention is accomplished by topical application or administration of the topical composition of the invention described herein to a person, animal or a patient in need of treatment or care. Topical delivery preferably means delivery to a keratinous layer such as the skin, hair and/or nails, but can also mean delivery to a body lumen lined with epithelial cells, for example the lungs or airways, the gastrointestinal tract, the buccal cavity. The effect may be confined to the surface of the skin or may be within the skin or a combination of both.

The topical composition of the invention is administered in a cosmetically or pharmaceutically effective amount. In other words, in an amount that is non-toxic but sufficient amount to provide the desired effect. It will be appreciated that a person skilled in the art would be capable of determining an appropriate dose of the topical compositions of the invention to administer without undue experimentation. Alternatively, a physician will determine the actual dose that is most suitable for a patient depending on the particular condition, disease or disorder to be treated or cared for and the age, body weight and/or health of the person. It will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can, of course, be individual instances where higher or lower dosage ranges are merited, and such are within the scope of this invention. For example, the composition may be administered at a dose of from 0.01 to 50 mg/kg body weight, such as from 0.1 to 30 mg/kg, more preferably from 0.1 to 20 mg/kg body weight, more preferably from 0.1 to 10 mg/kg body weight, preferably 0.1 to 5mg/kg body weight. In an exemplary embodiment, one or more doses of 10 to 300 mg/day or more preferably, 10 to 150 mg/day, will be administered to the patient. The amount and the frequency is as best suited to the purpose. The frequency of application or administration can vary greatly, depending on the needs of each subject, with a recommendation of an application or administration range from once a month to ten times a day, preferably from once a week to four times a day, more preferably from three times a week to three times a day, even more preferably once or twice a day.

In preferred embodiments, repeated use of the topical composition is provided.

The topical composition may be applied by, but not limited to, rubbing, or massaging into the keratinous tissue, skin or area of the body to be treated or cared for. In some embodiments, the composition is left on or not removed from the area of the body. In other embodiments, the composition is removed after a period of time, such as, but not limited to, from about 2 minutes to 60 minutes, from about 5 minutes to about 30 minutes, preferably from about 10 minutes to about 20 minutes. The composition may be removed immediately after application. In some embodiments of the current invention, the composition of the invention may be applied to an area to be treated by means to achieve a greater penetration of the composition and/or peptide of the invention, such as, but not limited to, iontophoresis, sonophoresis, electroporation, microelectric patches, mechanical pressure, osmotic pressure gradient, occlusive cure, microinjections or needle-free injections by means of pressure, such as injections by oxygen pressure, or any combination thereof.

The peptides of the invention are used in the topical cosmetic or pharmaceutical composition of this invention at cosmetically or pharmaceutically effective concentrations to achieve the desired effect; in a preferred form with regards to the total weight of the composition, between 0.00000001% (in weight) and 20% (in weight); preferably between 0.000001% (in weight) and 15% (in weight), more preferably between 0.0001% (in weight) and 10% (in weight) and even more preferably between 0.0001% (in weight) and 5% (in weight).

In some embodiments of the current invention, the composition may be delivered via any one of liposomes, mixed liposomes, oleosomes, niosomes, ethosomes, millicapsules, capsules, macrocapsules, nanocapsules, nanostructured lipid carriers, sponges, cyclodextrins, vesicles, micelles, mixed micelles of surfactants, surfactant-phospholipid mixed micelles, millispheres, spheres, lipospheres, particles, nanospheres, nanoparticles,milliparticles, solid nanopartciles as well as microemulsions including water-in-oil microemulsions with an internal structure of reverse micelle and nanoemulsions microspheres, microparticles.

A variety of methods are available for preparing liposomes. See, e.g., Szoka et al., Ann. Rev. Biophys. Bioeng. 9:467 (1980), U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication No. WO 91/17424, Deamer & Bangham, Biochim. Biophys. Acta 443:629-634 (1976); Fraley, et al., PNAS 76:3348-3352 (1979); Hope et al., Biochim. Biophys. Acta 812:55-65 (1985); Mayer et al., Biochim. Biophys. Acta 858:161-168 (1986); Williams et al., PNAS 85:242-246 (1988); Liposomes (Ostro (ed.), 1983, Chapter 1); Hope et al., Chem. Phys. Lip. 40:89 (1986); Gregoriadis, Liposome Technology (1984) and Lasic, Liposomes: from Physics to Applications (1993)). Suitable methods include, for example, sonication, extrusion, high pressure/homogenization, microfluidization, detergent dialysis, calcium-induced fusion of small liposome vehicles and ether fusion methods, all of which are well known in the art.

These delivery systems may be adapted to achieve a greater penetration of the compound and/or peptides of the invention. This may improve pharmacokinetic and pharmacodynamics properties. The delivery system may be a sustained release system wherein the compound or peptide of the invention is gradually released during a period of time and preferably with a constant release rate over a period of time. The delivery systems are prepared by methods known in the art. The amount of peptide contained in the sustained release system will depend on where the composition is to be delivered and the duration of the release as well as the type of the condition, disease and/or disorder to be treated or cared for.

The topical composition of the invention may be for human or animal usage in human and veterinary medicine.

The topical composition of the invention may be used for pharmaceutical, personal care and/or cosmetic uses.

The composition can be used to treat or care for any disease, disorder or condition of the skin, including but not limited to, psoriasis, dermatitis, allergic dermatitis, eczema, spongiosis, edema, skin cancer, ulcers, acne, scars, cellulitis, elastosis, keratosis, rosacea, varicose veins, inflammatory disorders.

The topical composition may be used to for treating or caring for visible signs of aging including but not limited to wrinkles, stretch marks and dark circles, dryness, fine lines, age spots, red blotches, sagging skin, and conditions caused by sun exposure including sunburn, stress, pollution and/diet. The topical composition may also be used for delaying, slowing or inhibiting the skins or the onset of aging. The composition may be administered by a medical device, such as a plaster or a patch as described herein.

The topical composition may be used to treat or care for a wound in a mammal. In another embodiment, the topical composition is for use in the treatment or prevention of a disease or condition characterised by damaged epithelial cells or tissue, and/or damaged dermal or epithelial cells or tissue. The disease may be but is not limited to cancer and trauma.

The topical composition may be used to treat or care for any muscle condition, to improve, muscle status in a mammal, to promote recovery of muscle, typically following exercise, to maintain or restore muscle health (for example lean tissue mass) in a mammal, to enhance physical performance, in treatment or prevention of a disease or condition characterised by lethargy or low energy levels.

The topical composition may be used to promote growth of a tissue, promote growth of epithelial tissue, promote growth of skin, promote growth of an organ, promote growth of an organism. The skin can have a normal pathology and/or an abnormal pathology.

The topical composition may also be used to treat or care for any inflammatory disorder.

A further aspect of the invention relates to a pharmaceutical composition comprising a peptide of the invention or a composition of peptides of the invention, admixed with one or more pharmaceutically acceptable diluents, excipients or carriers. Even though the peptides and compositions of the present invention can be administered alone, they will generally be administered in admixture with a pharmaceutical carrier, excipient or diluent, particularly for human therapy. The pharmaceutical compositions may be for human or animal usage in human and veterinary medicine. Examples of such suitable excipients for the various different forms of pharmaceutical compositions described herein may be found in the “Handbook of Pharmaceutical Excipients, 2nd Edition, (1994), Edited by A Wade and P J Weller. In particular, formulations for topical delivery are described in Topical drug delivery formulations edited by David Osborne and Antonio Aman, Taylor & Francis, the complete contents of which are incorporated herein by reference. Acceptable carriers or diluents for therapeutic use are well known in the pharmaceutical art, and are described, for example, in Remington's Pharmaceutical Sciences, Mack Publishing Co. (A. R. Gennaro edit. 1985). Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of p hydroxybenzoic acid. Antioxidants and suspending agents may be also used. The peptide or composition of the invention may be adapted for topical, oral, rectal, parenteral, intramuscular, intraperitoneal, intra-arterial, intrabronchial, subcutaneous, intradermal, intravenous, nasal, vaginal, buccal or sublingual routes of administration. For oral administration, particular use is made of compressed tablets, pills, tablets, gellules, drops, and capsules. Preferably, these compositions contain from 1 to 250 mg and more preferably from 10-100 mg, of active ingredient per dose. Other forms of administration comprise solutions or emulsions which may be injected intravenously, intra-arterial, subcutaneously, intradermally, intraperitoneally or intramuscularly, and which are prepared from sterile or sterilisable solutions. The pharmaceutical compositions of the present invention may also be in form of suppositories, vaginal rings, pessaries, suspensions, emulsions, lotions, ointments, creams, gels, sprays, solutions or dusting powders. The composition of the invention may be formulated for topical delivery. Topical delivery generally means delivery to the skin, but can also mean delivery to a body lumen lined with epithelial cells, for example the lungs or airways, the gastrointestinal tract, the buccal cavity. In particular, formulations for topical delivery are described in Topical drug delivery formulations edited by David Osborne and Antonio Aman, Taylor & Francis, the complete contents of which are incorporated herein by reference. Compositions or formulations for delivery to the airways are described in O'Riordan et al (Respir Care, 2002, Nov. 47), EP2050437, WO2005023290, US2010098660, and US20070053845. Composition and formulations for delivering active agents to the iluem, especially the proximal iluem, include microparticles and microencapsulates where the active agent is encapsulated within a protecting matrix formed of polymer or dairy protein that is acid resistant but prone to dissolution in the more alkaline environment of the ileum. Examples of such delivery systems are described in EP1072600.2 and EP13171757.1. An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention. Depending upon the need, the agent may be administered at a dose of from 0.01 to 30 mg/kg body weight, such as from 0.1 to 10 mg/kg, more preferably from 0.1 to 1 mg/kg body weight. In an exemplary embodiment, one or more doses of 10 to 300 mg/day or more preferably, 10 to 150 mg/day, will be administered to the patient for the treatment of an inflammatory disorder.

In a particularly preferred embodiment, the methods and uses of the invention involve administration of a peptide or composition of the invention in combination with one or more other active agents, for example, existing anti-inflammatory drugs or pharmacological enhancers available on the market. In such cases, the compounds of the invention may be administered consecutively, simultaneously or sequentially with the one or more other active agents.

In one embodiment of the invention, the peptide of the invention may be administered in the form of a conjugate comprising the peptide, and may optionally include a linker, and a partner molecule, for example a protein such as an antibody molecule intended to increase the half-life of the conjugate in-vivo. In one embodiment, the peptide may be modified to substitute one or more amino acids with amino acids employed to attach partner molecules. For example, an amino acid may be substituted with a lysine residue for the purpose of conjugating a partner molecule such as a PEG molecule.

### Definitions

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

The term “mammal” should be understood to mean a higher mammal, especially a human. However, the term also includes non-mammalian animals such as fish.

The term “composition” should be understood to mean a composition of matter made by the hand of man and not occurring in nature. Exemplary compositions include food compositions, beverage compositions, pharmaceutical compositions, nutritional supplement compositions, personal care compositions and healthcare compositions.

The term “peptide” used herein refers to a polymer composed of 3 to 50 (or 4-50, 5-50, or 6-50) amino acid monomers typically linked via peptide bond linkage. Peptides (including fragments and variants thereof) of and for use in the invention may be generated wholly or partly by chemical synthesis or by expression from nucleic acid. For example, the peptides of and for use in the present invention can be readily prepared according to well-established, standard liquid or, preferably, solid-phase peptide synthesis methods known in the art (see, for example, J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984). When necessary, any of the peptides employed in the invention can be chemically modified to increase their stability. A chemically modified peptide or a peptide analog includes any functional chemical equivalent of the peptide characterized by its increased stability and/or efficacy in vivo or in vitro in respect of the practice of the invention. The term peptide analog also refers to any amino acid derivative of a peptide as described herein. A peptide analog can be produced by procedures that include, but are not limited to, modifications to side chains, incorporation of unnatural amino acids and/or their derivatives during peptide synthesis and the use of cross-linkers and other methods that impose conformational constraint on the peptides or their analogs. Examples of side chain modifications include modification of amino groups, such as by reductive alkylation by reaction with an aldehyde followed by reduction with NaBH₄; amidation with methylacetimidate; acetylation with acetic anhydride; carbamylation of amino groups with cyanate; trinitrobenzylation of amino groups with 2, 4, 6, trinitrobenzene sulfonic acid (TNBS); alkylation of amino groups with succinic anhydride and tetrahydrophthalic anhydride; and pyridoxylation of lysine with pyridoxa-5′-phosphate followed by reduction with NABH₄. The guanidino group of arginine residues may be modified by the formation of heterocyclic condensation products with reagents such as 2,3-butanedione, phenylglyoxal and glyoxal. The carboxyl group may be modified by carbodiimide activation via o-acylisourea formation followed by subsequent derivatization, for example, to a corresponding amide. Sulfhydryl groups may be modified by methods, such as carboxymethylation with iodoacetic acid or iodoacetamide; performic acid oxidation to cysteic acid; formation of mixed disulphides with other thiol compounds; reaction with maleimide; maleic anhydride or other substituted maleimide; formation of mercurial derivatives using 4-chloromercuribenzoate, 4-chloromercuriphenyl sulfonic acid, phenylmercury chloride, 2-chloromercuric-4-nitrophenol and other mercurials; carbamylation with cyanate at alkaline pH. Tryptophan residues may be modified by, for example, oxidation with N-bromosuccinimide or alkylation of the indole ring with 2-hydroxy-5-nitrobenzyl bromide or sulphonyl halides. Tryosine residues may be altered by nitration with tetranitromethane to form a 3-nitrotyrosine derivative. Modification of the imidazole ring of a histidine residue may be accomplished by alkylation with iodoacetic acid derivatives or N-carbethoxylation with diethylpyrocarbonate. Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. Peptide structure modification includes the generation of retro-inverso peptides comprising the reversed sequence encoded by D-amino acids.

“Isolated peptide” as applied to a peptide of the invention or modified peptide of the invention typically refers to a peptide of the invention that is produced by man by means of a technical process. Thus, the peptide may be produced by means of a biotechnological process or by means of chemical synthesis.

The term “modified peptide” is used interchangeably with the term derivative of the peptide. The modified peptide includes a peptide which has been substituted with one or more groups as defined herein. The modification may be any modified that provides the peptides and or the composition of the invention with an increased ability to penetrate a cell. The modification may be any modification that increases the half-life of the composition or peptides of the invention. In one embodiment, the group is a protecting group. The protecting group may be an N-terminal protecting group, a C-terminal protecting group or a side-chain protecting group. The peptide may have one or more of these protecting groups. The person skilled in the art is aware of suitable techniques to react amino acids with these protecting groups. These groups can be added by preparation methods known in the art, for example the methods as outlined in paragraphs [0104] to [0107] of US2014120141. The groups may remain on the peptide or may be removed. The protecting group may be added during synthesis. In an embodiment of the invention the peptides may be substituted with a group selected from one or more straight chain or branched chain, long or short chain, saturated, or unsaturated, substituted with a hydroxyl, amino, amino acyl, sulfate or sulphide group or unsubstituted having from 1 to 29 carbon atoms. N-acyl derivatives include acyl groups derived from acetic acid, capric acid, lauric acid, myristic acid, octanoic acid, palmitic acid, stearic acid, behenic acid, linoleic acid, linolenic acid, lipoic acid, oleic acid, isosteric acid, elaidoic acid, 2-ethylhexaneic acid, coconut oil fatty acid, tallow fatty acid, hardened tallow fatty acid, palm kernel fatty acid, lanolin fatty acid or similar acids. These may be substituted or unsubstituted. When substituted they are preferably substituted with hydroxyl, or sulphur containing groups such as but not limited to SO₃H, SH, or S—S. In an embodiment of the current invention, the peptide is R₁—X—R₂. R₁ and/or R₂ groups respectively bound to the amino-terminal (N-terminal) and carboxyl-terminal (C-terminal) of the peptide sequence. In one embodiment, the peptide is R₁—X. Alternatively, the peptide is X—R₂. Preferably, R₁ is H, C₁₋₄ alkyl, acetyl, benzoyl or trifluoroacetyl; X is the peptide of the invention; R₂ is OH or NH₂.

In an embodiment, R ₁ is selected from the group formed by H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, substituted or unsubstituted aralkyl, Tert-butyloxycarbonyl, 9-fluorenylmethyloxycarbonyl (Fmoc) and R₅—CO—, wherein R₅ is selected from the group formed by H, a non-cyclic substituted or unsubstituted aliphatic group, substituted or unsubstituted alicyclyl, substituted or unsubstituted aryl, substituted or unsubstituted aralkyl, substituted or unsubstituted heterocyclyl and substituted or unsubstituted heteroarylalkyl; R₂ is selected from the group formed by —NR₃R₄, —OR₃ and —SR₃, wherein R₃ and R₄ are independently selected from the group formed by H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, and substituted or unsubstituted aralkyl; and with the condition that R₁ and R₂ are not a-amino acids. In accordance with another preferred embodiment, R₂ is —NR₃R₄, —OR ₃ or —SR₃ wherein R₃ and R₄ are independently selected from the group formed by H, substituted or unsubstituted C₁-C₂₄ alkyl, substituted or unsubstituted C₂-C₂₄ alkenyl, Tert-butyloxycarbonyl, 9-fluorenylmethyloxycarbonyl (Fmoc), substituted or unsubstituted C₂-C₂₄ alkynyl, substituted or unsubstituted C₃-C ₂₄ cycloalkyl, substituted or unsubstituted C₅-C₂₄ cycloalkenyl, substituted or unsubstituted C₈-C ₂₄ cycloalkynyl, substituted or unsubstituted C ₆-C₃₀ aryl, substituted or unsubstituted C₇-C₂₄ aralkyl, substituted or unsubstituted heterocyclyl ring of 3-10 members, and substituted or unsubstituted heteroarylalkyl of 2 to 24 carbon atoms and 1 to 3 atoms other than carbon wherein the alkyl chain is of 1 to 6 carbon atoms. Optionally, R ₃ and R ₄ can be bound by a saturated or unsaturated carbon-carbon bond, forming a cycle with the nitrogen atom. More preferably R ₂ is —NR₃R₄ or —OR ₃, wherein R₃ and R₄ are independently selected from the group formed by H, substituted or unsubstituted C₁-C₂₄ alkyl, substituted or unsubstituted C₂-C₂₄ alkenyl, substituted or unsubstituted C₂-C₂₄ alkynyl, substituted or unsubstituted C₃-C₁₀ cycloalkyl, substituted or unsubstituted C₆-C ₁₅ aryl and substituted or unsubstituted heterocyclyl of 3-10 members, substituted or unsubstituted heteroarylalkyl with a ring of 3 to 10 members and an alkyl chain of 1 to 6 carbon atoms. More preferably R₃ and R₄ are selected from the group formed by H, methyl, ethyl, hexyl, dodecyl, or hexadecyl. Even more preferably R₃ is H and R₄ is selected from the group formed by H, methyl, ethyl, hexyl, dodecyl, or hexadecyl. In accordance with an even more preferred embodiment, R₂ is selected from —OH and —NH₂.

In accordance with another embodiment of this invention R ᵢ is selected from the group formed by H, acetyl, lauroyl, myristoyl or palmitoyl, and R₂ is —NR₃R ₄ or —OR₃ wherein R₃ and R₄ are independently selected from H, methyl, ethyl, hexyl, dodecyl and hexadecyl, preferably R₂ is —OH or —NH₂ More preferably, R₁ is acetyl or palmitoyl and R₂ is —NH₂. In a preferred embodiment, the acyl group is bound to the N-terminal end of at least one amino acid of the peptide. In an embodiment of the invention, the peptide is modified to comprise a side chain protecting group. The side chain protecting group may be one or more of the group comprising benzyl or benzyl based groups, t-butyl-based groups, benzyloxy-carbonyl (Z) group, and allyloxycarbonyl (alloc) protecting group. The side chain protecting group may be derived from an achiral amino acid such as achiral glycine. The use of an achiral amino acid helps to stabilise the resultant peptide and also facilitate the facile synthesis route of the present invention. Preferably, the peptide further comprises a modified C-terminus, preferably an amidated C-terminus. The achiral residue may be alpha-aminoisobutyric acid (methylalaine). It will be appreciated that the specific side chain protecting groups used will depend on the sequence of the peptide and the type of N-terminal protecting group used.

“Conjugate”: In one embodiment of the invention the peptide is conjugated, linked or fused to a binding partner, for example one or more polyethylene glycol polymers or other compounds, such as molecular weight increasing compounds or lipophilic groups. The molecular weight increasing compound is any compound that will increase the molecular weight, typically by 10% to 90%, or 20% to 50% of the resulting conjugate and may have a molecular weight of between 200 and 20, 000, preferably between 500 and 10, 000. The molecular weight increasing compound may be PEG, any water-soluble(amphiphilic or hydrophilic) polymer moiety, homo or co-polymers of PEG, a monomethyl-subsitututed polymer of PEG (mPEG) and polyoxyethylene glycerol (POG), polyamino acids such as poly-lysine, poly-glutamic acid, poly-aspartic acid, particular those of L conformation, pharmacologically inactive proteins such as albumin, gelatin, a fatty acid, olysaccharide, a lipid amino acid and dextran. The polymer moiety may be straight chained or branched and it may have a molecular weight of 500 to 40000 Da, 5000 to 10000 Da, 10000 to 5000, Da. The compound (binding partner) may be any suitable cell penetrating compound, such as that peptide, penetratin, pep-1. The compound (binding partner) may be an antibody molecule. The compound (binding partner) may be a lipophilic moiety or a polymeric moiety. The lipophilic substituent and polymeric substituents are known in the art. The lipophilic substituent includes an acyl group, a sulphonyl group, an N atom, an O atom or an S atom which forms part of the ester, sulphonyl ester, thioester, amide or sulphonamide. The lipophilic moiety may include a hydrocarbon chain having 4 to 30 C atoms, preferably between 8 and 12 C atoms. It may be linear or branched, saturated or unsaturated. The hydrocarbon chain may be further substituted. It may be cycloalkane or heterocycloalkane. The peptide may be modified at the N-terminal, C-terminal or both. The polymer or compound (binding partner) is preferably linked to an amino, carboxyl or thio group and may be linked by N-termini or C-termini of side chains of any amino acid residue. The polymer or compound (binding partner) may be conjugated to the side chain of any suitable residue. The polymer or compound (binding partner) may be conjugated via a spacer. The spacer may be a natural or unnatural amino acid, succinic acid, lysyl, glutamyl, asparagyl, glycyl, beta-alanyl, gamma-amino butanoyl. The polymer or compound (binding partner) may be conjugated via an ester, a sulphonyl ester, a thioester, an amide, a carbamate, a urea, a sulphonamide. A person skilled in the art is aware of suitable means to prepare the described conjugate.

“Anti-inflammatory” or “anti-inflammatory activity” as applied to a peptide or fragment means a peptide or fragment that is capable of significantly reducing the secretion of TNFα by LPS-stimulated J774.2 macrophages (compared with untreated LPS-stimulated J774.2 macrophages) when the macrophages are treated with 100 μM of the peptide or fragment as described in the experimental section below.

“Glucose transport promoting” or “glucose transport promoting activity” as applied to a peptide or fragment means a peptide or fragment that is capable of increasing GLUT4 translocation into skeletal muscle compared with an untreated control when employed at a concentration of 2 μM in the in-vitro assay described below. Preferably the peptide or fragment is capable of increasing GLUT4 translocation compared with an untreated control by at least 50% (i.e a relative unit increase in GLUT4 translocation of 1% to 1.5%).

“Growth promoting” or “growth promoting activity” as applied to a peptide or fragment means a peptide or fragment that is capable of increasing elastin production or cellular proliferation of human skin treated with a 20μM solution of peptide or fragment as described in the assay below.

“Antibacterial” or “antibacterial activity” as applied to a peptide or fragment means a peptide or fragment that is capable of visibly inhibiting the growth of a bacteria in the agar-plate based growth inhibition studies described below.

A “variant” of a bioactive fragment shall be taken to mean a fragment having an amino acid sequence that is substantially identical to the reference fragment, and typically is bioactive. Thus, for example, the term should be taken to include fragments that are altered in respect of one or more amino acid residues. Preferably such alterations involve the insertion, addition, deletion and/or substitution of 5 or fewer amino acids, more preferably of 4 or fewer, even more preferably of 3 or fewer, most preferably of 1 or 2 amino acids only. Insertion, addition and substitution with natural and modified amino acids is envisaged. The variant may have conservative amino acid changes, wherein the amino acid being introduced is similar structurally, chemically, or functionally to that being substituted. Generally, the variant will have at least 70% amino acid sequence homology, preferably at least 80% sequence homology, more preferably at least 90% sequence homology, and ideally at least 95%, 96%, 97%, 98% or 99% sequence homology with the parent anti-inflammatory fragment. In this specification, the term “sequence identity” should be understand to comprise both sequence identity and similarity, i.e. a variant (or homolog) that shares 70% sequence identity with a reference sequence is one in which any 70% of aligned residues of the variant (or homolog) are identical to or conservative substitutions of the corresponding residues in the reference sequence across the entire length of the sequence. Sequence identity is the amount of characters which match exactly between two different sequences. Hereby, gaps are not counted and the measurement is relational to the shorter of the two sequences. In terms of “sequence homology”, the term should be understood to mean that a variant (or homolog) which shares a defined percent similarity or identity with a reference sequence when the percentage of aligned residues of the variant (or homolog) are either identical to, or conservative substitutions of, the corresponding residues in the reference sequence and where the variant (or homolog) shares the same function as the reference sequence. This alignment and the percent homology or sequence identity can be determined using software programs known in the art, for example, one alignment program is BLAST, using default parameters. Details of these programs can be found at the following Internet address: http://www.ncbi.nlm.nih.gov/blast/Blast.cgi.

Variants of SEQUENCE ID NO: 555 (muscle peptide E64_SP2)

Variants of SEQUENCE ID NO: 555 (VLDLAIPVNRPGQL) including variants having 1,2 or 3 conservative amino acid substitutions, 1, 2 to 3 non-conservative amino acid substitutions, 1-2 amino acid additions, 1, 2 or 3 amino acid deletions, are provided below:

One conservative amino acid substitution:

(SEQ ID NO'S 1133 TO 1139)

ILDLAIPVNRPGQL; VLELAIPVNRPGQL; VLDLAVPVNRPGQL;
VLDLAIPINRPGQL; VLDLAIPVNKPGQL; VLDLAIPVEKPGQL;
VLDLAIPVNKPGEL

Two conservative amino acid substitutions:

(SEQ ID NOS 1140 TO 1148)

ILELAIPVNRPGQL; ILDLAVPVNRPGQL; VLELAVPVNRPGQL;
VLELAIPVNKPGQL; ILDLAIPVNKPGQL; VLDLAVPVNKPGQL;
VLDLAIPVEKPGEL; ILDLAIPVNKPGEL; VLELAIPVEKPGQL.

Three conservative amino acid substitutions:

(SEQ ID NOS TO 1149 TO 1156)

ILELAVPVNRPGQL; ILELAIPVNKPGQL; VLELAVPVNKPGQL;
ILELAIPVNRPGEL; ILDLAIPVNKPGEL; VLDLAVPVEKPGQL;
VLDLAVPVERPGEL; VLELAIPVERPGEL.

One non-conservative amino acid substitution

(SEQ ID NOS 1157 to 1162)

KLDLAIIVNRPGQL; VLDLAIPVNRPGQK; VLDLAIPVNRPCQL;
VLDLWIPVNRPGQL; VLDLAIPVNRPGQL; VLYLAIPVNRPGQL.

Two non-conservative amino acid substitution

(SEQ ID 1163 to 1170)

VLDLYIPVGRPGQL; VKDLAIPWNRPGQL; VLDLAIPVNRPCCL;
VLDLAGGVNRPGQL; VLDLAIPKNEPGQL; PLDLAIPVNDPGQL;
VLDLAIPVNRPIQL; VLDHAIPVNRPGQL.

Three non-conservative amino acid substitution

(SEQ ID 1171 to 1177)

VLDLAIPVNRPGGG; VLDLHIPGNEPGQL; VYKLAIPVNEPGQL;
VLDLAIPVNRPYPG; VLDYAIPKNDPGQL; RRRLAIPVNRPGQL;
VLDLAIGVNRGPQL

One or two amino acid additions

(SEQ ID NO: 1178 TO 1185)

VLDLAIPVNRPGFQL; VLDLADIPVNRPGQL; VLDLAIPVGNRPGQL;
VLQQDLAIPVNRPGQL; VLDLAIPVNRGPGQKL;
VLDGLPLAIPVNRPGQL; VLDLAIPVNRPGQLLL;
VLDLFLGAIPVNRPGQL

One, two or three amino acid deletions

(SEQ ID NOS 1186 TO 1193)

VLDLAIPVNRGQL; VLDLAPVNRPGQL; LDLAIPVNRPGQL;
VLDLAIPVNRPGQ; DLAIPVNRPGQL; VLDLAIPVNRPG;
VLDLAINRPGQL; VLDAIVNPGQL

Variants of SEQUENCE ID NO: 41 (anti-inflammatory peptide (I_37)

Variants of SEQUENCE ID NO: 41 (RGPQQYAEWQINEK) including variants having 1,2 or 3 conservative amino acid substitutions, 1, 2 to 3 non-conservative amino acid substitutions, 1-2 amino acid additions, 1, 2 or 3 amino acid deletions, are provided below:

One conservative amino acid substitution:

(SEQ ID NO'S 1194 to 1201)

RGPQQYAEWQINER, RGPQQYAEWQINDK, RGPQQFAEWQINEK,
KGPQQYAEWQINEK, RGPEQYAEWQINEK, RGPQEYAEWQINEK,
RGPQQYADWQINEK, RGPQQYAEYQINEK

Two conservative amino acid substitutions:

(SEQ ID NO'S 1202 to 1209)

KGPEQYAEWQINEK, KGPQEYAEWQINEK, KGPQQFAEWQINEK,
RGPEQFAEWQINEK, KGPQQYAEWQINER, RGPQQYAEWQINDR,
RGPQQYADWQINDK, RGPQQFAEWQINER

Three conservative amino acid substitutions:

(SEQ ID NO'S 1210 to 1217)

RGPQQYAEWQVNEK, RGPQQFAEWQINEK, KGPQQFAEWQINER,
KGPQQFAEWQVNEK, RGPQQFAEWQVNDK, RGPQQYADWQINDR,
KGPQQYADWQINDK, RGPQQFADYQINEK

One non-conservative amino acid substitution

(SEQ ID NO'S 1218 to 1225)

RGPQQYARWQINEK, RGPQQYAEWQINEE, HGPQQYAEWQINEK,,
RGPYQYAEWQINEK, RGPQQYMEWQINEK, RGPQQYAEWQINEK,
RGPQQYAEWCINEK, RGPQPYAEWQINEK

Two non-conservative amino acid substitution

(SEQ ID NO'S 1226 to 1232)

RGGQQYAEWQINED, RGPQQYARWKINEK, RGGQQYAETQINEK,
RGPLQYAEWQNNEK, EGPQQYAEWQINED, RGPQQYAEWQINLL,
RGPQQGGEWQINEK

Three non-conservative amino acid substitution

(SEQ ID NO'S 1233 to 1204)

RGPQQYAEWQIGGG, RGPQQKYEWQINEK, RGPQAQYEWQINEK,
RPHQQYAEWQINEK, RGPQHHHEWQINEK, RGPPQYAPPQINEK,
RGPQCYYEWCINEK, RGPTQYAEGQINEG

One or two amino acid additions

(SEQ ID NO'S 1241 to 1246)

RGPQQYAEWQINEKG, RGPQQYAEWQINEKY,
RGPQQYAFTEWQINEK, RGPQSQYAEWQINEKPM,
RGPQQYAEWQINEKKK, RRRRGPQQYAEWQINEK

One, two or three amino acid deletions

(SEQ ID NO'S 1247 to 1257)

RGPQQYAEWQINE, RGPQQYAEWQIN, RGPQQYAEWQI,
GPQQYAEWQINEK, PQQYAEWQINEK, QQYAEWQINEK,
QQYAEWQI, PQQYAEWQINE, PQQYAEWQIN, RGPQQYA,
EWQINEK

Variants of SEQUENCE ID NO: 701 (anti-ageing peptide E_1_788)

Variants of SEQUENCE ID NO: 701 (QSFLLSGNQ) including variants having 1 or 2 conservative amino acid substitutions, 1, 2 to 3 non-conservative amino acid substitutions, 1-2 amino acid additions, 1, 2 or 3 amino acid deletions, are provided below:

One conservative amino acid substitution:

(SEQ ID NO'S 1258 to 1261)

QSFILSGNE, ESFLLSGNQ, QSYLLSGNQ, QSFLLSGDQ

Two conservative amino acid substitutions:

(SEQ ID NO'S 1262 to 1266)

QSYLLSGNE, ESFLLSGNE, ESYLLSGNQ, QSFLLSGDE,
QSYLLSGDQ

One non-conservative amino acid substitution

(SEQ ID NO'S 1267 to 1271)

QSFRLSGNQ, QSFLLSYNQ, QFFLLSGNQ, QSFLLSGAQ,
QSFLLSGNP

Two non-conservative amino acid substitution

(SEQ ID NO'S 1272 to 1276)

QSFRRSGNQ, QSFLLSYIQ, QFFLLSGNL, QSFLLSGAQ,
QSFLLSGNP

One or two amino acid additions

(SEQ ID NO'S 1277 to 1281)

QSFLLSGNQQ, QSFLLLSGNQ, AQSFGLLSGNQ, RQSFLLISGNQ,
QSFLLSGNQK

One, two or three amino acid deletions

(SEQ ID NO'S 1282 to 1287)

QFLLSGNQ, SFLLSGNQ, QSFLLSGN, QSFLLGNQ, QSFLSGNQ,
QSLLSGNQ

“Fragment” means a fragment of a peptide of the invention that typically has a bioactivity, for example anti-inflammatory activity, anti-ageing activity, glucose transport promoting activity, or anti-bacterial activity. In one embodiment, the fragment has at least 3, 4, 5, 6, 7, 8, 9, 10, 11, 12, 13, 14, 15, 16, 17, 18, 19, 20, 21 or 22 amino acids. In one embodiment, the fragment consists of at least 30%, 40%, 50%, 60%, 70%, 80%, or 90% of the reference sequence. Examples of fragments of the invention are provided in SEQUENCE ID NO'S 708 to 751. Examples of fragments of SEQ ID NO: 555 include:

(SEQ ID 1288)

VLDLAIPVNRPGQ;

(SEQ ID 1289)

VLDLAIPVNRPG;

(SEQ ID 1290)

VLDLAIPVNRP;

(SEQ ID 1291)

LDLAIPVNRPGQL;

(SEQ ID 1292)

DLAIPVNRPGQL;

(SEQ ID 1293)

LAIPVNRPGQL;

(SEQ ID 1294)

LDLAIPVNRPGQ;

(SEQ ID 1295)

DLAIPVNRPG;

(SEQ ID 1296)

LAIPVNRP;

(SEQ ID 1297)

VLDLAIPVN;

(SEQ ID 1298)

AIPVNRPGQL;

(SEQ ID 1299)

VNRPGQL;

(SEQ ID 1300)

VLDLAIPV,
and

(SEQ ID 1301)

VLDLAIPVNR.

Examples of fragments of SEQ ID NO:701 include:

(SEQ ID NO'S 1302 to 1308).

QSFLLSGN, QSFLLSG, QSFLLS, SFLLSGNQ, FLLSGNQ,
LLSGNQ SFLLSGN.

“Enriched in peptides having a molecular weight of less than 10 KD” as applied to a composition of the invention means that the dry weight % of peptides in the composition having a molecular weight of less than 10 KD is greater than the dry weight % of polypeptide/protein in the composition having a molecular weight of 10 KD or greater.

“Inflammatory disorder” means an immune-mediated inflammatory condition that affects humans and is generally characterised by dysregulated expression of one or more cytokines. Examples of inflammatory disorders include skin inflammatory disorders, inflammatory disorders of the joints, inflammatory disorders of the cardiovascular system, certain autoimmune diseases, lung and airway inflammatory disorders, intestinal inflammatory disorders. Examples of skin inflammatory disorders include dermatitis, for example atopic dermatitis and contact dermatitis, acne vulgaris, and psoriasis. Examples of inflammatory disorders of the joints include rheumatoid arthritis. Examples of inflammatory disorders of the cardiovascular system are cardiovascular disease and atherosclerosis. Examples of autoimmune diseases include Type 1 diabetes, Graves disease, Guillain-Barre disease, Lupus, Psoriatic arthritis, and Ulcerative colitis. Examples of lung and airway inflammatory disorders include asthma, cystic fibrosis, COPD, emphysema, and acute respiratory distress syndrome. Examples of intestinal inflammatory disorders include colitis and inflammatory bowel disease. Other inflammatory disorders include cancer, hay fever, periodontitis, allergies, hypersensitivity, ischemia, depression, systemic diseases, post infection inflammation and bronchitis.

The peptides and compositions of the invention may also be employed in the non-therapeutic treatment of inflammation. Examples of non-therapeutic treatment of inflammation include use to relieve normal, non-pathological, inflammation, for example inflammation in the muscles and joints following exercise.

In this specification, the term “Metabolic disorder” should be understood to include pre-diabetes, diabetes; Type-1 diabetes; Type-2 diabetes; metabolic syndrome; obesity; diabetic dyslipidemia; hyperlipidemia; hypertension; hypertriglyceridemia; hyperfattyacidemia; hypercholerterolemia; hyperinsulinemia, and MODY.

“Ani-ageing” means inhibiting or slowing the appearance of ageing of a human's skin and/or reversing the appearance of ageing. “Slowing or inhibiting ageing of the skin” means slowing or inhibiting the ageing process in the skin, and/or reversing the appearance of ageing.

“Disease or condition characterised by damaged dermal or epithelial cells or tissue” means any condition or disease that results in damaged dermal or epithelial tissue or cells or organs. One example is trauma which often results in damaged skin. Another example is an inflammatory skin condition such as psoriasis or excezma which often results in damaged skin. Another example is an inflammatory disorder of the lower intestines which can result in damaged epithelial cells/tissue lining the lower intestines. Another example is damaged epithelial cells/tissue lining the lower intestines caused by ingestion of a toxic or damaging substance, for example toxic chemicals or drugs. Another example is cancer, for example bowel cancer, which can result in damaged epithelial tissue in the bowel. Another condition is a peripheral inflammatory disorder such as atopic dermatitis which can result in damage to the skin in humans.

“Disease or condition characterised by bacterial infection” means any condition or disease characterised having a pathology caused by growth of bacteria or by bacterial infection, including for example MRSA, salmonella, listeria, bacterial pneumonia, Staphylococcal food poisoning, bacterial memingitis. Specific examples are provided on the web page of Wikipedia™ under the section “List of infectious diseases”.

“Man-made” as applied to comestible products should be understood to mean made by a human being and not existing in nature.

“Maintaining or restoring gut health” means reducing and/or regulating the pro-inflammatory response in the gut and more specifically the epithelial cells. The healthy microbiome offers some protection against pathogenic viruses and bacteria, and their presence is needed to guide the development of our immune system. It has been shown that these bacteria can react to human signals of stress, sickness, or age which can be manifested by inflammation and as a consequence switch on their virulence genes and cause or contribute to disease. Having the ability to reduce and maintain at healthy levels the inflammatory response can help maintain the healthy bacteria. Digestive problems, which comprise the number one health problem in North America, appear to be occurring with more frequency in recent years. One way to maintain digestive health is to maintain proper inflammation and intestinal flora.

“Improving muscle status” means improving the muscle health, for example promoting skeletal muscle protein synthesis, skeletal glucose absorbtion, improving lean tissue mass in therapeutic or non-therapeutic context, promoting muscle recovery generally after activity exercise, or improving muscle performance. The methods or uses may be therapeutic or non-therapeutic. The term “improving lean tissue mass status” should be understood to mean increasing lean tissue mass, or inhibiting or preventing the rate of lean tissue mass degradation.

“Promoting muscle recovery” means causing an increase in absorbtion of glucose in skeletal muscle compared with untreated skeletal muscel.

“Disease or condition characterised by lethargy or low energy levels” means any condition or disease characterised by a feeling or tiredness or low energy. Examples include allergies, asthma, anemia, cancer and its treatments, chronic pain, heart disease, infection, depression, eating disorders, grief, sleeping disorders, thyroid problems, medication side effects, alcohol use, or drug use.

“Maintaining or restoring muscle health” means helping retain or restore mammalian muscle health resulting from damage incurred during exercise. By promoting glucose transport in skeletal muscle the peptides promote recovery from exercise, and relieve muscle soreness/pain and injury connected with exercise. They can also be used to decrease and prevent muscle cramping, and to allow a faster recovery from muscle cramping. Cramping can result from physical stress, mental stress, and or Repetitive Strain Injury stress. By promoting glucose transport the peptides help reduce Myopathy of the muscle, and help prevent Sarcopenia in mammals, promote recovery from injuries during exercise, and relieve muscle soreness/pain and injury connected with exercise. The invention also relates to a peptide or composition of the invention for use in maintaining or restoring muscle health in a mammal.

In this specification, the term “substantially all” as applied to a list of peptides should be understood to mean at least 60%, 70%, 80%, 90% or 95% of the peptides.

In this specification, the term “personal care product” should be understood to mean a composition formulated for use by humans in cleaning or treating the human body, particularly the skin, teeth, nails, feet and hair. Examples include shampoo, conditioner, skin creams and lotions, powders, dentrifice, shower gel or creams, body lotion, deodorant, and anti-perspirant.

In this specification, the term “nutritional supplement” should be understood to mean a product formulated for ingestion by a mammal and intended to confer a health benefit on the recipient. The supplement can take any form, for example a solid, liquid, or powder. Examples of supplements include powders, tablets, capsules, and drinks.

A further aspect of the invention relates to a pharmaceutical composition comprising a peptide of the invention or a composition of peptides of the invention, admixed with one or more pharmaceutically acceptable diluents, excipients or carriers. Even though the peptides and compositions of the present invention can be administered alone, they will generally be administered in admixture with a pharmaceutical carrier, excipient or diluent, particularly for human therapy. The pharmaceutical compositions may be for human or animal usage in human and veterinary medicine. Examples of such suitable excipients for the various different forms of pharmaceutical compositions described herein may be found in the “Handbook of Pharmaceutical Excipients, 2^{nd} Edition, (1994), Edited by A Wade and P J Weller. In particular, formulations for topical delivery are described in Topical drug delivery formulations edited by David Osborne and Antonio Aman, Taylor & Francis, the complete contents of which are incorporated herein by reference. Acceptable carriers or diluents for therapeutic use are well known in the pharmaceutical art, and are described, for example, in Remington's Pharmaceutical Sciences, Mack Publishing Co. (A. R. Gennaro edit. 1985). Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

The peptide or composition of the invention may be adapted for topical, oral, rectal, parenteral, intramuscular, intraperitoneal, intra-arterial, intrabronchial, subcutaneous, intradermal, intravenous, nasal, vaginal, buccal or sublingual routes of administration. For oral administration, particular use is made of compressed tablets, pills, tablets, gellules, drops, and capsules. Preferably, these compositions contain from 1 to 250 mg and more preferably from 10-100 mg, of active ingredient per dose. Other forms of administration comprise solutions or emulsions which may be injected intravenously, intra-arterial, subcutaneously, intradermally, intraperitoneally or intramuscularly, and which are prepared from sterile or sterilisable solutions. The pharmaceutical compositions of the present invention may also be in form of suppositories, vaginal rings, pessaries, suspensions, emulsions, lotions, ointments, creams, gels, sprays, solutions or dusting powders. The composition of the invention may be formulated for topical delivery. Topical delivery generally means delivery to the skin, but can also mean delivery to a body lumen lined with epithelial cells, for example the lungs or airways, the gastrointestinal tract, the buccal cavity. In particular, formulations for topical delivery are described in Topical drug delivery formulations edited by David Osborne and Antonio Aman, Taylor & Francis, the complete contents of which are incorporated herein by reference. Compositions or formulations for delivery to the airways are described in O'Riordan et al (Respir Care, 2002, November 47), EP2050437, WO2005023290, US2010098660, and US20070053845. Composition and formulations for delivering active agents to the iluem, especially the proximal iluem, include microparticles and microencapsulates where the active agent is encapsulated within a protecting matrix formed of polymer or dairy protein that is acid resistant but prone to dissolution in the more alkaline environment of the ileum. Examples of such delivery systems are described in EP1072600.2 and EP13171757.1. An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required. Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention. Depending upon the need, the agent may be administered at a dose of from 0.01 to 30 mg/kg body weight, such as from 0.1 to 10 mg/kg, more preferably from 0.1 to 1 mg/kg body weight. In an exemplary embodiment, one or more doses of 10 to 300 mg/day or more preferably, 10 to 150 mg/day, will be administered to the patient for the treatment of an inflammatory disorder.

In a particularly preferred embodiment, the methods and uses of the invention involve administration of a peptide or composition of the invention in combination with one or more other active agents, for example, existing anti-inflammatory drugs or pharmacological enhancers available on the market. In such cases, the compounds of the invention may be administered consecutively, simultaneously or sequentially with the one or more other active agents.

### BRIEF DESCRIPTION OF THE FIGURES

### Section A

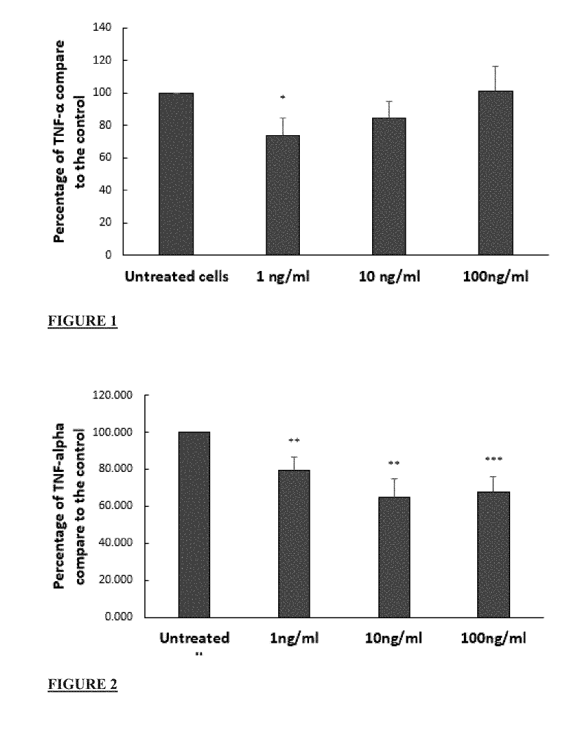

FIGS. 1 to 18: The effect of eighteen synthetic peptides of the invention on TNF-secretion in THP1 cells. All experiments were prepared in duplicate on three plates (6 wells/conditions). Significance was calculated using Student's t-test (*p<0.05 compared to control, **p<0.01 compared to control, *** p<0.001 compared to control)

FIG. 19. Viability of J774.2 macrophages after treatment with synthetic peptides. J774.2 macrophages were treated 100 μM of synthetic peptide for 24 hours before an alamar blue assay was performed. Data are presented as an average of n=3 +/−SEM.

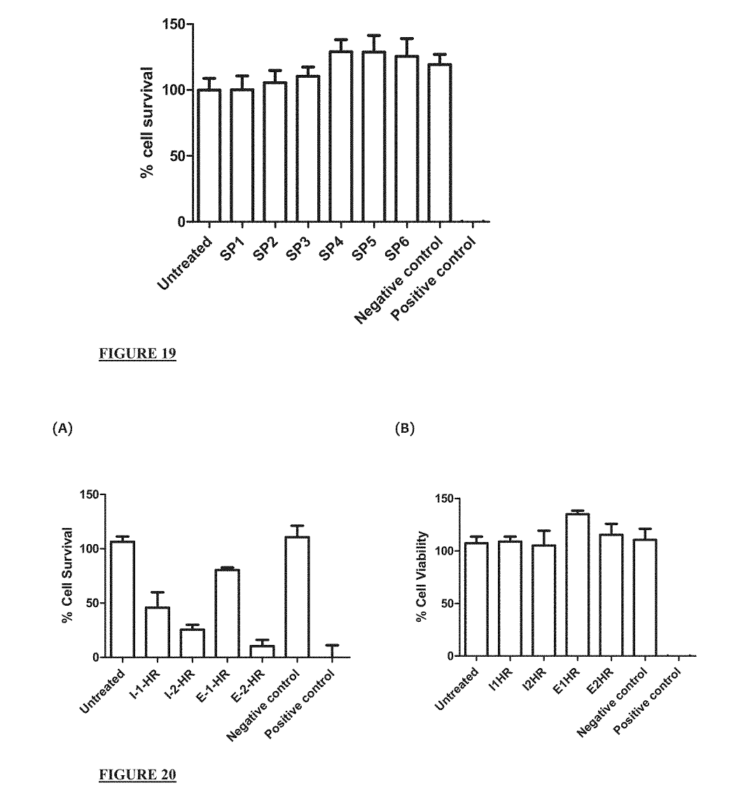

FIG. 20. The effects of hydrolysates on cell survival. J774.2 macrophages were treated with (A) 1 mg/ml or (B) 0.5 mg/ml of hydrolysates for 24 hours before an alamar blue assay was performed. Data is shown as (A) n=1 +/−SEM and (B) n=3 +/−SEM.

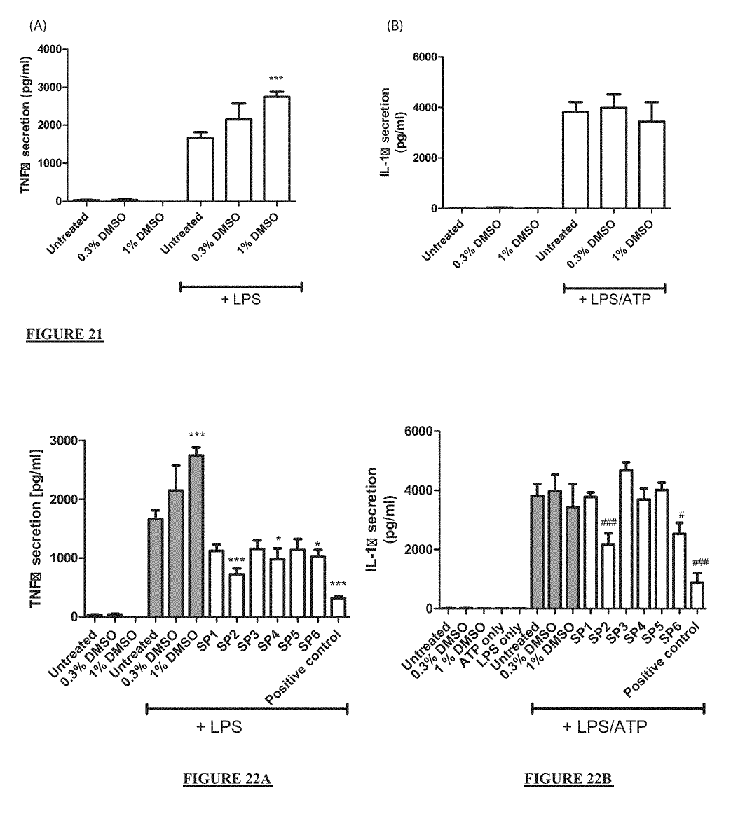

FIG. 21. The effect of DMSO vehicle on TNFα and IL-β (3 secretion from J774.2 macrophages. J774.2 macrophages were treated with a final concentration of 0.3% and 1% DMSO (equivalent to the amounts used to dissolve the peptides) for 24 hours and the effect on TNFα and IL-β after stimulation was established. Data are presented as an average of n=3 +/−SEM. (***p<0.001 w.r.t LPS).

FIG. 22. The effect of six peptides of the invention on TNFα and IL-β secretion from J774.2 macrophages. J774.2 macrophages were treated with 100 μM of synthetic peptide for 24 hours and then stimulated with (A) LPS (long/ml) for five hours or (B) LPS (long/ml) for 5 hours followed by ATP (5 mM) for one hour. Supernatant was collected and levels of (A) TNFα and (B) IL-β were determined by ELISA. (***p<0.001 w.r.t LPS, **p<0.01 w.r.t LPS, *p<0.05, ###p<0.001 w.r.t. LPS/ATP, ##p<0.01 w.r.t LPS/ATP and #p<0.05 w.r.t LPS/ATP). Final concentration of DMSO in well: SP1-0.3%, SP2-0%, SP3-0.3%, SP4-1%, SP5-1%, SP6-0.3%, Positive Control—0%. Data are presented as an average of n=3 +/−SEM.

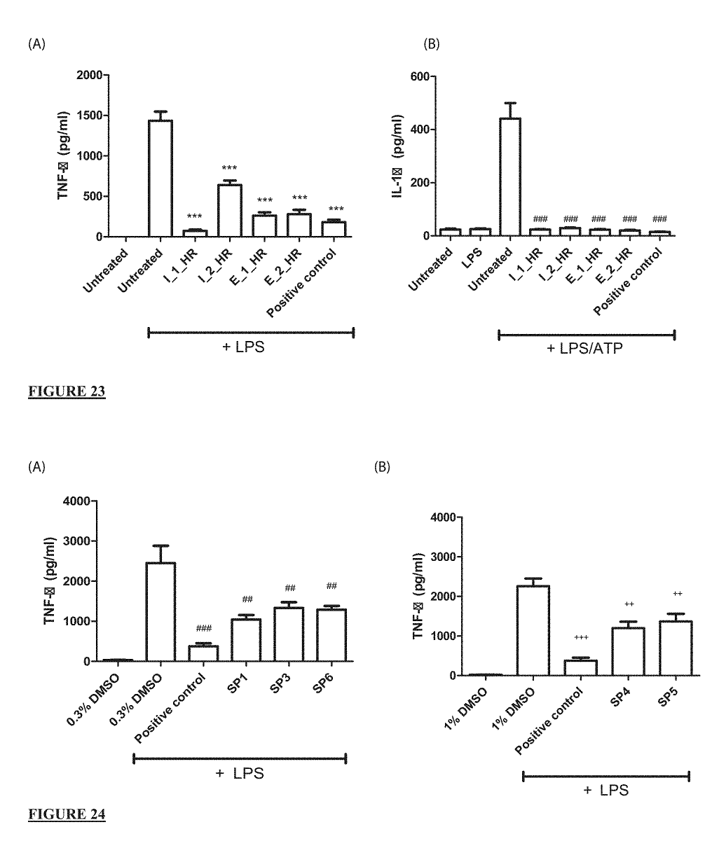

FIG. 23. The effect a peptide composition of the invention on TNFα and IL-β secretion. J774.2 macrophages were treated with 0.5 mg/ml of hydrolysate for 24 hours and then stimulated with (A) LPS (long/ml) for five hours or (B) LPS (long/ml) for 5 hours followed by ATP (5 mM) for one hour. Supernatant was collected and levels of (A) TNFα and (B) IL-β were determined by ELISA. (***p<0.001 w.r.t untreated+LPS, ###p<0.001 w.r.t. untreated+LPS/ATP). Data are presented as an average of n=3 +/−SEM.

FIG. 24. The effects of synthetic peptides with DMSO vehicle on TNFα J774.2 macrophages were treated with 100 μM of synthetic peptide for 24 hours and then LPS (long/ml) for five hours. Supernatant was collected and levels of TNFα were determined by ELISA. ###p<0.001 w.r.t 0.3% DMSO+LPS, ##p<0.01 w.r.t. 0.3% DMSO+LPS,+++p<0.001 w.r.t 1% DMSO+LPS, ++p<0.01 w.r.t 1% DMSO+LPS/ATP). Final concentration of DMSO in well: positive control-0% , SP1-0.3%, SP2-0%, SP3-0.3%, SP4-1%, SP5-1%, SP6-0.3%.

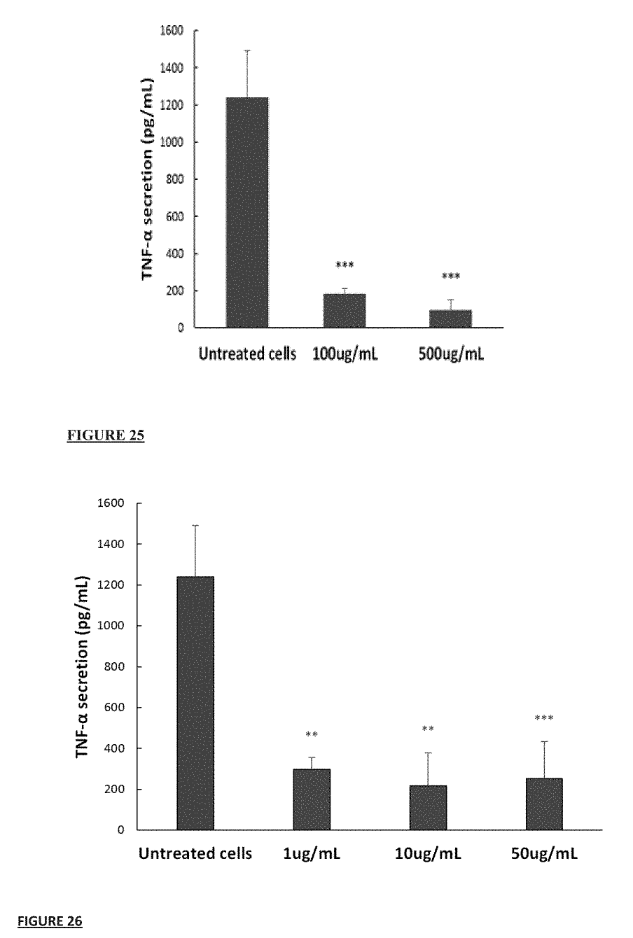

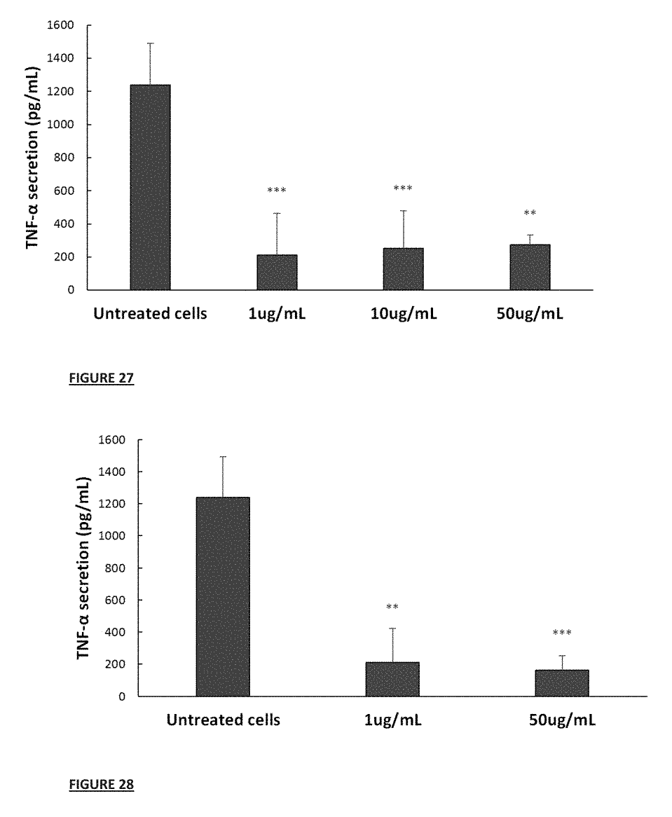

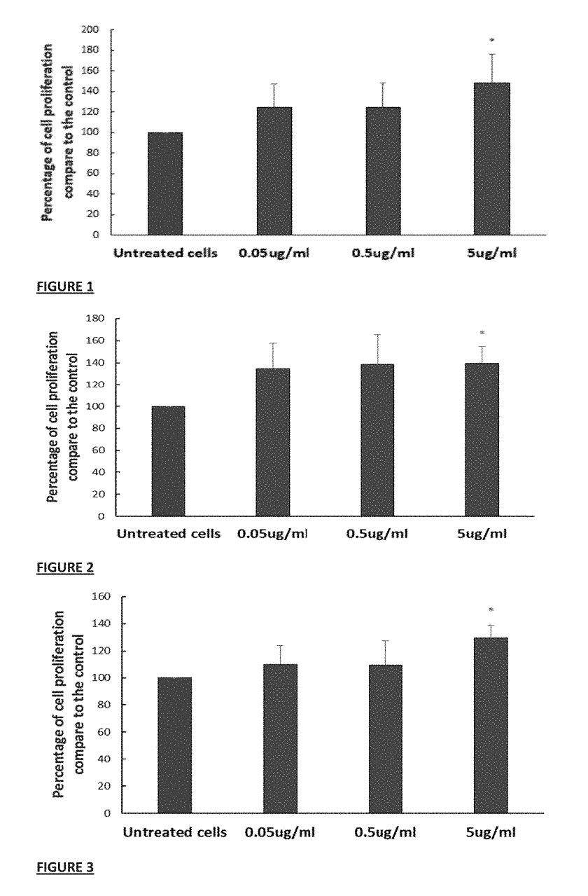

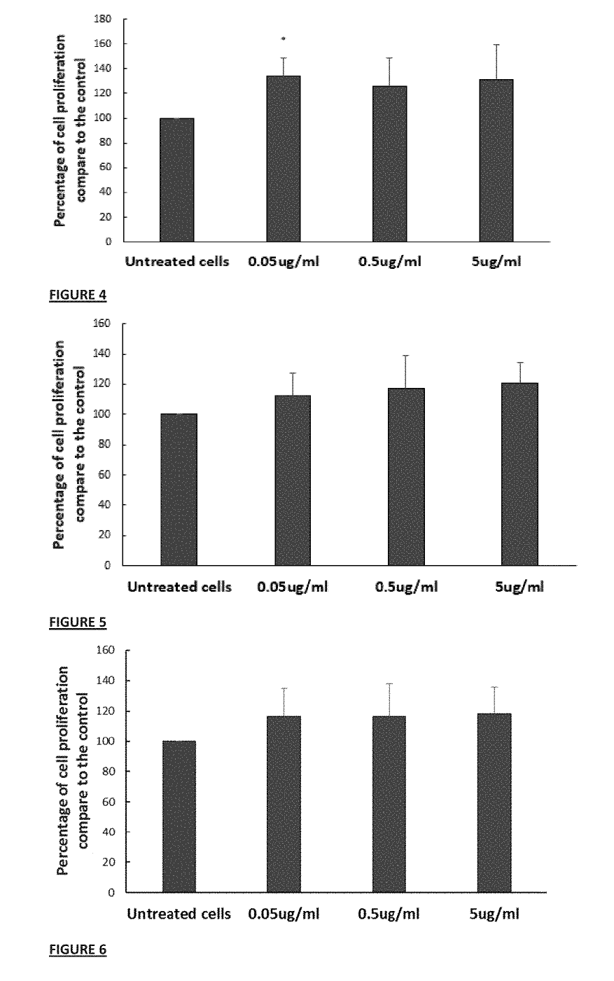

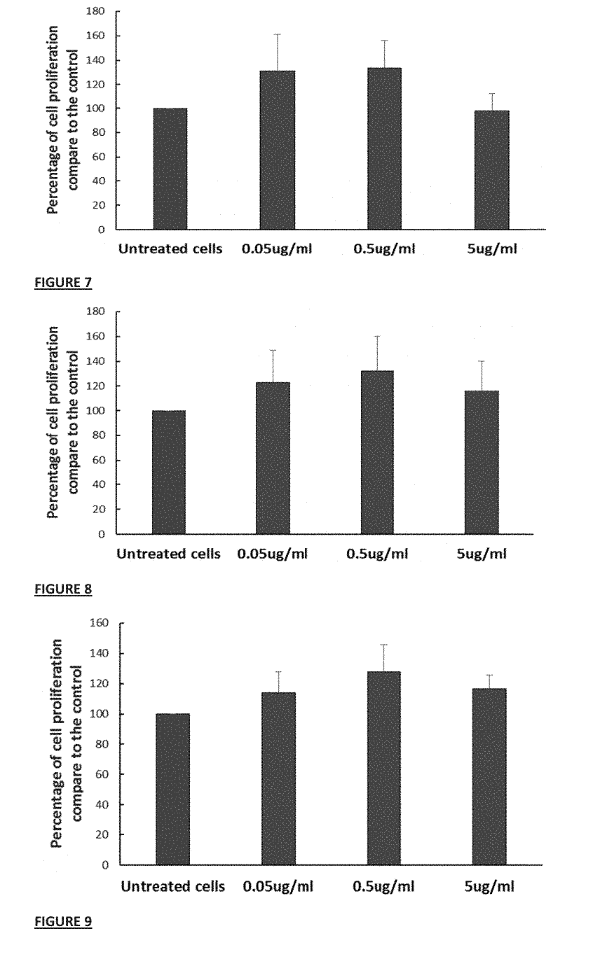

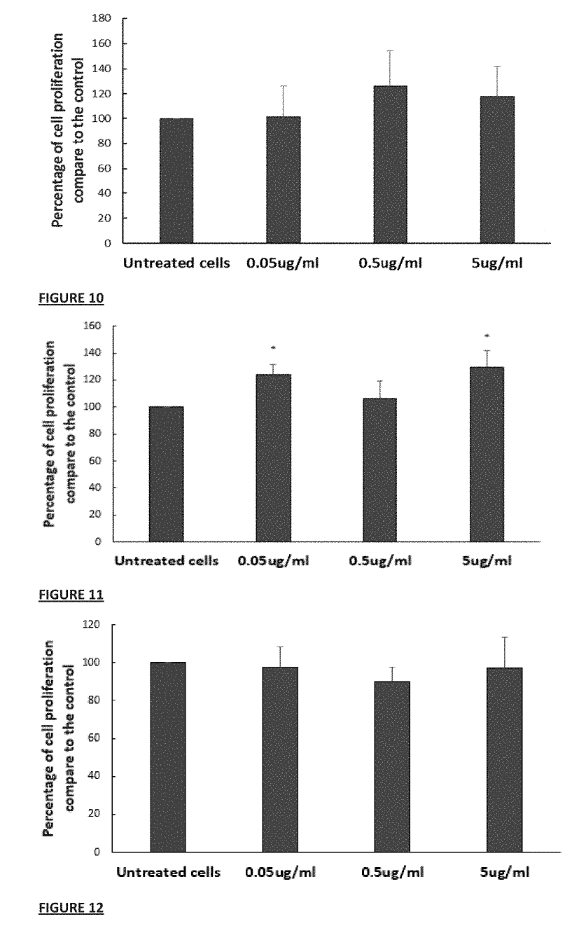

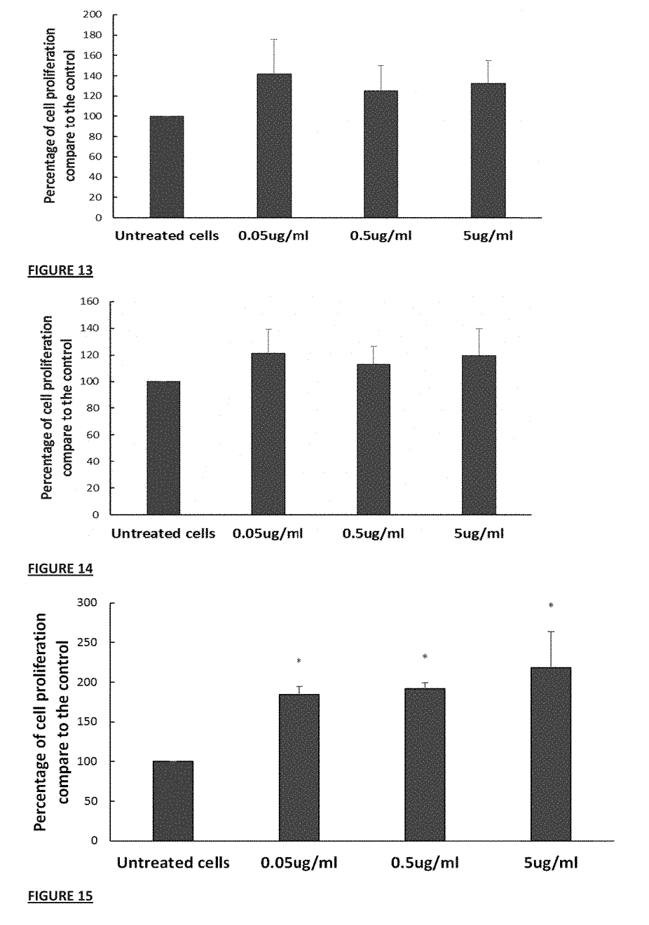

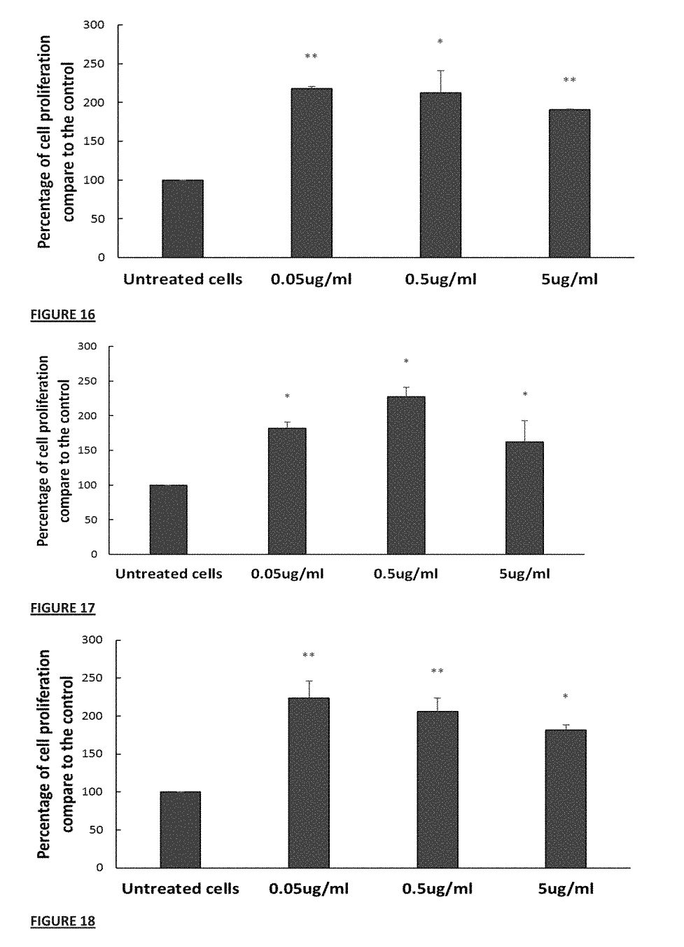

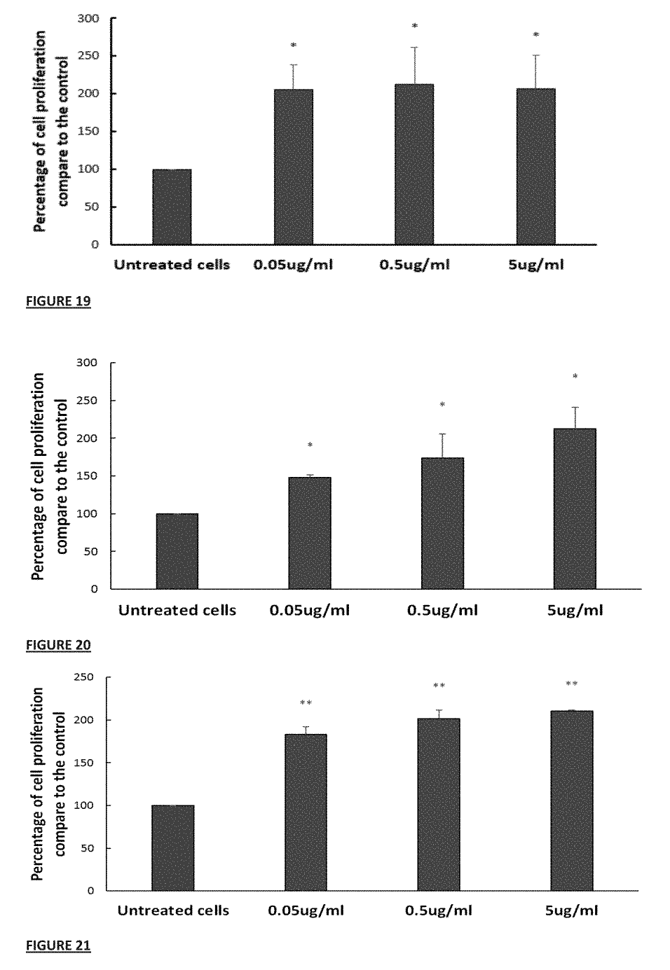

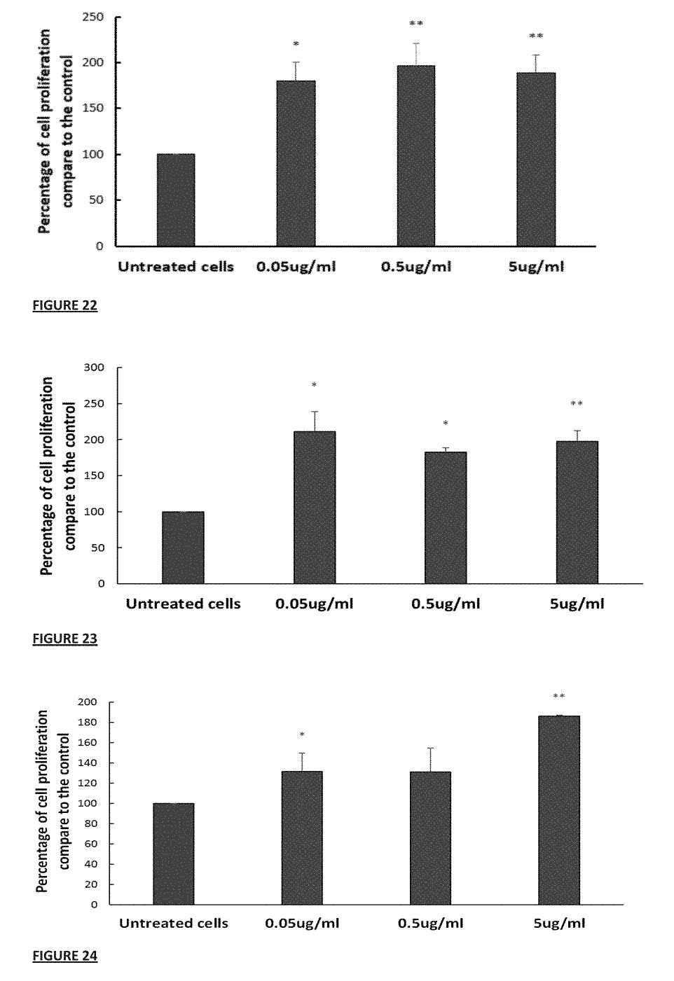

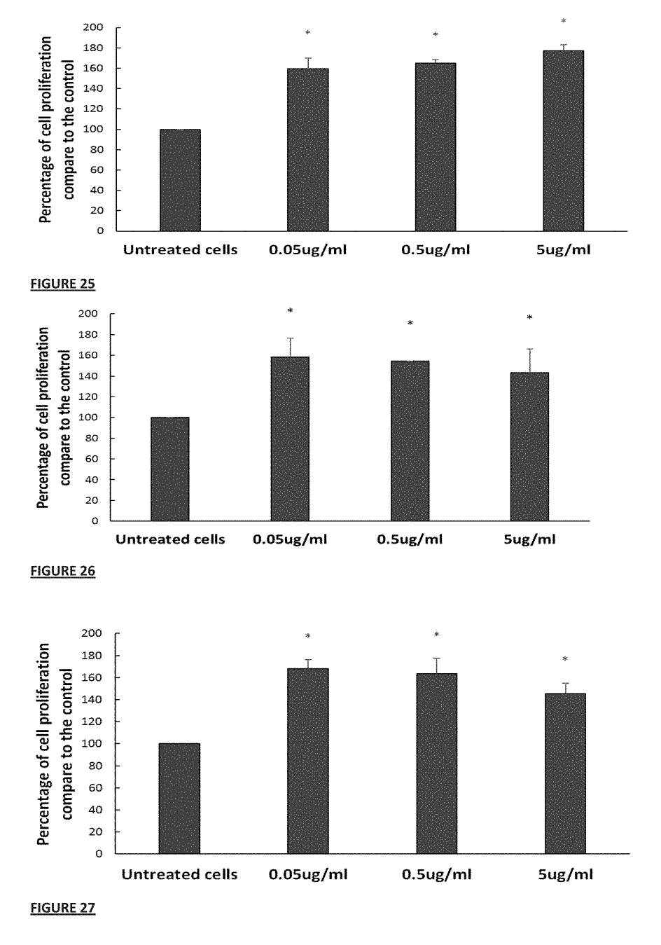

FIG. 25. THP-1 differentiated macrophages treated with a composition of rice peptides of the invention (I_2_HR) for 24 hrs. prior to LPS stimulation were compared to untreated cells. TNF-α secretion in I_2_HR treated cells is reduced by 92% vs. untreated cells. Significant results are observed at 100 ug/ml and 500 ug/ml concentrations of I_2_HR, indicating the potency of I_2_HR.

FIG. 26. THP-1 differentiated macrophages treated with E_41_PJ for 24 hrs. prior to LPS stimulation were compared to untreated cells. TNF-α secretion in E_41_PJ treated cells is reduced by 80% vs. untreated cells. At all tested concentrations of E_41_PJ a significant reduction in TNF-α is seen.

FIG. 27. THP-1 differentiated macrophages treated with E_1_788 for 24 hrs. prior to LPS stimulation were compared to untreated cells. TNF-α secretion in E_1_788 treated cells is reduced by 80% vs. untreated cells. At all tested concentrations of E_1_788 a significant reduction in TNF-α is seen.

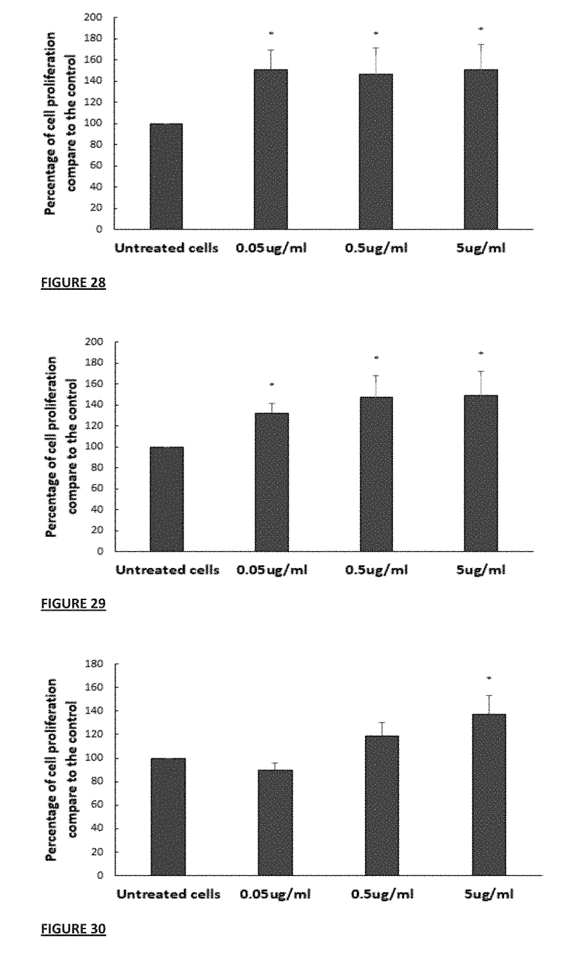

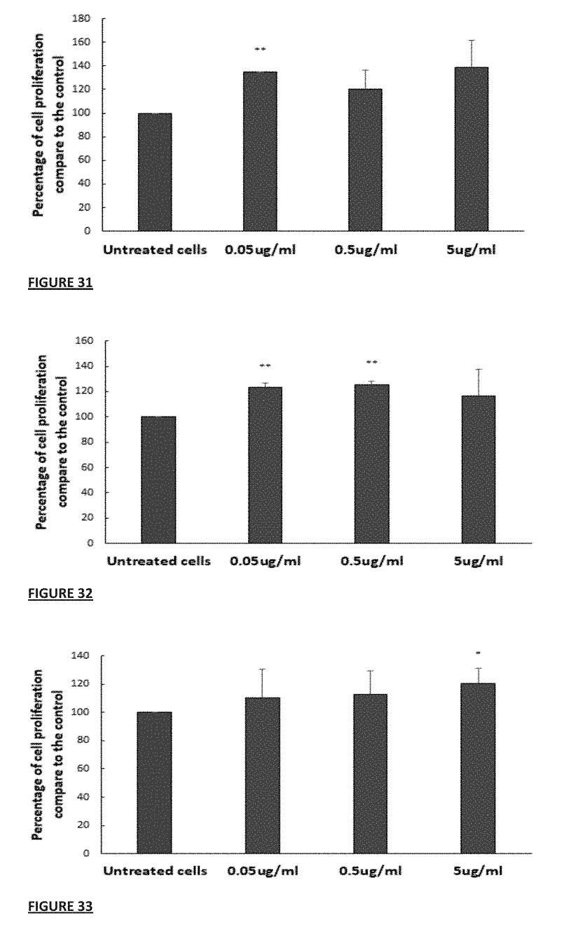

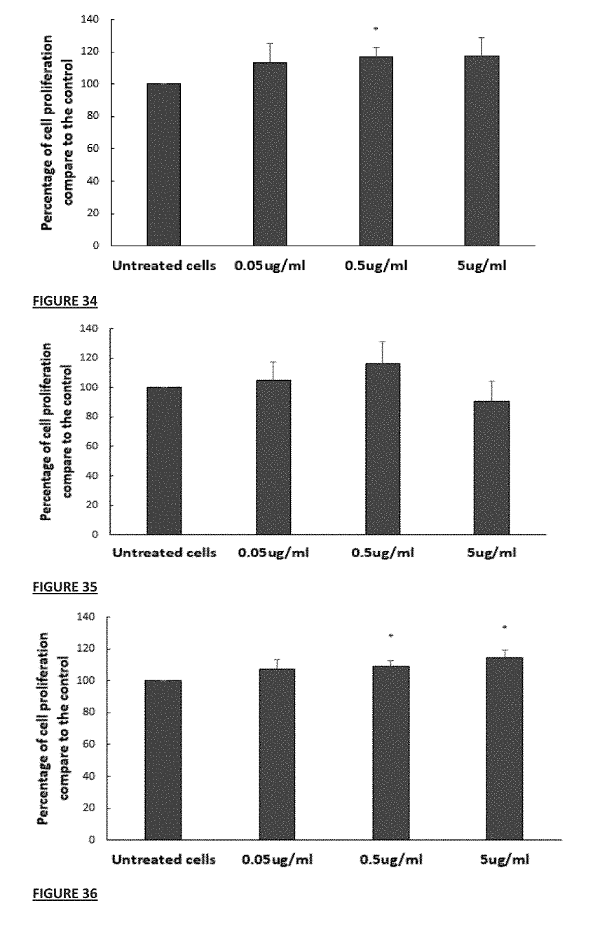

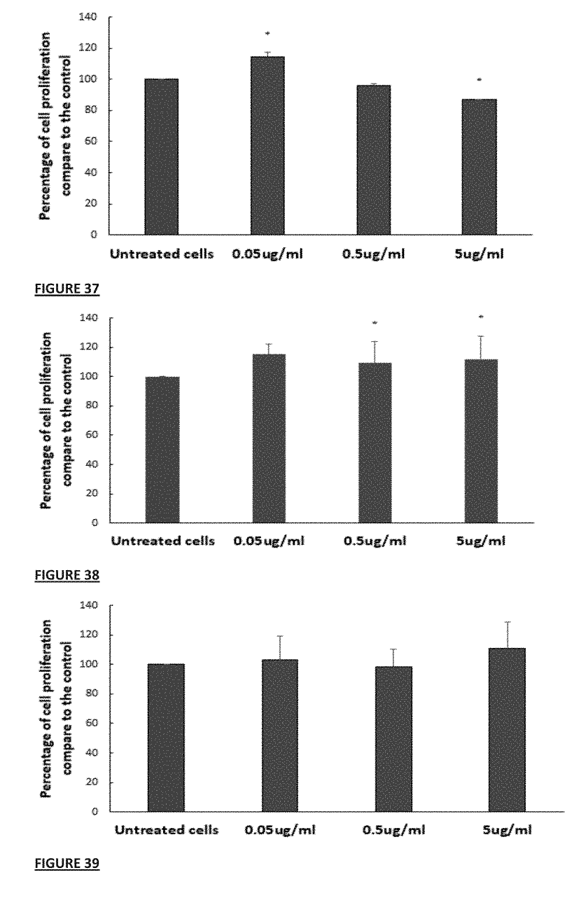

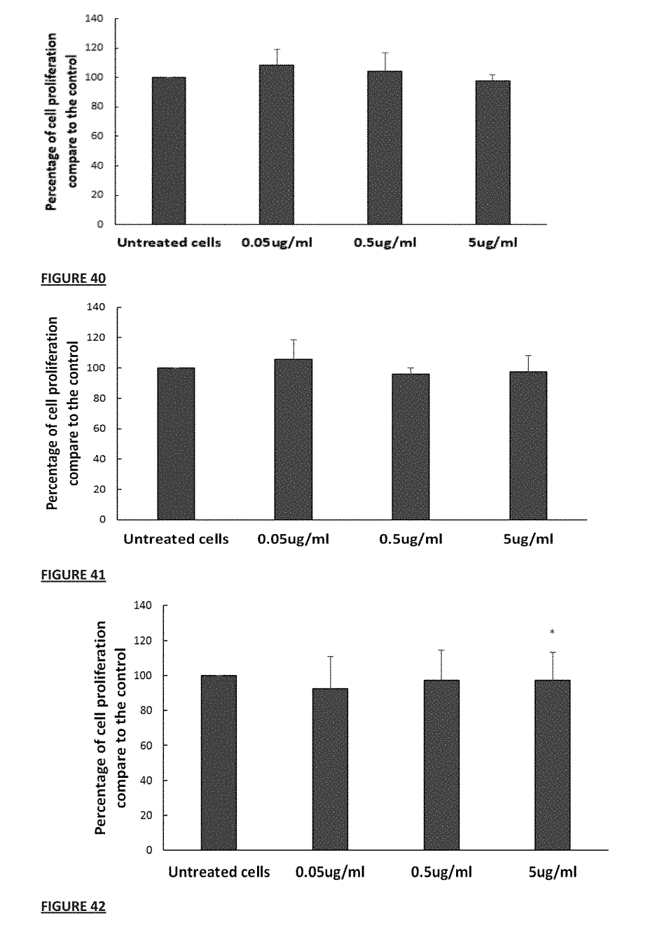

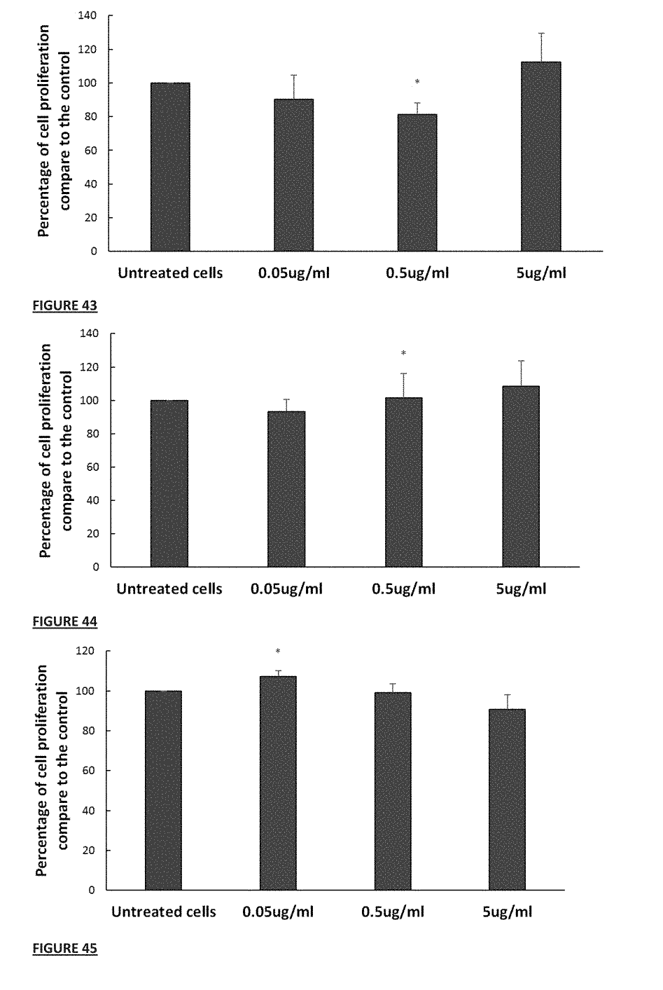

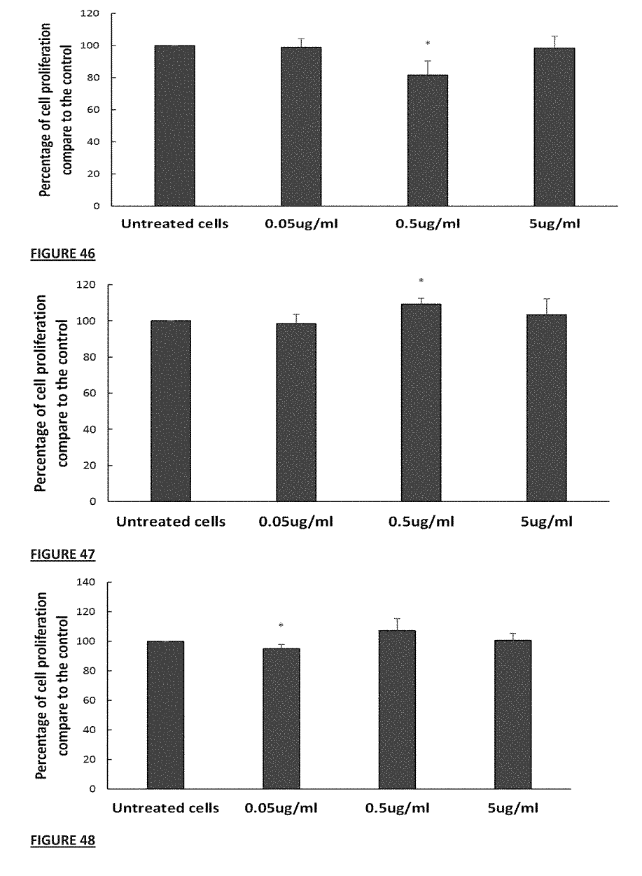

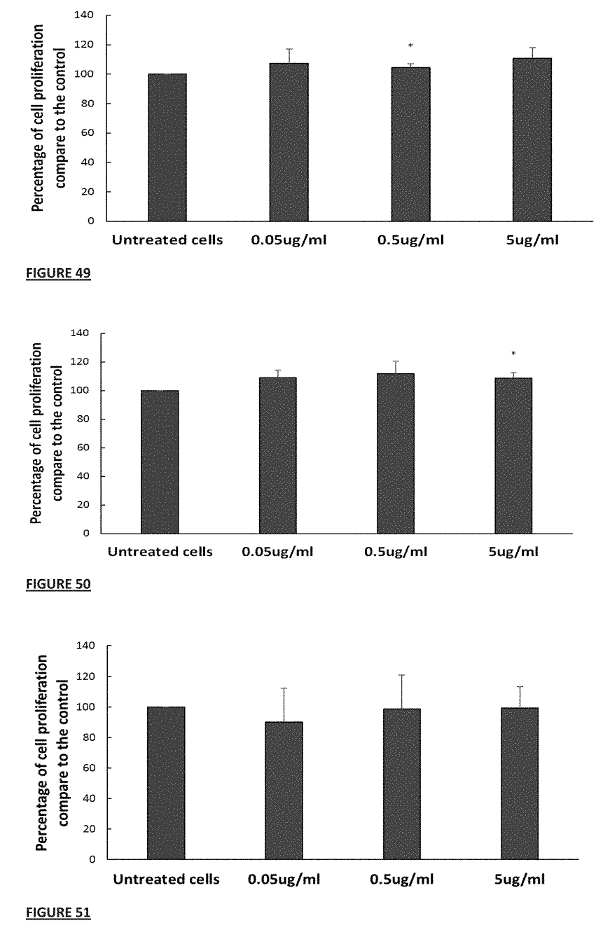

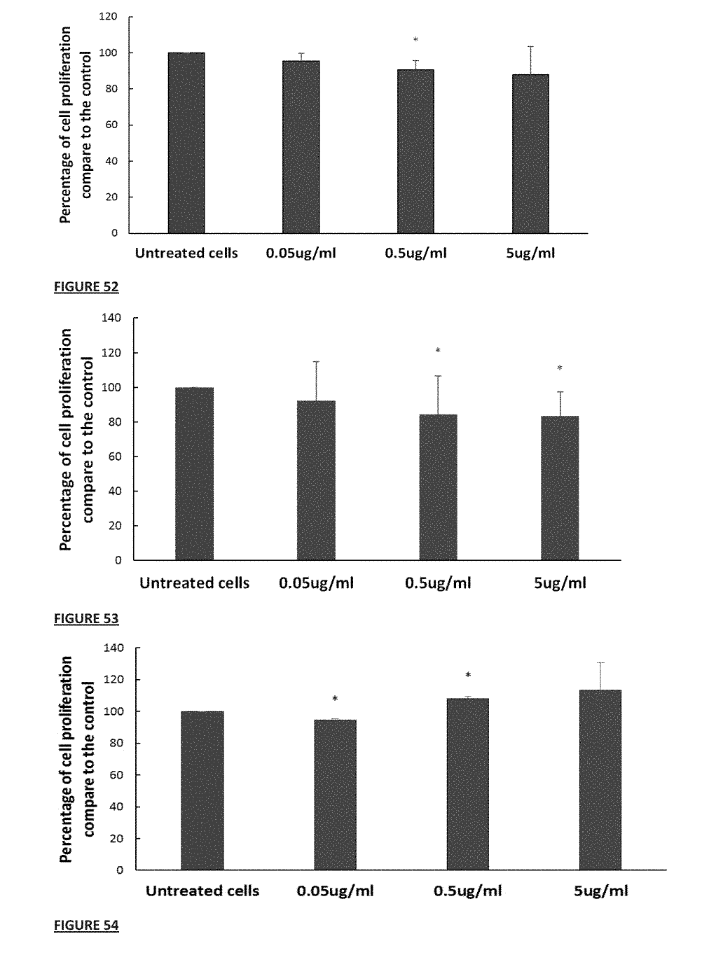

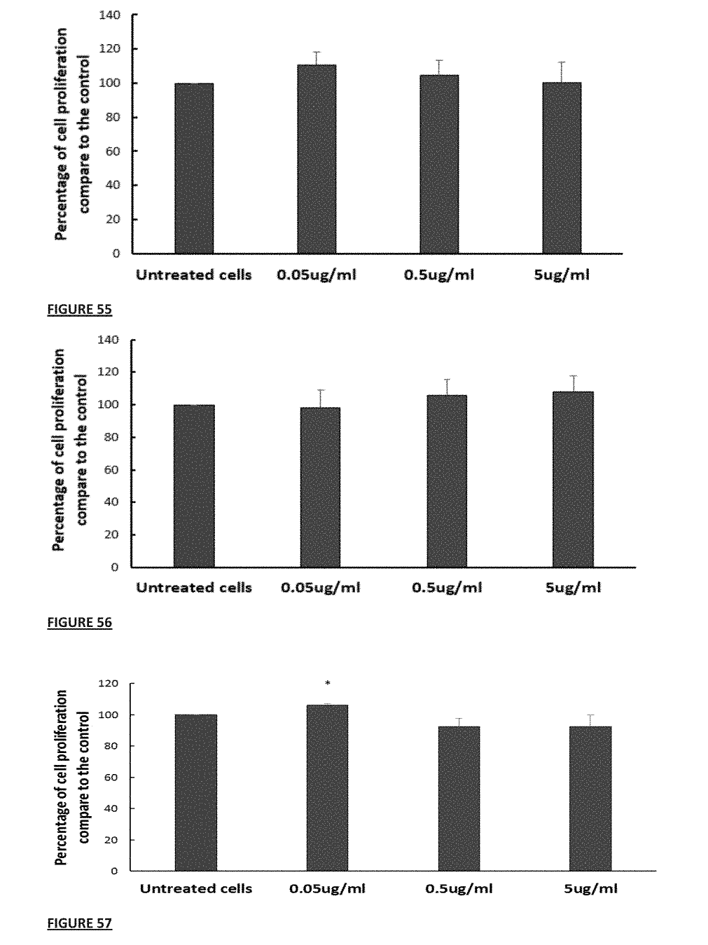

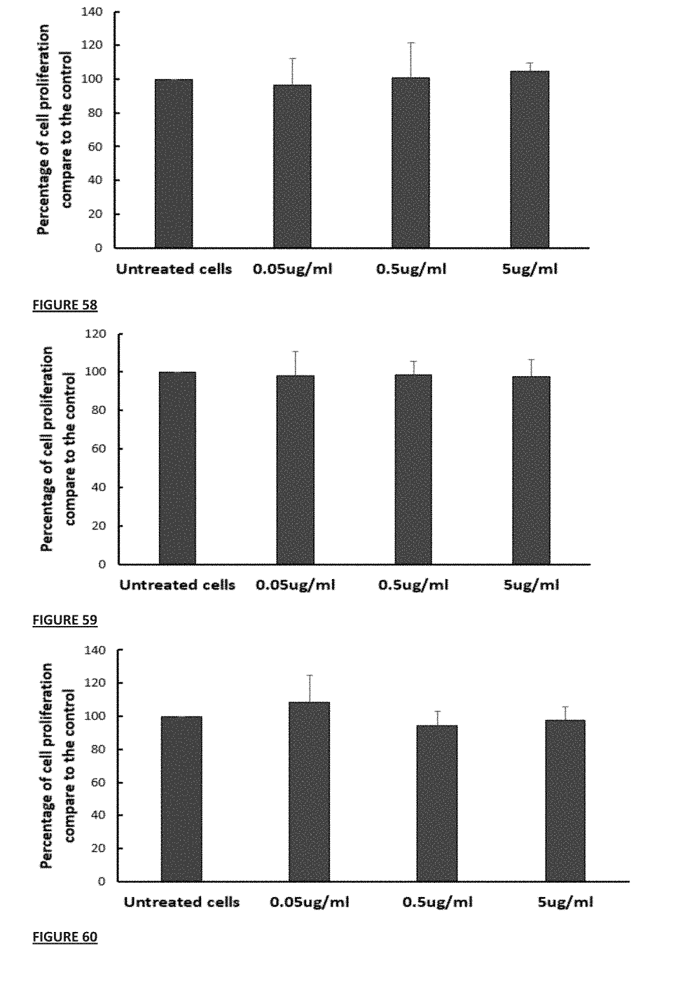

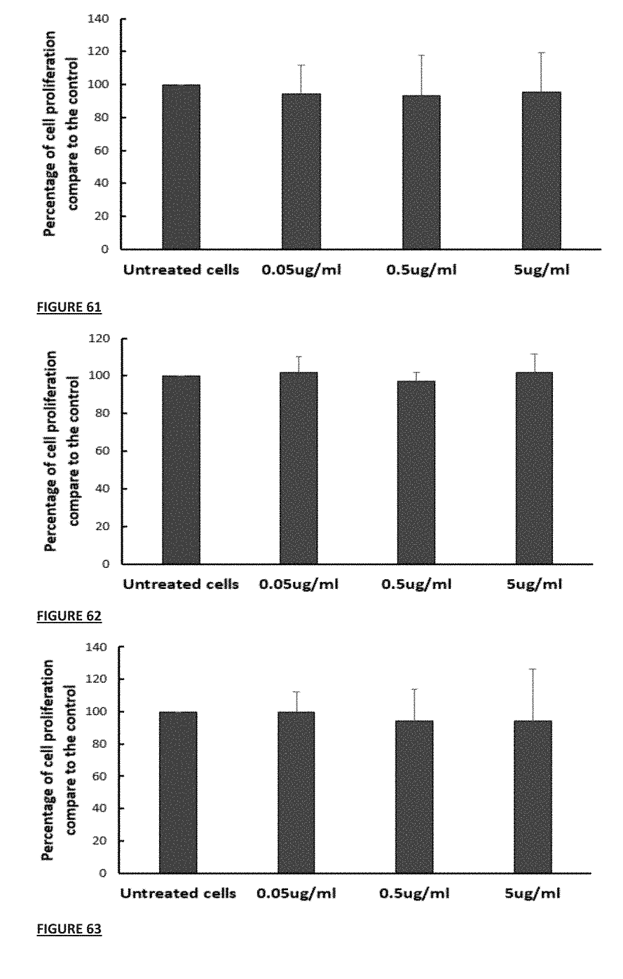

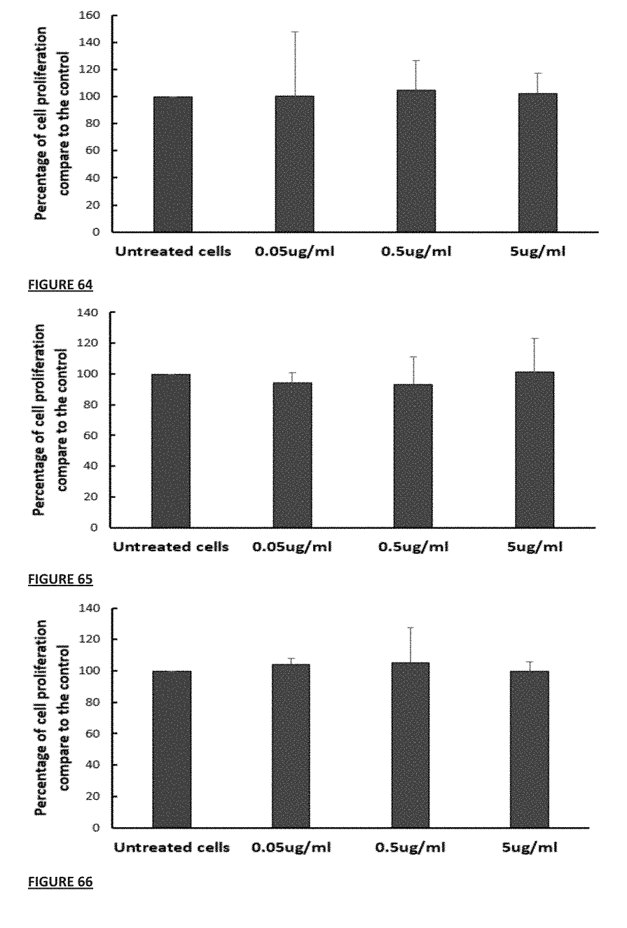

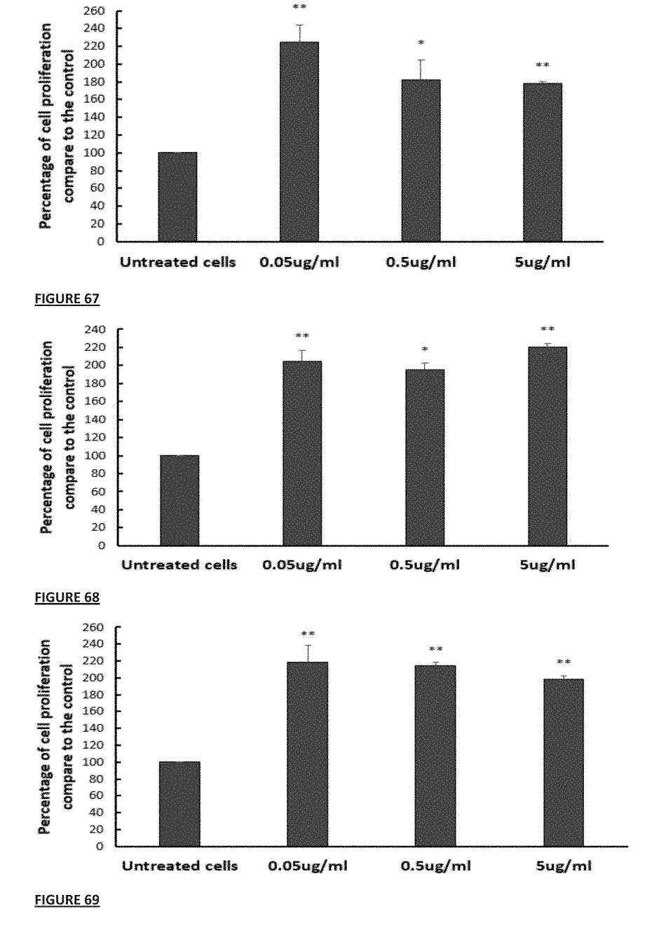

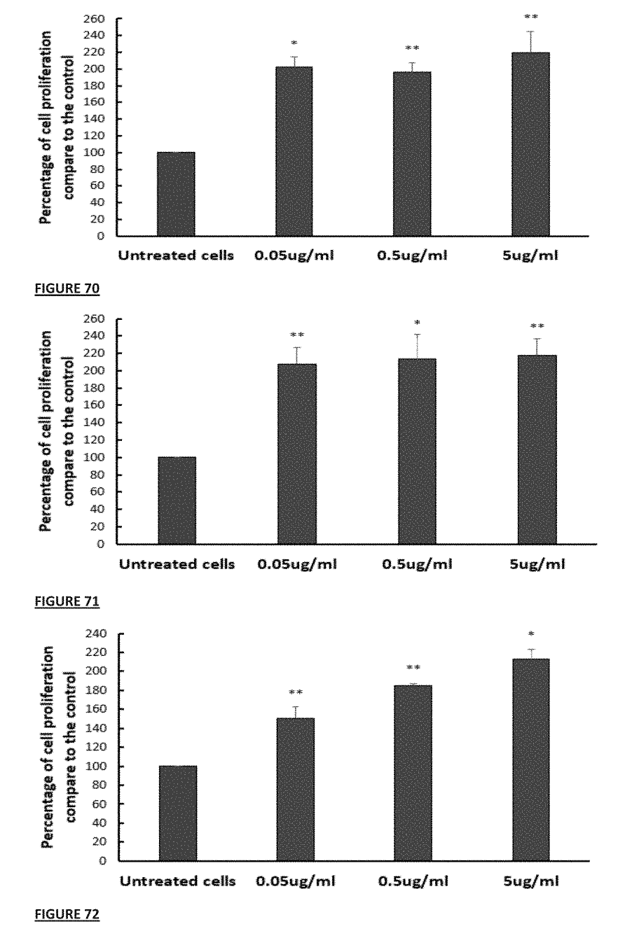

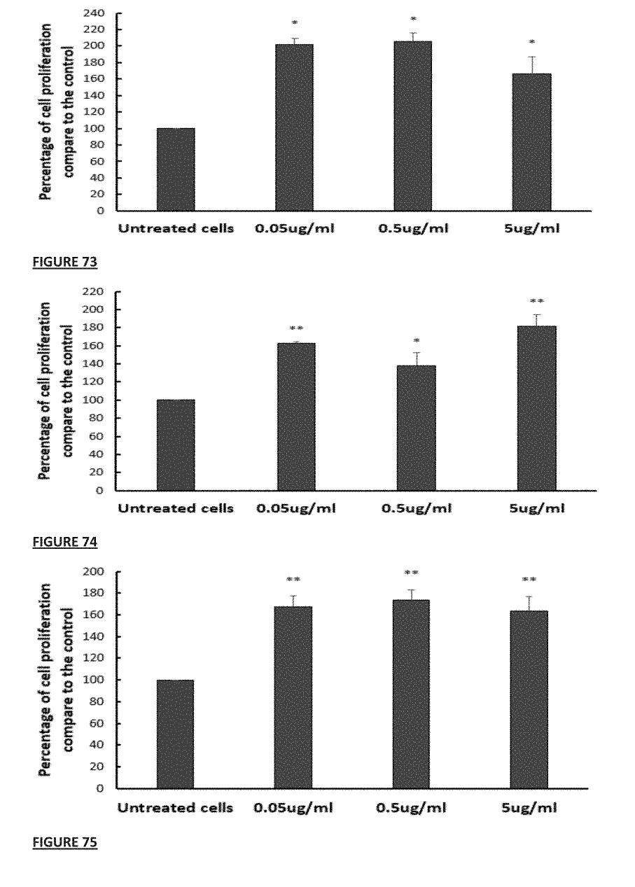

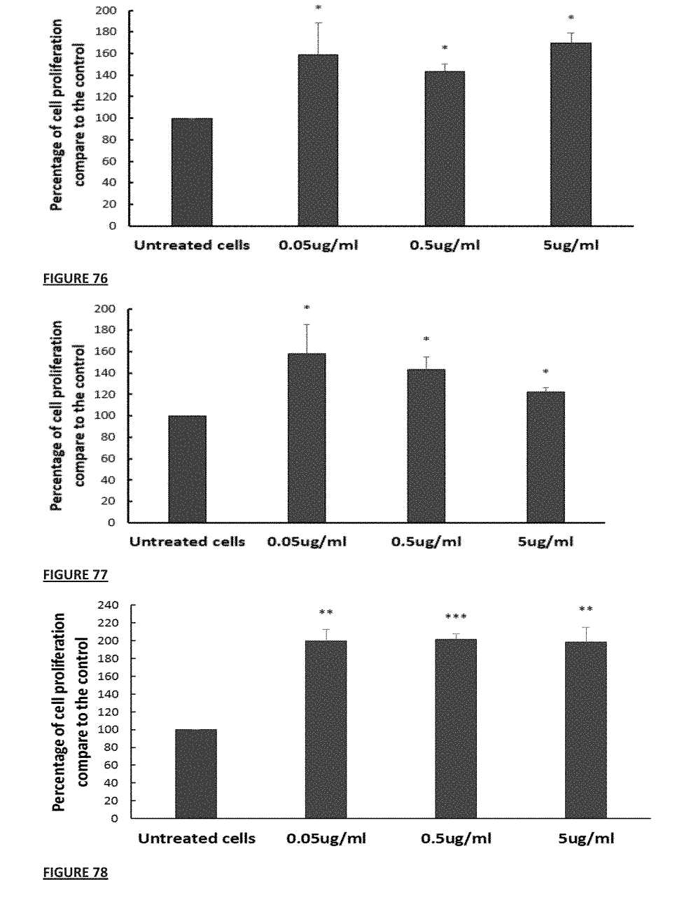

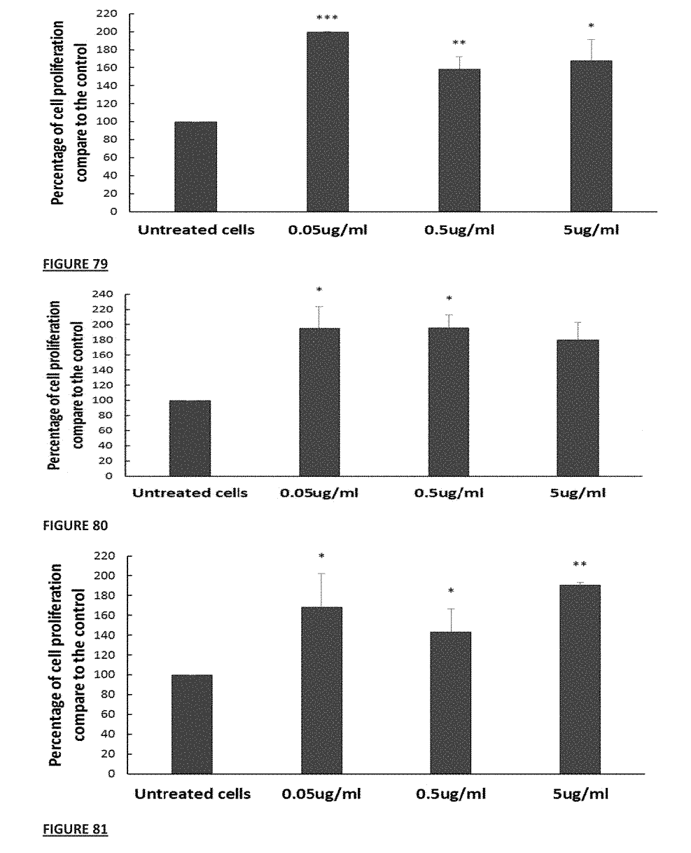

FIG. 28. THP-1 differentiated macrophages treated with I_222two for 24 hrs. prior to LPS stimulation were compared to untreated cells. TNF-α secretion in I_222two treated cells is reduced by 80% vs. untreated cells. Equivalent results are observed at 1 ug/ml and 50 ug/ml concentrations of I_222two, indicating the potency of I_222two.

### Section B

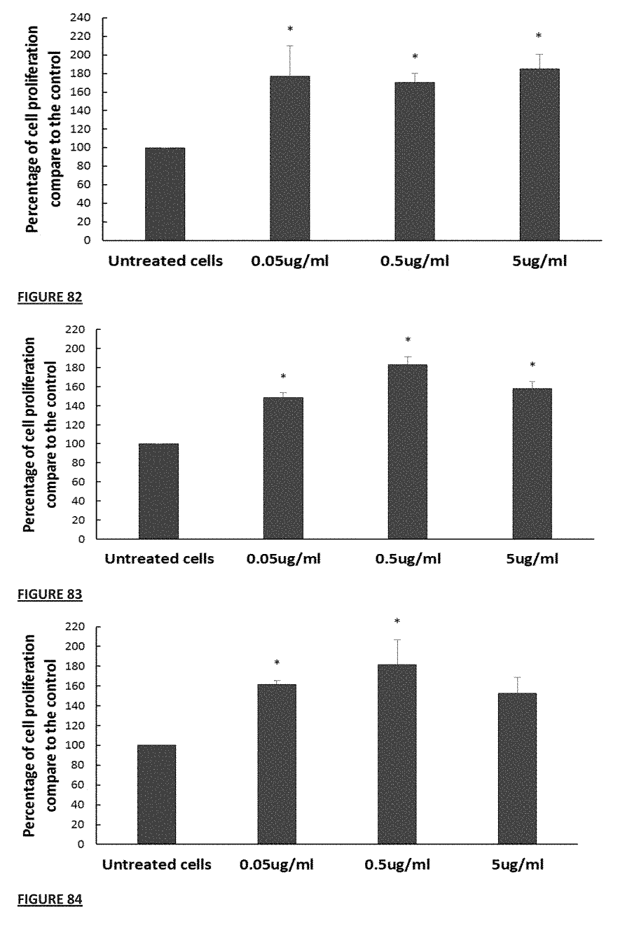

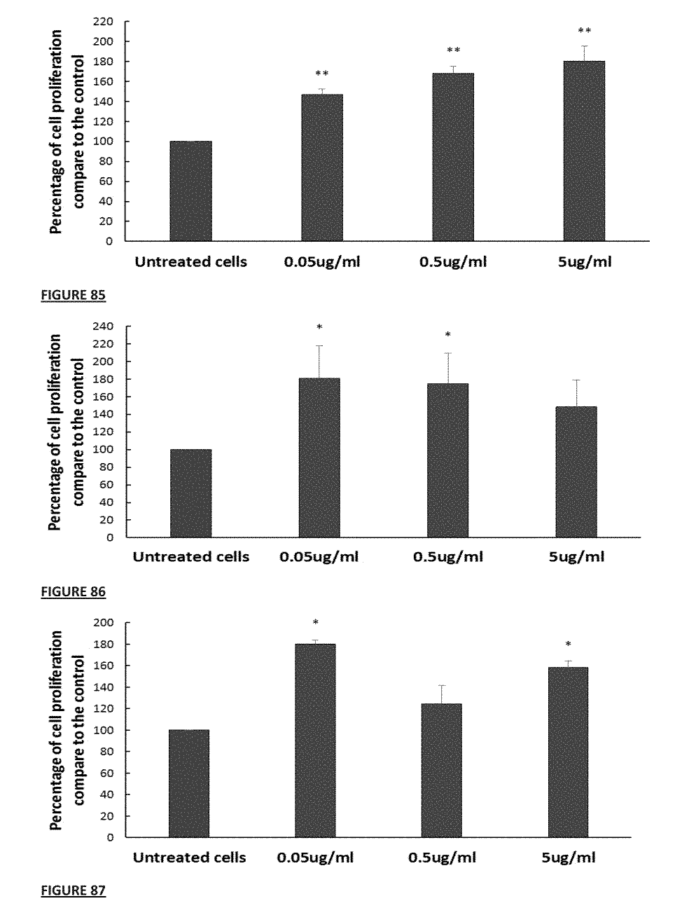

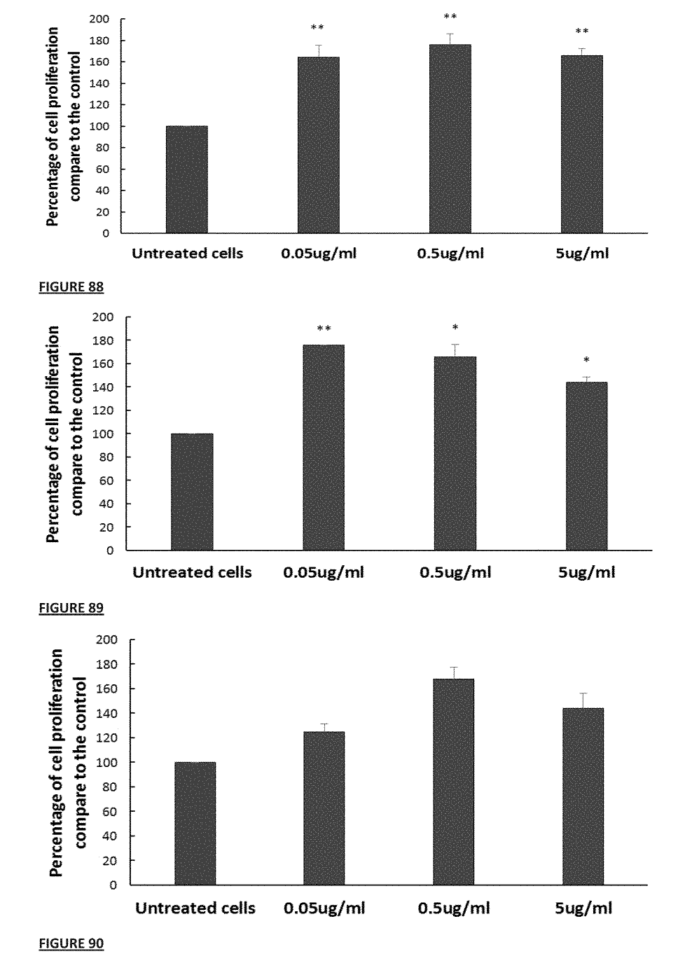

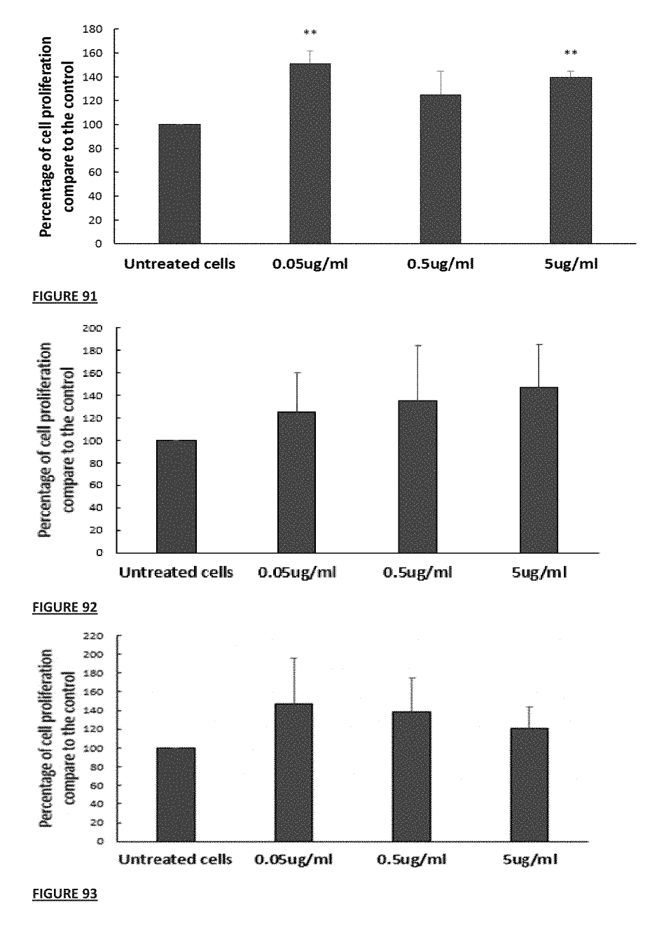

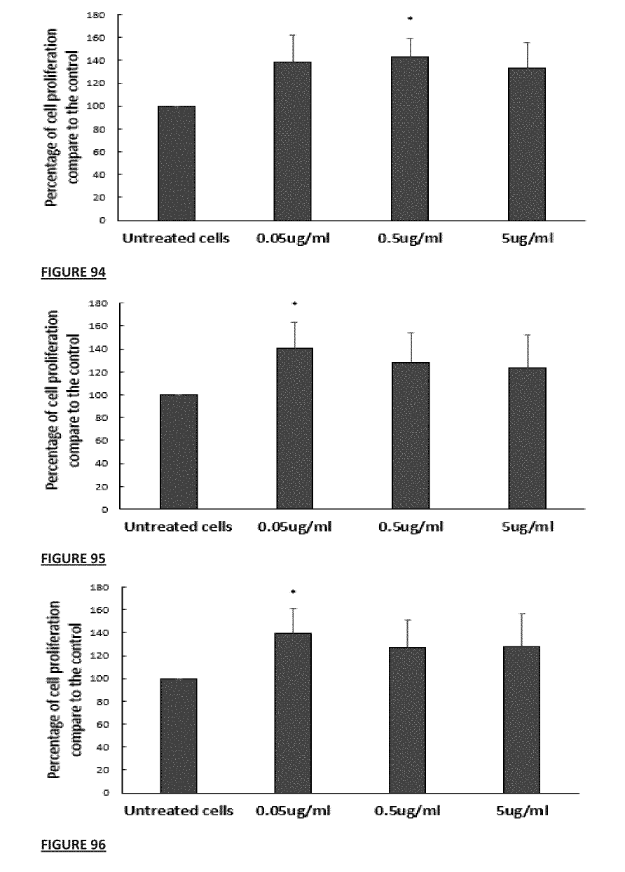

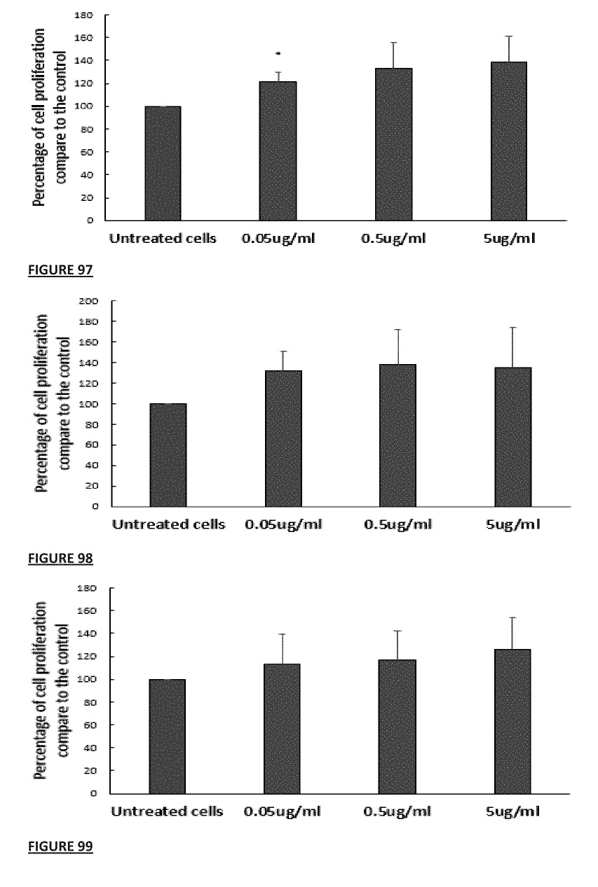

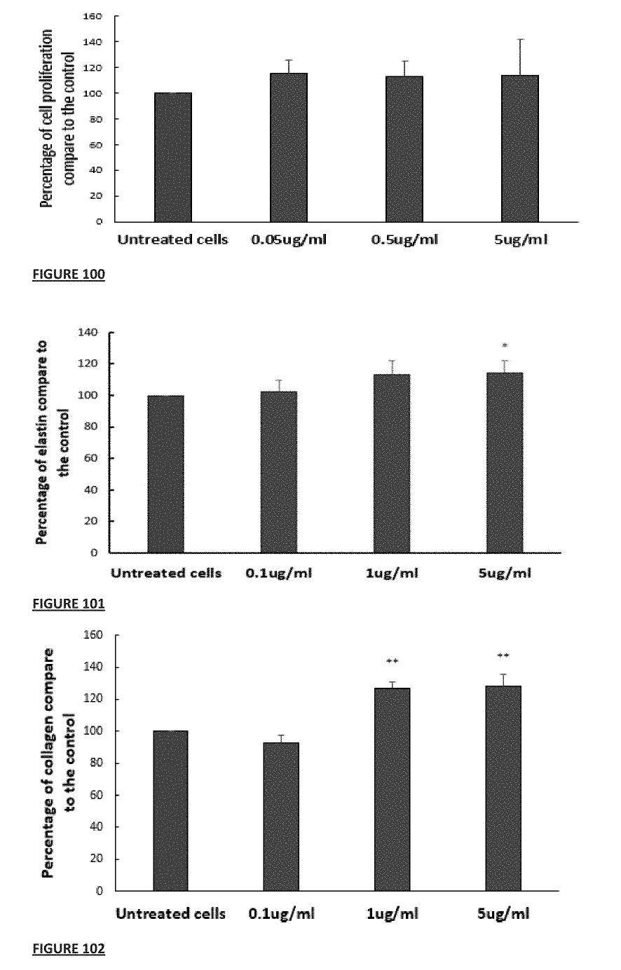

FIGS. 1 to 100: Effect of synthetic peptides of the invention on proliferation of Human Dermal Fibroblasts (HDF).

FIG. 101: Effect of synthetic peptide of the invention (SEQ ID 42) on elastin synthesis of Human Dermal Fibroblasts (HDF).

FIG. 102: Effect of synthetic peptide of the invention (SEQ ID 42) on collagen synthesis of Human Dermal Fibroblasts (HDF).

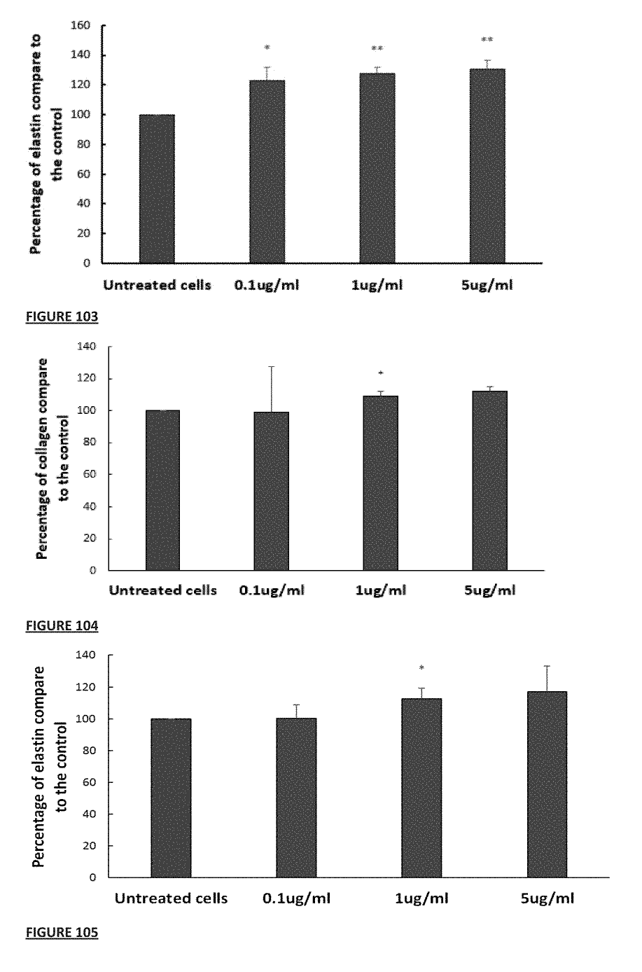

FIG. 103: Effect of synthetic peptide of the invention (SEQ ID 701) on elastin synthesis of Human Dermal Fibroblasts (HDF).

FIG. 104: Effect of synthetic peptide of the invention (SEQ ID 701) on collagen synthesis of Human Dermal Fibroblasts (HDF).

FIG. 105: Effect of synthetic peptide of the invention (SEQ ID 246) on elastin synthesis of Human Dermal Fibroblasts (HDF).

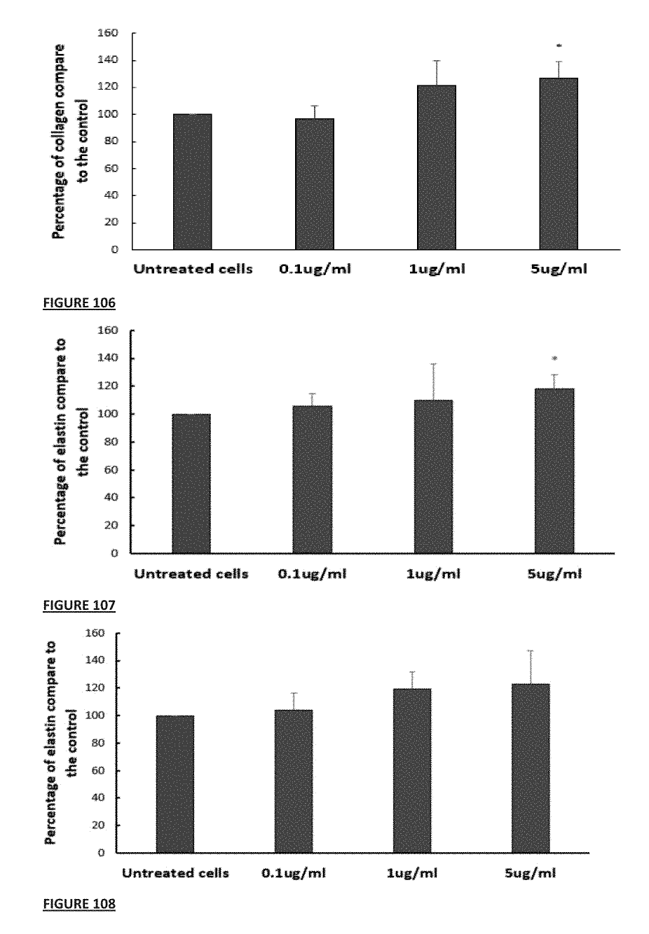

FIG. 106: Effect of synthetic peptide of the invention (SEQ ID 246) on collagen synthesis of Human Dermal Fibroblasts (HDF).

FIG. 107: Effect of synthetic peptide of the invention (SEQ ID 284) on elastin synthesis of Human Dermal Fibroblasts (HDF).

FIG. 108: Effect of synthetic peptide of the invention (SEQ ID 245) on elastin synthesis of Human Dermal Fibroblasts (HDF).

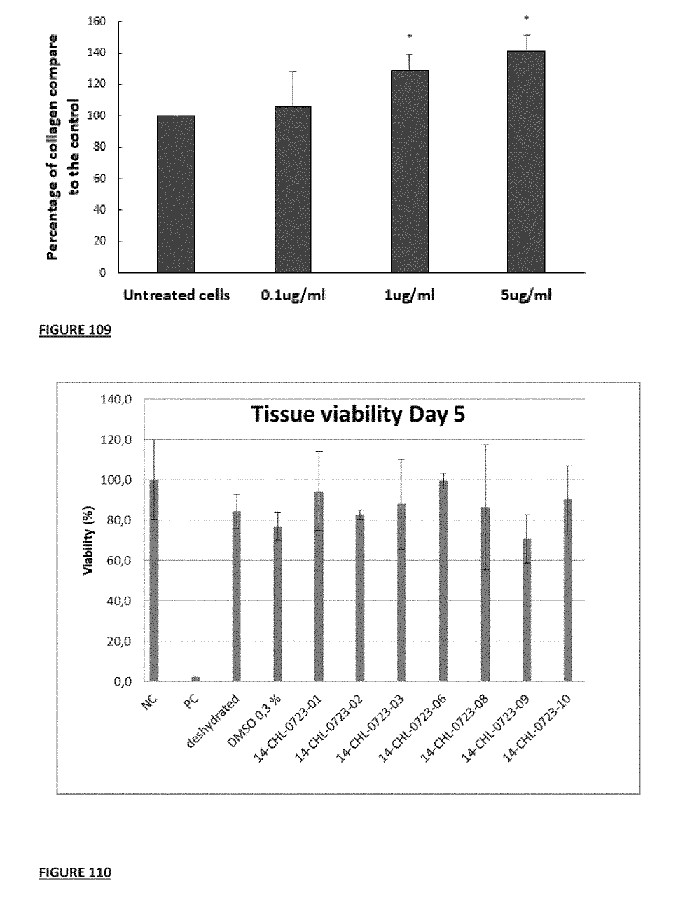

FIG. 109: Effect of synthetic peptide of the invention (SEQ ID 245) on collagen synthesis of Human Dermal Fibroblasts (HDF).

FIG. 110. shows the integrity controls and viability controls for the assay system.

FIG. 111. % of elastin expression in superficial dermis compared to control (water or DMSO) for peptides P1, P2 and P3 * shows significant increases of elastin expression in superficial AND middle dermis.

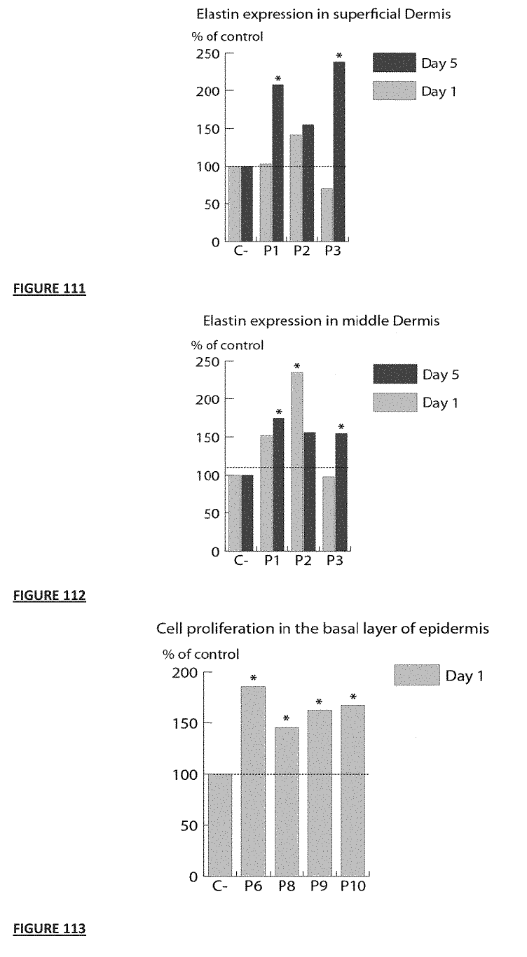

FIG. 112. % of elastin expression in middle dermis compared to control (water or DMSO) for peptides P1, P2 and P3. * shows significant increases of elastin expression in superficial AND middle dermis.

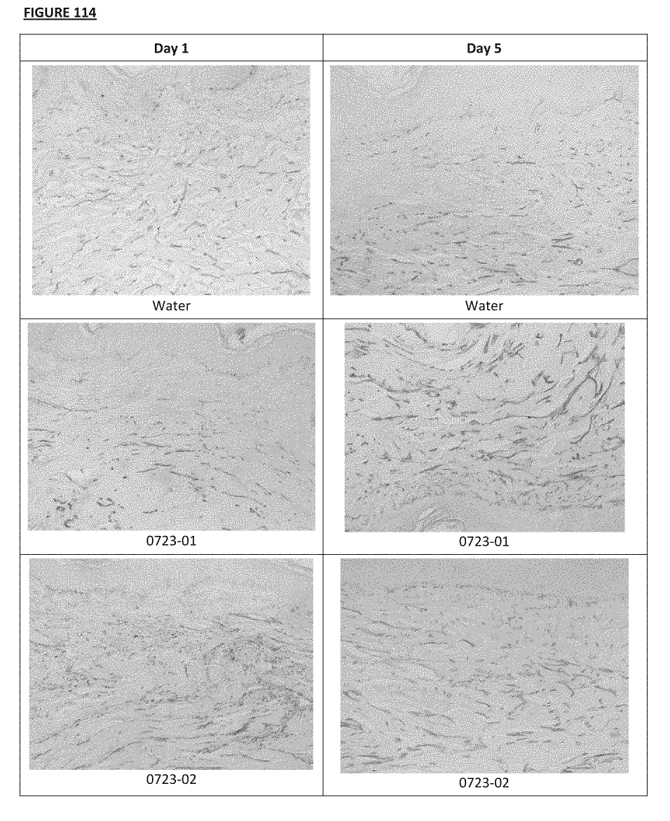

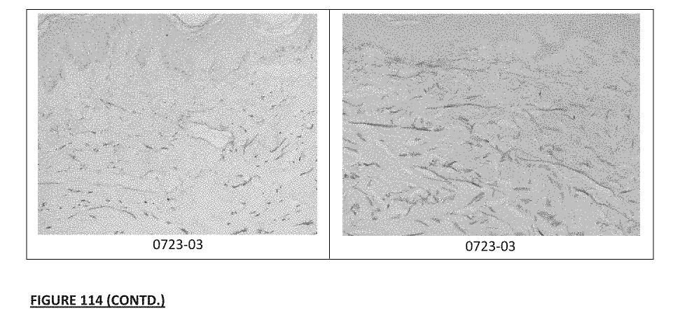

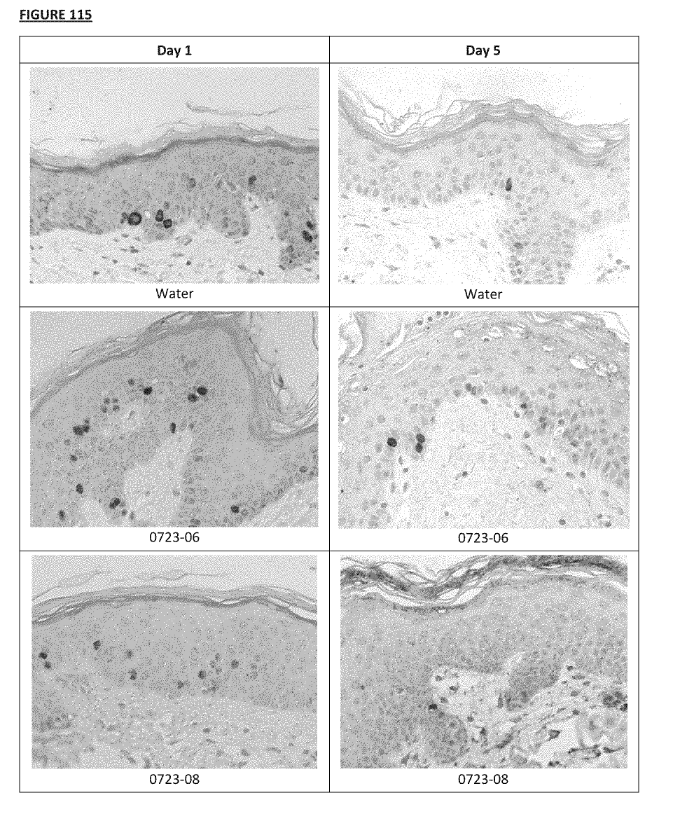

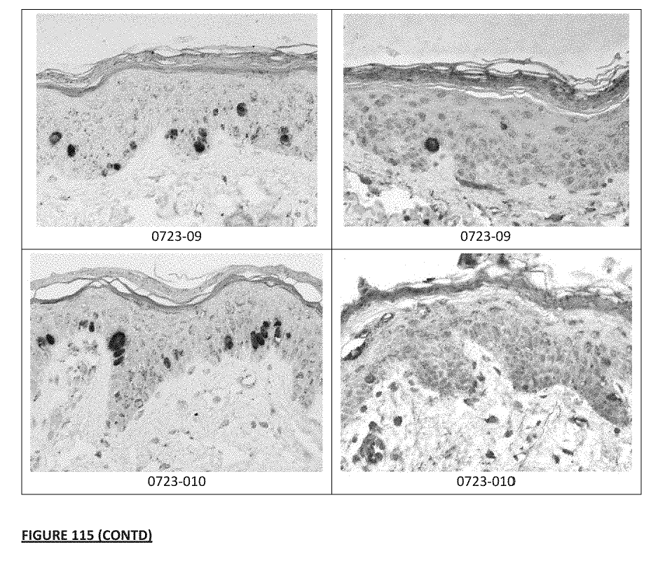

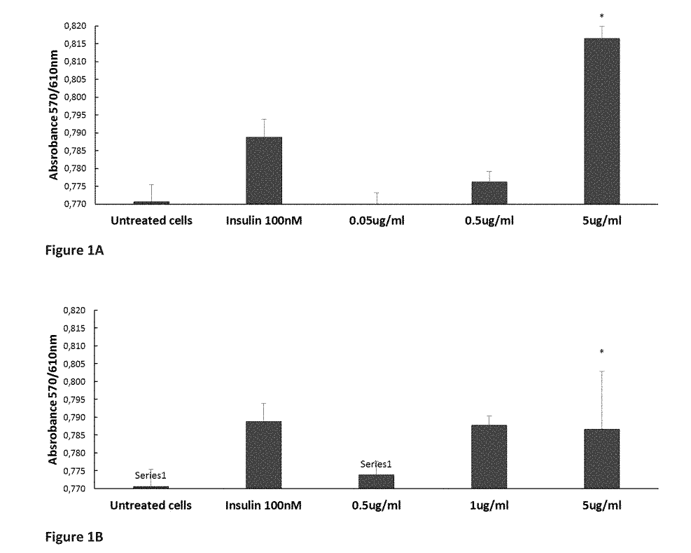

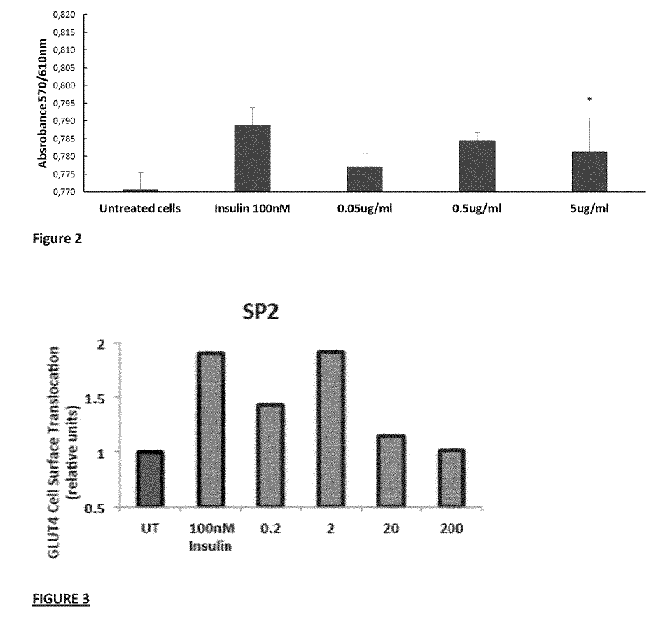

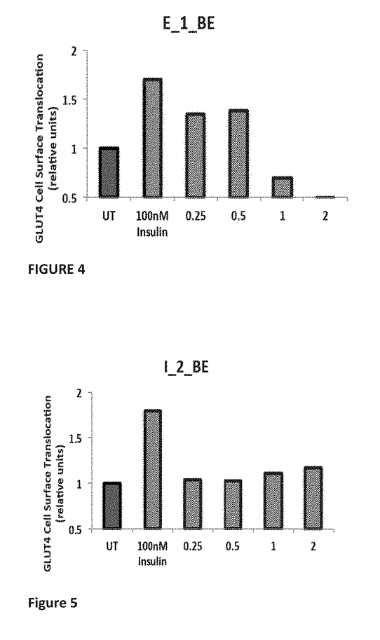

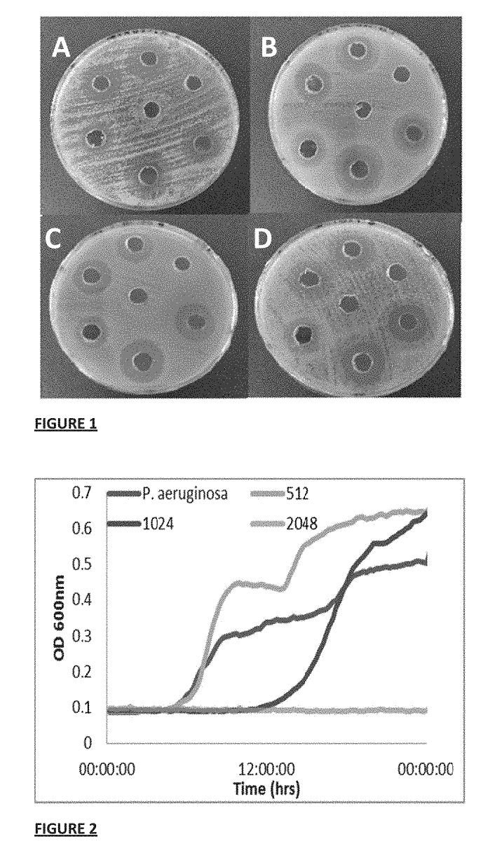

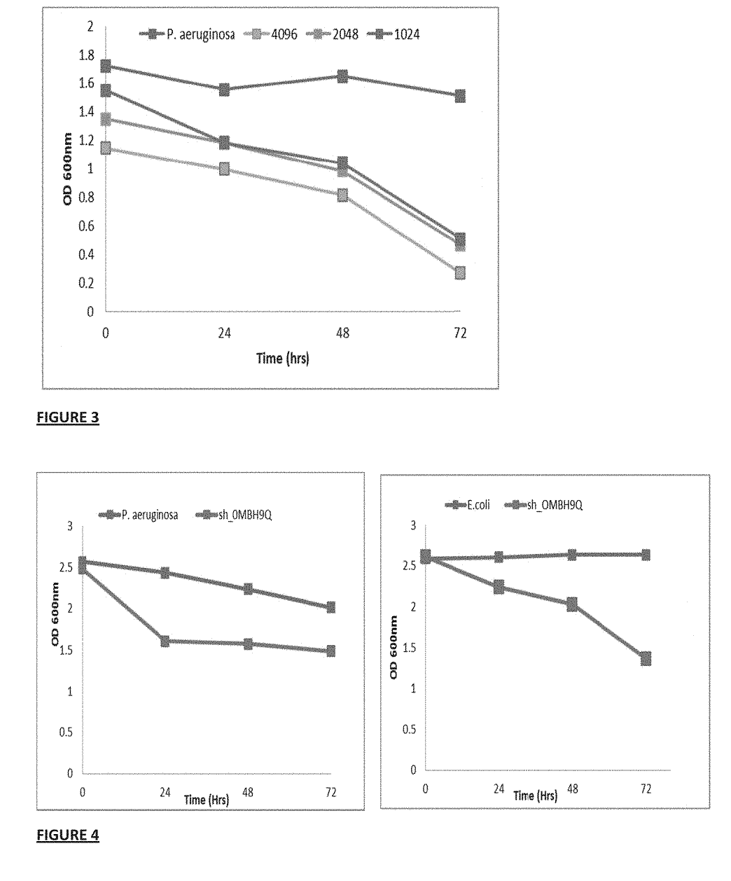

FIG. 113. % of cell proliferation in the basal layer of epidermis compared to control (water or DMSO) for peptides P6 and P8, and peptide compositions P9 and P10 * shows significant increases.

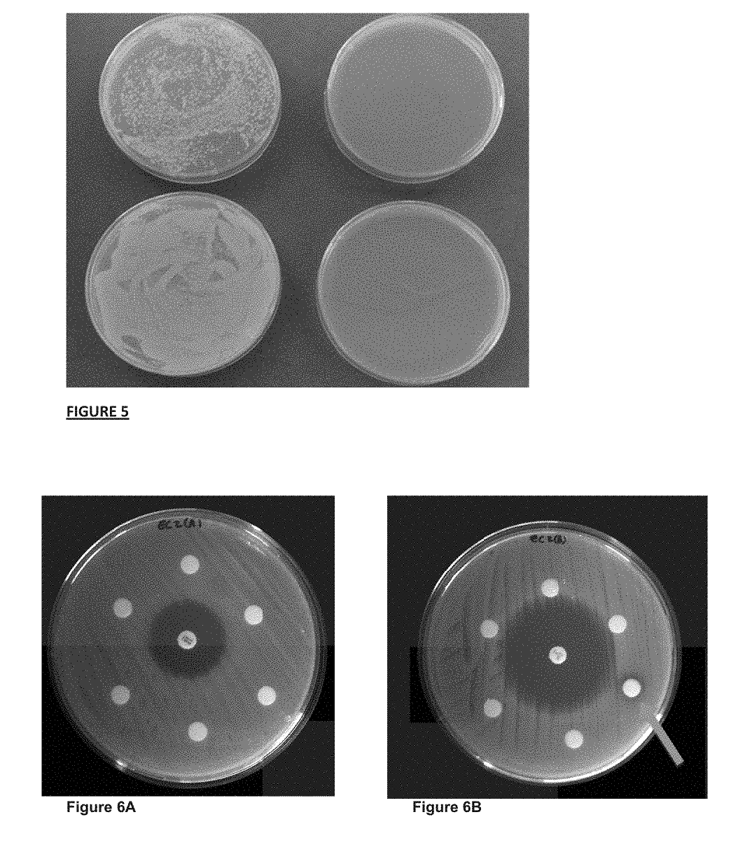

FIG. 114. Histological analysis of the elastic fibers (+catechin, ×200).

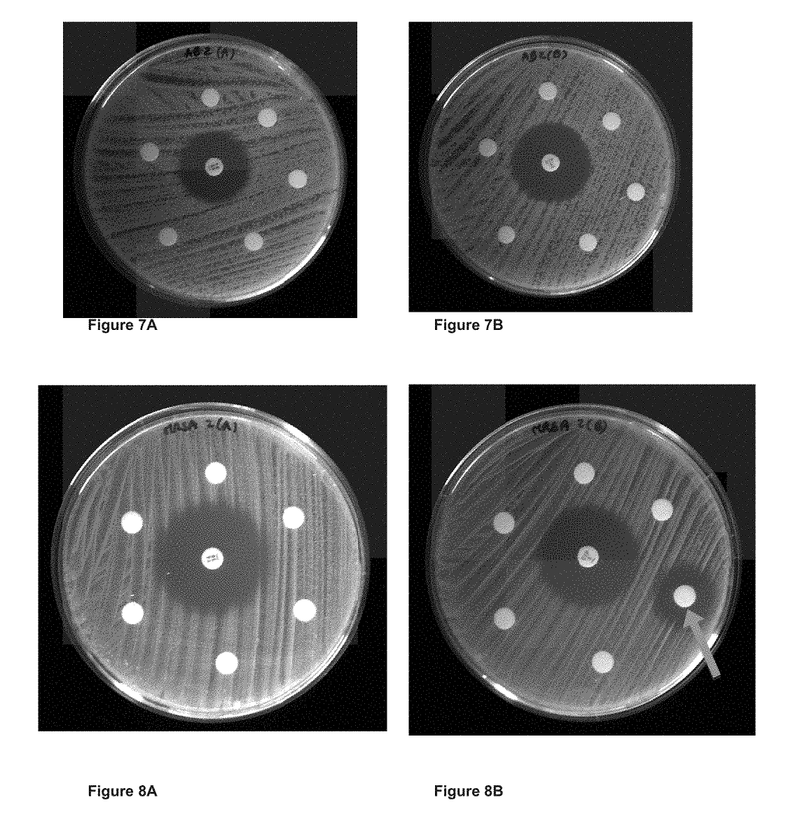

FIG. 115. Immunohistochemical evaluation of the mitotic index (Ki67, ×400).

### Section C

FIG. 1A: The effect of synthetic peptide SEQ ID 51 (Rice) on glucose uptake in skeletal muscle cells.

FIG. 1B: The effect of synthetic peptides—SEQ ID 13 (Pea) on glucose uptake in skeletal muscle cells.

FIG. 2: The effect of synthetic peptide SEQ ID 66 (Rice) on glucose uptake in skeletal muscle cells. FIG. 3: The effect of synthetic peptide SEQ ID 7 (Rice) on GLUT4 translocation in L6-GLUT4myc skeletal muscle cells.

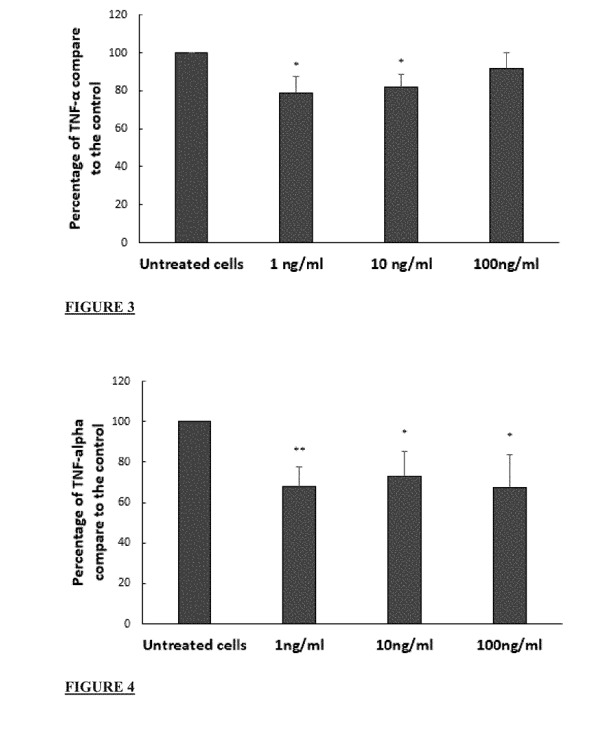

FIG. 4: The effect of peptide composition E_1_BE on GLUT4 translocation in L6-GLUT4myc skeletal muscle cells.

FIG. 5: The effect of peptide composition I_2_BE on GLUT4 translocation in L6-GLUT4myc skeletal muscle cells.

### Section D

FIG. 1. Agar Diffusion Assay. The activity of the peptide composition WHICH ONE was determined against S. aureus (A), Salmonella Typhimurium (B), P. aeruginosa (C) and E. coli (D). The arrow highlights the position of the peptide on the disk.

FIG. 2. Effect of peptide composition E_2_AM on the growth of P. aeruginosa. Growth curves were conducted in Mueller Hinton at pH7 over 24 hours using a Synergy H1 plate reader. Data was then analysed using the Gen5 Software.

FIG. 3. Total viable counts after 72 hours in P. aeruginosa inoculated orange juice. The red line shows the inoculated control and the purple, blue and orange show decreasing concentrations of peptide composition E_2_AM. Plates were read in a Synergy H1 plate reader

FIG. 4: Total viable counts after 72 hours in P. aeruginosa (left) and E. coli (right) inoculated milk at 37 degrees. Peptide composition E_2_AM is included at 5 mg/mL in blue. Plates were read in a Synergy H1 plate reader every 24 hours.

FIG. 5: Plate Count assay with Minced beef. Plates 1 & 2 show a bacterial count of 1×10 −1 CFU/mL and plates 3 & 4 are 1×10 −4 CFU/mL dilution with control (left) and peptide (right) at 37 degrees after 72 hours.

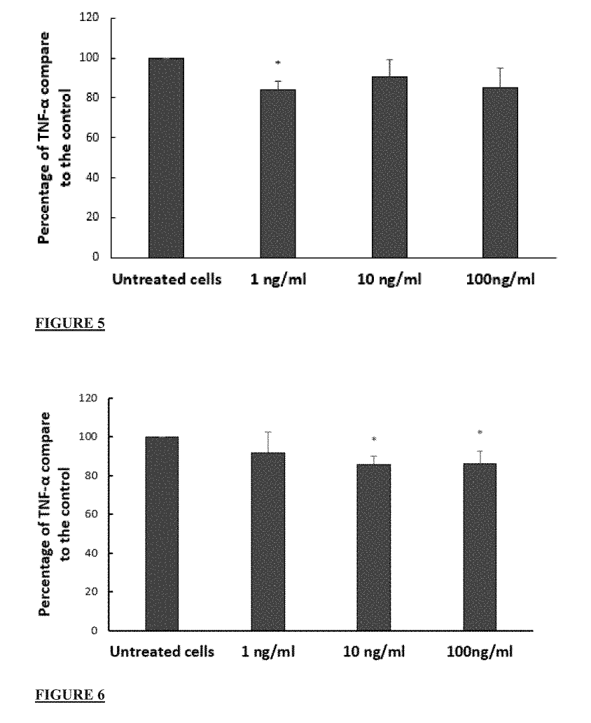

FIGS. 6A and 6B are pictures of agar plates in which E Coli ATCC25922 is growing showing an inhibition zone (9-10 mm) obtained with peptide I_87_SF (arrow). Control antibiotic discs (TET and CIP) were placed in the centre of each plate.

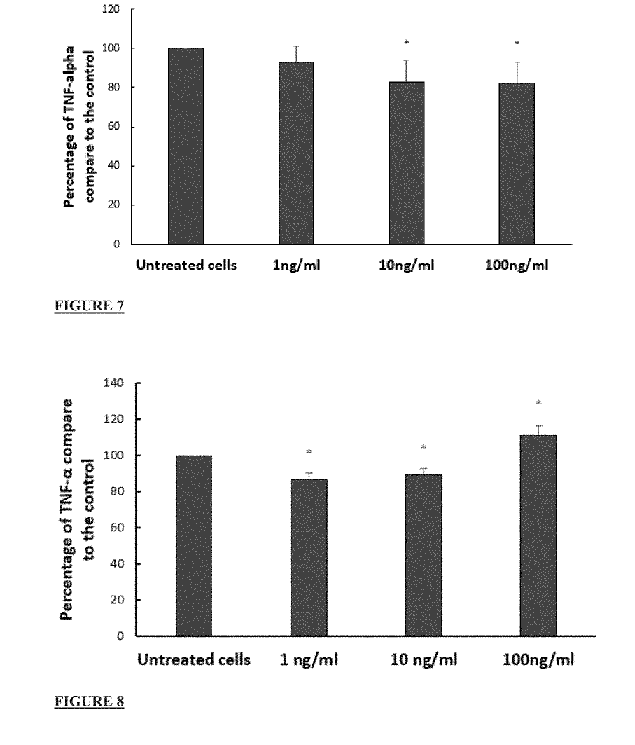

FIGS. 7A and 7B are pictures of agar plates in which Acinetobacter baumannii 19606 is growing, No antibacterial activity was detected. Control antibiotic discs (TET and CIP) were placed in the centre of each plate.

FIGS. 8A and 8B are pictures of agar plates in which MRSA 4330 is growing showing an inhibition zone (18 mm) obtained with peptide I_87_SF (arrow). Control antibiotic discs (TET and CIP) were placed in the centre of each plate.

### DETAILED DESCRIPTION OF THE INVENTION

### Section A

### EXAMPLE 1

### Inflammatory Response

TNF-α is secreted by macrophages in response to stimulation by endotoxins such as lipopolysaccharides (LPS). TNF-α is thought to be involved in systemic inflammation and dysregulation of TNF-α production is thought to be involved in many diseases. The Biolegend assay is a sandwich ELISA kit that is designed for the accurate quantitation of human TNF-α from cell culture supernatant, serum or plasma.

THP-1 monocytes were seeded in a 96 well plate at 10,000 cells per well in RPMI containing 10% fetal calf serum (FCS), 1% Pen/strep, 1% L-glutamine, 100 nM PMA and allowed to differentiated for 72 h prior to experimentation.

Following differentiation the cells were incubated with 100 ng/ml, 10 ng/ml or 1 ng/ml synthetic peptide for 24 h respectively.

Following treatment the cells were stimulated with 10 ng/ml LPS for 5 h and the quantity of TNF-α in the supernatant determined using the Biolegend assay ELISA kit.

Results were calculated as a percentage of the untreated control. An increase in optical density reading indicates greater quantity of TNF-α release into cell culture supernatant.

The results are provided in FIGS. 1 to 21 and summarised in Table 1 below. All experiments were prepared in dublicate on three plates (6 wells/conditions). Significance was calculated using Stsudents t-test (*p<0.05 compared to control, **p<0.01 compared to control, *** p<0.001 compared to control).

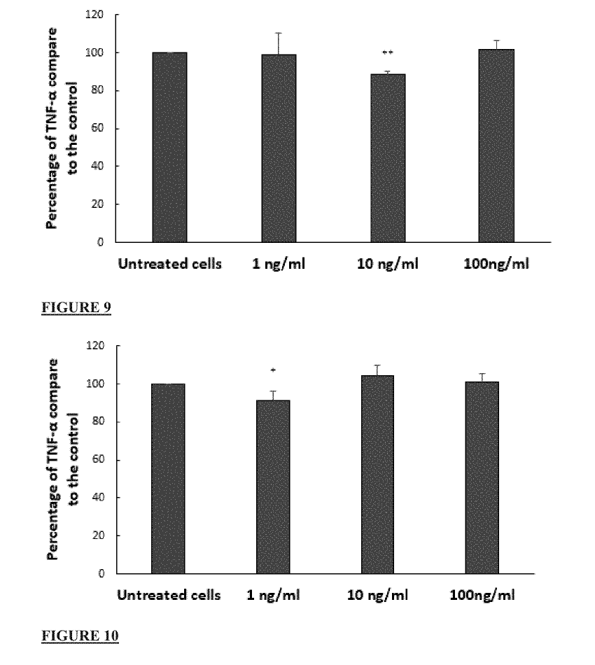

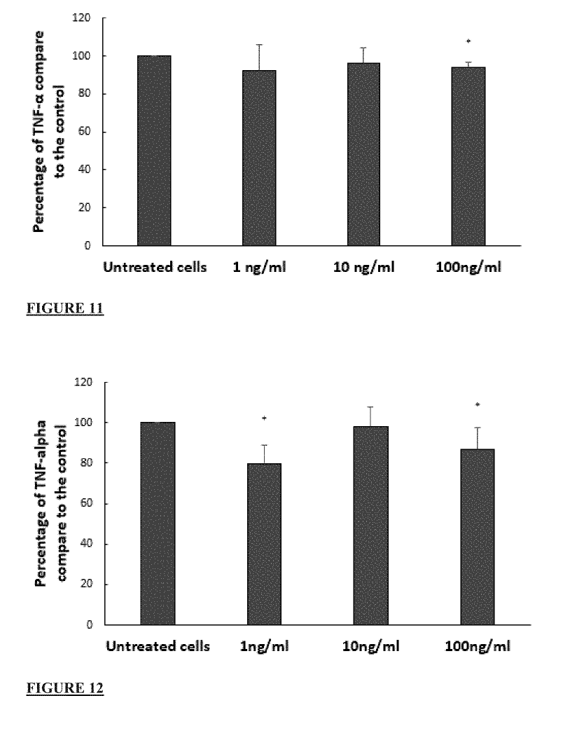

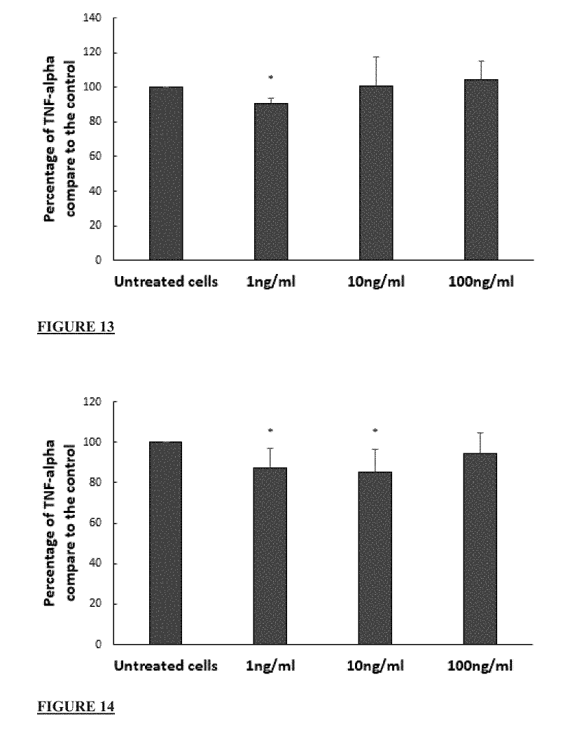

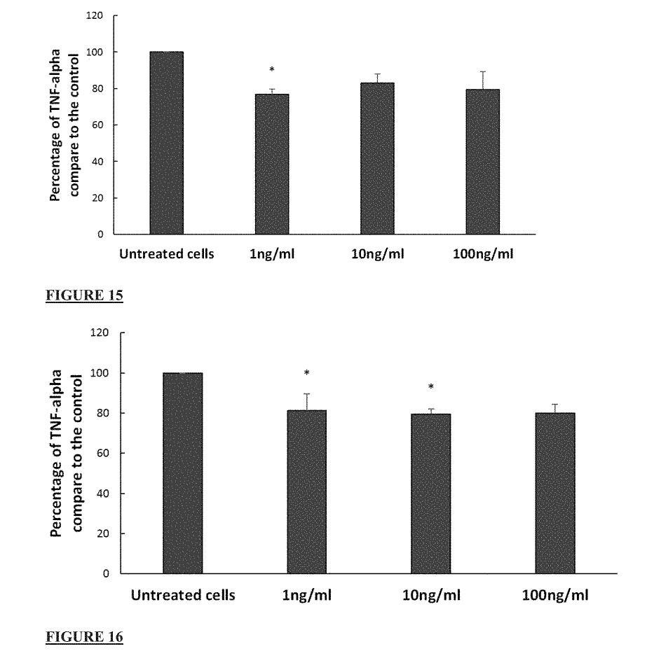

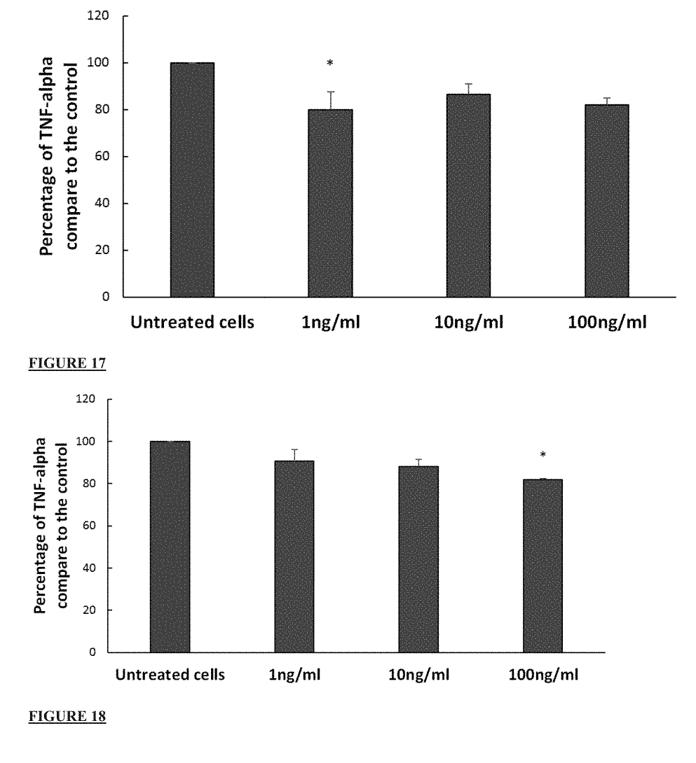

TABLE 1

| FIG. NUMBER | SEQ ID | TNF-α DECREASE |
|---|---|---|
| FIG. 1 | 339 | 26% |
| FIG. 2 | 352 | 23% |
| FIG. 3 | 341 | 21% |
| FIG. 4 | 351 | 18% |
| FIG. 5 | 144 | 16% |
| FIG. 6 | 93 | 14% |
| FIG. 7 | 320 | 13% |
| FIG. 8 | 92 | 13% |
| FIG. 9 | 75 | 11% |
| FIG. 10 | 76 | 9% |
| FIG. 11 | 349 | 6% |
| FIG. 12 | 350 | |
| FIG. 13 | 105 | 9% |
| FIG. 14 | 177 | |
| FIG. 15 | 345 | 23% |
| FIG. 16 | 353 | 20% |
| FIG. 17 | 344 | 20% |
| FIG. 18 | 346 | 18% |
| FIG. 19 | 85 | 80% |
| FIG. 20 | 91 | 80% |
| FIG. 21 | 350 | 80% |

### EXAMPLE 2

### Inflammatory Response

The effect of six synthetic peptides of the invention, SP1 to SP6 (SEQUENCE ID NO: 108, 109, 110, 111, 85 and 91) and four peptide compositions on the inflammatory response in vitro using a cell line was determined.

Peptide composition I_1_HR (Rice) contained the followings peptides (identified by SEQ ID): 116, 197, 207, 112, 211, 158, 201, 203, 114, 183, 130, 113, 182, 167, 166, 152, 220, 213, 215, 154, 219, 218, 165, 123, 185, 190, 209, 181, 198, 200, 147, 172, 184, 124, 153, 205, 115, 196, 151, 161, 160, 216, 210, 208, 146, 133, 204, 212, 206.

Peptide composition I_2_HR (Rice) contained the followings peptides (identified by SEQ ID): 189, 177, 174, 129, 176, 202, 193, 195, 194, 192, 182, 128, 220, 127, 134, 136, 135, 180, 179, 178, 219, 218, 145, 120, 175, 190, 149, 126, 187, 191, 121, 122, 159, 132, 162, 137, 150, 186, 188, 164, 118, 125, 163, 157, 156, 117.

Peptide composition E_1_HR (Pea) contained the followings peptides (identified by SEQ 76, 106, 102, 101, 100, 92, 96, 83, 89, 90, 104, 82, 75, 79, 78, 77, 99, 103, 72, 86, 105, 94, 93, 81, 97, 80, 88, 85, 87, 71, 107, 73, 84, 98, 95.

Peptide composition E_2_HR contained homologs of the peptides of the invention.

A J774.2 mouse macrophage cell line was treated with 100 μM of each synthetic peptide (SP) and 0.5 mg/ml of each peptide composition and the effect on two pro-inflammatory markers—tumour necrosis factor α (TNFα) and interleukin-1β (IL-1β) was determined after inflammation was induced using lipopolysaccharide (LPS) as an inflammatory stimulus. A one way anova was used with the dunnett test which is a multiple comparison and compares every mean with a single control mean.

### EXAMPLE 3

### Synthetic Peptides: Cell Viability

Synthetic peptides were first diluted in a suitable solvent. Dimethyl sulfoxide (DMSO) was the solvent of choice for peptides with poor predicted water solubility. Final concentration of DMSO in each well: SP1 (1_155_HR)-0.3%, SP2 (1_374_HR)-0%, SP3 (E_155_HR)-0.3%, SP4 (E_54_HR)-1%, SP5 (E_41_HR)-1%, SP6 (E_788_HR)-0.3%, positive Control-0%. Cells were first treated with 100 μM of each SP for 24 hours before an alamar blue assay was performed. No viability issues were seen with any of the peptides.

### EXAMPLE 4

### Peptide Compositions: Preparations and Toxicity

The peptide compositions were prepared by adjusting the pH to between 6-7 and sterile filtering. The effects of the peptide compositions on cell viability was determined. J774.2 macrophages were treated with 1 mg/ml and 0.5mg/ml of each peptide composition, hydrogen peroxide to induce cell death as a positive control, and a peptide known to be non-toxic as a negative control. An alamar blue assay was then performed and cell survival is shown in FIG. 19 as a percentage of untreated (100%). As cell survival was compromised with lmg/ml of peptide, 0.5 mg/ml of peptide composition was used for further assays.

### EXAMPLE 5

### Inflammatory Markers

The effect of the DMSO on TNFα and IL-1β secretion was determined. 1% DMSO significantly increased levels of TNFα (FIG. 3A. ***p<0.001 w.r.t LPS) and this was taken into account when analysing the effect of the peptides on TNFα. No significant effect was seen with regards DMSO and IL-1β secretion.

### EXAMPLE 6

### Inflammatory Response

THP-1 differentiated macrophages were treated with a composition of rice peptides of the invention (I_2_HR) for 24 hrs. prior to LPS stimulation were compared to untreated cells. TNF-α secretion in I_2_HR treated cells is reduced by 92% vs. untreated cells. Significant results are observed at 100 ug/ml and 500 ug/ml concentrations of I_2_HR, indicating the potency of I_2_HR.

### Section B

### EXAMPLE 1

### Cell Proliferation Assay

BrDu is incorporated into newly synthesised DNA strands of actively proliferating cells. Following partial denaturation of double stranded DNA, Brdu is detected immunochemically allowing the assessment of the population of cells which are synthesizing DNA.

Human Dermal Fibroblasts (HDF—Sigma 10605a) were seeded in a 96 well plate at 10,000 cells per well in DMEM containing 10% fetal calf serum (FCS), 1% Pen/strep, 1% L-glutamine and allowed to adhere for 24 h.

Following the initial 24 h incubation the cells were incubated with 5 μg/ml, 0.5 μg/ml or 0.05 μg/ml synthetic peptide for 24 h respectively.

After 18 h incubation with synthetic peptides 20 μl BrDu reagent was added to each well. At 24 h incubation the cell were fixed and the amount of 2-DG6P was measured using the BrdU Cell Proliferation Assay, all steps were carried out according to the manufacturer's instructions. Results were calculated as a percentage of the untreated control. An increase in optical density reading indicates greater incorporation of BrDu and increase cell proliferation. The results are shown in FIGS. 1-100 and Table 2 below.

TABLE 2

| FIG. | SEQ | INCREASE IN | FIG. | SEQ | INCREASE IN |
|---|---|---|---|---|---|
| NO | ID | PROLIFERATION | NO | ID | PROLIFERATION |
| 1 | 121 | 48% | 29 | 98 | 49% |
| 2 | 105 | 40% | 30 | 454 | 38% |
| 3 | 249 | 30% | 31 | 85 | 35% |
| 4 | 226 | 30% | 32 | 453 | 25% |
| 5 | 84 | 20% | 33 | 158 | 21% |
| 6 | 330 | 18% | 34 | 464 | 18% |
| 7 | 181 | 33% | 35 | 73 | 16% |
| 8 | 83 | 32% | 36 | 359 | 15% |
| 9 | 247 | 28% | 37 | 124 | 15% |
| 10 | 97 | 26% | 38 | 112 | 15% |
| 11 | 74 | 29% | 39 | 733 | |
| 12 | | | 40 | 728 | |
| 13 | 168 | | 41 | 727 | |
| 14 | 151 | | 42 | 730 | |
| 15 | 470 | 119% | 43 | 731 | |
| 16 | 257 | 118% | 44 | 148 | |
| 17 | 256 | 117% | 45 | 343 | |
| 18 | 457 | 114% | 46 | 345 | |
| 19 | 499 | 113% | 47 | 484 | |
| 20 | 253 | 112% | 48 | 729 | |
| 21 | 222 | 110% | 49 | 456 | |
| 22 | 272 | 97% | 50 | 494 | |
| 23 | 252 | 111% | 51 | 723 | |
| 24 | 248 | 86% | 52 | 722 | |
| 25 | 472 | 77% | 53 | 498 | |
| 26 | 365 | 58% | 54 | 475 | 13% |
| 27 | 502 | 68% | 55 | 718 | |
| 28 | 496 | 51% | 56 | 337 | 8% |
| 57 | 500 | 6% | 79 | 463 | 100% |
| 58 | 717 | | 80 | 465 | 96% |
| 59 | 297 | | 81 | 467 | 90% |
| 60 | 340 | | 82 | 461 | 85% |
| 61 | 719 | | 83 | 341 | 83% |
| 62 | 726 | | 84 | 468 | 82% |
| 63 | 725 | | 85 | 285 | 81% |
| 64 | 724 | | 86 | 496 | 81% |
| 65 | 720 | | 87 | 146 | 80% |
| 66 | 721 | | 88 | 478 | 76% |
| 67 | 503 | 125% | 89 | 452 | 76% |
| 68 | 474 | 121% | 90 | 495 | 68% |
| 69 | 504 | 119% | 91 | 403 | 51% |
| 70 | 114 | 119% | 92 | 455 | 47% |
| 71 | 505 | 118% | 93 | 270 | 47% |
| 72 | 482 | 113% | 94 | 501 | 43% |
| 73 | 479 | 106% | 95 | 473 | 41% |
| 74 | 477 | 81% | 96 | | 39% |
| 75 | 410 | 73% | 97 | 471 | 38% |
| 76 | 475 | 69% | 98 | 460 | 38% |
| 77 | 497 | 58% | 99 | 93 | 26% |
| 78 | 480 | 102% | 100 | 462 | 15% |

### EXAMPLE 2

### Collagen Production Assay

Hydroxyproline in tissue preparations is a direct measure of the amount of collagen present. FIRELISA Human Hydroxyproline ELISA kit assay is designed to measure hydroxyproline in tissue or peptide compositions.

Human Dermal Fibroblasts (HDF Sigma 10605a) were seeded in 24 well plates at 50,000 cells per well in DMEM containing 10% fetal calf serum (FCS), 1% Pen/strep, 1% L-glutamine and allowed to adhere for 24 h.

Following the initial 24 h incubation the cells were incubated with 5 μg/ml, 1 μg/ml or 0.1 μg/ml synthetic peptide for 96 h respectively.

After treatment the cells were lysed using 4 freeze thaw cycles in liquid nitrogen. The lysed cells were centrifuged and 50 μl/ml of each supernatant was assayed using the FIRELISA Human Hydroxyproline ELISA kit. All steps were carried out according to the manufacturer's instructions.

Results were calculated as a percentage of the untreated control. An increase in optical density reading indicates an increase collagen content. The results are shown in FIGS. 102, 104, 106 and 109

### EXAMPLE 3

### Elastin Production Assay

Elastin is a highly elastic protein in connective tissue and allows many tissues in the body to resume their shape after stretching or contracting. FIRELISA Human Elastin ELISA kit assay is designed to measure Elastin in tissue or protein/peptide compositions.

Human Dermal Fibroblasts (HDF) were seeded in 24 well plates at 50,000 cells per well in DMEM containing 10% fetal calf serum (FCS), 1% Pen/strep, 1% L-glutamine and allowed to adhere for 24 h.

Following the initial 24 h incubation the cells were incubated with 5 μg/ml, 1 μg/ml or 0.1 μg/ml synthetic peptide for 96 h respectively.

After treatment the cells were lysed using 4 freeze thaw cycles in liquid nitrogen. The lysed cells were centrifuged and 50 μl/ml of each supernatant was assayed using the FIRELISA Human Elastin ELISA kit. All steps were carried out according to the manufacturer's instructions.

Results were calculated as a percentage of the untreated control. An increase in optical density reading indicates an increase collagen content.

The results are shown in FIGS. 101, 103, 105, 107, 108 and 109.

### EXAMPLE 4

### Elastin and Cell Proliferation Assays

### Equipment

Incubator, Flow Laminar Chamber, Sterile Polished Plastic Rod, Pipettor, Maintenance medium, Plate 6 well, Plate 24 well.

### Reagents

MTT, PBS, SDS, Formaldehyde, Xylene, Ethanol absolute, Dulbecco's phosphate-buffered saline (DPBS), Metal Enhanced DAB substrate kit, ABC peroxidase staining kit, Citric acid, Sodium hydroxide 2N, Hydrogen peroxide 30%, Anti-Filaggrin, Anti-rabbit IgG-Biotin, Tween 20.

### Test System

Nature: Human skin tissue 5 mm diameter

Batch number: EXP004050B009 and EXP004050B011

Provider: Laboratoire Biopredic International—8-18 rue Jean Pecker—35000 Rennes—France.

Tel: +33 (0)2.99.14.36.14—Fax: +33 (0)2.99.54.44.72.

Certificates of analysis are present in Annex 1.

Two batches are used for the assay. Batch EXP004050B005 is used for experiment day 1, and Batch EXP004050B006 is used for experiment day 5.

### Maintenance Medium

Maintenance Medium: Batch n°: MIL 218C

Provider: Laboratoire Biopredic International—8-18 rue Jean Pecker—35000 Rennes—France.

### Peptides Tested

SEQ ID NO: 283

P1:

SEQ ID NO: 246

P2:

SEQ ID NO: 284

P3:

(SEQ ID 1319)

P4: RPYYSNAPQEIF

(SEQ ID 1320)

P5: VLLEQQEQEPQH

SEQ ID NO: 245

P6:

(SEQ ID 1321)

P7: QQYGIAASPFLQSAA

SEQ ID NO: 42

P8:

### Compositions Tested

P9 (14-CHL-0723-09) is the Pea composition (SEQ ID Numbers:50, 85, 74, 140, 82, 136, 189, 77, 169, 149, 171, 178, 143, 127, 190, 141, 147, 133, 186, 125, 122, 119, 87, 90, 86, 89, 138, 129, 123, 120, 117, 113, 110, 121, 105, 98, 55, 161, 19, 317, 135, 130, 146, 177, 160, 170, 188, 83, 78, 36, 96, 159, 26, 330, 168, 148, 184, 151, 151, 165, 114, 284)

P10 (14-CHL-0723-010) is the Rice composition (SEQ ID Numbers: 245, 246, 263, 250, 257, 259, 276, 255, 251, 264, 256, 266, 274, 270, 269, 356, 245, 380, 262, 258, 356, 218, 252, 358, 271, 253, 344, 275, 272, 226, 224, 220, 248, 261, 265, 373, 375, 247, 249, 363, 273, 343, 273, 362)

### Application Method

Skin explants were prepared from abdominal plastic surgery. Some explants were delipidated with alcohol to obtain a dehydrated skin.

These explants were maintained in maintenance medium supplied by the provider Bioprédic International for 5 days. Test items are applied twice per day with 5 μL per explant. At the end of the test, viabilities controls are realized with the MTT on two explants, the third explant is fixed in the formaldehyde 4% for histology and cell staining.

For each time of analysis (D1 and D5), histologies on delipidated explants, treated explants with test items, the DMSO 0.3% control and water control, are performed.

After receipt in the laboratory, each skin explant in the maintenance medium is delipidated with 5 μL alcohol during 3 hours.

After 3 hours, all skin explants are treated two per day with test items, and they are incubated at 37° C. +/−2° C., 5% CO2 for 1 day or 5 days.

Integrity of the system is realized at day 1 and day 5 with a viability control with MTT.

### Immunostaining

Histology is realized by the laboratory Gredeco and the immunostaining to elastin and Ki67 are realized by the same laboratory. Immunostaining to filaggrin is realized by the laboratory Intertek.

The detection of elastin (rabbit monoclonal antibody, clone P15502, LSBio) is performed using an immunoperoxidase technique two layers (ABC kit, Vector Laboratories) and revealed by AEC (3-amino-9-ethylcarbazole). The immunohistochemical staining intensity in the elastic fibers is evaluated using a semi-quantitative histological score. Epithelial proliferation was analyzed by immunohistochemistry using anti-Ki67 antibody.

Immunodetection was performed using an indirect immunoperoxidase technique three layers, amplified (DAKO kit) and revealed by AEC (3-Amino-9-ethylcarbazole). Counting the number of labeled cells (keratinocytes of the basal layer of the epidermis) is performed and provides the total number of basal cells to calculate the % of labeled cells.

The specific staining of filaggrin is performed with an immunoperoxidase staining (ABC kit, Fisher). The intensity of immunohistochemical marker in the epidermis is evaluated relative to the negative control of the solvent (Water or DMSO 0.3%).

### C: Results

### Viability Control

The integrity control and the viability control are present in FIG. 1. These controls do allow to validate the assay system. The viability is >50% for test items, and they do not show a cytotoxicity according to the test.

### Immunostaining

### Elastin Expression

The elastic fibers of the dermis were revealed by staining with the catechin and morphometrically quantified by analysis by computer-assisted image. The percentage area taken up by elastic fibers in the dermis was calculated in the dermis and the average superficial dermis. Results are presents in Table 4, FIG. 2 and FIG. 3.

Under the experimental conditions of the study, 0723-1 and 0723-3 samples show an increase by twice of elastic fibers in the superficial dermis compared to control water (Error! Reference source not found.), and an increase in the middle dermis compared to the water control at D5. The 0723-2 sample shows an increase doubled in the middle dermis at day 1 compared to control water and an increase at day 5.

### Ki67 Expression

The results of the immunohistochemical analysis of Ki67 are reported in Table 5 and expressed as % of labelled at the basal layer of the epidermis. The Error! Reference source not found. shows the percentage of Ki 67 cells compared to negative controls (water or DMSO). Immunohistochemical analysis of mitotic activity is shown in annex 4 with a reminder of the average for each analysed conditions.

Table 5. % of Ki67 positive cells in the basal layer of the epidermis. Orange bands correspond to samples dissolved into DMSO 0.3% instead of water.

Under the experimental conditions of the study, test item 0723-06, 0723-08, 0723-09 and 0723-010 show an increase in the number of mitotic cells compared to EGF at day 1. A decrease in the mitotic index was observed on day 5 compared to day 1 for all analysed conditions.

The decrease in this cell staining on day 5 is caused by the model. Indeed, after approximately 3 days cell turnover is exhausted on this model.

### Section C

### EXAMPLE 1

Measuring glucose uptake using 2-deoxyglucose (2-DG) is a widely accepted method used to investigate glucose uptake in skeletal muscle cells. 2-DG is taken up by glucose transporters and metabolized to 2-DG-6-phosphate (2-DG6P). The amount of accumulated non-metabolized 2-DG6P is proportional to glucose uptake by cells.

### Method:

1. Human skeletal myoblasts (Sigma 150-05a) were seeded in a 96 well plate at 10,000 cells per well in Skeletal Muscle Differentiation medium and allowed to differentiated for 72 h prior to experimentation.

2. The differentiated cells were serum starved for 24 h prior to stimulation with insulin or synthetic peptides. After starvation, the serum free media was removed, cells rinsed with Phosphate Buffered Saline (PBS) and media replaced with 100 μl of Krebs-Ringer-Phosphate-HEPES (KRPH) and incubated for 1 h.

3. The cells were then stimulated with 100 nM insulin for 30 minutes or 5 μg/ml, 0.5 μg/ml or 0.05 m/ml synthetic peptide for 3 h respectively.

4. Following stimulation the cells were incubated with 10 μl/well of 2-DG solution for 40 min and glucose uptake was measured using the PrismColor Glucose Uptake Assay Kit' (Molecutools), all steps were carried out according to the manufacturer's instructions.

5. Results were calculated as a percentage of the untreated control. An increase in optical density reading indicates greater incorporation of 2-DG6P and increase in glucose uptake. All experiments were carried out in duplicate on three plates (6 wells/condition). Significance was determined using the Students t-test (*p<0.05 compared to control, **p<0.01 compared to control, *** p<0.001 compared to control)

The results are shown in FIGS. 1 and 2—all synthetic peptides caused a significant increase in glucose uptake in the cells.

### EXAMPLE 2

### Study Description

Skeletal muscle is the predominant site of glucose disposal (80%) under insulin-stimulated or post-prandial conditions. Under these conditions, transport of glucose into skeletal muscle is facilitated principally by the insulin-responsive glucose transport protein GLUT4, which translocates to the cell surface upon insulin or contractile stimulation.

We determined the effect of six synthetic peptides (SP1-6) and four peptide compositions on in vitro GLUT4 translocation using the L6 rat skeletal muscle cell line. A clone of the L6 cell line containing overexpression of GLUT4 tagged with a c-myc epitope (courtesy of Prof. Amira Klip, Hospital for Sick Children, Toronto) was used to investigate the efficacy of each synthetic peptide and peptide composition for effects on GLUT4 translocation in a dose-response design.

SP2 [SEQ ID 555] is a glucose transport promoting fragment of Pea Protein P13918, whereas peptides SP1 and SP3-SP6 are comparative peptides.

SP1 (E_685two_BE)

[SEQ ID 1309]

DTFYNAAWDPSNR
SP2 (E_64two_BE)

[SEQ ID 555]

VLDLAIPVNRPGQL
SP3 (E_93_BE)

[SEQ ID 25]

YQHQQGGKQEQENEGNNIFSGFK
SP4 (I_641_BE)

[SEQ ID 1310]

ALDWAIANLLR
SP5 (I_1021_BE)

[SEQ ID 1311]

YDYENVDAGAAK
SP6 (I_24_BE)

[SEQ ID 1312]

EVQDSPLDACR

The following compositions of peptides were tested for skeletal muscle glucose transport activity in an in-vitro test:

I_2_BE (comprises peptides of SEQ ID NO: 55 and 10)

E_1_BE (comprises peptides of SEQ ID NO: 48, 49, 50, 51, 54, 58, 60, 61, 62, 63)

Cell culture

L6-GLUT4myc cells were grown in 10% FBS and 2 μg/ml blasticidin. Cells were grown for 48-72 hours before being seeded in 24-well plates at 15,000 cells per well in 2% FBS and allowed to differentiate for 6 to 8 days prior to experimentation.

L6-GLUT4myc cells were serum-starved for three hours prior to incubation with 100 nM of insulin for 30 mins, or 200, 20, 2.0 and 0.2 μM of SP, and 2, 1, 0.5 and 0.25mg/ml of peptide composition for 3 hours respectively. A 3 hour incubation period was selected based on previous findings identifying that incubation with branch chain amino acid containing di-peptides for 3 hours increases glucose uptake in L6 myotubes 1. Treatments were staggered in order to determine GLUT4myc translocation at the same time point.

### Measurement of GLUT4myc Translocation in L6 Myotubes

The quantity of myc-tagged GLUT4 at the cell surface was measured by antibody-coupled colorimetric assay. Briefly, after incubation with either insulin for 30 mins or synthetic peptide or peptide composition for 3 hours respectively, L6-GLUT4myc cells were fixed via incubation with 3% paraformaldehyde (PFA). A 0.1 M glycine solution was then added to quench PFA and cells were blocked with 5% goat serum. The myotube monolayer was exposed to anti-myc antibody and then incubated with peroxidase conjugated donkey anti-mouse IgG. 1mL of o-phenylenediamine dihydrochloride (OPD) reagent was added to each well and this reaction was stopped by adding 250 μl/well of 3 M HCL. To determine GLUT4 translocation to cell surface, a measured aliquot of each condition was determined spectrophotometrically on a plate reader using absorbance at 492 nm.

### Synthetic Peptides

Peptides were first diluted in a suitable solvent. Dimethyl sulfoxide (DMSO) was the solvent of choice for peptides with poor predicted water solubility. Final concentration of DMSO in each well at 200, 20, 2 and 0.2 μM for each synthetic peptide are shown in Table 6.

### Peptide Compositions

Peptide compositions were prepared by adjusting the pH to between 6-7 using 1 M NaOH or HCL and subsequently sterile filtered.

TABLE 6
Concentration of DMSO per well for each synthetic peptide

| | Concentration of DMSO per well (%) | | | |
|---|---|---|---|---|
| Peptide | 200 μM | 20 μM | 2 μM | 0.2 μM |
| SP1 (E_685two_BE) | 4.0 | 0.4 | 0.04 | 0.004 |
| SP2 (E_64two_BE) | 0.9 | 0.09 | 0.009 | 0.0009 |
| SP3 (E_93_BE) | 0.8 | 0.08 | 0.008 | 0.0008 |
| SP4 (I_641_BE) | 0.2 | 0.02 | 0.002 | 0.0002 |
| SP5 (I_1021_BE) | 3.0 | 0.3 | 0.03 | 0.003 |
| SP6 (I_24_BE) | 0.0 | 0.0 | 0.0 | 0.0 |

Synthetic peptides

In addition to an untreated control, 100 nM insulin was utilised to stimulate a maximal GLUT4 translocation response i.e. a positive control in each experiment. The average increase in cell surface GLUT4 translocation in response to 100 nM insulin was 1.72-fold when compared to untreated control (FIG. 3). Treatments were staggered so that all conditions (untreated, insulin and sample) were processed at the same time-point. There was a trend for SP2 to increase GLUT4 translocation at a concentration ranging from 0.2-2 μM. SP1 at 200 μM tended to decrease translocation due to poor cell viability.

### Peptide Compositions

Peptide composition E_1_BE tended to increase GLUT4 translocation at a concentration ranging from 0.25-0.5 mg/ml, however 1 and 2 mg/ml induced progressive cell death. Furthermore, there was a trend for composition I_2_BE to increase GLUT4 translocation in a dose-dependent manner (FIGS. 4-6).

### Conclusion of the Experiment

SP2 and compositions E_1_BE and I_2_BE displayed a trend for stimulatory effect on skeletal muscle GLUT4 translocation and warrant further investigation for their potential to facilitate glucose transport in skeletal muscle.

### EXAMPLE 3

### Anti-Hyperglycaemic Properties of Peptide Compositions I_2 BE and E_1_BE in db/db Mice

### Preparation

I_2_BE or E_1_BE is administered as a solution or suspension in Purified Water. According to stability data, test item formulations at 10 mg/ml in Purified Water are stable for 10 hours at +2-+8° C. protected from light. Therefore test item formulations are kept at +2-+8° C. protected from light and used within 10 hours after preparation. Aspect of formulations and maximal duration of storage are detailed below.

### Material

### Species: Mouse.

Strain: BKS.Cg-Dock7m+/+Leprdb/J (db/db diabetic mice) (souche JAXTM Mice strain). Choice of species: The mouse was chosen because of its acceptance as a predictor of pharmacological effects of drugs in man and the recognition by regulatory authorities that this species is suitable for pharmacodynamic studies.

Age: 8-9 weeks on the day of randomisation.

Weight: On the day of randomisation, a maximum range of 2.5 g between each group should be achieved. The body weight of the animals on the day of randomisation will be mentioned in the report. About 10% excess animals will be ordered to allow selection of animals on the basis of body weight; if unassigned to groups, these will be available as spare animals, in case of unforeseen events.

### Study Design

The study involves 3 groups of 12 animals each. Groups will be as follows:
Group 1: control group dosed with the vehicle (Purified Water), po
Group 2: I_2_BE at 100 mg/kg, po
Group 3: E_1_BE at 100 mg/kg, po

Allocation of treatment to each animal is randomly determined before the start of the study. Homogeneity of groups will be validated on the criterion of body weight and glycaemia measured on the day of randomisation.

Justification of the number of animals per group:

The number of animals per group is the minimum number enabling an accurate assessment of the pharmacokinetics profile.

### Study Calendar

D-4 : Weighing, glycaemia measurements, inclusion and randomisation of animals
D1: Weighing of animals, start of daily oral administrations of test items or vehicle, and glycaemia measurements
D8: Weighing of animals and glycaemia measurements
D15: Weighing of animals and glycaemia measurements
D16-D18: Oral Glucose Tolerance Test (OGTT)
D22: Weighing of animals and glycaemia measurements
D29: Weighing of animals and glycaemia measurements
D29-D31: Blood sampling followed by the organs sampling

### Glycaemia Measurements

Blood glucose level is measured weekly from D1 up to D29, 90 ±30 minutes after the daily treatment. A drop of blood is collected from the tail vein of non fasted db/db mice and is put on the extremity of a glucose strip (Nova Biomedical) placed into the Glucose Meter (Nova Biomedical).

### Oral Glucose Tolerance Test (OGTT)

Over the third week (D16-D18) and after an overnight fasting period, the OGTT is performed. After a blood glucose level measurement (predose value) and 30 minutes after the daily oral administrations of test items or vehicle, animals are dosed by the oral route with 10 mL/kg of a glucose solution at 0.2 g/mL (2 g/kg) in Purified Water. Afterwards, blood glucose level are measured following the same procedure described above, at times 15, 30, 60, 90 and 120 minutes after the glucose overload.

### Intermediate Results

The effects of I_2_BE and E_1_BE on body weight and glycaemia are compared with those of the vehicle and the delta corresponding to the evolution of blood sugar in each group is calculated from D1 to D15. Evolution of blood glucose from D-5 to D1 and therefor prior to treatment shows that progression of the disease is the same in all three groups. Strong trends of activity were observed for both peptide compositions compare to control between D1 and D15 showing that both peptide compositions are able to control the evolution of blood sugar in diabetic animals.

### Results

The effects of I_2_BE and E_1_BE on body weight and glycaemia are compared with those of the vehicle using an analysis of variance for repeated measurements with a Dunnett's test in case of significance (P≦0.05). For OGTT, the results of glycemia after the glucose overload in treated animals is compared with those of the vehicle animals using an analysis of variance for repeated measurements with a Dunnett's test in case of significance (P=0.05).

Biochemical results (plasma glucose, HbA1c and insulin) are expressed as absolute values. The effects of I_2_BE and E_1_BE on biochemical parameters are compared with those of the vehicle using an analysis of variance with a Dunnett's test in case of significance (P≦0.05).

### Section D

### EXAMPLE 1

The anti-bacterial effects of peptide compositions of the invention were tested. The compositions are:

E_1_AM Contains substantially all of SEQ ID 106-251, 81, 68, 66, 106 and 107

E_2_AM Contains substantially all of SEQ ID 106-251, 81 and 68

Minimum Inhibitory Concentrations and Zone of Inhibition

MIC and MBC assays were carried out in Mueller Hinton broth previously adjusted to pH5, 7 and 9 and inoculated with 1×105 CFU/mL of each bacteria. The values shown represent the mean of three replicates performed on three independent days. Concentrations necessary to inhibit and completely halt growth are consistently lower in all strains at pH5. As the pH increases so too does the MIC and MBC suggesting that the bioactivity is improved in acidic conditions. This may be as a result of these conditions inducing a favourable isoelectric point an therefore, and enhanced electrostatic interaction between the positively charged hydrolysate and the negatively charged bacterial membrane. The zones of inhibition shown are the mean of three independent replicate experiments with the standard deviation. Values range from ˜11 mm to ˜21 mm with the best activity observed in P. aeruginosa. Each well is 8 mm in diameter alone and studies were conducted in Mueller Hinton agar at pH7.

Growth Curve in Mueller Hinton Broth pH7 at 37 degrees over 24 hours (FIG. 2) and Total viable counts of P. aeruginosa in peptide treated orange juice over 72 hours (FIG. 3)

The peptide composition interferes with the growth of P. aeruginosa. At a concentration of 1024 μg/mL, sh_0MBH9Q extended the lag-time of P. aeruginosa by ˜10 hours. Concentrations above this resulted in complete cell death. This value corresponds to the MBC determined at pH7.

Fresh orange juice was inoculated with P. aeruginosa at 1×105 CFU/mL and plates were read at selected time points. Increased reduction in the microbial population appears to be linear with the increasing concentration of the peptide composition continues to reduce counts of P. aeruginosa over time and induces a ˜1.2 log reduction at 4096 μg/mL after 72 hours.

Total viable counts after 72 hours in P. aeruginosa (FIG. 4 left) and E. coli (FIG. 4 right) inoculated milk at 37 degrees.

The plate count study was conducted with peptide compositions at a concentration of 5 mg/mL. The meat used was fresh beef mince with 5% fat content. FIG. 5 highlights the complete reduction in microbial counts of the microflora and pathogens present in the meat after 72 hours at dilutions of 1×10 −1 CFU/mL and 1×10 −4 CFU/mL when treated with 5 times the MIC identified in standard conditions. This study suggests the compound could be a suitable natural ingredient for extending the shelf life and control pathogenic populations in fresh minced meat.

### EXAMPLE 2

The following peptides were tested for bacterial inhibition activity in solid and liquid media test. I_87_SF [SEQ ID 640] is an antibacterial fragment of Rice Protein P14614, whereas the remaining six peptides are comparative peptides.

I_45_SF

(SEQ ID 1313)

ITSVNSQKFPILNLIQMSATR
I_77_SF

(SEQ ID 1314)

SRVQVVSNFGK
E_31_SF

(SEQ ID 1315)

VLDLAIPVNKPGQLQSFLLSGTQNQPSLLSGFSK
E_376_SF

(SEQ ID 1316)

NAMFVPHYNLNANSIIYALKGR
E_78_SF

(SEQ ID 1317)

LRPGVMFVVPAGHPFVNIASK
E_354_SF

(SEQ ID 1318)

GLFDLGHPLVNR
I_87_SF

[SEQ ID 640]

LNSQKFPILNLVQLSATR

TABLE 7
UCD_CFS_Strains used for Determination of antibacterial activity

| | Strain | Type | Hospital/Isolation | Source | ARP (Resistant to) | Reference |
|---|---|---|---|---|---|---|
| Gram- | E. coli 25922 | Reference | FDA strain Seattle 1946 | — | PEN; VAN; AMP; CLI; | http://www.atcc.org/ATCCAdvanced |
| negative | | | [DSM 1103, NCIB | | CL | CatalogSearch/ProductDetails/tabid/4 |
| | | | 12210] | | | 52/Default.aspx?ATCCNum=25922& |
| | | | | | | Template=bacteria |
| | Acinetobacter | Reference | Deposit in ATCC as | Urine | — | Hugh R, Reese R. Designation of the |
| | baumannii | | Bacterium anitratum | | | type strain for Bacterium anitratum |
| | ATCC19606 | | Schaub and Hauber | | | Schaub and Hauber 1948. Int. J. Syst. |
| | | | 2208 [81, DSM 6974] | | | Bacteriol. 17: 245-254, 1967. |
| Gram- | MRSA ATCC | Reference | Kansas | Human | AMP; PEN; OXA; | http://www.atcc.org/ATCCAdvanced |
| positive | 43300 | | | | MET; AXO; CIP; | CatalogSearch/ProductDetails/tabid/4 |
| | | | | | LEVO; GAT; ERY; CLI | 52/Default.aspx?ATCCNum=43300& |
| | | | | | | Template=bacteria |

Abbreviations:
MRSA - Methicillin-Resistant Staphylococcus aureus;
ARP - Antibiotic Resistance Profile;
AMC - Amoxicillin-Clavulanic acid;
C-Chloramphenicol;
F- Furazolidone;
Fc- Florfenicol
Gm-Gentamycin;
N-Neomycin;
NAL-Nalidixic acid;
S-Streptomycin;
Su-Sulfonamides;
TET- Tetracycline;
TMP-Trimetoprim;
AMP-ampicillin;
PEN - penicillin;
OXA - oxacillin;
MET-methicillin;
AXO - ceftriaxone;
CIP- ciprofloxacin;
LEVO-levofloxacin;
GAT - gatifloxacin;
ERY-erythromycin;
CLI- clindamycin;
CL- Cephalexin

Compounds tested. A total of 1 compound (1 peptide) was tested. Peptide stock=5 mg/mL dissolved in DMSO.

Preparation of the peptide. The powder was reconstituted with 1.04 mL of DMSO to achieve a final concentration of 5 mg/mL. The peptide was in high purity. No precipitation problems. Antibacterial activity testing (in solid media). Bacterial inoculums were adjusted to McFarland 0.5 standard and MHA plates swabbed. Blank disks were placed in the plates and 10 μL of each compound (at 64 μg/mL—maximum concentration tested) added. Plates were incubated at 37° C. for 16-18 hours. Appropriate controls (DMSO; Mueller-Hinton media alone; and two antibiotic discs—ciprofloxacin and tetracycline) were also performed.

Results from the Testing in Agar-Plates

Determination of antibacterial activity (inhibition of growth) was performed in Mueller-Hinton plates. After incubation period results were registered and plates photographed.

TABLE 8

| | Inhibition zone diameter (mm) | | | | | | | | |
|---|---|---|---|---|---|---|---|---|---|
| Compounds | E. coli ATCC 25922 | | | A. baumannii ATCC 19606 | | | MRSA ATCC 43300 | | |
| ID | Rep 1 | Rep 2 | Rep 3 | Rep 1 | Rep 2 | Rep 3 | Rep 1 | Rep 2 | Rep 3 |
| I_87_SF | 10 | 10 | 9 | NI | NI | NI | 18 | 18 | 18 |
| TET | 28 | 26 | 26 | 23 | 24 | 24 | 31 | 31 | 32 |
| CIP | 42 | 40 | 42 | 27 | 28 | 27 | 27 | 28 | 29 |
| DMSO | NI | NI | NI | NI | NI | NI | NI | NI | NI |
| MH | NI | NI | NI | NI | NI | NI | NI | NI | NI |

Legend: TET—tetracycline; CIP—Ciprofloxacin; MH—Mueller-Hinton (control); NI—No Inhibition of growth

Final result: Peptide I_87_SF showed some inhibitory activity against E. coli and MRSA, but not at the levels of susceptibility. No activity was obtained against A. baumannii.

Results from the Testing in Agar-Plates (Photos)

Note: Only one set is shown since the other two sets had the same results. Control antibiotic discs (TET and CIP) were placed in the centre of each plate.

The invention is not limited to the embodiments hereinbefore described which may be varied in construction and detail without departing from the spirit of the invention.

The invention is not limited to the embodiments hereinbefore described which may be varied in construction and detail without departing from the spirit of the invention.

SEQUENCES
PEPTIDE:

[SEQ ID 1]

SRGPIYSNEFGK
PEPTIDE:

[SEQ ID 2]

NSFNLER
PEPTIDE:

[SEQ ID 3]

VLDLAIPVNR
PEPTIDE:

[SEQ ID 4]

DDNEELR
PEPTIDE:

[SEQ ID 5]

LSSGDVFVIPAGHPVAVK
PEPTIDE:

[SEQ ID 6]

EDDEEEEQGEEEINK
PEPTIDE:

[SEQ ID 7]

NILEASFNTDYEEIEKVLLEEHEKETQHR
PEPTIDE:

[SEQ ID 8]

NILEASFNTDYEEIEKVLLEEHEK
PEPTIDE:

[SEQ ID 9]

NILEASFNTDYEEIEK
PEPTIDE:

[SEQ ID 10]

RQQSQEENVIVK
PEPTIDE:

[SEQ ID 11]

QQSQEENVIVK
PEPTIDE:

[SEQ ID 12]

LSRGQIEELSK
PEPTIDE:

[SEQ ID 13]

GQIEELSK
PEPTIDE:

[SEQ ID 14]

VLLEEHEK
PEPTIDE:

[SEQ ID 15]

SKPHTIFLPQHTDADYILVVLSGK
PEPTIDE:

[SEQ ID 16]

PHTIFLPQHTDADYILVVLSGK
PEPTIDE:

[SEQ ID 17]

SNKFQTLFENENGHIR
PEPTIDE:

[SEQ ID 18]

SKIFENLQNYR
PEPTIDE:

[SEQ ID 19]

IFENLQNYR
PEPTIDE:

[SEQ ID 20]

ILENQKQSHFADAQPQQR
PEPTIDE:

[SEQ ID 21]

PGQLQSFLLSGNQNQQNYLSGFSK
PEPTIDE:

[SEQ ID 22]

FFELTPEKNQQLQDLDLFVNSVDLK
PEPTIDE:

[SEQ ID 23]

QLEELSK
PEPTIDE:

[SEQ ID 24]

QEEDEDEDEER
PEPTIDE:

[SEQ ID 25]

YQHQQGGKQEQENEGNNIFSGFK
PEPTIDE:

[SEQ ID 26]

GDTIKLPAGTTSYLVNQDDEEDLR
PEPTIDE:

[SEQ ID 27]

RQQGEETDAIVK
PEPTIDE:

[SEQ ID 28]

VLLEEQEKDRK
PEPTIDE:

[SEQ ID 29]

NILEASYNTR
PEPTIDE:

[SEQ ID 30]

FEAFDLAK
PEPTIDE:

[SEQ ID 31]

EQIEELKK
PEPTIDE:

[SEQ ID 32]

EQIEELK
PEPTIDE:

[SEQ ID 33]

NKNQYLR
PEPTIDE:

[SEQ ID 34]

LSPGDVVIIPAGHPVAITASSNLNLLGFGINAENNER
PEPTIDE:

[SEQ ID 35]

PSYEKQEDEEEKQK
PEPTIDE:

[SEQ ID 36]

EEDEEEGQR
PEPTIDE:

[SEQ ID 37]

TLFLPQYTDADFILVVLSGK
PEPTIDE:

[SEQ ID 38]

LVDLVIPVNGPGKFEAFDLAK
PEPTIDE:

[SEQ ID 39]

KNPQLQDLDIFVNYVEIK
PEPTIDE:

[SEQ ID 40]

GYVGLTFPGCPATHQQQFQLFEQR
PEPTIDE:

[SEQ ID 41]

RGPQQYAEWQINEK
PEPTIDE:

[SEQ ID 42]

DEHQKIHQFR
PEPTIDE:

[SEQ ID 43]

FRDEHQK
PEPTIDE:

[SEQ ID 44]

FPILNLIQMSATR
PEPTIDE:

[SEQ ID 45]

TNANAFVSHLAGK
PEPTIDE:

[SEQ ID 46]

ALPVDVVANAYR
PEPTIDE:

[SEQ ID 47]

YVYDVNNNANQLEPRQKEFL
PEPTIDE:

[SEQ ID 48]

VYVYDVNNNANQLEPRQKEFL
PEPTIDE:

[SEQ ID 49]

ADSYNPR
PEPTIDE:

[SEQ ID 50]

KPTLTQQQEQAQAQDQ
PEPTIDE:

[SEQ ID 51]

QAQAQDQYQQVQY
PEPTIDE:

[SEQ ID 52]

QAQDQYQQVQY
PEPTIDE:

[SEQ ID 53]

LQAFEPLR
PEPTIDE:

[SEQ ID 54]

SRVQVVSNFGK
PEPTIDE:

[SEQ ID 55]

WNVNAHSLVY
PEPTIDE:

[SEQ ID 56]

NVNAHSLVY
PEPTIDE:

[SEQ ID 57]

IQGRSRVQVVSNFGK
PEPTIDE:

[SEQ ID 58]

GKTVFDGVLRPGQL
PEPTIDE:

[SEQ ID 59]

FGKTVFDGVLRPGQL
PEPTIDE:

[SEQ ID 60]

FQQQYYPGLSNESESETSE
PEPTIDE:

[SEQ ID 61]

QQYYPGLSN
PEPTIDE:

[SEQ ID 62]

QQQYYPGLSN
PEPTIDE:

[SEQ ID 63]

VTNLNTQNFPILSLVQMSAVK
PEPTIDE:

[SEQ ID 64]

ITQGRARVQVVNNNGKTVF
PEPTIDE:

[SEQ ID 65]

ITQGRARVQVVNNNGKTVFNGE
PEPTIDE:

[SEQ ID 66]

ITQGRARVQVVNNNGKTVFNG
PEPTIDE:

[SEQ ID 67]

RVQVVNNNGKTVF
PEPTIDE:

[SEQ ID 68]

RALPNDVLANAYRISREE
PEPTIDE:

[SEQ ID 69]

SIFRALPNDVLANAYR
PEPTIDE:

[SEQ ID 70]

SIFRALPNDVLANAY
PEPTIDE:

[SEQ ID 71]

SIFRALPNDVLAN
PEPTIDE:

[SEQ ID 72]

SSIFRALPNDVLANAYR
PEPTIDE:

[SEQ ID 73]

SIFRALPNDVLANAYRISREE
PEPTIDE:

[SEQ ID 74]

SIFRALPND
PEPTIDE:

[SEQ ID 75]

IYVTDLNNGANQLDPRQRD
PEPTIDE:

[SEQ ID 76]

VTNLNSQNFPILNLVQMSAVK
PEPTIDE:

[SEQ ID 77]

QNIDNPNR
PEPTIDE:

[SEQ ID 78]

ADTYNPR
PEPTIDE:

[SEQ ID 79]

NIDNPNRADTYNPRAGRVTNL
PEPTIDE:

[SEQ ID 80]

RVRQNIDNPNRADTYNPRAGRVTNL
PEPTIDE:

[SEQ ID 81]

TNPNSMVSHIAGKSSIFR
PEPTIDE:

[SEQ ID 82]

HNRGDEFGAFTPLQYK
PEPTIDE:

[SEQ ID 83]

SYQDVYNVAESS
PEPTIDE:

[SEQ ID 84]

ISREEAQR
PEPTIDE:

[SEQ ID 85]

SIFRALPTDVLANAYRISREE
PEPTIDE:

[SEQ ID 86]

YRISREEAQRLKHNRGDEF
PEPTIDE:

[SEQ ID 87]

YRISREEAQRLKHNRGDE
PEPTIDE:

[SEQ ID 88]

FKDEHQKIHR
PEPTIDE:

[SEQ ID 89]

QGDVIALPAGVAHW
PEPTIDE:

[SEQ ID 90]

TVFNGELRR
PEPTIDE:

[SEQ ID 91]

TVFNGELR
PEPTIDE:

[SEQ ID 92]

QVQVVNNNGKTVF
PEPTIDE:

[SEQ ID 93]

YIIQGRGITGPTF
PEPTIDE:

[SEQ ID 94]

VYIIQGRGITGPTF
PEPTIDE:

[SEQ ID 95]

LQAFEPIRSVR
PEPTIDE:

[SEQ ID 96]

GLSLLQPYASLQEQEQGQMQSR
PEPTIDE:

[SEQ ID 97]

GEIVRVER
PEPTIDE:

[SEQ ID 98]

RGLSLLQPYASLQ
PEPTIDE:

[SEQ ID 99]

RGLSLLQPYASLQEQ
PEPTIDE:

[SEQ ID 100]

RGLSLLQPYASLQEQE
PEPTIDE:

[SEQ ID 101]

RGLSLLQPYASLQE
PEPTIDE:

[SEQ ID 102]

RNPQAYR
PEPTIDE:

[SEQ ID 103]

FLLAGNKRNPQAY
PEPTIDE:

[SEQ ID 104]

EVEEWSQNIF
PEPTIDE:

[SEQ ID 105]

LAGNKRNPQAYR
PEPTIDE:

[SEQ ID 106]

FLLAGNKRNPQA
PEPTIDE:

[SEQ ID 107]

ELGAPDVGHPMSE
PEPTIDE:

[SEQ ID 108]

IVQGHARVQVVSNLGK
PEPTIDE:

[SEQ ID 109]

IVQGHARVQVVSNL
PEPTIDE:

[SEQ ID 110]

IVQGHARVQVVSN
PEPTIDE:

[SEQ ID 111]

NNRGEELGAFTPR
PEPTIDE:

[SEQ ID 112]

GEELGAFTPR
PEPTIDE:

[SEQ ID 113]

FPILNLVQLSATR
PEPTIDE:

[SEQ ID 114]

SIEQHSGQNIFSGFNNELLSEALGVNALVAK
PEPTIDE:

[SEQ ID 115]

LQGQNDQR
PEPTIDE:

[SEQ ID 116]

SGFNNELLSEALGVNALVAK
PEPTIDE:

[SEQ ID 117]

PAFAQQQEQAQQQEQAQAQY
PEPTIDE:

[SEQ ID 118]

VAKRLQGQNDQRGEI
PEPTIDE:

[SEQ ID 119]

ALVAKRLQGQNDQRGEI
PEPTIDE:

[SEQ ID 120]

LQGQNDQRGEIIR
PEPTIDE:

[SEQ ID 121]

PNVNPWHNPRQGGF
PEPTIDE:

[SEQ ID 122]

FYNEGDAPVVALY
PEPTIDE:

[SEQ ID 123]

FYNEGDAPVV
PEPTIDE:

[SEQ ID 124]

FYNEGDAPVVAL
PEPTIDE:

[SEQ ID 125]

FYNEGDAPVVA
PEPTIDE:

[SEQ ID 126]

TNANSMVSHLAGK
PEPTIDE:

[SEQ ID 127]

AMPVDVIANAYR
PEPTIDE:

[SEQ ID 128]

NWENVLLGLGVAGSAPGIEGDEIAPLAK
PEPTIDE:

[SEQ ID 129]

NVLLGLGVAGSAPGIEGDE
PEPTIDE:

[SEQ ID 130]

NWENVLLGLGVAGSAPGIEGDEIAPLAK
PEPTIDE:

[SEQ ID 131]

FNAPLAHLIMAGADVLAVPSR
PEPTIDE:

[SEQ ID 132]

FNAPLAHLIM
PEPTIDE:

[SEQ ID 133]

FNAPLAHLIMAGADVLAVPSR
PEPTIDE:

[SEQ ID 134]

VVGTPAYEEMVR
PEPTIDE:

[SEQ ID 135]

TGGLGDVLGGLPPAMAANGHR
PEPTIDE:

[SEQ ID 136]

YDQYKDAWDTSVVAEIK
PEPTIDE:

[SEQ ID 137]

DAWDTSVVAEIK
PEPTIDE:

[SEQ ID 138]

VMVISPR
PEPTIDE:

[SEQ ID 139]

LTGITGIVNGMDVSEWDPSKDK
PEPTIDE:

[SEQ ID 140]

VLTVSPYYAEELISGIAR
PEPTIDE:

[SEQ ID 141]

EALQAEAGLPVDRK
PEPTIDE:

[SEQ ID 142]

YDATTAIEAK
PEPTIDE:

[SEQ ID 143]

IPLIAFIGR
PEPTIDE:

[SEQ ID 144]

AGILEADR
PEPTIDE:

[SEQ ID 145]

IPLIAFIGR
PEPTIDE:

[SEQ ID 146]

VFIDHPSFLEK
PEPTIDE:

[SEQ ID 147]

GPDTGVDYKDNQM
PEPTIDE:

[SEQ ID 148]

IYGPDTGVDYKDNQMR
PEPTIDE:

[SEQ ID 149]

IYGPDTGVDYK
PEPTIDE:

[SEQ ID 150]

ILNLNNNPYFK
PEPTIDE:

[SEQ ID 151]

APTGTFIASGVVVGKD
PEPTIDE:

[SEQ ID 152]

QNYLSGFSKNILE
PEPTIDE:

[SEQ ID 153]

TIKLPAGTIAYLVNRDDNEE
PEPTIDE:

[SEQ ID 154]

LAIPVNRPGQLQSFL
PEPTIDE:

[SEQ ID 155]

AIPVNRPGQLQ
PEPTIDE:

[SEQ ID 156]

PAGHPVAVK
PEPTIDE:

[SEQ ID 157]

VQNYKAKLSSGDVFVIPAG
PEPTIDE:

[SEQ ID 158]

NNQRNFLAGDEDNVISQIQRPVKE
PEPTIDE:

[SEQ ID 159]

INKQVQNYKAKLSSGDVFVIPAG
PEPTIDE:

[SEQ ID 160]

LAIPVNRPGQ
PEPTIDE:

[SEQ ID 161]

NFLAGDEDNVISQIQRPVKE
PEPTIDE:

[SEQ ID 162]

DLAIPVNRPGQLQSF
PEPTIDE:

[SEQ ID 163]

VIPAGHPVAVK
PEPTIDE:

[SEQ ID 164]

DTIKLPAGTIAYLVNRDDNEE
PEPTIDE:

[SEQ ID 165]

LAIPVNRPGQLQSF
PEPTIDE:

[SEQ ID 166]

KQVQNYKAKLSSGDVFVIPAG
PEPTIDE:

[SEQ ID 167]

RGDTIKLPAGTIAYLVNRDDNEE
PEPTIDE:

[SEQ ID 168]

FLAGDEDNVISQIQRPVKE
PEPTIDE:

[SEQ ID 169]

LAIPVNRPGQLQS
PEPTIDE:

[SEQ ID 170]

VLDLAIPVNRPGQLQ
PEPTIDE:

[SEQ ID 171]

DLAIPVNRPGQLQ
PEPTIDE:

[SEQ ID 172]

VFVIPAGHPVAVK
PEPTIDE:

[SEQ ID 173]

TIFLPQHTDADYILVVLSGK
PEPTIDE:

[SEQ ID 174]

NQRNFLAGDEDNVISQIQRPVKE
PEPTIDE:

[SEQ ID 175]

LAIPVNRPGQLQ
PEPTIDE:

[SEQ ID 176]

HPVAVKASSNLDLLGFG
PEPTIDE:

[SEQ ID 177]

LAIPVNRPGQL
PEPTIDE:

[SEQ ID 178]

DLAIPVNRPGQL
PEPTIDE:

[SEQ ID 179]

SKPHTIFLPQHTDADYILVVLSGK
PEPTIDE:

[SEQ ID 180]

FVIPAGHPVAVK
PEPTIDE:

[SEQ ID 181]

DLAIPVNRPGQLQS
PEPTIDE:

[SEQ ID 182]

SGDVFVIPAGHPVAVKASSNLD
PEPTIDE:

[SEQ ID 183]

AIPVNRPGQLQSF
PEPTIDE:

[SEQ ID 184]

ELAFPGSAQEVDR
PEPTIDE:

[SEQ ID 185]

LAIPVNRPGQLQSFLLSG
PEPTIDE:

[SEQ ID 186]

VFVIPAGHPVAVKASSNLDLLGFG
PEPTIDE:

[SEQ ID 187]

AGHPVAVK
PEPTIDE:

[SEQ ID 188]

HPVAVKASSNLDLLGFGINAE
PEPTIDE:

[SEQ ID 189]

LAIPVNRPGQLQSFLLSGNQNQ
PEPTIDE:

[SEQ ID 190]

SGDVFVIPAG
PEPTIDE:

[SEQ ID 191]

GSLLLPHYNSRAIVIVTVNE
PEPTIDE:

[SEQ ID 192]

NFLAGDEDNVISQIQRPVK
PEPTIDE:

[SEQ ID 193]

SGDVFVIPAGHPVA
PEPTIDE:

[SEQ ID 194]

GSLLLPHYNSRAIVIV
PEPTIDE:

[SEQ ID 195]

RGDTIKLPAGTIAYLVNRDD
PEPTIDE:

[SEQ ID 196]

SGDVFVIPAGHPVAVK
PEPTIDE:

[SEQ ID 197]

LSSGDVFVIPAGHPVAVK
PEPTIDE:

[SEQ ID 198]

LDLAIPVNRPGQL
PEPTIDE:

[SEQ ID 199]

AIPVNRPGQL
PEPTIDE:

[SEQ ID 200]

LAIPVNRPGQLQSFLL
PEPTIDE:

[SEQ ID 201]

PHTIFLPQHTDADYILVVLSGK
PEPTIDE:

[SEQ ID 202]

VFVIPAGHPVAVKASSNLD
PEPTIDE:

[SEQ ID 203]

LAIPVNRPGQLQSFLLS
PEPTIDE:

[SEQ ID 204]

VLDLAIPVNRPGQLQSF
PEPTIDE:

[SEQ ID 205]

AIPVNRPGQLQS
PEPTIDE:

[SEQ ID 206]

DTIKLPAGTIAYLVNRDDNE
PEPTIDE:

[SEQ ID 207]

NYKAKLSSGDVFVIPAG
PEPTIDE:

[SEQ ID 208]

GKAILTVLKPDDRNSFNLE
PEPTIDE:

[SEQ ID 209]

YKSKPHTIFLPQHTDAD
PEPTIDE:

[SEQ ID 210]

ASSNLDLLGFG
PEPTIDE:

[SEQ ID 211]

DEEEEQGEEEINK
PEPTIDE:

[SEQ ID 212]

YKSKPHTIFLPQHTD
PEPTIDE:

[SEQ ID 213]

VLDLAIPVNR
PEPTIDE:

[SEQ ID 214]

FFEITPEKNPQLQDLDIFVNSVEIK
PEPTIDE:

[SEQ ID 215]

TIFLPQHTDADYIL
PEPTIDE:

[SEQ ID 216]

SFLLSGNQNQQNYLSG
PEPTIDE:

[SEQ ID 217]

SFLLSGNQNQQNYLSGFS
PEPTIDE:

[SEQ ID 218]

NQQEQRKEDDEEEEQGEEE
PEPTIDE:

[SEQ ID 219]

EEQGEEEINK
PEPTIDE:

[SEQ ID 220]

SRGPIYSNE
PEPTIDE:

[SEQ ID 221]

EDDEEEEQGEEEINK
PEPTIDE:

[SEQ ID 222]

DDEEEEQGEEEINK
PEPTIDE:

[SEQ ID 223]

KEDDEEEEQGEEEIN
PEPTIDE:

[SEQ ID 224]

KEDDEEEEQGEE
PEPTIDE:

[SEQ ID 225]

QRKEDDEEEEQGEEE
PEPTIDE:

[SEQ ID 226]

KEDDEEEEQGEEEINK
PEPTIDE:

[SEQ ID 227]

KEDDEEEEQGEEE
PEPTIDE:

[SEQ ID 228]

HPVAITASSNLNLLG
PEPTIDE:

[SEQ ID 229]

ASSNLNLLGFG
PEPTIDE:

[SEQ ID 230]

ITASSNLNLLGFG
PEPTIDE:

[SEQ ID 231]

ITASSNLNLLGFGINAE
PEPTIDE:

[SEQ ID 232]

SSNLNLLGFG
PEPTIDE:

[SEQ ID 233]

VDLVIPVNGPGKF
PEPTIDE:

[SEQ ID 234]

LVIPVNGPGKFE
PEPTIDE:

[SEQ ID 235]

LVIPVNGPGKFEA
PEPTIDE:

[SEQ ID 236]

LRLVDLVIPVNGPGKFE
PEPTIDE:

[SEQ ID 237]

YRAKPHTIFLPQHIDAD
PEPTIDE:

[SEQ ID 238]

HPVAITASSNLNLLGFGINAE
PEPTIDE:

[SEQ ID 239]

SNLNLLGFG
PEPTIDE:

[SEQ ID 240]

HPVAITASSNLNLLGFGINAENNE
PEPTIDE:

[SEQ ID 241]

LVDLVIPVNGPGKFE
PEPTIDE:

[SEQ ID 242]

LVIPVNGPGKF
PEPTIDE:

[SEQ ID 243]

TIKLPAGTTSYLVNQDDE
PEPTIDE:

[SEQ ID 244]

DLRLVDLVIPVNGPGKFE
PEPTIDE:

[SEQ ID 245]

EDLRLVDLVIPVNGPGKFE
PEPTIDE:

[SEQ ID 246]

HPVAITASSNLNLLGFG
PEPTIDE:

[SEQ ID 247]

LVDLVIPVNGPGKFEAFDLAK
PEPTIDE:

[SEQ ID 248]

DNVISQIENPVKE
PEPTIDE:

[SEQ ID 249]

VVIIPAGHPVAITASSNLNLLGFG
PEPTIDE:

[SEQ ID 250]

LVDLVIPVNGPGKFEAF
PEPTIDE:

[SEQ ID 251]

YPQLQDLDL
PEPTIDE:

[SEQ ID 252]

VIPVNGPGKF
PEPTIDE:

[SEQ ID 253]

SKKSLPSE
PEPTIDE:

[SEQ ID 254]

LPQHIDADLILVVLSGK
PEPTIDE:

[SEQ ID 255]

RGDTIKLPAGTTSYLVNQD
PEPTIDE:

[SEQ ID 256]

IPVNGPGKF
PEPTIDE:

[SEQ ID 257]

LPQHIDADL
PEPTIDE:

[SEQ ID 258]

LVIPVNGPGK
PEPTIDE:

[SEQ ID 259]

IFLPQHIDAD
PEPTIDE:

[SEQ ID 260]

LPQHIDAD
PEPTIDE:

[SEQ ID 261]

VIPVNGPGK
PEPTIDE:

[SEQ ID 262]

IFLPQHIDA
PEPTIDE:

[SEQ ID 263]

TIKLPAGTTSYLVNQDDEE
PEPTIDE:

[SEQ ID 264]

HGEWRPSYEKEEDEEEGQRER
PEPTIDE:

[SEQ ID 265]

EKRHGEWRPSYEKEEDEEEGQRE
PEPTIDE:

[SEQ ID 266]

LPAGTTSYLVNQDDEEDLR
PEPTIDE:

[SEQ ID 267]

PSYEKEEDEEEGQRER
PEPTIDE:

[SEQ ID 268]

EKRHGEWRPSYE
PEPTIDE:

[SEQ ID 269]

TIKLPAGTTSYLVNQDDEED
PEPTIDE:

[SEQ ID 270]

HGEWRPSYEKQEDEEEK
PEPTIDE:

[SEQ ID 271]

EWRPSYEKEEDEEE
PEPTIDE:

[SEQ ID 272]

PSYEKEEDEEEGQR
PEPTIDE:

[SEQ ID 273]

EKEEDEEEGQR
PEPTIDE:

[SEQ ID 274]

EWRPSYEKEEDEEEGQRE
PEPTIDE:

[SEQ ID 275]

KEEDEEEGQR
PEPTIDE:

[SEQ ID 276]

VQPGRERWEREEDEEQVDE
PEPTIDE:

[SEQ ID 277]

DVVIIPAGHPVA
PEPTIDE:

[SEQ ID 278]

HGEWRPSYEKQEDE
PEPTIDE:

[SEQ ID 279]

EEDEEEGQR
PEPTIDE:

[SEQ ID 280]

HGEWRPSYEKEEDEEEGQR
PEPTIDE:

[SEQ ID 281]

EEWRGSQRREDPEE
PEPTIDE:

[SEQ ID 282]

REEDEEQVDEEWRGSQRREDPEE
PEPTIDE:

[SEQ ID 283]

RHGEWRPSY
PEPTIDE:

[SEQ ID 284]

HGEWRPSYEKQEDEE
PEPTIDE:

[SEQ ID 285]

VVIIPAGHPVA
PEPTIDE:

[SEQ ID 286]

HGEWRPSYE
PEPTIDE:

[SEQ ID 287]

KEEDEEEGQRER
PEPTIDE:

[SEQ ID 288]

VVIIPAGHPVAIT
PEPTIDE:

[SEQ ID 289]

EKRHGEWRPSYEKEEDE
PEPTIDE:

[SEQ ID 290]

QVDEEWRGSQRREDPEE
PEPTIDE:

[SEQ ID 291]

GDTIKLPAGTTSYLVNQDDEEDLR
PEPTIDE:

[SEQ ID 292]

GSEPRVPAQRE
PEPTIDE:

[SEQ ID 293]

EEKRHGEWRPSYEKE
PEPTIDE:

[SEQ ID 294]

EWRPSYEKEEDEE
PEPTIDE:

[SEQ ID 295]

NYDEGSEPRVPAQRE
PEPTIDE:

[SEQ ID 296]

VIIPAGHPVAIT
PEPTIDE:

[SEQ ID 297]

RHGEWRPSYEK
PEPTIDE:

[SEQ ID 298]

NYDEGSEPR
PEPTIDE:

[SEQ ID 299]

WRPSYEKEEDEE
PEPTIDE:

[SEQ ID 300]

WRPSYEKQEDEEE
PEPTIDE:

[SEQ ID 301]

EKRHGEWRPSYEKQEDEEE
PEPTIDE:

[SEQ ID 302]

VVIIPAGHPVAITA
PEPTIDE:

[SEQ ID 303]

KRHGEWRPSYE
PEPTIDE:

[SEQ ID 304]

GSDDNVISQIENPVKE
PEPTIDE:

[SEQ ID 305]

VVIIPAGHPV
PEPTIDE:

[SEQ ID 306]

HGEWRPSY
PEPTIDE:

[SEQ ID 307]

RPSYEKEEDEEEGQR
PEPTIDE:

[SEQ ID 308]

HGEWRPSYEK
PEPTIDE:

[SEQ ID 309]

KRHGEWRPSYEKEE
PEPTIDE:

[SEQ ID 310]

VVIIPAGHPVAITAS
PEPTIDE:

[SEQ ID 311]

RGDTIKLPAGTTSYLVNQDDEED
PEPTIDE:

[SEQ ID 312]

KRHGEWRPSYEKQEDEEE
PEPTIDE:

[SEQ ID 313]

DEEQVDEEWRGSQRREDPEE
PEPTIDE:

[SEQ ID 314]

RHGEWRPSYE
PEPTIDE:

[SEQ ID 315]

HGEWRPSYEKE
PEPTIDE:

[SEQ ID 316]

KRHGEWRPSYEKEEDEEE
PEPTIDE:

[SEQ ID 317]

EKRHGEWRPSYEKEEDEEE
PEPTIDE:

[SEQ ID 318]

TIKLPAGTTSYLVNQDDEEDLRLVD
PEPTIDE:

[SEQ ID 319]

WRPSYEKEEDEEEGQRE
PEPTIDE:

[SEQ ID 320]

KRHGEWRPSYEKEEDEE
PEPTIDE:

[SEQ ID 321]

VVIIPAGHPVAI
PEPTIDE:

[SEQ ID 322]

EWRGSQRREDPEE
PEPTIDE:

[SEQ ID 323]

HGEWRPSYEKQEDEEEKQK
PEPTIDE:

[SEQ ID 324]

SGSDDNVISQIENPVKE
PEPTIDE:

[SEQ ID 325]

RPSYEKEEDEEEGQRER
PEPTIDE:

[SEQ ID 326]

EKEEDEEEGQRER
PEPTIDE:

[SEQ ID 327]

HGEWRPSYEKQ
PEPTIDE:

[SEQ ID 328]

WRPSYEKEEDEEE
PEPTIDE:

[SEQ ID 329]

LAKNKNQYLRGFS
PEPTIDE:

[SEQ ID 330]

NKNQYLRGFS
PEPTIDE:

[SEQ ID 331]

LRGFSKNILE
PEPTIDE:

[SEQ ID 332]

LAKNKNQYLRGFSKN
PEPTIDE:

[SEQ ID 333]

TVLSPNDRNSY
PEPTIDE:

[SEQ ID 334]

QYLRGFSKNILE
PEPTIDE:

[SEQ ID 335]

GKAILTVLSPNDRNSYNLE
PEPTIDE:

[SEQ ID 336]

RGFSKNILE
PEPTIDE:

[SEQ ID 337]

NKNQYLRGFSKNILE
PEPTIDE:

[SEQ ID 338]

ASSNLNLLGFGINAE
PEPTIDE:

[SEQ ID 339]

ASSNLNLLGF
PEPTIDE:

[SEQ ID 340]

LAKNKNQYLRGFSK
PEPTIDE:

[SEQ ID 341]

RGDTIKLPAGTTSYLVNQDDEE
PEPTIDE:

[SEQ ID 342]

ARLSPGDVVIIPAGHPVAITASSN
PEPTIDE:

[SEQ ID 343]

VQRYEARLSPGD
PEPTIDE:

[SEQ ID 344]

ARLSPGDVVIIPAGHPVAIT
PEPTIDE:

[SEQ ID 345]

RGDTIKLPAGTTSYLVNQDDE
PEPTIDE:

[SEQ ID 346]

ARLSPGDVVIIPAGHPVA
PEPTIDE:

[SEQ ID 347]

GALMLPHYNSRAIVVLLVNE
PEPTIDE:

[SEQ ID 348]

ARLSPGDVVIIPAGHPVAITASS
PEPTIDE:

[SEQ ID 349]

LSPGDVVIIPAGHPVAITASSNLNLLGFGINAENNER
PEPTIDE:

[SEQ ID 350]

ARLSPGDVVIIPAGHPVAITAS
PEPTIDE:

[SEQ ID 351]

LSPGDVVIIPAGHPVAITASSNL
PEPTIDE:

[SEQ ID 352]

ARLSPGDVVIIPAGHPVAITA
PEPTIDE:

[SEQ ID 353]

HGPVEMPYTLLYPSSK
PEPTIDE:

[SEQ ID 354]

LDALEPDNR
PEPTIDE:

[SEQ ID 355]

DALEPDNR
PEPTIDE:

[SEQ ID 356]

HGSLHKNAMFVPHYNLNANSIIYA
PEPTIDE:

[SEQ ID 357]

LAGTSSVINNLPLDVVAATF
PEPTIDE:

[SEQ ID 358]

FREGDIIAVPTGIVFW
PEPTIDE:

[SEQ ID 359]

GTSSVINNLPLDVVAATFNLQRNE
PEPTIDE:

[SEQ ID 360]

KGAIVKVKGGLSIISPPE
PEPTIDE:

[SEQ ID 361]

RLAGTSSVINNLPLD
PEPTIDE:

[SEQ ID 362]

AGTSSVINNLPLDVVAATFNLQRNE
PEPTIDE:

[SEQ ID 363]

AGTSSVINNLPL
PEPTIDE:

[SEQ ID 364]

LAGTSSVINNLPLDVVA
PEPTIDE:

[SEQ ID 365]

AGTSSVINNLPLDV
PEPTIDE:

[SEQ ID 366]

AGRIKTVTSLDLPVLRW
PEPTIDE:

[SEQ ID 367]

AGRIKTVTSLDLPVLR
PEPTIDE:

[SEQ ID 368]

FREGDIIAVPTGIVF
PEPTIDE:

[SEQ ID 369]

AGTSSVINNLPLD
PEPTIDE:

[SEQ ID 370]

LAGTSSVINNLPL
PEPTIDE:

[SEQ ID 371]

LAGTSSVINNLPLDVV
PEPTIDE:

[SEQ ID 372]

EGDIIAVPTGIVF
PEPTIDE:

[SEQ ID 373]

LAGTSSVINNLPLDV
PEPTIDE:

[SEQ ID 374]

AGRALTVPQNYAVAAKSLSD
PEPTIDE:

[SEQ ID 375]

AGRALTVPQNYA
PEPTIDE:

[SEQ ID 376]

LAGTSSVINNLPLD
PEPTIDE:

[SEQ ID 377]

RAGIARLAGTSSVINNLPLDVVA
PEPTIDE:

[SEQ ID 378]

RASSNLNLLGFGINAE
PEPTIDE:

[SEQ ID 379]

VTVNEGKGDFEL
PEPTIDE:

[SEQ ID 380]

VRASSNLNLLGFGINAE
PEPTIDE:

[SEQ ID 381]

VRASSNLNLLGFG
PEPTIDE:

[SEQ ID 382]

HPVAVRASSNLNLLGFG
PEPTIDE:

[SEQ ID 383]

TKNQVQSYKAKLTPGD
PEPTIDE:

[SEQ ID 384]

HPVAVRASSNLNLLG
PEPTIDE:

[SEQ ID 385]

KAKLTPGDVFVIPAG
PEPTIDE:

[SEQ ID 386]

DLTFPGSAQEVDRLLENQK
PEPTIDE:

[SEQ ID 387]

PAGHPVAVR
PEPTIDE:

[SEQ ID 388]

AKLTPGDVFVIPAGHPVA
PEPTIDE:

[SEQ ID 389]

SYKAKLTPGDVFVIPAGHPVA
PEPTIDE:

[SEQ ID 390]

LTPGDVFVIPAGHPVAVR
PEPTIDE:

[SEQ ID 391]

VQSYKAKLTPGDVFVIPAG
PEPTIDE:

[SEQ ID 392]

YKAKLTPGDVFVIPAGHPVA
PEPTIDE:

[SEQ ID 393]

FVIPAGHPVAVR
PEPTIDE:

[SEQ ID 394]

YKAKLTPGDVFVIPAG
PEPTIDE:

[SEQ ID 395]

DLTFPGSAQEVDR
PEPTIDE:

[SEQ ID 396]

AKLTPGDVFVIPAGHPVAVR
PEPTIDE:

[SEQ ID 397]

LTPGDVFVIPAG
PEPTIDE:

[SEQ ID 398]

SYKAKLTPGDVFVIPAG
PEPTIDE:

[SEQ ID 399]

SYKAKLTPGDVFVIPAGHPVAVR
PEPTIDE:

[SEQ ID 400]

VIPAGHPVAVR
PEPTIDE:

[SEQ ID 401]

QVQSYKAKLTPGDVFVIPAG
PEPTIDE:

[SEQ ID 402]

AKLTPGDVFVIPAG
PEPTIDE:

[SEQ ID 403]

HPVAVRASSNLNLLGFGINAE
PEPTIDE:

[SEQ ID 404]

YKAKLTPGDVFVIPAGHPVAVR
PEPTIDE:

[SEQ ID 405]

TKNQVQSYKAKLTPGDVFVIPAG
PEPTIDE:

[SEQ ID 406]

PFNLKSSDPIYS
PEPTIDE:

[SEQ ID 407]

IEKILLEE
PEPTIDE:

[SEQ ID 408]

SRSEPFNLKSSDPIYS
PEPTIDE:

[SEQ ID 409]

HPVAVRASSNLNL
PEPTIDE:

[SEQ ID 410]

TLFLPQYTDADFILVVLSGK
PEPTIDE:

[SEQ ID 411]

NWENVLLGLGVAGSAPGIEGDEIAPLAK
PEPTIDE:

[SEQ ID 412]

YDQYKDAWDTSVVAEIK
PEPTIDE:

[SEQ ID 413]

SSFDFIDGYDTPVEGR
PEPTIDE:

[SEQ ID 414]

GPDTGVDYKDNQM
PEPTIDE:

[SEQ ID 415]

ILNLNNNPYFK
PEPTIDE:

[SEQ ID 416]

VVGTPAYEE
PEPTIDE:

[SEQ ID 417]

IDGYDTPVEGR
PEPTIDE:

[SEQ ID 418]

VVGTPAYE
PEPTIDE:

[SEQ ID 419]

IYGPDTGVDYK
PEPTIDE:

[SEQ ID 420]

VAGSAPGIEGDE
PEPTIDE:

[SEQ ID 421]

IYGPDTGVDYKDNQMR
PEPTIDE:

[SEQ ID 422]

VVGTPAYEEMVR
PEPTIDE:

[SEQ ID 423]

DFIDGYDTPVEGR
PEPTIDE:

[SEQ ID 424]

LGLGVAGSAPGIEGDEIAPLAK
PEPTIDE:

[SEQ ID 425]

FNAPLAHLIMAGADVLAVPSR
PEPTIDE:

[SEQ ID 426]

LGLGVAGSAPGIEGDE
PEPTIDE:

[SEQ ID 427]

LGLGVAGSAPGIEGDEIAPL
PEPTIDE:

[SEQ ID 428]

VLTVSPYYAEELISGIAR
PEPTIDE:

[SEQ ID 429]

EALQAEAGLPVDR
PEPTIDE:

[SEQ ID 430]

LGLGVAGSAPGIEGD
PEPTIDE:

[SEQ ID 431]

IMAGADVLAVPSR
PEPTIDE:

[SEQ ID 432]

GLGVAGSAPGIEGDE
PEPTIDE:

[SEQ ID 433]

EALQAEAGLPVDRK
PEPTIDE:

[SEQ ID 434]

TGGLGDVLGGLPPAMAANGHR
PEPTIDE:

[SEQ ID 435]

LEEQKGPDVMA
PEPTIDE:

[SEQ ID 436]

LGVAGSAPGIEGDEIAPLAK
PEPTIDE:

[SEQ ID 437]

GLGVAGSAPGIEGDEIAPLAK
PEPTIDE:

[SEQ ID 438]

TGGLGDVLGGLPPAM
PEPTIDE:

[SEQ ID 439]

NVLLGLGVAGSAPGIEGDE
PEPTIDE:

[SEQ ID 440]

TVFDGVLRPGQL
PEPTIDE:

[SEQ ID 441]

RLQSQNDQRGEIIHVK
PEPTIDE:

[SEQ ID 442]

EGYYGEQQQQPGMTR
PEPTIDE:

[SEQ ID 443]

GYYGEQQQQPGMTR
PEPTIDE:

[SEQ ID 444]

EEGYYGEQQQQPGMTR
PEPTIDE:

[SEQ ID 445]

YYGGEGSSSEQGYYGEGSSE
PEPTIDE:

[SEQ ID 446]

YGGEGSSSEQGYYGEGSSE
PEPTIDE:

[SEQ ID 447]

SSEEGYYGEQQQQPGMTR
PEPTIDE:

[SEQ ID 448]

SEEGYYGEQQQQPGMTR
PEPTIDE:

[SEQ ID 449]

QGYYGEGSSEE
PEPTIDE:

[SEQ ID 450]

YGGEGSSSEQGYYGEGSSEEGY
PEPTIDE:

[SEQ ID 451]

YGEQQQQPGMTR
PEPTIDE:

[SEQ ID 452]

YYGEQQQQPGMTR
PEPTIDE:

[SEQ ID 453]

SYEESMPMPLEQGWSSSSSE
PEPTIDE:

[SEQ ID 454]

YYGEGSSEEGYYGEQQQQPGMTR
PEPTIDE:

[SEQ ID 455]

QQQQPGMTRV
PEPTIDE:

[SEQ ID 456]

GEQQQQPGMTR
PEPTIDE:

[SEQ ID 457]

SYEESMPMPLEQGWSSSSSEY
PEPTIDE:

[SEQ ID 458]

YYGGEGSSSEQGYYGEGSSEEGY
PEPTIDE:

[SEQ ID 459]

YGEQQQQPGMTRVR
PEPTIDE:

[SEQ ID 460]

GEGSSEEGYYGEQQQQPGMTR
PEPTIDE:

[SEQ ID 461]

YGEGSSEEGYYGEQQQQPGMTR
PEPTIDE:

[SEQ ID 462]

QQQQPGMTRVR
PEPTIDE:

[SEQ ID 463]

QYAAQLPSMCRVEPQQCSIFAAGQY
PEPTIDE:

[SEQ ID 464]

TVFNGVLRPGQL
PEPTIDE:

[SEQ ID 465]

TVFNGVLRPGQLL
PEPTIDE:

[SEQ ID 466]

SGFNNELLSEALGVNALVAK
PEPTIDE:

[SEQ ID 467]

NGVLRPGQL
PEPTIDE:

[SEQ ID 468]

ALVAKRLQGQNDQRGEI
PEPTIDE:

[SEQ ID 469]

VPRYSNTPGM
PEPTIDE:

[SEQ ID 470]

PRYSNTPGMV
PEPTIDE:

[SEQ ID 471]

YSNTPGMVY
PEPTIDE:

[SEQ ID 472]

LVPRYSNTPGM
PEPTIDE:

[SEQ ID 473]

FYNEGDAPVV
PEPTIDE:

[SEQ ID 474]

FYNEGDAPVVAL
PEPTIDE:

[SEQ ID 475]

FEPLRRVRSEAGVTE
PEPTIDE:

[SEQ ID 476]

FYNEGDAPVVALY
PEPTIDE:

[SEQ ID 477]

FYNEGDAPVVA
PEPTIDE:

[SEQ ID 478]

EVEERSQNIF
PEPTIDE:

[SEQ ID 479]

VEERSQNIFSGF
PEPTIDE:

[SEQ ID 480]

ASLQEQEQGQVQ
PEPTIDE:

[SEQ ID 481]

QEQEQGQVQSR
PEPTIDE:

[SEQ ID 482]

ASLQEQEQGQVQSR
PEPTIDE:

[SEQ ID 483]

EVEERSQNIFSGF
PEPTIDE:

[SEQ ID 484]

VTDLNNGANQLDPRQRD
PEPTIDE:

[SEQ ID 485]

VEHGLSLLQPYASL
PEPTIDE:

[SEQ ID 486]

IYVTDLNNGANQLDPRQRDFL
PEPTIDE:

[SEQ ID 487]

VTDLNNGANQLDPRQRDFL
PEPTIDE:

[SEQ ID 488]

VEHGLSLLQPYASLQEQEQGQVQSR
PEPTIDE:

[SEQ ID 489]

VTDLNNGANQLDPR
PEPTIDE:

[SEQ ID 490]

IYVTDLNNGANQLDPRQRD
PEPTIDE:

[SEQ ID 491]

YVTDLNNGANQLDPRQRDFL
PEPTIDE:

[SEQ ID 492]

YVTDLNNGANQLDPR
PEPTIDE:

[SEQ ID 493]

STELLSEALGVSSQVAR
PEPTIDE:

[SEQ ID 494]

HGLSLLQPYASLQEQE
PEPTIDE:

[SEQ ID 495]

SGFSTELLSEALGVSSQVAR
PEPTIDE:

[SEQ ID 496]

GAFTPLQYKSYQD
PEPTIDE:

[SEQ ID 497]

GLLLPHYTNGASLVY
PEPTIDE:

[SEQ ID 498]

FLLAGNKRNPQAYRRE
PEPTIDE:

[SEQ ID 499]

ALPTDVLANAYR
PEPTIDE:

[SEQ ID 500]

DFLLAGNK
PEPTIDE:

[SEQ ID 501]

DVLANAYR
PEPTIDE:

[SEQ ID 502]

GAFTPLQYK
PEPTIDE:

[SEQ ID 503]

QGDVIALPAGVAHW
PEPTIDE:

[SEQ ID 504]

FGAFTPLQYKSY
PEPTIDE:

[SEQ ID 505]

FLLAGNKRNPQAYR
PEPTIDE:

[SEQ ID 506]

FGAFTPLQYKSYQ
PEPTIDE:

[SEQ ID 507]

GLSLLQPYASLQEQE
PEPTIDE:

[SEQ ID 508]

AFTPLQYK
PEPTIDE:

[SEQ ID 509]

FGAFTPLQYKSYQD
PEPTIDE:

[SEQ ID 510]

GDEFGAFTPLQYK
PEPTIDE:

[SEQ ID 511]

FGAFTPLQYKSYQDV
PEPTIDE:

[SEQ ID 512]

FGAFTPLQYK
PEPTIDE:

[SEQ ID 513]

FGAFTPLQYKS
PEPTIDE:

[SEQ ID 514]

VYIIQGRGITGPTF
PEPTIDE:

[SEQ ID 515]

YIIQGRGITGPTF
PEPTIDE:

[SEQ ID 516]

KTNPNSMVSHIAGK
PEPTIDE:

[SEQ ID 517]

TNPNSMVSHIAGK
PEPTIDE:

[SEQ ID 518]

PNSMVSHIAGKS
PEPTIDE:

[SEQ ID 519]

NIDNPNRADTYNPRAGRVTN
PEPTIDE:

[SEQ ID 520]

QRDFLLAGNKR
PEPTIDE:

[SEQ ID 521]

LLQPYASLQEQE
PEPTIDE:

[SEQ ID 522]

QRDFLLAGNK
PEPTIDE:

[SEQ ID 523]

QEQEQGQMQSR
PEPTIDE:

[SEQ ID 524]

SLLQPYASLQEQE
PEPTIDE:

[SEQ ID 525]

ASLQEQEQGQM
PEPTIDE:

[SEQ ID 526]

ASLQEQEQGQMQSR
PEPTIDE:

[SEQ ID 527]

DFLLAGNKR
PEPTIDE:

[SEQ ID 528]

QAFEPIRSVRSQAGTTEF
PEPTIDE:

[SEQ ID 529]

KTNPNSMVSHIAGKSSIF
PEPTIDE:

[SEQ ID 530]

VRRVIEPRGLLLPHYTNGASL
PEPTIDE:

[SEQ ID 531]

FGAFTPLQYKSYQDVYN
PEPTIDE:

[SEQ ID 532]

IALPAGVAHW
PEPTIDE:

[SEQ ID 533]

RVRQNIDNPNRADTYNPRAGRVTNL
PEPTIDE:

[SEQ ID 534]

NIDNPNRADTYNPRAGRVTNL
PEPTIDE:

[SEQ ID 535]

GAFTPLQYKSYQDVYN
PEPTIDE:

[SEQ ID 536]

PNSMVSHIAGKSSIFR
PEPTIDE:

[SEQ ID 537]

RLQAFEPIRSVRSQAGTTE
PEPTIDE:

[SEQ ID 538]

TNPNSMVSHIAGKSSIFR
PEPTIDE:

[SEQ ID 539]

ELGAPDVGHPM
PEPTIDE:

[SEQ ID 540]

LGAPDVGHPM
PEPTIDE:

[SEQ ID 541]

ELGAPDVGHPMSEVF
PEPTIDE:

[SEQ ID 542]

ELGAPDVGHPMS
PEPTIDE:

[SEQ ID 543]

ELGAPDVGHPMSEVFR
PEPTIDE:

[SEQ ID 544]

ELGAPDVGHPMSEV
PEPTIDE:

[SEQ ID 545]

ELGAPDVGHPMSE
PEPTIDE:

[SEQ ID 546]

LGAPDVGHPMSE
PEPTIDE:

[SEQ ID 547]

YRELGAPDVGHPMSE
PEPTIDE:

[SEQ ID 548]

LGAPDVGHPMSEV
PEPTIDE:

[SEQ ID 549]

RELGAPDVGHPMSE
PEPTIDE:

[SEQ ID 550]

APTGTFIASGVVVGKD
PEPTIDE:

[SEQ ID 551]

LAIVKFSPNEQNKHIGE
PEPTIDE:

[SEQ ID 552]

RHQITDTTNGHYAPVTYIQVE
PEPTIDE:

[SEQ ID 553]

GDLAIVKFSPNEQNKHIGE
PEPTIDE:

[SEQ ID 554]

NPDNPDNPNNPDNPNNPD
PEPTIDE:

[SEQ ID 555]

VLDLAIPVNRPGQL
PEPTIDE:

[SEQ ID 556]

VLDLAIPVNRPGQLQSF
PEPTIDE:

[SEQ ID 557]

SFLLSGNQNQQNYLS
PEPTIDE:

[SEQ ID 558]

VLDLAIPVNR
PEPTIDE:

[SEQ ID 559]

SFLLSGNQNQQNYLSGFS
PEPTIDE:

[SEQ ID 560]

LAIPVNRPGQLQSFLLSG
PEPTIDE:

[SEQ ID 561]

SFLLSGNQNQQNYLSG
PEPTIDE:

[SEQ ID 562]

LDLAIPVNRPGQL
PEPTIDE:

[SEQ ID 563]

VLDLAIPVNRPGQLQ
PEPTIDE:

[SEQ ID 564]

LAIPVNRPGQLQSFLLSGNQNQ
PEPTIDE:

[SEQ ID 565]

SFLLSGNQNQQNYLSGFSKNILE
PEPTIDE:

[SEQ ID 566]

GSLLLPHYN
PEPTIDE:

[SEQ ID 567]

GSLLLPHYNS
PEPTIDE:

[SEQ ID 568]

SSNLDLLGFG
PEPTIDE:

[SEQ ID 569]

AFDLAKNKNQYLRGFS
PEPTIDE:

[SEQ ID 570]

QYLRGFSKNILE
PEPTIDE:

[SEQ ID 571]

NLLGFGINAE
PEPTIDE:

[SEQ ID 572]

SNLNLLGFG
PEPTIDE:

[SEQ ID 573]

LAKNKNQYLRGFSKN
PEPTIDE:

[SEQ ID 574]

LAKNKNQYLRGFSK
PEPTIDE:

[SEQ ID 575]

LRGFSKNILE
PEPTIDE:

[SEQ ID 576]

YSNKFGKLFE
PEPTIDE:

[SEQ ID 577]

AFDLAKNKNQYLRGF
PEPTIDE:

[SEQ ID 578]

AFDLAKNKNQYLRGFSK
PEPTIDE:

[SEQ ID 579]

NKNQYLRGFS
PEPTIDE:

[SEQ ID 580]

NKNQYLRGFSKNILE
PEPTIDE:

[SEQ ID 581]

SSNLNLLGFG
PEPTIDE:

[SEQ ID 582]

EYSNKFGKLFE
PEPTIDE:

[SEQ ID 583]

ASSNLNLLG
PEPTIDE:

[SEQ ID 584]

LNLLGFGI
PEPTIDE:

[SEQ ID 585]

NKFGKLFE
PEPTIDE:

[SEQ ID 586]

VQPGRERWEREEDEEQVDE
PEPTIDE:

[SEQ ID 587]

RERWEREEDEEQVDE
PEPTIDE:

[SEQ ID 588]

ASSNLNLLGF
PEPTIDE:

[SEQ ID 589]

LAKNKNQYLRGFS
PEPTIDE:

[SEQ ID 590]

ELLGLKNE
PEPTIDE:

[SEQ ID 591]

ASSNLNLL
PEPTIDE:

[SEQ ID 592]

YPQLQDLDL
PEPTIDE:

[SEQ ID 593]

LLGLKNEQQE
PEPTIDE:

[SEQ ID 594]

LVVLSGKAIL
PEPTIDE:

[SEQ ID 595]

YDATTAIEAK
PEPTIDE:

[SEQ ID 596]

NGLQLLKPTL
PEPTIDE:

[SEQ ID 597]

GLQLLKPTL
PEPTIDE:

[SEQ ID 598]

GVLRPGQLL
PEPTIDE:

[SEQ ID 599]

DGVLRPGQLL
PEPTIDE:

[SEQ ID 600]

LQLLKPTLTQQQE
PEPTIDE:

[SEQ ID 601]

FLLAGNNNR
PEPTIDE:

[SEQ ID 602]

EFLLAGNNNR
PEPTIDE:

[SEQ ID 603]

FLLAGNNNRAQQQQVYGSSIE
PEPTIDE:

[SEQ ID 604]

FLLAGNNNRAQQQQ
PEPTIDE:

[SEQ ID 605]

FLLAGNNNRAQQQQVYG
PEPTIDE:

[SEQ ID 606]

FLLAGNNNRAQQQQVY
PEPTIDE:

[SEQ ID 607]

FQQQYYPGLSNESESETSE
PEPTIDE:

[SEQ ID 608]

LSEALGVNAL
PEPTIDE:

[SEQ ID 609]

LRPAFAQQQEQAQQQEQA
PEPTIDE:

[SEQ ID 610]

LRPAFAQQQE
PEPTIDE:

[SEQ ID 611]

LRPAFAQQQEQAQQQE
PEPTIDE:

[SEQ ID 612]

HGLSLLQPYA
PEPTIDE:

[SEQ ID 613]

HGLSLLQPYASL
PEPTIDE:

[SEQ ID 614]

HGLSLLQPY
PEPTIDE:

[SEQ ID 615]

GSLLLPHYN
PEPTIDE:

[SEQ ID 616]

GSLLLPHYNS
PEPTIDE:

[SEQ ID 617]

SSNLDLLGFG
PEPTIDE:

[SEQ ID 618]

ELLGLKNE
PEPTIDE:

[SEQ ID 619]

SNLNLLGFG
PEPTIDE:

[SEQ ID 620]

ASSNLNLL
PEPTIDE:

[SEQ ID 621]

YPQLQDLDL
PEPTIDE:

[SEQ ID 622]

SSNLNLLGFG
PEPTIDE:

[SEQ ID 623]

ASSNLNLLG
PEPTIDE:

[SEQ ID 624]

LLGLKNEQQE
PEPTIDE:

[SEQ ID 625]

LVVLSGKAIL
PEPTIDE:

[SEQ ID 626]

LNLLGFGI
PEPTIDE:

[SEQ ID 627]

ASSNLNLLGF
PEPTIDE:

[SEQ ID 628]

LQNYRLLE
PEPTIDE:

[SEQ ID 629]

LLSGTQNQPSLL
PEPTIDE:

[SEQ ID 630]

LLLPNYNSR
PEPTIDE:

[SEQ ID 631]

NLQNYRLLE
PEPTIDE:

[SEQ ID 632]

GSLLLPNYNS
PEPTIDE:

[SEQ ID 633]

NLQNYRLL
PEPTIDE:

[SEQ ID 634]

NGLQLLKPTL
PEPTIDE:

[SEQ ID 635]

GLQLLKPTL
PEPTIDE:

[SEQ ID 636]

GVLRPGQLL
PEPTIDE:

[SEQ ID 637]

DGVLRPGQLL
PEPTIDE:

[SEQ ID 638]

LQLLKPTLTQQQE
PEPTIDE:

[SEQ ID 639]

LSEALGVNAL
PEPTIDE:

[SEQ ID 640]

LNSQKFPILNLVQLSATR
PEPTIDE:

[SEQ ID 641]

HGLSLLQPYA
PEPTIDE:

[SEQ ID 642]

HGLSLLQPYASL
PEPTIDE:

[SEQ ID 643]

HGLSLLQPY
PEPTIDE:

[SEQ ID 644]

DKIILGPK
PEPTIDE:

[SEQ ID 645]

DRKRRQQGEETDAIVK
PEPTIDE:

[SEQ ID 646]

IGINGFGRIGRLVAR
PEPTIDE:

[SEQ ID 647]

ILNRGHKIKGTVVL
PEPTIDE:

[SEQ ID 648]

INGFGRIGRLVAR
PEPTIDE:

[SEQ ID 649]

KKNEPWWPK
PEPTIDE:

[SEQ ID 650]

LLLLGIIFLASVV
PEPTIDE:

[SEQ ID 651]

RLLQKFDQRSKIF
PEPTIDE:

[SEQ ID 652]

TIKSRFPLLLLLG
PEPTIDE:

[SEQ ID 653]

TKAVKNTVGRAL
PEPTIDE:

[SEQ ID 654]

VIVKLSR
PEPTIDE:

[SEQ ID 655]

HSPR
PEPTIDE:

[SEQ ID 656]

HWF
PEPTIDE:

[SEQ ID 657]

IFEDAITIPGR
PEPTIDE:

[SEQ ID 658]

ELTFPGSVQE
PEPTIDE:

[SEQ ID 659]

ELTFPGSVQ
PEPTIDE:

[SEQ ID 660]

DSINALEPDHR
PEPTIDE:

[SEQ ID 661]

LDALEPDNRIESE
PEPTIDE:

[SEQ ID 662]

EEGIQLVAEAIR
PEPTIDE:

[SEQ ID 663]

TFAEETWGK
PEPTIDE:

[SEQ ID 664]

LVSHPIAAHEGR
PEPTIDE:

[SEQ ID 665]

NLAQAPAQALL
PEPTIDE:

[SEQ ID 666]

ILVDGSHDIER
PEPTIDE:

[SEQ ID 667]

LDVTPLSLGL
PEPTIDE:

[SEQ ID 668]

WTIVQGLPIDE
PEPTIDE:

[SEQ ID 669]

KTLDYWPSLR
PEPTIDE:

[SEQ ID 670]

RHGEWGPSY
PEPTIDE:

[SEQ ID 671]

HMPP
PEPTIDE:

[SEQ ID 672]

HMPS
PEPTIDE:

[SEQ ID 673]

MPPSS
PEPTIDE:

[SEQ ID 674]

PRRF
PEPTIDE:

[SEQ ID 675]

FHMP
PEPTIDE:

[SEQ ID 676]

NNPF
PEPTIDE:

[SEQ ID 677]

FWM
PEPTIDE:

[SEQ ID 678]

PHMP
PEPTIDE:

[SEQ ID 679]

FHMPP
PEPTIDE:

[SEQ ID 680]

HMPSS
PEPTIDE:

[SEQ ID 681]

HRRS
PEPTIDE:

[SEQ ID 682]

MPPS
PEPTIDE:

[SEQ ID 683]

MPRR
PEPTIDE:

[SEQ ID 684]

PHMPS
PEPTIDE:

[SEQ ID 685]

HGGEGGRPY
PEPTIDE:

[SEQ ID 686]

GYPMYPLPR
PEPTIDE:

[SEQ ID 687]

LQQAPPPPQR
PEPTIDE:

[SEQ ID 688]

VGWGEQPWSPY
PEPTIDE:

[SEQ ID 689]

HPRPPKPDAPR
PEPTIDE:

[SEQ ID 690]

FWN
PEPTIDE:

[SEQ ID 691]

MRFR
PEPTIDE:

[SEQ ID 692]

WHT
PEPTIDE:

[SEQ ID 693]

FRRP
PEPTIDE:

[SEQ ID 694]

HRFR
PEPTIDE:

[SEQ ID 695]

MFRR
PEPTIDE:

[SEQ ID 696]

MFRRP
PEPTIDE:

[SEQ ID 697]

NMPS
PEPTIDE:

[SEQ ID 698]

WMK
PEPTIDE:

[SEQ ID 699]

YSLKPLVPR
PEPTIDE:

[SEQ ID 700]

PVEMPTLLYPS
PEPTIDE:

[SEQ ID 701]

QSFLLSGNQ
PEPTIDE:

[SEQ ID 702]

SLTLEDVPNHGTIR
PEPTIDE:

[SEQ ID 703]

LSLTDLK
PEPTIDE:

[SEQ ID 704]

THPMNFLNER
PEPTIDE:

[SEQ ID 705]

LGLSPQDALK
PEPTIDE:

[SEQ ID 706]

TRPPVPSTIPTK
PEPTIDE:

[SEQ ID 707]

RGPQQYAEWQINE
PEPTIDE:

[SEQ ID 708]

GIARLAGTSSVIN
PEPTIDE:

[SEQ ID 709]

YLRGFS
PEPTIDE:

[SEQ ID 710]

LRGFSK
PEPTIDE:

[SEQ ID 711]

GALMLPHYNSR
PEPTIDE:

[SEQ ID 712]

GALMLPHYN
PEPTIDE:

[SEQ ID 713]

RSQNIF
PEPTIDE:

[SEQ ID 714]

GHPM
PEPTIDE:

[SEQ ID 715]

WDP
PEPTIDE:

[SEQ ID 716]

WHN
PEPTIDE:

[SEQ ID 717]

PMPL
PEPTIDE:

[SEQ ID 718]

HNPR
PEPTIDE:

[SEQ ID 719]

HPSF
PEPTIDE:

[SEQ ID 720]

PNSM
PEPTIDE:

[SEQ ID 721]

HPMS
PEPTIDE:

[SEQ ID 722]

MPMP
PEPTIDE:

[SEQ ID 723]

VFDGVLRPG
PEPTIDE:

[SEQ ID 724]

RLQSQNDQRG
PEPTIDE:

[SEQ ID 725]

LQSQND
PEPTIDE:

[SEQ ID 726]

LEPDNR
PEPTIDE:

[SEQ ID 727]

QSQNDQRGEIIHVK
PEPTIDE:

[SEQ ID 728]

RGEIIHVK
PEPTIDE:

[SEQ ID 729]

RLQSQNDQ
PEPTIDE:

[SEQ ID 730]

RLQSQNDQRGEIIH
PEPTIDE:

[SEQ ID 731]

LQSQNDQRGEI
PEPTIDE:

[SEQ ID 732]

FLPQHTD
PEPTIDE:

[SEQ ID 733]

PQQYAEWQ
PEPTIDE:

[SEQ ID 734]

QSFLLSGNQNQQ
PEPTIDE:

[SEQ ID 735]

QSFLLSGNQ
PEPTIDE:

[SEQ ID 736]

PGQLQSFLLSGN
PEPTIDE:

[SEQ ID 737]

PGQLQSFLLSGNQNQQNYLSGF
PEPTIDE:

[SEQ ID 738]

QLQSFLLSGNQNQQNYLSGFSK
PEPTIDE:

[SEQ ID 739]

QNQQNYLSGFSK
PEPTIDE:

[SEQ ID 740]

GPQQYAEWQINEK
PEPTIDE:

[SEQ ID 741]

RGPQQYA
PEPTIDE:

[SEQ ID 742]

EWQINEK
PEPTIDE:

[SEQ ID 743]

GKIKIGINGFGRIGRLVA
PEPTIDE:

[SEQ ID 744]

MMAP
PEPTIDE:

[SEQ ID 745]

MAPH
PEPTIDE:

[SEQ ID 746]

ERGVLY
PEPTIDE:

[SEQ ID 747]

RVLNGL
PEPTIDE:

[SEQ ID 748]

SLLSGE
PEPTIDE:

[SEQ ID 749]

LLSGED
PEPTIDE:

[SEQ ID 750]

LSGEDA
PEPTIDE:

[SEQ ID 751]

HRHA
PEPTIDE:

[SEQ ID 752]

SRAIVIVTVNE
PEPTIDE:

[SEQ ID 753]

AKLTPGDV
PEPTIDE:

[SEQ ID 754]

IVIVTVNEGK
PEPTIDE:

[SEQ ID 755]

LDALEPDNRIESEGGL (also in P02857)
PEPTIDE:

[SEQ ID 756]

RPYYSNAPQE (also in P02857)
PEPTIDE:

[SEQ ID 757]

LDALEPDNRIESEGGLIETWNPNNK (also in P02857)
PEPTIDE:

[SEQ ID 758]

AIVIVTVNEGK (also in P13918)
PEPTIDE:

[SEQ ID 759]

LQVVNCNGNTVFDGEL
PEPTIDE:

[SEQ ID 760]

QVVNCNGNTVFDGEL
PEPTIDE:

[SEQ ID 761]

IIAVPTGIVF
PEPTIDE:

[SEQ ID 762]

GRRYRDRHQKVNRFRE
PEPTIDE:

[SEQ ID 763]

RPYYSNAPQEI
PEPTIDE:

[SEQ ID 764]

RLDALEPDNRIE
PEPTIDE:

[SEQ ID 765]

RLDALEPDNRIESE
PEPTIDE:

[SEQ ID 766]

LDALEPDNRIESEGGLIETW
PEPTIDE:

[SEQ ID 767]

LDALEPDNRIE
PEPTIDE:

[SEQ ID 768]

LDALEPDNRIESEGGLIE
PEPTIDE:

[SEQ ID 769]

LDALEPDNRIESEGGL (also in P02855)
PEPTIDE:

[SEQ ID 770]

RPYYSNAPQE (also in P02857)
PEPTIDE:

[SEQ ID 771]

LDALEPDNRIESEGGLIETWNPNNK (also in P02855)
PEPTIDE:

[SEQ ID 772]

VEHGLSLLQPYASLQEQEQGQVQSRER
PEPTIDE:

[SEQ ID 773]

RSQNIFSGF
PEPTIDE:

[SEQ ID 774]

GITGPTFPGCPESY
PEPTIDE:

[SEQ ID 775]

CNGS
PEPTIDE:

[SEQ ID 776]

SPREC
PEPTIDE:

[SEQ ID 777]

PREC
PEPTIDE:

[SEQ ID 778]

PRECR
PEPTIDE:

[SEQ ID 779]

CPES
PEPTIDE:

[SEQ ID 780]

SGCS
PEPTIDE:

[SEQ ID 781]

CSNG
PEPTIDE:

[SEQ ID 782]

RSQNIFSGFSTE
PEPTIDE:

[SEQ ID 783]

VEEWSQNIFSGFST
PEPTIDE:

[SEQ ID 784]

WSQNIFSGFSTEL
PEPTIDE:

[SEQ ID 785]

WSQNIFSGFSTE
PEPTIDE:

[SEQ ID 786]

STSQWQSSRR
PEPTIDE:

[SEQ ID 787]

NRPI
PEPTIDE:

[SEQ ID 788]

CDGS
PEPTIDE:

[SEQ ID 789]

PRGC
PEPTIDE:

[SEQ ID 790]

PRGCR
PEPTIDE:

[SEQ ID 791]

RGCR
PEPTIDE:

[SEQ ID 792]

GCRF
PEPTIDE:

[SEQ ID 793]

PTFP
PEPTIDE:

[SEQ ID 794]

PGCPE
PEPTIDE:

[SEQ ID 795]

GCPE
PEPTIDE:

[SEQ ID 796]

CPET
PEPTIDE:

[SEQ ID 797]

AHWC
PEPTIDE:

[SEQ ID 798]

HWCY
PEPTIDE:

[SEQ ID 799]

SGCP
PEPTIDE:

[SEQ ID 800]

SGCPN
PEPTIDE:

[SEQ ID 801]

GCPN
PEPTIDE:

[SEQ ID 802]

CPNG
PEPTIDE:

[SEQ ID 803]

TFCTM
PEPTIDE:

[SEQ ID 804]

FCTM
PEPTIDE:

[SEQ ID 805]

FCTMR
PEPTIDE:

[SEQ ID 806]

CTMR
PEPTIDE:

[SEQ ID 807]

EGCA
PEPTIDE:

[SEQ ID 808]

SQNIFSGFSTELL
PEPTIDE:

[SEQ ID 809]

SQNIFSGFSTE
PEPTIDE:

[SEQ ID 810]

QNDQRGEIVR
PEPTIDE:

[SEQ ID 811]

SQNIFSGFSTEL (also in P07730)
PEPTIDE:

[SEQ ID 812]

QLQCQNDQRGEI (also in P07730)
PEPTIDE:

[SEQ ID 813]

LGQSTSQWQSSR (also in P07730)
PEPTIDE:

[SEQ ID 814]

QQLLGQSTSQWQSSR (also in P07730)
PEPTIDE:

[SEQ ID 815]

LLGQSTSQWQSSR (also in P07730)
PEPTIDE:

[SEQ ID 816]

NDQRGEIVR
PEPTIDE:

[SEQ ID 817]

GQSTSQWQSSR
PEPTIDE:

[SEQ ID 818]

STSQWQSSR
PEPTIDE:

[SEQ ID 819]

GITGPTFPGCPET
PEPTIDE:

[SEQ ID 820]

GITGPTFPGCPETY
PEPTIDE:

[SEQ ID 821]

SQNIFSGFSTEL (also in P07728)
PEPTIDE:

[SEQ ID 822]

QLQCQNDQRGEI (also in P07728)
PEPTIDE:

[SEQ ID 823]

LGQSTSQWQSSR (also in P07728)
PEPTIDE:

[SEQ ID 824]

QQLLGQSTSQWQSSR (also in P07728)
PEPTIDE:

[SEQ ID 825]

LLGQSTSQWQSSR (also in P07728)
PEPTIDE:

[SEQ ID 826]

IFFANQTYL
PEPTIDE:

[SEQ ID 827]

EHLEPNLEGLTVEE
PEPTIDE:

[SEQ ID 828]

IFFANQTYLPSETPAPLVHYREEELNNLRGDGTGER
PEPTIDE:

[SEQ ID 829]

IHFEWDDDMGIPGAFYIK
PEPTIDE:

[SEQ ID 830]

IFFANQTYLPSETPAPLVHYREEELNNLR
PEPTIDE:

[SEQ ID 831]

TEQALPADLIK
PEPTIDE:

[SEQ ID 832]

EHLEPNLEGLTVEEAIQNKK
PEPTIDE:

[SEQ ID 833]

ISKEHLEPNLEGLTVEEAIQNKK
PEPTIDE:

[SEQ ID 834]

LSLPHPQGDEHGAVSY
PEPTIDE:

[SEQ ID 835]

ISKEHLEPNLEGLTVEEAIQNK
PEPTIDE:

[SEQ ID 836]

EHLEPNLEGLTVEEAIQNK
PEPTIDE:

[SEQ ID 837]

LSTTGGNSGSPVFNEKNE
PEPTIDE:

[SEQ ID 838]

QSFLLSGNQNQQNYLSG
PEPTIDE:

[SEQ ID 839]

VLDLAIPVNRPGQLQS
PEPTIDE:

[SEQ ID 840]

VLDLAIPVNRPGQLQSFL
PEPTIDE:

[SEQ ID 841]

FLLSGNQNQQNYLSG
PEPTIDE:

[SEQ ID 842]

FLLSGNQNQQNYLSGFSK
PEPTIDE:

[SEQ ID 843]

DPQNPFIFKSNKFQTLFE
PEPTIDE:

[SEQ ID 844]

ELAFPGSAQEVDRILENQK
PEPTIDE:

[SEQ ID 845]

AIVIVTVNEGK (also in P02855)
PEPTIDE:

[SEQ ID 846]

INAVAAKRLQSQNDQRGE
PEPTIDE:

[SEQ ID 847]

NRAQQQQVYGSSIE
PEPTIDE:

[SEQ ID 848]

PSTNPWHSPR
PEPTIDE:

[SEQ ID 849]

CHGS
PEPTIDE:

[SEQ ID 850]

CHGSM
PEPTIDE:

[SEQ ID 851]

PWHS
PEPTIDE:

[SEQ ID 852]

FREC
PEPTIDE:

[SEQ ID 853]

RECR
PEPTIDE:

[SEQ ID 854]

ECRF
PEPTIDE:

[SEQ ID 855]

CRFD
PEPTIDE:

[SEQ ID 856]

CRFDR
PEPTIDE:

[SEQ ID 857]

CTGT
PEPTIDE:

[SEQ ID 858]

FPGC
PEPTIDE:

[SEQ ID 859]

FPGCP
PEPTIDE:

[SEQ ID 860]

PGCP
PEPTIDE:

[SEQ ID 861]

PGCPA
PEPTIDE:

[SEQ ID 862]

PGCPAT
PEPTIDE:

[SEQ ID 863]

GCPA
PEPTIDE:

[SEQ ID 864]

CPAT
PEPTIDE:

[SEQ ID 865]

ENFC
PEPTIDE:

[SEQ ID 866]

NFCT
PEPTIDE:

[SEQ ID 867]

NFCTI
PEPTIDE:

[SEQ ID 868]

FCTI
PEPTIDE:

[SEQ ID 869]

AQQQQVYGSSIEQH
PEPTIDE:

[SEQ ID 870]

AQQQQVYGSSIEQHSGQNIFSGF
PEPTIDE:

[SEQ ID 871]

AAKRLQSQNDQRGE
PEPTIDE:

[SEQ ID 872]

QARSLKNNRGEE (also in P14614)
PEPTIDE:

[SEQ ID 873]

FNPSTNPWHSPRQGS (also in Q0DEV5)
PEPTIDE:

[SEQ ID 874]

QARSLKNNRGEE (also in P14323)
PEPTIDE:

[SEQ ID 875]

AAASLPAFCNVDIPNGGGGVCYWLAR
PEPTIDE:

[SEQ ID 876]

VAGSAPGIEGDEIAPLAK
PEPTIDE:

[SEQ ID 877]

LGVAGSAPGIEGDEIAPLAKEN
PEPTIDE:

[SEQ ID 878]

GSAPGIEGDEIAPLAKE
PEPTIDE:

[SEQ ID 879]

VAGSAPGIEGDEIAP
PEPTIDE:

[SEQ ID 880]

GVAGSAPGIEGDEIAPLAK
PEPTIDE:

[SEQ ID 881]

GVAGSAPGIEGDEIAPLAKEN
PEPTIDE:

[SEQ ID 882]

VAGSAPGIEGDEIAPLAKEN
PEPTIDE:

[SEQ ID 883]

SAPGIEGDEIAPLAK
PEPTIDE:

[SEQ ID 884]

GSAPGIEGDEIAPLAK
PEPTIDE:

[SEQ ID 885]

SAPGIEGDEIAPLAKEN
PEPTIDE:

[SEQ ID 886]

FNPSTNPWHSPRQGS (also in P14323)
PEPTIDE:

[SEQ ID 887]

VDLVIPVNGPGK
PEPTIDE:

[SEQ ID 888]

LVDLVIPVNGPGK
PEPTIDE:

[SEQ ID 889]

IKLPAGTTSY
PEPTIDE:

[SEQ ID 890]

IKLPAGTTSYL
PEPTIDE:

[SEQ ID 891]

RRNPFLFKSNKF
PEPTIDE:

[SEQ ID 892]

IENPVKELTFPGSVQEINR
PEPTIDE:

[SEQ ID 893]

RRNPFLFKSNKFLT
PEPTIDE:

[SEQ ID 894]

AKPHTIFLPQHIDA
PEPTIDE:

[SEQ ID 895]

AKPHTIFLPQHIDAD
PEPTIDE:

[SEQ ID 896]

KQKYRYQRE
PEPTIDE:

[SEQ ID 897]

KQKYQYQRE
PEPTIDE:

[SEQ ID 898]

MLPH
PEPTIDE:

[SEQ ID 899]

RRNPFLFKSNKFLTLFENE
PEPTIDE:

[SEQ ID 900]

PFLFKSNKFLTLFE
PEPTIDE:

[SEQ ID 901]

SQERRNPFLFKSNKFLTLFE
PEPTIDE:

[SEQ ID 902]

RRNPFLFKSNKFLTLFE
PEPTIDE:

[SEQ ID 903]

SQERRNPFLFKSNKFLTLFENE
PEPTIDE:

[SEQ ID 904]

LTFPGSVQE
PEPTIDE:

[SEQ ID 905]

ELTFPGSVQEINR
PEPTIDE:

[SEQ ID 906]

KNPQLQDLDI
PEPTIDE:

[SEQ ID 907]

GQSTSQWQSSR
PEPTIDE:

[SEQ ID 908]

QSTSQWQSSR (also in P07728)
PEPTIDE:

[SEQ ID 909]

QSTSQWQSSR (also in P07730)
PEPTIDE:

[SEQ ID 910]

EEEEQGEEEINK
PEPTIDE:

[SEQ ID 911]

PSTNPWHSPR
PEPTIDE:

[SEQ ID 912]

AQAQDQYQQVQYSE
PEPTIDE:

[SEQ ID 913]

SEAGVTEYFDEKNELFQCTGTFVIRR
PEPTIDE:

[SEQ ID 914]

QAQAQDQYQQVQYSE
PEPTIDE:

[SEQ ID 915]

GSMGLTFPGCPAT (also in P14614)
PEPTIDE:

[SEQ ID 916]

GSMGLTFPGCPATY (also in P14614)
PEPTIDE:

[SEQ ID 917]

LGAFTPRY
PEPTIDE:

[SEQ ID 918]

LGAFTPRYQQ
PEPTIDE:

[SEQ ID 919]

ALGVNALVAKRLQGQN
PEPTIDE:

[SEQ ID 920]

LGAFTPRYQ
PEPTIDE:

[SEQ ID 921]

GSMGLTFPGCPAT (also in P14323)
PEPTIDE:

[SEQ ID 922]

GSMGLTFPGCPATY (also in P14323)
PEPTIDE:

[SEQ ID 923]

SNNPFKFLVPARQS
PEPTIDE:

[SEQ ID 924]

CAGVFVIRR
PEPTIDE:

[SEQ ID 925]

GSPLQSPRGF
PEPTIDE:

[SEQ ID 926]

RSSWQQQSY
PEPTIDE:

[SEQ ID 927]

SFGGSPLQSPR
PEPTIDE:

[SEQ ID 928]

YLPTKQLQPTW
PEPTIDE:

[SEQ ID 929]

GKPRSSWQQQ
PEPTIDE:

[SEQ ID 930]

FGGSPLQSPRG
PEPTIDE:

[SEQ ID 931]

LNLLGFGINAENNE
PEPTIDE:

[SEQ ID 932]

SGPFNLRSRNPIYSNKFGKFFE
PEPTIDE:

[SEQ ID 933]

TSKQVQLYKAKLSPGDVFVIPAG
PEPTIDE:

[SEQ ID 934]

GHIRLLQKFDKRSKIFE
PEPTIDE:

[SEQ ID 935]

TSKQVQLYKAKLSPGDVFVIPAGHP
PEPTIDE:

[SEQ ID 936]

TSKQVQLYKAKLSPGDVFVIPAGHPV
PEPTIDE:

[SEQ ID 937]

TSKQVQLYKAKLSPGDVFVIPAGHPVA
PEPTIDE:

[SEQ ID 938]

TSKQVQLYKAKLSPGDVFVIPAGHPVAI
PEPTIDE:

[SEQ ID 939]

TSKQVQLYKAKLSPGDVFVIPAGHPVAIN
PEPTIDE:

[SEQ ID 940]

KFKDEHQKIHRFRQGDVIALPAGVAHW
PEPTIDE:

[SEQ ID 941]

KFKDEHQKIHRFRQGDVIALPAGVAHWC
PEPTIDE:

[SEQ ID 942]

KFKDEHQKIHRFRQGDVIALPAGVAHWCY
PEPTIDE:

[SEQ ID 943]

SPFWNINAHSVVYITQGRARVQVVNNNGK
PEPTIDE:

[SEQ ID 944]

QKIHRFRQGDVIALPAGVAHW
PEPTIDE:

[SEQ ID 945]

VVRRVIEPRGLLLPHYTNG
PEPTIDE:

[SEQ ID 946]

VRRVIEPRGLLLPHYTNG
PEPTIDE:

[SEQ ID 947]

IRLLQKFDQRSKIFE
PEPTIDE:

[SEQ ID 948]

AVAAKRLQSQNDQRGEIIHVKNGLQ
PEPTIDE:

[SEQ ID 949]

VAAKRLQSQNDQRGEIIHVKNGLQ
PEPTIDE:

[SEQ ID 950]

TVFDGVLRPGQLLIIPQHYAVLKK
PEPTIDE:

[SEQ ID 951]

PSTNPWHSPR
PEPTIDE:

[SEQ ID 952]

NPSTNPWHSPRQGSFR
PEPTIDE:

[SEQ ID 953]

QLFNPSTNPWHSPRQGSFR
PEPTIDE:

[SEQ ID 954]

SMAQLFNPSTNPWHSPRQGSFR
PEPTIDE:

[SEQ ID 955]

QLFNPSTNPWHSPRQGSFRECRF
PEPTIDE:

[SEQ ID 956]

CHGSMAQLFNPSTNPWHSPRQGSFR
PEPTIDE:

[SEQ ID 957]

CHGSMAQLFNPSTNPWHSPRQGSFRECRF
PEPTIDE:

[SEQ ID 958]

LLLCHGSMAQLFNPSTNPWHSPRQGSFR
PEPTIDE:

[SEQ ID 959]

PSTNPWHSPRQGSFRE
PEPTIDE:

[SEQ ID 960]

PWHSPRQGSFRE
PEPTIDE:

[SEQ ID 961]

QLFNPSTNPWHSPRQGSF
PEPTIDE:

[SEQ ID 962]

RVIQPQGLLVPR
PEPTIDE:

[SEQ ID 963]

FNPSTNPWHSPRQGSF
PEPTIDE:

[SEQ ID 964]

FNPSTNPWHSPRQGS
PEPTIDE:

[SEQ ID 965]

GPNVNPWHNPRQGGFRECR
PEPTIDE:

[SEQ ID 966]

QLFGPNVNPWHNPRQGGFR
PEPTIDE:

[SEQ ID 967]

QLFGPNVNPWHNPRQGGFRECR
PEPTIDE:

[SEQ ID 968]

SMAQLFGPNVNPWHNPRQGGFR
PEPTIDE:

[SEQ ID 969]

CHGSMAQLFGPNVNPWHNPRQGGFR
PEPTIDE:

[SEQ ID 970]

SMAQLFGPNVNPWHNPRQGGFRECR
PEPTIDE:

[SEQ ID 971]

LLLCHGSMAQLFGPNVNPWHNPRQGGFR
PEPTIDE:

[SEQ ID 972]

WRPSYEKEE
PEPTIDE:

[SEQ ID 973]

AKPHTIFLPQHIDADLILVVLSGKAILTVLSPNDR
PEPTIDE:

[SEQ ID 974]

PRVPAQRERGRQEGEKEEKRHGEWRP
PEPTIDE:

[SEQ ID 975]

PRVPAQRERGRQEGEKEEKRHGEWRPS
PEPTIDE:

[SEQ ID 976]

PRVPAQRERGRQEGEKEEKRHGEWRPSY
PEPTIDE:

[SEQ ID 977]

PRVPAQRERGRQEGEKEEKRHGEWR
PEPTIDE:

[SEQ ID 978]

GSEPRVPAQRERGRQEGEKEEKRHGEWRP
PEPTIDE:

[SEQ ID 979]

KRHGEWRPSYEK
PEPTIDE:

[SEQ ID 980]

KKEQKEVQPGRERW
PEPTIDE:

[SEQ ID 981]

KKEQKEVQPGRERWER
PEPTIDE:

[SEQ ID 982]

REKKEQKEVQPGRERW
PEPTIDE:

[SEQ ID 983]

QREKKEQKEVQPGRERW
PEPTIDE:

[SEQ ID 984]

REKKEQKEVQPGRERWER
PEPTIDE:

[SEQ ID 985]

QREKKEQKEVQPGRERWER
PEPTIDE:

[SEQ ID 986]

QYQREKKEQKEVQPGRERW
PEPTIDE:

[SEQ ID 987]

YQYQREKKEQKEVQPGRERW
PEPTIDE:

[SEQ ID 988]

QYQREKKEQKEVQPGRERWER
PEPTIDE:

[SEQ ID 989]

QKYQYQREKKEQKEVQPGRERW
PEPTIDE:

[SEQ ID 990]

YQYQREKKEQKEVQPGRERWER
PEPTIDE:

[SEQ ID 991]

KQKYQYQREKKEQKEVQPGRERW
PEPTIDE:

[SEQ ID 992]

QKYQYQREKKEQKEVQPGRERWE
PEPTIDE:

[SEQ ID 993]

KQKYQYQREKKEQKEVQPGRERWE
PEPTIDE:

[SEQ ID 994]

QKYQYQREKKEQKEVQPGRERWER
PEPTIDE:

[SEQ ID 995]

EEKQKYQYQREKKEQKEVQPGRERW
PEPTIDE:

[SEQ ID 996]

KQKYQYQREKKEQKEVQPGRERWER
PEPTIDE:

[SEQ ID 997]

QKYQYQREKKEQKEVQPGRERWERE
PEPTIDE:

[SEQ ID 998]

KQKYQYQREKKEQKEVQPGRERWERE
PEPTIDE:

[SEQ ID 999]

EEKQKYQYQREKKEQKEVQPGRERWER
PEPTIDE:

[SEQ ID 1000]

KQKYQYQREKKEQKEVQPGRERWEREE
PEPTIDE:

[SEQ ID 1001]

PINLRSHKPEYSNKFGKLFEITPEKKYP
PEPTIDE:

[SEQ ID 1002]

KRHGEWRPSY
PEPTIDE:

[SEQ ID 1003]

PAQRERGRQEGEKEEKRHGEWRP
PEPTIDE:

[SEQ ID 1004]

PAQRERGRQEGEKEEKRHGEWRPS
PEPTIDE:

[SEQ ID 1005]

PRVPAQRERGRQEGEKEEKRHGEW
PEPTIDE:

[SEQ ID 1006]

EWRGSQRREDPEERARLRHREERTK
PEPTIDE:

[SEQ ID 1007]

PAQRERGRQEGEKEEKRHGEWRPSY
PEPTIDE:

[SEQ ID 1008]

EWRGSQRREDPEERARLRHREERTKR
PEPTIDE:

[SEQ ID 1009]

PAQRERGRQEGEKEEKRHGEWRPSYE
PEPTIDE:

[SEQ ID 1010]

EEWRGSQRREDPEERARLRHREERTKR
PEPTIDE:

[SEQ ID 1011]

EWRGSQRREDPEERARLRHREERTKRD
PEPTIDE:

[SEQ ID 1012]

GSEPRVPAQRERGRQEGEKEEKRHGEW
PEPTIDE:

[SEQ ID 1013]

PAQRERGRQEGEKEEKRHGEWRPSYEK
PEPTIDE:

[SEQ ID 1014]

EWRGSQRREDPEERARLRHREERTKRDR
PEPTIDE:

[SEQ ID 1015]

GSEPRVPAQRERGRQEGEKEEKRHGEWR
PEPTIDE:

[SEQ ID 1016]

PAQRERGRQEGEKEEKRHGEWRPSYEKE
PEPTIDE:

[SEQ ID 1017]

EEWRGSQRREDPEERARLRHREERTKRDR
PEPTIDE:

[SEQ ID 1018]

EWRGSQRREDPEERARLRHREERTKRDRR
PEPTIDE:

[SEQ ID 1019]

KEEKRHGEWRP
PEPTIDE:

[SEQ ID 1020]

KEEKRHGEWRPS
PEPTIDE:

[SEQ ID 1021]

GEKEEKRHGEWRP
PEPTIDE:

[SEQ ID 1022]

KEEKRHGEWRPSY
PEPTIDE:

[SEQ ID 1023]

KEQKEVQPGRERW
PEPTIDE:

[SEQ ID 1024]

KRHGEWRPSYEKE
PEPTIDE:

[SEQ ID 1025]

EEKRHGEWRPSYEK
PEPTIDE:

[SEQ ID 1026]

GEKEEKRHGEWRPS
PEPTIDE:

[SEQ ID 1027]

KEEKRHGEWRPSYE
PEPTIDE:

[SEQ ID 1028]

KEQKEVQPGRERWE
PEPTIDE:

[SEQ ID 1029]

EEKRHGEWRPSYEKQ
PEPTIDE:

[SEQ ID 1030]

GEKEEKRHGEWRPSY
PEPTIDE:

[SEQ ID 1031]

KEEKRHGEWRPSYEK
PEPTIDE:

[SEQ ID 1032]

KEQKEVQPGRERWER
PEPTIDE:

[SEQ ID 1033]

KKEQKEVQPGRERWE
PEPTIDE:

[SEQ ID 1034]

KEEKRHGEWRPSYEKE
PEPTIDE:

[SEQ ID 1035]

KEQKEVQPGRERWERE
PEPTIDE:

[SEQ ID 1036]

RQEGEKEEKRHGEWRP
PEPTIDE:

[SEQ ID 1037]

GEKEEKRHGEWRPSYEK
PEPTIDE:

[SEQ ID 1038]

GRQEGEKEEKRHGEWRP
PEPTIDE:

[SEQ ID 1039]

KKEQKEVQPGRERWERE
PEPTIDE:

[SEQ ID 1040]

REKKEQKEVQPGRERWE
PEPTIDE:

[SEQ ID 1041]

RQEGEKEEKRHGEWRPS
PEPTIDE:

[SEQ ID 1042]

GEKEEKRHGEWRPSYEKQ
PEPTIDE:

[SEQ ID 1043]

GRQEGEKEEKRHGEWRPS
PEPTIDE:

[SEQ ID 1044]

KKEQKEVQPGRERWEREE
PEPTIDE:

[SEQ ID 1045]

QREKKEQKEVQPGRERWE
PEPTIDE:

[SEQ ID 1046]

RQEGEKEEKRHGEWRPSY
PEPTIDE:

[SEQ ID 1047]

ERGRQEGEKEEKRHGEWRP
PEPTIDE:

[SEQ ID 1048]

GRQEGEKEEKRHGEWRPSY
PEPTIDE:

[SEQ ID 1049]

REKKEQKEVQPGRERWERE
PEPTIDE:

[SEQ ID 1050]

ERGRQEGEKEEKRHGEWRPS
PEPTIDE:

[SEQ ID 1051]

QREKKEQKEVQPGRERWERE
PEPTIDE:

[SEQ ID 1052]

QYQREKKEQKEVQPGRERWE
PEPTIDE:

[SEQ ID 1053]

REKKEQKEVQPGRERWEREE
PEPTIDE:

[SEQ ID 1054]

RERGRQEGEKEEKRHGEWRP
PEPTIDE:

[SEQ ID 1055]

RQEGEKEEKRHGEWRPSYEK
PEPTIDE:

[SEQ ID 1056]

ERGRQEGEKEEKRHGEWRPSY
PEPTIDE:

[SEQ ID 1057]

GRQEGEKEEKRHGEWRPSYEK
PEPTIDE:

[SEQ ID 1058]

PAQRERGRQEGEKEEKRHGEW
PEPTIDE:

[SEQ ID 1059]

QREKKEQKEVQPGRERWEREE
PEPTIDE:

[SEQ ID 1060]

RERGRQEGEKEEKRHGEWRPS
PEPTIDE:

[SEQ ID 1061]

RQEGEKEEKRHGEWRPSYEKQ
PEPTIDE:

[SEQ ID 1062]

YQYQREKKEQKEVQPGRERWE
PEPTIDE:

[SEQ ID 1063]

GQRERGRQEGEKEEKRHGEWRP
PEPTIDE:

[SEQ ID 1064]

PAQRERGRQEGEKEEKRHGEWR
PEPTIDE:

[SEQ ID 1065]

QYQREKKEQKEVQPGRERWERE
PEPTIDE:

[SEQ ID 1066]

RERGRQEGEKEEKRHGEWRPSY
PEPTIDE:

[SEQ ID 1067]

ERGRQEGEKEEKRHGEWRPSYEK
PEPTIDE:

[SEQ ID 1068]

GQRERGRQEGEKEEKRHGEWRPS
PEPTIDE:

[SEQ ID 1069]

QYQREKKEQKEVQPGRERWEREE
PEPTIDE:

[SEQ ID 1070]

RERGRQEGEKEEKRHGEWRPSYE
PEPTIDE:

[SEQ ID 1071]

YQYQREKKEQKEVQPGRERWERE
PEPTIDE:

[SEQ ID 1072]

ERGRQEGEKEEKRHGEWRPSYEKQ
PEPTIDE:

[SEQ ID 1073]

GQRERGRQEGEKEEKRHGEWRPSY
PEPTIDE:

[SEQ ID 1074]

RERGRQEGEKEEKRHGEWRPSYEK
PEPTIDE:

[SEQ ID 1075]

YQYQREKKEQKEVQPGRERWEREE
PEPTIDE:

[SEQ ID 1076]

GQRERGRQEGEKEEKRHGEWRPSYE
PEPTIDE:

[SEQ ID 1077]

RERGRQEGEKEEKRHGEWRPSYEKE
PEPTIDE:

[SEQ ID 1078]

RHGEWRPSYEKQEDEEEKQKYRYQR
PEPTIDE:

[SEQ ID 1079]

EEEKQKYQYQREKKEQKEVQPGRERW
PEPTIDE:

[SEQ ID 1080]

GQRERGRQEGEKEEKRHGEWRPSYEK
PEPTIDE:

[SEQ ID 1081]

QKYQYQREKKEQKEVQPGRERWEREE
PEPTIDE:

[SEQ ID 1082]

EEEKQKYQYQREKKEQKEVQPGRERWE
PEPTIDE:

[SEQ ID 1083]

GQRERGRQEGEKEEKRHGEWRPSYEKQ
PEPTIDE:

[SEQ ID 1084]

QKYQYQREKKEQKEVQPGRERWEREED
PEPTIDE:

[SEQ ID 1085]

RHGEWRPSYEKQEDEEEKQKYRYQREK
PEPTIDE:

[SEQ ID 1086]

EEEKQKYQYQREKKEQKEVQPGRERWER
PEPTIDE:

[SEQ ID 1087]

EEKQKYQYQREKKEQKEVQPGRERWERE
PEPTIDE:

[SEQ ID 1088]

GQRERGRQEGEKEEKRHGEWRPSYEKQE
PEPTIDE:

[SEQ ID 1089]

KQKYQYQREKKEQKEVQPGRERWEREED
PEPTIDE:

[SEQ ID 1090]

PSEFEPINLRSHKPEYSNKFGKLFEITP
PEPTIDE:

[SEQ ID 1091]

EEEKQKYQYQREKKEQKEVQPGRERWERE
PEPTIDE:

[SEQ ID 1092]

KEDEEEKQKYQYQREKKEQKEVQPGRERW
PEPTIDE:

[SEQ ID 1093]

KQKYQYQREKKEQKEVQPGRERWEREEDE
PEPTIDE:

[SEQ ID 1094]

LPSEFEPINLRSHKPEYSNKFGKLFEITP
PEPTIDE:

[SEQ ID 1095]

EEKRHGEWRP
PEPTIDE:

[SEQ ID 1096]

KEVQPGRERW
PEPTIDE:

[SEQ ID 1097]

QKEVQPGRERW
PEPTIDE:

[SEQ ID 1098]

EEKRHGEWRPSY
PEPTIDE:

[SEQ ID 1099]

RHGEWRPSYEKQ
PEPTIDE:

[SEQ ID 1100]

KEVQPGRERWERE
PEPTIDE:

[SEQ ID 1101]

RHGEWRPSYEKQE
PEPTIDE:

[SEQ ID 1102]

QKEVQPGRERWERE
PEPTIDE:

[SEQ ID 1103]

KKSLPSEFEPINLRSHKP
PEPTIDE:

[SEQ ID 1104]

EWRGSQRREDPEERARLRHR
PEPTIDE:

[SEQ ID 1105]

SSKKSLPSEFEPINLRSHKP
PEPTIDE:

[SEQ ID 1106]

KPEYSNKFGKLFEITPEKKYP
PEPTIDE:

[SEQ ID 1107]

SSSKKSLPSEFEPINLRSHKP
PEPTIDE:

[SEQ ID 1108]

HKPEYSNKFGKLFEITPEKKYP
PEPTIDE:

[SEQ ID 1109]

AKSSSKKSLPSEFEPINLRSHKP
PEPTIDE:

[SEQ ID 1110]

INLRSHKPEYSNKFGKLFEITPEKKYP
PEPTIDE:

[SEQ ID 1111]

AKSSSKKSLPSEFEPINLRSHKPEYSNKF
PEPTIDE:

[SEQ ID 1112]

RRNPFLFKSNKFLTLFE
PEPTIDE:

[SEQ ID 1113]

PINLRSHKPEYSNKFGKLFEITPEKKYPQ
PEPTIDE:

[SEQ ID 1114]

KPEYSNKFGKLFEITPEKKYPQLQ
PEPTIDE:

[SEQ ID 1115]

HKPEYSNKFGKLFEITPEKKYPQLQ
PEPTIDE:

[SEQ ID 1116]

SHKPEYSNKFGKLFEITPEKKYPQLQ
PEPTIDE:

[SEQ ID 1117]

FEPINLRSHKPEYSNKFGKLFEITPEKK
PEPTIDE:

[SEQ ID 1118]

MASINRPIVFFTVCLFLLCNGSLA
PEPTIDE:

[SEQ ID 1119]

FLLAGNKRNPQ
PEPTIDE:

[SEQ ID 1120]

FLLAGNKRN
PEPTIDE:

[SEQ ID 1121]

VLDLAIPVNRPGQLQSFLLSGNQNQQNYLSGFSK
PEPTIDE:

[SEQ ID 1122]

QSFLLSGNQNQQNYLSG
PEPTIDE:

[SEQ ID 1123]

RLSSV
PEPTIDE:

[SEQ ID 1124]

QKEFLLAGNNNR (also in P14614)
PEPTIDE:

[SEQ ID 1125]

LLRPAFAQQQEQAQQQEQA
PEPTIDE:

[SEQ ID 1126]

VKNGLKLLRPAF
PEPTIDE:

[SEQ ID 1127]

FLLAGNNNRE
PEPTIDE:

[SEQ ID 1128]

GLKLLRPAFAQQQE
PEPTIDE:

[SEQ ID 1129]

LKLLRPAFAQQQE
PEPTIDE:

[SEQ ID 1130]

LLRPAFAQQQE
PEPTIDE:

[SEQ ID 1131]

QKEFLLAGNNNR (also in P14323)
PEPTIDE:

[SEQ ID 1132]

LRGFSKN
PEPTIDE:

[SEQ ID NO: 1133]

ILDLAIPVNRPGQL
PEPTIDE:

[SEQ ID NO: 1134]

VLELAIPVNRPGQL
PEPTIDE:

[SEQ ID NO: 1135]

VLDLAVPVNRPGQL
PEPTIDE:

[SEQ ID NO: 1136]

VLDLAIPINRPGQL
PEPTIDE:

[SEQ ID NO: 1137]

VLDLAIPVNKPGQL
PEPTIDE:

[SEQ ID NO: 1138]

VLDLAIPVEKPGQL
PEPTIDE:

[SEQ ID NO: 1139]

VLDLAIPVNKPGEL
PEPTIDE:

[SEQ ID NO: 1140]

ILELAIPVNRPGQL
PEPTIDE:

[SEQ ID NO: 1141]

ILDLAVPVNRPGQL
PEPTIDE:

[SEQ ID NO: 1142]

VLELAVPVNRPGQL
PEPTIDE:

[SEQ ID NO: 1143]

VLELAIPVNKPGQL
PEPTIDE:

[SEQ ID NO: 1144]

ILDLAIPVNKPGQL
PEPTIDE:

[SEQ ID NO: 1145]

VLDLAVPVNKPGQL
PEPTIDE:

[SEQ ID NO: 1146]

VLDLAIPVEKPGEL
PEPTIDE:

[SEQ ID NO: 1147]

ILDLAIPVNKPGEL
PEPTIDE:

[SEQ ID NO: 1148]

VLELAIPVEKPGQL
PEPTIDE:

[SEQ ID NO: 1149]

ILELAVPVNRPGQL
PEPTIDE:

[SEQ ID NO: 1150]

ILELAIPVNKPGQL
PEPTIDE:

[SEQ ID NO: 1151]

VLELAVPVNKPGQL
PEPTIDE:

[SEQ ID NO: 1152]

ILELAIPVNRPGEL
PEPTIDE:

[SEQ ID NO: 1153]

ILDLAIPVNKPGEL
PEPTIDE:

[SEQ ID NO: 1154]

VLDLAVPVEKPGQL
PEPTIDE:

[SEQ ID NO: 1155]

VLDLAVPVERPGEL
PEPTIDE:

[SEQ ID NO: 1156]

VLELAIPVERPGEL
PEPTIDE:

[SEQ ID NO: 1157]

KLDLAIIVNRPGQL
PEPTIDE:

[SEQ ID NO: 1158]

VLDLAIPVNRPGQK
PEPTIDE:

[SEQ ID NO: 1159]

VLDLAIPVNRPCQL
PEPTIDE:

[SEQ ID NO: 1160]

VLDLWIPVNRPGQL
PEPTIDE:

[SEQ ID NO: 1161]

VLDLAIPVNRPGQL
PEPTIDE:

[SEQ ID NO: 1162]

VLYLAIPVNRPGQL
PEPTIDE:

[SEQ ID NO: 1163]

VLDLYIPVGRPGQL
PEPTIDE:

[SEQ ID NO: 1164]

VKDLAIPWNRPGQL
PEPTIDE:

[SEQ ID NO: 1165]

VLDLAIPVNRPCCL
PEPTIDE:

[SEQ ID NO: 1166]

VLDLAGGVNRPGQL
PEPTIDE:

[SEQ ID NO: 1167]

VLDLAIPKNEPGQL
PEPTIDE:

[SEQ ID NO: 1168]

PLDLAIPVNDPGQL
PEPTIDE:

[SEQ ID NO: 1169]

VLDLAIPVNRPIQL
PEPTIDE:

[SEQ ID NO: 1170]

VLDHAIPVNRPGQL
PEPTIDE:

[SEQ ID NO: 1171]

VLDLAIPVNRPGGG
PEPTIDE:

[SEQ ID NO: 1172]

VLDLHIPGNEPGQL
PEPTIDE:

[SEQ ID NO: 1173]

VYKLAIPVNEPGQL
PEPTIDE:

[SEQ ID NO: 1174]

VLDLAIPVNRPYPG
PEPTIDE:

[SEQ ID NO: 1175]

VLDYAIPKNDPGQL
PEPTIDE:

[SEQ ID NO: 1176]

RRRLAIPVNRPGQL
PEPTIDE:

[SEQ ID NO: 1177]

VLDLAIGVNRGPQL
PEPTIDE:

[SEQ ID NO: 1178]

VLDLAIPVNRPGFQL
PEPTIDE:

[SEQ ID NO: 1179]

VLDLADIPVNRPGQL
PEPTIDE:

[SEQ ID NO: 1180]

VLDLAIPVGNRPGQL
PEPTIDE:

[SEQ ID NO: 1181]

VLQQDLAIPVNRPGQL
PEPTIDE:

[SEQ ID NO: 1182]

VLDLAIPVNRGPGQKL
PEPTIDE:

[SEQ ID NO: 1183]

VLDGLPLAIPVNRPGQL
PEPTIDE:

[SEQ ID NO: 1184]

VLDLAIPVNRPGQLLL
PEPTIDE:

[SEQ ID NO: 1185]

VLDLFLGAIPVNRPGQL
PEPTIDE:

[SEQ ID NO: 1186]

VLDLAIPVNRGQL
PEPTIDE:

[SEQ ID NO: 1187]

VLDLAPVNRPGQL
PEPTIDE:

[SEQ ID NO: 1188]

LDLAIPVNRPGQL
PEPTIDE:

[SEQ ID NO: 1189]

VLDLAIPVNRPGQ
PEPTIDE:

[SEQ ID NO: 1190]

DLAIPVNRPGQL
PEPTIDE:

[SEQ ID NO: 1191]

VLDLAIPVNRPG
PEPTIDE:

[SEQ ID NO: 1192]

VLDLAINRPGQL
PEPTIDE:

[SEQ ID NO: 1193]

VLDAIVNPGQL
PEPTIDE:

[SEQ ID NO: 1194]

RGPQQYAEWQINER
PEPTIDE:

[SEQ ID NO: 1195]

RGPQQYAEWQINDK
PEPTIDE:

[SEQ ID NO: 1196]

RGPQQFAEWQINEK
PEPTIDE:

[SEQ ID NO: 1197]

KGPQQYAEWQINEK
PEPTIDE:

[SEQ ID NO: 1198]

RGPEQYAEWQINEK
PEPTIDE:

[SEQ ID NO: 1199]

RGPQEYAEWQINEK
PEPTIDE:

[SEQ ID NO: 1200]

RGPQQYADWQINEK
PEPTIDE:

[SEQ ID NO: 1201]

RGPQQYAEYQINEK
PEPTIDE:

[SEQ ID NO: 1202]

KGPEQYAEWQINEK
PEPTIDE:

[SEQ ID NO: 1203]

KGPQEYAEWQINEK
PEPTIDE:

[SEQ ID NO: 1204]

KGPQQFAEWQINEK
PEPTIDE:

[SEQ ID NO: 1205]

RGPEQFAEWQINEK
PEPTIDE:

[SEQ ID NO: 1206]

KGPQQYAEWQINER
PEPTIDE:

[SEQ ID NO: 1207]

RGPQQYAEWQINDR
PEPTIDE:

[SEQ ID NO: 1208]

RGPQQYADWQINDK
PEPTIDE:

[SEQ ID NO: 1209]

RGPQQFAEWQINER
PEPTIDE:

[SEQ ID NO: 1210]

RGPQQYAEWQVNEK
PEPTIDE:

[SEQ ID NO: 1211]

RGPQQFAEWQINEK
PEPTIDE:

[SEQ ID NO: 1212]

KGPQQFAEWQINER
PEPTIDE:

[SEQ ID NO: 1213]

KGPQQFAEWQVNEK
PEPTIDE:

[SEQ ID NO: 1214]

RGPQQFAEWQVNDK
PEPTIDE:

[SEQ ID NO: 1215]

RGPQQYADWQINDR
PEPTIDE:

[SEQ ID NO: 1216]

KGPQQYADWQINDK
PEPTIDE:

[SEQ ID NO: 1217]

RGPQQFADYQINEK
PEPTIDE:

[SEQ ID NO: 1218]

RGPQQYARWQINEK
PEPTIDE:

[SEQ ID NO: 1219]

RGPQQYAEWQINEE
PEPTIDE:

[SEQ ID NO: 1220]

HGPQQYAEWQINEK
PEPTIDE:

[SEQ ID NO: 1221]

RGPYQYAEWQINEK
PEPTIDE:

[SEQ ID NO: 1222]

RGPQQYMEWQINEK
PEPTIDE:

[SEQ ID NO: 1223]

RGPQQYAEWQINEK
PEPTIDE:

[SEQ ID NO: 1224]

RGPQQYAEWCINEK
PEPTIDE:

[SEQ ID NO: 1225]

RGPQPYAEWQINEK
PEPTIDE:

[SEQ ID NO: 1226]

RGGQQYAEWQINED
PEPTIDE:

[SEQ ID NO: 1227]

RGPQQYARWKINEK
PEPTIDE:

[SEQ ID NO: 1228]

RGGQQYAETQINEK
PEPTIDE:

[SEQ ID NO: 1229]

RGPLQYAEWQNNEK
PEPTIDE:

[SEQ ID NO: 1230]

EGPQQYAEWQINED
PEPTIDE:

[SEQ ID NO: 1231]

RGPQQYAEWQINLL
PEPTIDE:

[SEQ ID NO: 1232]

RGPQQGGEWQINEK
PEPTIDE:

[SEQ ID NO: 1233]

RGPQQYAEWQIGGG
PEPTIDE:

[SEQ ID NO: 1234]

RGPQQKYEWQINEK
PEPTIDE:

[SEQ ID NO: 1235]

RGPQAQYEWQINEK
PEPTIDE:

[SEQ ID NO: 1236]

RPHQQYAEWQINEK
PEPTIDE:

[SEQ ID NO: 1237]

RGPQHHHEWQINEK
PEPTIDE:

[SEQ ID NO: 1238]

RGPPQYAPPQINEK
PEPTIDE:

[SEQ ID NO: 1239]

RGPQCYYEWCINEK
PEPTIDE:

[SEQ ID NO: 1240]

RGPTQYAEGQINEG
PEPTIDE:

[SEQ ID NO: 1241]

RGPQQYAEWQINEKG
PEPTIDE:

[SEQ ID NO: 1242]

RGPQQYAEWQINEKY
PEPTIDE:

[SEQ ID NO: 1243]

RGPQQYAFTEWQINEK
PEPTIDE:

[SEQ ID NO: 1244]

RGPQSQYAEWQINEKPM
PEPTIDE:

[SEQ ID NO: 1245]

RGPQQYAEWQINEKKK
PEPTIDE:

[SEQ ID NO: 1246]

RRRRGPQQYAEWQINEK
PEPTIDE:

[SEQ ID NO: 1247]

RGPQQYAEWQINE
PEPTIDE:

[SEQ ID NO: 1248]

RGPQQYAEWQIN
PEPTIDE:

[SEQ ID NO: 1249]

RGPQQYAEWQI
PEPTIDE:

[SEQ ID NO: 1250]

GPQQYAEWQINEK
PEPTIDE:

[SEQ ID NO: 1251]

PQQYAEWQINEK
PEPTIDE:

[SEQ ID NO: 1252]

QQYAEWQINEK
PEPTIDE:

[SEQ ID NO: 1253]

QQYAEWQI
PEPTIDE:

[SEQ ID NO: 1254]

PQQYAEWQINE
PEPTIDE:

[SEQ ID NO: 1255]

PQQYAEWQIN
PEPTIDE:

[SEQ ID NO: 1256]

RGPQQYA
PEPTIDE:

[SEQ ID NO: 1257]

EWQINEK
PEPTIDE:

[SEQ ID NO: 1258]

QSFILSGNE
PEPTIDE:

[SEQ ID NO: 1259]

ESFLLSGNQ
PEPTIDE:

[SEQ ID NO: 1260]

QSYLLSGNQ
PEPTIDE:

[SEQ ID NO: 1261]

QSFLLSGDQ
PEPTIDE:

[SEQ ID NO: 1262]

QSYLLSGNE
PEPTIDE:

[SEQ ID NO: 1263]

ESFLLSGNE
PEPTIDE:

[SEQ ID NO: 1264]

ESYLLSGNQ
PEPTIDE:

[SEQ ID NO: 1265]

QSFLLSGDE
PEPTIDE:

[SEQ ID NO: 1266]

QSYLLSGDQ
PEPTIDE:

[SEQ ID NO: 1267]

QSFRLSGNQ
PEPTIDE:

[SEQ ID NO: 1268]

QSFLLSYNQ
PEPTIDE:

[SEQ ID NO: 1269]

QFFLLSGNQ
PEPTIDE:

[SEQ ID NO: 1270]

QSFLLSGAQ
PEPTIDE:

[SEQ ID NO: 1271]

QSFLLSGNP
PEPTIDE:

[SEQ ID NO: 1272]

QSFRRSGNQ
PEPTIDE:

[SEQ ID NO: 1273]

QSFLLSYIQ
PEPTIDE:

[SEQ ID NO: 1274]

QFFLLSGNL
PEPTIDE:

[SEQ ID NO: 1275]

QSFLLSGAQ
PEPTIDE:

[SEQ ID NO: 1276]

QSFLLSGNP
PEPTIDE:

[SEQ ID NO: 1277]

QSFLLSGNQQ
PEPTIDE:

[SEQ ID NO: 1278]

QSFLLLSGNQ
PEPTIDE:

[SEQ ID NO: 1279]

AQSFGLLSGNQ
PEPTIDE:

[SEQ ID NO: 1280]

RQSFLLISGNQ
PEPTIDE:

[SEQ ID NO: 1281]

QSFLLSGNQK
PEPTIDE:

[SEQ ID NO: 1282]

QFLLSGNQ
PEPTIDE:

[SEQ ID NO: 1283]

SFLLSGNQ
PEPTIDE:

[SEQ ID NO: 1284]

QSFLLSGN
PEPTIDE:

[SEQ ID NO: 1285]

QSFLLGNQ
PEPTIDE:

[SEQ ID NO: 1286]

QSFLSGNQ
PEPTIDE:

[SEQ ID NO: 1287]

QSLLSGNQ
PEPTIDE:

[SEQ ID NO: 1288]

VLDLAIPVNRPGQ
PEPTIDE:

[SEQ ID NO: 1289]

VLDLAIPVNRPG
PEPTIDE:

[SEQ ID NO: 1290]

VLDLAIPVNRP
PEPTIDE:

[SEQ ID NO: 1291]

LDLAIPVNRPGQL
PEPTIDE:

[SEQ ID NO: 1292]

DLAIPVNRPGQL
PEPTIDE:

[SEQ ID NO: 1293]

LAIPVNRPGQL
PEPTIDE:

[SEQ ID NO: 1294]

LDLAIPVNRPGQ
PEPTIDE:

[SEQ ID NO: 1295]

DLAIPVNRPG
PEPTIDE:

[SEQ ID NO: 1296]

LAIPVNRP
PEPTIDE:

[SEQ ID NO: 1297]

VLDLAIPVN
PEPTIDE:

[SEQ ID NO: 1298]

AIPVNRPGQL
PEPTIDE:

[SEQ ID NO: 1299]

VNRPGQL
PEPTIDE:

[SEQ ID NO: 1300]

VLDLAIPV
PEPTIDE:

[SEQ ID NO: 1301]

VLDLAIPVNR
PEPTIDE:

[SEQ ID NO: 1302]

QSFLLSGN
PEPTIDE:

[SEQ ID NO: 1303]

QSFLLSG
PEPTIDE:

[SEQ ID NO: 1304]

QSFLLS
PEPTIDE:

[SEQ ID NO: 1305]

SFLLSGNQ
PEPTIDE:

[SEQ ID NO: 1306]

FLLSGNQ
PEPTIDE:

[SEQ ID NO: 1307]

LLSGNQ
PEPTIDE:

[SEQ ID NO: 1308]

SFLLSGN
PEPTIDE:

[SEQ ID NO: 1309]

DTFYNAAWDPSNR
PEPTIDE:

[SEQ ID NO: 1310]

ALDWAIANLLR
PEPTIDE:

[SEQ ID NO: 1311]

YDYENVDAGAAK
PEPTIDE:

[SEQ ID NO: 1312]

EVQDSPLDACR
PEPTIDE:

[SEQ ID NO: 1313]

ITSVNSQKFPILNLIQMSATR
PEPTIDE:

[SEQ ID NO: 1314]

SRVQVVSNFGK
PEPTIDE:

[SEQ ID NO: 1315]

VLDLAIPVNKPGQLQSFLLSGTQNQPSLLSGFSK
PEPTIDE:

[SEQ ID NO: 1316]

NAMFVPHYNLNANSIIYALKGR
PEPTIDE:

[SEQ ID NO: 1317]

LRPGVMFVVPAGHPFVNIASK
PEPTIDE:

[SEQ ID NO: 1318]

GLFDLGHPLVNR
PEPTIDE:

[SEQ ID NO: 1319]

RPYYSNAPQEIF
PEPTIDE:

[SEQ ID NO: 1320]

VLLEQQEQEPQH
PEPTIDE:

[SEQ ID NO: 1321]

QQYGIAASPFLQSAA

1. A man made composition comprising a plurality of peptides, in which at least one of the peptides includes SEQUENCE ID NO 555 and another of the peptides includes SEQUENCE ID NO: 701.

2. A man-made composition according to claim 1 in which the peptides are selected from SEQUENCE ID NO 555 and SEQUENCE IS NO: 701.

3. A man-made composition according to claim 1 including a further peptide that comprises a sequence selected from SEQUENCE ID NO's 5, 23, 22, 38, 39, 21, 258, 242, 261, 211, 222, 249, 235, 295, 283, 284, and 216.

4. A man-made composition according to claim 1 including peptides comprising SEQUENCE ID NO's 5, 23, 22, 38, 39, 21, 258, 242, 261, 211, 222, 249, 235, 295, 283, 284, and 216.

5. A man-made composition according to claim 1 including peptides of SEQUENCE ID NO's 5, 23, 22, 38, 39, 21, 258, 242, 261, 211, 222, 249, 235, 295, 283, 284, and 216.

6. A man-made composition according to claim 1 that is enriched in peptides having a molecular weight of less than 10 kD.

7. A man-made composition according to claim 1 that is a powder.

8. A man-made composition according to claim 1 that is a liquid or cream, in which the liquid or cream has a PH of 6-8.

9. A food product comprising a composition of claim 1.

10. A man-made composition according to claim 1 that is a dietary supplement.

11. A method of treating inflammation in a mammal comprising a step of topically or orally administering a therapeutically effective amount of a composition of claim 1 to the mammal.

12. A method of preventing ageing of skin in a mammal, comprising a step of topically administering a therapeutically effective amount of the composition of claim 1 to the skin of the mammal.

13. A method of preventing growth of bacteria on a surface comprising a step of contacting the surface with the composition of claim 1.

14. An isolated peptide having 6 to 50 amino acids and comprising a sequence selected from SEQUENCE ID NO: 5, 21, 22, 23, 38, 39, 40, 41 and 74.

15. An isolated peptide according to claim 14 selected from SEQUENCE ID NO: 5, 21, 22, 23, 38, 39, 40, 41 and 74.

16. An isolated peptide according to claim 14, which is a modified peptide, in which the modification is configured to increase the blood plasma half-life of the modified peptide relative to the unmodified peptide; the lipophilicity of the modified peptide relative to the unmodified peptide, or the stability of the modified peptide relative to the unmodified peptide.

17. A conjugate comprising an isolated peptide of claim 14 conjugated to a binding partner.


SEQUENCE LISTING (1321 sequences)

SEQ ID NO: 1 - protein, 12 aa, Artificial Sequence
  Description of Artificial Sequence Synthetic peptide
SRGPIYSNEFGK

SEQ ID NO: 2 - protein, 7 aa, Artificial Sequence
  Description of Artificial Sequence Synthetic peptide
NSFNLER

SEQ ID NO: 3 - protein, 10 aa, Artificial Sequence
  Description of Artificial Sequence Synthetic peptide
VLDLAIPVNR

SEQ ID NO: 4 - protein, 7 aa, Artificial Sequence
  Description of Artificial Sequence Synthetic peptide
DDNEELR

SEQ ID NO: 5 - protein, 18 aa, Artificial Sequence
  Description of Artificial Sequence Synthetic peptide
LSSGDVFVIPAGHPVAVK

SEQ ID NO: 6 - protein, 15 aa, Artificial Sequence
  Description of Artificial Sequence Synthetic peptide
EDDEEEEQGEEEINK

SEQ ID NO: 7 - protein, 29 aa, Artificial Sequence
  Description of Artificial Sequence Synthetic peptide
NILEASFNTDYEEIEKVLLEEHEKETQHR

SEQ ID NO: 8 - protein, 24 aa, Artificial Sequence
  Description of Artificial Sequence Synthetic peptide
NILEASFNTDYEEIEKVLLEEHEK

SEQ ID NO: 9 - protein, 16 aa, Artificial Sequence
  Description of Artificial Sequence Synthetic peptide
NILEASFNTDYEEIEK

SEQ ID NO: 10 - protein, 12 aa, Artificial Sequence
  Description of Artificial Sequence Synthetic peptide
RQQSQEENVIVK

SEQ ID NO: 11 - protein, 11 aa, Artificial Sequence
  Description of Artificial Sequence Synthetic peptide
QQSQEENVIVK

SEQ ID NO: 12 - protein, 11 aa, Artificial Sequence
  Description of Artificial Sequence Synthetic peptide
LSRGQIEELSK

SEQ ID NO: 13 - protein, 8 aa, Artificial Sequence
  Description of Artificial Sequence Synthetic peptide
GQIEELSK

SEQ ID NO: 14 - protein, 8 aa, Artificial Sequence
  Description of Artificial Sequence Synthetic peptide
VLLEEHEK

SEQ ID NO: 15 - protein, 24 aa, Artificial Sequence
  Description of Artificial Sequence Synthetic peptide
SKPHTIFLPQHTDADYILVVLSGK

SEQ ID NO: 16 - protein, 22 aa, Artificial Sequence
  Description of Artificial Sequence Synthetic peptide
PHTIFLPQHTDADYILVVLSGK

SEQ ID NO: 17 - protein, 16 aa, Artificial Sequence
  Description of Artificial Sequence Synthetic peptide
SNKFQTLFENENGHIR

SEQ ID NO: 18 - protein, 11 aa, Artificial Sequence
  Description of Artificial Sequence Synthetic peptide
SKIFENLQNYR

SEQ ID NO: 19 - protein, 9 aa, Artificial Sequence
  Description of Artificial Sequence Synthetic peptide
IFENLQNYR

SEQ ID NO: 20 - protein, 18 aa, Artificial Sequence
  Description of Artificial Sequence Synthetic peptide
ILENQKQSHFADAQPQQR

SEQ ID NO: 21 - protein, 24 aa, Artificial Sequence
  Description of Artificial Sequence Synthetic peptide
PGQLQSFLLSGNQNQQNYLSGFSK

SEQ ID NO: 22 - protein, 25 aa, Artificial Sequence
  Description of Artificial Sequence Synthetic peptide
FFELTPEKNQQLQDLDLFVNSVDLK

SEQ ID NO: 23 - protein, 7 aa, Artificial Sequence
  Description of Artificial Sequence Synthetic peptide
QLEELSK

SEQ ID NO: 24 - protein, 11 aa, Artificial Sequence
  Description of Artificial Sequence Synthetic peptide
QEEDEDEDEER

SEQ ID NO: 25 - protein, 23 aa, Artificial Sequence
  Description of Artificial Sequence Synthetic peptide
YQHQQGGKQEQENEGNNIFSGFK

SEQ ID NO: 26 - protein, 24 aa, Artificial Sequence
  Description of Artificial Sequence Synthetic peptide
GDTIKLPAGTTSYLVNQDDEEDLR

SEQ ID NO: 27 - protein, 12 aa, Artificial Sequence
  Description of Artificial Sequence Synthetic peptide
RQQGEETDAIVK

SEQ ID NO: 28 - protein, 11 aa, Artificial Sequence
  Description of Artificial Sequence Synthetic peptide
VLLEEQEKDRK

SEQ ID NO: 29 - protein, 10 aa, Artificial Sequence
  Description of Artificial Sequence Synthetic peptide
NILEASYNTR

SEQ ID NO: 30 - protein, 8 aa, Artificial Sequence
  Description of Artificial Sequence Synthetic peptide
FEAFDLAK

SEQ ID NO: 31 - protein, 8 aa, Artificial Sequence
  Description of Artificial Sequence Synthetic peptide
EQIEELKK

SEQ ID NO: 32 - protein, 7 aa, Artificial Sequence
  Description of Artificial Sequence Synthetic peptide
EQIEELK

SEQ ID NO: 33 - protein, 7 aa, Artificial Sequence
  Description of Artificial Sequence Synthetic peptide
NKNQYLR

SEQ ID NO: 34 - protein, 37 aa, Artificial Sequence
  Description of Artificial Sequence Synthetic polypeptide
LSPGDVVIIPAGHPVAITASSNLNLLGFGINAENNER

SEQ ID NO: 35 - protein, 14 aa, Artificial Sequence
  Description of Artificial Sequence Synthetic peptide
PSYEKQEDEEEKQK

SEQ ID NO: 36 - protein, 9 aa, Artificial Sequence
  Description of Artificial Sequence Synthetic peptide
EEDEEEGQR

SEQ ID NO: 37 - protein, 20 aa, Artificial Sequence
  Description of Artificial Sequence Synthetic peptide
TLFLPQYTDADFILVVLSGK

SEQ ID NO: 38 - protein, 21 aa, Artificial Sequence
  Description of Artificial Sequence Synthetic peptide
LVDLVIPVNGPGKFEAFDLAK

SEQ ID NO: 39 - protein, 18 aa, Artificial Sequence
  Description of Artificial Sequence Synthetic peptide
KNPQLQDLDIFVNYVEIK

SEQ ID NO: 40 - protein, 24 aa, Artificial Sequence
  Description of Artificial Sequence Synthetic peptide
GYVGLTFPGCPATHQQQFQLFEQR

SEQ ID NO: 41 - protein, 14 aa, Artificial Sequence
  Description of Artificial Sequence Synthetic peptide
RGPQQYAEWQINEK

SEQ ID NO: 42 - protein, 10 aa, Artificial Sequence
  Description of Artificial Sequence Synthetic peptide
DEHQKIHQFR

SEQ ID NO: 43 - protein, 7 aa, Artificial Sequence
  Description of Artificial Sequence Synthetic peptide
FRDEHQK

SEQ ID NO: 44 - protein, 13 aa, Artificial Sequence
  Description of Artificial Sequence Synthetic peptide
FPILNLIQMSATR

SEQ ID NO: 45 - protein, 13 aa, Artificial Sequence
  Description of Artificial Sequence Synthetic peptide
TNANAFVSHLAGK

SEQ ID NO: 46 - protein, 12 aa, Artificial Sequence
  Description of Artificial Sequence Synthetic peptide
ALPVDVVANAYR

SEQ ID NO: 47 - protein, 20 aa, Artificial Sequence
  Description of Artificial Sequence Synthetic peptide
YVYDVNNNANQLEPRQKEFL

SEQ ID NO: 48 - protein, 21 aa, Artificial Sequence
  Description of Artificial Sequence Synthetic peptide
VYVYDVNNNANQLEPRQKEFL

SEQ ID NO: 49 - protein, 7 aa, Artificial Sequence
  Description of Artificial Sequence Synthetic peptide
ADSYNPR

SEQ ID NO: 50 - protein, 16 aa, Artificial Sequence
  Description of Artificial Sequence Synthetic peptide
KPTLTQQQEQAQAQDQ

SEQ ID NO: 51 - protein, 13 aa, Artificial Sequence
  Description of Artificial Sequence Synthetic peptide
QAQAQDQYQQVQY

SEQ ID NO: 52 - protein, 11 aa, Artificial Sequence
  Description of Artificial Sequence Synthetic peptide
QAQDQYQQVQY

SEQ ID NO: 53 - protein, 8 aa, Artificial Sequence
  Description of Artificial Sequence Synthetic peptide
LQAFEPLR

SEQ ID NO: 54 - protein, 11 aa, Artificial Sequence
  Description of Artificial Sequence Synthetic peptide
SRVQVVSNFGK

SEQ ID NO: 55 - protein, 10 aa, Artificial Sequence
  Description of Artificial Sequence Synthetic peptide
WNVNAHSLVY

SEQ ID NO: 56 - protein, 9 aa, Artificial Sequence
  Description of Artificial Sequence Synthetic peptide
NVNAHSLVY

SEQ ID NO: 57 - protein, 15 aa, Artificial Sequence
  Description of Artificial Sequence Synthetic peptide
IQGRSRVQVVSNFGK

SEQ ID NO: 58 - protein, 14 aa, Artificial Sequence
  Description of Artificial Sequence Synthetic peptide
GKTVFDGVLRPGQL

SEQ ID NO: 59 - protein, 15 aa, Artificial Sequence
  Description of Artificial Sequence Synthetic peptide
FGKTVFDGVLRPGQL

SEQ ID NO: 60 - protein, 19 aa, Artificial Sequence
  Description of Artificial Sequence Synthetic peptide
FQQQYYPGLSNESESETSE

SEQ ID NO: 61 - protein, 9 aa, Artificial Sequence
  Description of Artificial Sequence Synthetic peptide
QQYYPGLSN

SEQ ID NO: 62 - protein, 10 aa, Artificial Sequence
  Description of Artificial Sequence Synthetic peptide
QQQYYPGLSN

SEQ ID NO: 63 - protein, 21 aa, Artificial Sequence
  Description of Artificial Sequence Synthetic peptide
VTNLNTQNFPILSLVQMSAVK

SEQ ID NO: 64 - protein, 19 aa, Artificial Sequence
  Description of Artificial Sequence Synthetic peptide
ITQGRARVQVVNNNGKTVF

SEQ ID NO: 65 - protein, 22 aa, Artificial Sequence
  Description of Artificial Sequence Synthetic peptide
ITQGRARVQVVNNNGKTVFNGE

SEQ ID NO: 66 - protein, 21 aa, Artificial Sequence
  Description of Artificial Sequence Synthetic peptide
ITQGRARVQVVNNNGKTVFNG

SEQ ID NO: 67 - protein, 13 aa, Artificial Sequence
  Description of Artificial Sequence Synthetic peptide
RVQVVNNNGKTVF

SEQ ID NO: 68 - protein, 18 aa, Artificial Sequence
  Description of Artificial Sequence Synthetic peptide
RALPNDVLANAYRISREE

SEQ ID NO: 69 - protein, 16 aa, Artificial Sequence
  Description of Artificial Sequence Synthetic peptide
SIFRALPNDVLANAYR

SEQ ID NO: 70 - protein, 15 aa, Artificial Sequence
  Description of Artificial Sequence Synthetic peptide
SIFRALPNDVLANAY

SEQ ID NO: 71 - protein, 13 aa, Artificial Sequence
  Description of Artificial Sequence Synthetic peptide
SIFRALPNDVLAN

SEQ ID NO: 72 - protein, 17 aa, Artificial Sequence
  Description of Artificial Sequence Synthetic peptide
SSIFRALPNDVLANAYR

SEQ ID NO: 73 - protein, 21 aa, Artificial Sequence
  Description of Artificial Sequence Synthetic peptide
SIFRALPNDVLANAYRISREE

SEQ ID NO: 74 - protein, 9 aa, Artificial Sequence
  Description of Artificial Sequence Synthetic peptide
SIFRALPND

SEQ ID NO: 75 - protein, 19 aa, Artificial Sequence
  Description of Artificial Sequence Synthetic peptide
IYVTDLNNGANQLDPRQRD

SEQ ID NO: 76 - protein, 21 aa, Artificial Sequence
  Description of Artificial Sequence Synthetic peptide
VTNLNSQNFPILNLVQMSAVK

SEQ ID NO: 77 - protein, 8 aa, Artificial Sequence
  Description of Artificial Sequence Synthetic peptide
QNIDNPNR

SEQ ID NO: 78 - protein, 7 aa, Artificial Sequence
  Description of Artificial Sequence Synthetic peptide
ADTYNPR

SEQ ID NO: 79 - protein, 21 aa, Artificial Sequence
  Description of Artificial Sequence Synthetic peptide
NIDNPNRADTYNPRAGRVTNL

SEQ ID NO: 80 - protein, 25 aa, Artificial Sequence
  Description of Artificial Sequence Synthetic peptide
RVRQNIDNPNRADTYNPRAGRVTNL

SEQ ID NO: 81 - protein, 18 aa, Artificial Sequence
  Description of Artificial Sequence Synthetic peptide
TNPNSMVSHIAGKSSIFR

SEQ ID NO: 82 - protein, 16 aa, Artificial Sequence
  Description of Artificial Sequence Synthetic peptide
HNRGDEFGAFTPLQYK

SEQ ID NO: 83 - protein, 12 aa, Artificial Sequence
  Description of Artificial Sequence Synthetic peptide
SYQDVYNVAESS

SEQ ID NO: 84 - protein, 8 aa, Artificial Sequence
  Description of Artificial Sequence Synthetic peptide
ISREEAQR

SEQ ID NO: 85 - protein, 21 aa, Artificial Sequence
  Description of Artificial Sequence Synthetic peptide
SIFRALPTDVLANAYRISREE

SEQ ID NO: 86 - protein, 19 aa, Artificial Sequence
  Description of Artificial Sequence Synthetic peptide
YRISREEAQRLKHNRGDEF

SEQ ID NO: 87 - protein, 18 aa, Artificial Sequence
  Description of Artificial Sequence Synthetic peptide
YRISREEAQRLKHNRGDE

SEQ ID NO: 88 - protein, 10 aa, Artificial Sequence
  Description of Artificial Sequence Synthetic peptide
FKDEHQKIHR

SEQ ID NO: 89 - protein, 14 aa, Artificial Sequence
  Description of Artificial Sequence Synthetic peptide
QGDVIALPAGVAHW

SEQ ID NO: 90 - protein, 9 aa, Artificial Sequence
  Description of Artificial Sequence Synthetic peptide
TVFNGELRR

SEQ ID NO: 91 - protein, 8 aa, Artificial Sequence
  Description of Artificial Sequence Synthetic peptide
TVFNGELR

SEQ ID NO: 92 - protein, 13 aa, Artificial Sequence
  Description of Artificial Sequence Synthetic peptide
QVQVVNNNGKTVF

SEQ ID NO: 93 - protein, 13 aa, Artificial Sequence
  Description of Artificial Sequence Synthetic peptide
YIIQGRGITGPTF

SEQ ID NO: 94 - protein, 14 aa, Artificial Sequence
  Description of Artificial Sequence Synthetic peptide
VYIIQGRGITGPTF

SEQ ID NO: 95 - protein, 11 aa, Artificial Sequence
  Description of Artificial Sequence Synthetic peptide
LQAFEPIRSVR

SEQ ID NO: 96 - protein, 22 aa, Artificial Sequence
  Description of Artificial Sequence Synthetic peptide
GLSLLQPYASLQEQEQGQMQSR

SEQ ID NO: 97 - protein, 8 aa, Artificial Sequence
  Description of Artificial Sequence Synthetic peptide
GEIVRVER

SEQ ID NO: 98 - protein, 13 aa, Artificial Sequence
  Description of Artificial Sequence Synthetic peptide
RGLSLLQPYASLQ

SEQ ID NO: 99 - protein, 15 aa, Artificial Sequence
  Description of Artificial Sequence Synthetic peptide
RGLSLLQPYASLQEQ

SEQ ID NO: 100 - protein, 16 aa, Artificial Sequence
  Description of Artificial Sequence Synthetic peptide
RGLSLLQPYASLQEQE

SEQ ID NO: 101 - protein, 14 aa, Artificial Sequence
  Description of Artificial Sequence Synthetic peptide
RGLSLLQPYASLQE

SEQ ID NO: 102 - protein, 7 aa, Artificial Sequence
  Description of Artificial Sequence Synthetic peptide
RNPQAYR

SEQ ID NO: 103 - protein, 13 aa, Artificial Sequence
  Description of Artificial Sequence Synthetic peptide
FLLAGNKRNPQAY

SEQ ID NO: 104 - protein, 10 aa, Artificial Sequence
  Description of Artificial Sequence Synthetic peptide
EVEEWSQNIF

SEQ ID NO: 105 - protein, 12 aa, Artificial Sequence
  Description of Artificial Sequence Synthetic peptide
LAGNKRNPQAYR

SEQ ID NO: 106 - protein, 12 aa, Artificial Sequence
  Description of Artificial Sequence Synthetic peptide
FLLAGNKRNPQA

SEQ ID NO: 107 - protein, 13 aa, Artificial Sequence
  Description of Artificial Sequence Synthetic peptide
ELGAPDVGHPMSE

SEQ ID NO: 108 - protein, 16 aa, Artificial Sequence
  Description of Artificial Sequence Synthetic peptide
IVQGHARVQVVSNLGK

SEQ ID NO: 109 - protein, 14 aa, Artificial Sequence
  Description of Artificial Sequence Synthetic peptide
IVQGHARVQVVSNL

SEQ ID NO: 110 - protein, 13 aa, Artificial Sequence
  Description of Artificial Sequence Synthetic peptide
IVQGHARVQVVSN

SEQ ID NO: 111 - protein, 13 aa, Artificial Sequence
  Description of Artificial Sequence Synthetic peptide
NNRGEELGAFTPR

SEQ ID NO: 112 - protein, 10 aa, Artificial Sequence
  Description of Artificial Sequence Synthetic peptide
GEELGAFTPR

SEQ ID NO: 113 - protein, 13 aa, Artificial Sequence
  Description of Artificial Sequence Synthetic peptide
FPILNLVQLSATR

SEQ ID NO: 114 - protein, 31 aa, Artificial Sequence
  Description of Artificial Sequence Synthetic polypeptide
SIEQHSGQNIFSGFNNELLSEALGVNALVAK

SEQ ID NO: 115 - protein, 8 aa, Artificial Sequence
  Description of Artificial Sequence Synthetic peptide
LQGQNDQR

SEQ ID NO: 116 - protein, 20 aa, Artificial Sequence
  Description of Artificial Sequence Synthetic peptide
SGFNNELLSEALGVNALVAK

SEQ ID NO: 117 - protein, 20 aa, Artificial Sequence
  Description of Artificial Sequence Synthetic peptide
PAFAQQQEQAQQQEQAQAQY

SEQ ID NO: 118 - protein, 15 aa, Artificial Sequence
  Description of Artificial Sequence Synthetic peptide
VAKRLQGQNDQRGEI

SEQ ID NO: 119 - protein, 17 aa, Artificial Sequence
  Description of Artificial Sequence Synthetic peptide
ALVAKRLQGQNDQRGEI

SEQ ID NO: 120 - protein, 13 aa, Artificial Sequence
  Description of Artificial Sequence Synthetic peptide
LQGQNDQRGEIIR

SEQ ID NO: 121 - protein, 14 aa, Artificial Sequence
  Description of Artificial Sequence Synthetic peptide
PNVNPWHNPRQGGF

SEQ ID NO: 122 - protein, 13 aa, Artificial Sequence
  Description of Artificial Sequence Synthetic peptide
FYNEGDAPVVALY

SEQ ID NO: 123 - protein, 10 aa, Artificial Sequence
  Description of Artificial Sequence Synthetic peptide
FYNEGDAPVV

SEQ ID NO: 124 - protein, 12 aa, Artificial Sequence
  Description of Artificial Sequence Synthetic peptide
FYNEGDAPVVAL

SEQ ID NO: 125 - protein, 11 aa, Artificial Sequence
  Description of Artificial Sequence Synthetic peptide
FYNEGDAPVVA

SEQ ID NO: 126 - protein, 13 aa, Artificial Sequence
  Description of Artificial Sequence Synthetic peptide
TNANSMVSHLAGK

SEQ ID NO: 127 - protein, 12 aa, Artificial Sequence
  Description of Artificial Sequence Synthetic peptide
AMPVDVIANAYR

SEQ ID NO: 128 - protein, 28 aa, Artificial Sequence
  Description of Artificial Sequence Synthetic peptide
NWENVLLGLGVAGSAPGIEGDEIAPLAK

SEQ ID NO: 129 - protein, 19 aa, Artificial Sequence
  Description of Artificial Sequence Synthetic peptide
NVLLGLGVAGSAPGIEGDE

SEQ ID NO: 130 - protein, 28 aa, Artificial Sequence
  Description of Artificial Sequence Synthetic peptide
NWENVLLGLGVAGSAPGIEGDEIAPLAK

SEQ ID NO: 131 - protein, 21 aa, Artificial Sequence
  Description of Artificial Sequence Synthetic peptide
FNAPLAHLIMAGADVLAVPSR

SEQ ID NO: 132 - protein, 10 aa, Artificial Sequence
  Description of Artificial Sequence Synthetic peptide
FNAPLAHLIM

SEQ ID NO: 133 - protein, 21 aa, Artificial Sequence
  Description of Artificial Sequence Synthetic peptide
FNAPLAHLIMAGADVLAVPSR

SEQ ID NO: 134 - protein, 12 aa, Artificial Sequence
  Description of Artificial Sequence Synthetic peptide
VVGTPAYEEMVR

SEQ ID NO: 135 - protein, 21 aa, Artificial Sequence
  Description of Artificial Sequence Synthetic peptide
TGGLGDVLGGLPPAMAANGHR

SEQ ID NO: 136 - protein, 17 aa, Artificial Sequence
  Description of Artificial Sequence Synthetic peptide
YDQYKDAWDTSVVAEIK

SEQ ID NO: 137 - protein, 12 aa, Artificial Sequence
  Description of Artificial Sequence Synthetic peptide
DAWDTSVVAEIK

SEQ ID NO: 138 - protein, 7 aa, Artificial Sequence
  Description of Artificial Sequence Synthetic peptide
VMVISPR

SEQ ID NO: 139 - protein, 22 aa, Artificial Sequence
  Description of Artificial Sequence Synthetic peptide
LTGITGIVNGMDVSEWDPSKDK

SEQ ID NO: 140 - protein, 18 aa, Artificial Sequence
  Description of Artificial Sequence Synthetic peptide
VLTVSPYYAEELISGIAR

SEQ ID NO: 141 - protein, 14 aa, Artificial Sequence
  Description of Artificial Sequence Synthetic peptide
EALQAEAGLPVDRK

SEQ ID NO: 142 - protein, 10 aa, Artificial Sequence
  Description of Artificial Sequence Synthetic peptide
YDATTAIEAK

SEQ ID NO: 143 - protein, 9 aa, Artificial Sequence
  Description of Artificial Sequence Synthetic peptide
IPLIAFIGR

SEQ ID NO: 144 - protein, 8 aa, Artificial Sequence
  Description of Artificial Sequence Synthetic peptide
AGILEADR

SEQ ID NO: 145 - protein, 9 aa, Artificial Sequence
  Description of Artificial Sequence Synthetic peptide
IPLIAFIGR

SEQ ID NO: 146 - protein, 11 aa, Artificial Sequence
  Description of Artificial Sequence Synthetic peptide
VFIDHPSFLEK

SEQ ID NO: 147 - protein, 13 aa, Artificial Sequence
  Description of Artificial Sequence Synthetic peptide
GPDTGVDYKDNQM

SEQ ID NO: 148 - protein, 16 aa, Artificial Sequence
  Description of Artificial Sequence Synthetic peptide
IYGPDTGVDYKDNQMR

SEQ ID NO: 149 - protein, 11 aa, Artificial Sequence
  Description of Artificial Sequence Synthetic peptide
IYGPDTGVDYK

SEQ ID NO: 150 - protein, 11 aa, Artificial Sequence
  Description of Artificial Sequence Synthetic peptide
ILNLNNNPYFK

SEQ ID NO: 151 - protein, 16 aa, Artificial Sequence
  Description of Artificial Sequence Synthetic peptide
APTGTFIASGVVVGKD

SEQ ID NO: 152 - protein, 13 aa, Artificial Sequence
  Description of Artificial Sequence Synthetic peptide
QNYLSGFSKNILE

SEQ ID NO: 153 - protein, 20 aa, Artificial Sequence
  Description of Artificial Sequence Synthetic peptide
TIKLPAGTIAYLVNRDDNEE

SEQ ID NO: 154 - protein, 15 aa, Artificial Sequence
  Description of Artificial Sequence Synthetic peptide
LAIPVNRPGQLQSFL

SEQ ID NO: 155 - protein, 11 aa, Artificial Sequence
  Description of Artificial Sequence Synthetic peptide
AIPVNRPGQLQ

SEQ ID NO: 156 - protein, 9 aa, Artificial Sequence
  Description of Artificial Sequence Synthetic peptide
PAGHPVAVK

SEQ ID NO: 157 - protein, 19 aa, Artificial Sequence
  Description of Artificial Sequence Synthetic peptide
VQNYKAKLSSGDVFVIPAG

SEQ ID NO: 158 - protein, 24 aa, Artificial Sequence
  Description of Artificial Sequence Synthetic peptide
NNQRNFLAGDEDNVISQIQRPVKE

SEQ ID NO: 159 - protein, 23 aa, Artificial Sequence
  Description of Artificial Sequence Synthetic peptide
INKQVQNYKAKLSSGDVFVIPAG

SEQ ID NO: 160 - protein, 10 aa, Artificial Sequence
  Description of Artificial Sequence Synthetic peptide
LAIPVNRPGQ

SEQ ID NO: 161 - protein, 20 aa, Artificial Sequence
  Description of Artificial Sequence Synthetic peptide
NFLAGDEDNVISQIQRPVKE

SEQ ID NO: 162 - protein, 15 aa, Artificial Sequence
  Description of Artificial Sequence Synthetic peptide
DLAIPVNRPGQLQSF

SEQ ID NO: 163 - protein, 11 aa, Artificial Sequence
  Description of Artificial Sequence Synthetic peptide
VIPAGHPVAVK

SEQ ID NO: 164 - protein, 21 aa, Artificial Sequence
  Description of Artificial Sequence Synthetic peptide
DTIKLPAGTIAYLVNRDDNEE

SEQ ID NO: 165 - protein, 14 aa, Artificial Sequence
  Description of Artificial Sequence Synthetic peptide
LAIPVNRPGQLQSF

SEQ ID NO: 166 - protein, 21 aa, Artificial Sequence
  Description of Artificial Sequence Synthetic peptide
KQVQNYKAKLSSGDVFVIPAG

SEQ ID NO: 167 - protein, 23 aa, Artificial Sequence
  Description of Artificial Sequence Synthetic peptide
RGDTIKLPAGTIAYLVNRDDNEE

SEQ ID NO: 168 - protein, 19 aa, Artificial Sequence
  Description of Artificial Sequence Synthetic peptide
FLAGDEDNVISQIQRPVKE

SEQ ID NO: 169 - protein, 13 aa, Artificial Sequence
  Description of Artificial Sequence Synthetic peptide
LAIPVNRPGQLQS

SEQ ID NO: 170 - protein, 15 aa, Artificial Sequence
  Description of Artificial Sequence Synthetic peptide
VLDLAIPVNRPGQLQ

SEQ ID NO: 171 - protein, 13 aa, Artificial Sequence
  Description of Artificial Sequence Synthetic peptide
DLAIPVNRPGQLQ

SEQ ID NO: 172 - protein, 13 aa, Artificial Sequence
  Description of Artificial Sequence Synthetic peptide
VFVIPAGHPVAVK

SEQ ID NO: 173 - protein, 20 aa, Artificial Sequence
  Description of Artificial Sequence Synthetic peptide
TIFLPQHTDADYILVVLSGK

SEQ ID NO: 174 - protein, 23 aa, Artificial Sequence
  Description of Artificial Sequence Synthetic peptide
NQRNFLAGDEDNVISQIQRPVKE

SEQ ID NO: 175 - protein, 12 aa, Artificial Sequence
  Description of Artificial Sequence Synthetic peptide
LAIPVNRPGQLQ

SEQ ID NO: 176 - protein, 17 aa, Artificial Sequence
  Description of Artificial Sequence Synthetic peptide
HPVAVKASSNLDLLGFG

SEQ ID NO: 177 - protein, 11 aa, Artificial Sequence
  Description of Artificial Sequence Synthetic peptide
LAIPVNRPGQL

SEQ ID NO: 178 - protein, 12 aa, Artificial Sequence
  Description of Artificial Sequence Synthetic peptide
DLAIPVNRPGQL

SEQ ID NO: 179 - protein, 24 aa, Artificial Sequence
  Description of Artificial Sequence Synthetic peptide
SKPHTIFLPQHTDADYILVVLSGK

SEQ ID NO: 180 - protein, 12 aa, Artificial Sequence
  Description of Artificial Sequence Synthetic peptide
FVIPAGHPVAVK

SEQ ID NO: 181 - protein, 14 aa, Artificial Sequence
  Description of Artificial Sequence Synthetic peptide
DLAIPVNRPGQLQS

SEQ ID NO: 182 - protein, 22 aa, Artificial Sequence
  Description of Artificial Sequence Synthetic peptide
SGDVFVIPAGHPVAVKASSNLD

SEQ ID NO: 183 - protein, 13 aa, Artificial Sequence
  Description of Artificial Sequence Synthetic peptide
AIPVNRPGQLQSF

SEQ ID NO: 184 - protein, 13 aa, Artificial Sequence
  Description of Artificial Sequence Synthetic peptide
ELAFPGSAQEVDR

SEQ ID NO: 185 - protein, 18 aa, Artificial Sequence
  Description of Artificial Sequence Synthetic peptide
LAIPVNRPGQLQSFLLSG

SEQ ID NO: 186 - protein, 24 aa, Artificial Sequence
  Description of Artificial Sequence Synthetic peptide
VFVIPAGHPVAVKASSNLDLLGFG

SEQ ID NO: 187 - protein, 8 aa, Artificial Sequence
  Description of Artificial Sequence Synthetic peptide
AGHPVAVK

SEQ ID NO: 188 - protein, 21 aa, Artificial Sequence
  Description of Artificial Sequence Synthetic peptide
HPVAVKASSNLDLLGFGINAE

SEQ ID NO: 189 - protein, 22 aa, Artificial Sequence
  Description of Artificial Sequence Synthetic peptide
LAIPVNRPGQLQSFLLSGNQNQ

SEQ ID NO: 190 - protein, 10 aa, Artificial Sequence
  Description of Artificial Sequence Synthetic peptide
SGDVFVIPAG

SEQ ID NO: 191 - protein, 20 aa, Artificial Sequence
  Description of Artificial Sequence Synthetic peptide
GSLLLPHYNSRAIVIVTVNE

SEQ ID NO: 192 - protein, 19 aa, Artificial Sequence
  Description of Artificial Sequence Synthetic peptide
NFLAGDEDNVISQIQRPVK

SEQ ID NO: 193 - protein, 14 aa, Artificial Sequence
  Description of Artificial Sequence Synthetic peptide
SGDVFVIPAGHPVA

SEQ ID NO: 194 - protein, 16 aa, Artificial Sequence
  Description of Artificial Sequence Synthetic peptide
GSLLLPHYNSRAIVIV

SEQ ID NO: 195 - protein, 20 aa, Artificial Sequence
  Description of Artificial Sequence Synthetic peptide
RGDTIKLPAGTIAYLVNRDD

SEQ ID NO: 196 - protein, 16 aa, Artificial Sequence
  Description of Artificial Sequence Synthetic peptide
SGDVFVIPAGHPVAVK

SEQ ID NO: 197 - protein, 18 aa, Artificial Sequence
  Description of Artificial Sequence Synthetic peptide
LSSGDVFVIPAGHPVAVK

SEQ ID NO: 198 - protein, 13 aa, Artificial Sequence
  Description of Artificial Sequence Synthetic peptide
LDLAIPVNRPGQL

SEQ ID NO: 199 - protein, 10 aa, Artificial Sequence
  Description of Artificial Sequence Synthetic peptide
AIPVNRPGQL

SEQ ID NO: 200 - protein, 16 aa, Artificial Sequence
  Description of Artificial Sequence Synthetic peptide
LAIPVNRPGQLQSFLL

SEQ ID NO: 201 - protein, 22 aa, Artificial Sequence
  Description of Artificial Sequence Synthetic peptide
PHTIFLPQHTDADYILVVLSGK

SEQ ID NO: 202 - protein, 19 aa, Artificial Sequence
  Description of Artificial Sequence Synthetic peptide
VFVIPAGHPVAVKASSNLD

SEQ ID NO: 203 - protein, 17 aa, Artificial Sequence
  Description of Artificial Sequence Synthetic peptide
LAIPVNRPGQLQSFLLS

SEQ ID NO: 204 - protein, 17 aa, Artificial Sequence
  Description of Artificial Sequence Synthetic peptide
VLDLAIPVNRPGQLQSF

SEQ ID NO: 205 - protein, 12 aa, Artificial Sequence
  Description of Artificial Sequence Synthetic peptide
AIPVNRPGQLQS

SEQ ID NO: 206 - protein, 20 aa, Artificial Sequence
  Description of Artificial Sequence Synthetic peptide
DTIKLPAGTIAYLVNRDDNE

SEQ ID NO: 207 - protein, 17 aa, Artificial Sequence
  Description of Artificial Sequence Synthetic peptide
NYKAKLSSGDVFVIPAG

SEQ ID NO: 208 - protein, 19 aa, Artificial Sequence
  Description of Artificial Sequence Synthetic peptide
GKAILTVLKPDDRNSFNLE

SEQ ID NO: 209 - protein, 17 aa, Artificial Sequence
  Description of Artificial Sequence Synthetic peptide
YKSKPHTIFLPQHTDAD

SEQ ID NO: 210 - protein, 11 aa, Artificial Sequence
  Description of Artificial Sequence Synthetic peptide
ASSNLDLLGFG

SEQ ID NO: 211 - protein, 13 aa, Artificial Sequence
  Description of Artificial Sequence Synthetic peptide
DEEEEQGEEEINK

SEQ ID NO: 212 - protein, 15 aa, Artificial Sequence
  Description of Artificial Sequence Synthetic peptide
YKSKPHTIFLPQHTD

SEQ ID NO: 213 - protein, 10 aa, Artificial Sequence
  Description of Artificial Sequence Synthetic peptide
VLDLAIPVNR

SEQ ID NO: 214 - protein, 25 aa, Artificial Sequence
  Description of Artificial Sequence Synthetic peptide
FFEITPEKNPQLQDLDIFVNSVEIK

SEQ ID NO: 215 - protein, 14 aa, Artificial Sequence
  Description of Artificial Sequence Synthetic peptide
TIFLPQHTDADYIL

SEQ ID NO: 216 - protein, 16 aa, Artificial Sequence
  Description of Artificial Sequence Synthetic peptide
SFLLSGNQNQQNYLSG

SEQ ID NO: 217 - protein, 18 aa, Artificial Sequence
  Description of Artificial Sequence Synthetic peptide
SFLLSGNQNQQNYLSGFS

SEQ ID NO: 218 - protein, 19 aa, Artificial Sequence
  Description of Artificial Sequence Synthetic peptide
NQQEQRKEDDEEEEQGEEE

SEQ ID NO: 219 - protein, 10 aa, Artificial Sequence
  Description of Artificial Sequence Synthetic peptide
EEQGEEEINK

SEQ ID NO: 220 - protein, 9 aa, Artificial Sequence
  Description of Artificial Sequence Synthetic peptide
SRGPIYSNE

SEQ ID NO: 221 - protein, 15 aa, Artificial Sequence
  Description of Artificial Sequence Synthetic peptide
EDDEEEEQGEEEINK

SEQ ID NO: 222 - protein, 14 aa, Artificial Sequence
  Description of Artificial Sequence Synthetic peptide
DDEEEEQGEEEINK

SEQ ID NO: 223 - protein, 15 aa, Artificial Sequence
  Description of Artificial Sequence Synthetic peptide
KEDDEEEEQGEEEIN

SEQ ID NO: 224 - protein, 12 aa, Artificial Sequence
  Description of Artificial Sequence Synthetic peptide
KEDDEEEEQGEE

SEQ ID NO: 225 - protein, 15 aa, Artificial Sequence
  Description of Artificial Sequence Synthetic peptide
QRKEDDEEEEQGEEE

SEQ ID NO: 226 - protein, 16 aa, Artificial Sequence
  Description of Artificial Sequence Synthetic peptide
KEDDEEEEQGEEEINK

SEQ ID NO: 227 - protein, 13 aa, Artificial Sequence
  Description of Artificial Sequence Synthetic peptide
KEDDEEEEQGEEE

SEQ ID NO: 228 - protein, 15 aa, Artificial Sequence
  Description of Artificial Sequence Synthetic peptide
HPVAITASSNLNLLG

SEQ ID NO: 229 - protein, 11 aa, Artificial Sequence
  Description of Artificial Sequence Synthetic peptide
ASSNLNLLGFG

SEQ ID NO: 230 - protein, 13 aa, Artificial Sequence
  Description of Artificial Sequence Synthetic peptide
ITASSNLNLLGFG

SEQ ID NO: 231 - protein, 17 aa, Artificial Sequence
  Description of Artificial Sequence Synthetic peptide
ITASSNLNLLGFGINAE

SEQ ID NO: 232 - protein, 10 aa, Artificial Sequence
  Description of Artificial Sequence Synthetic peptide
SSNLNLLGFG

SEQ ID NO: 233 - protein, 13 aa, Artificial Sequence
  Description of Artificial Sequence Synthetic peptide
VDLVIPVNGPGKF

SEQ ID NO: 234 - protein, 12 aa, Artificial Sequence
  Description of Artificial Sequence Synthetic peptide
LVIPVNGPGKFE

SEQ ID NO: 235 - protein, 13 aa, Artificial Sequence
  Description of Artificial Sequence Synthetic peptide
LVIPVNGPGKFEA

SEQ ID NO: 236 - protein, 17 aa, Artificial Sequence
  Description of Artificial Sequence Synthetic peptide
LRLVDLVIPVNGPGKFE

SEQ ID NO: 237 - protein, 17 aa, Artificial Sequence
  Description of Artificial Sequence Synthetic peptide
YRAKPHTIFLPQHIDAD

SEQ ID NO: 238 - protein, 21 aa, Artificial Sequence
  Description of Artificial Sequence Synthetic peptide
HPVAITASSNLNLLGFGINAE

SEQ ID NO: 239 - protein, 9 aa, Artificial Sequence
  Description of Artificial Sequence Synthetic peptide
SNLNLLGFG

SEQ ID NO: 240 - protein, 24 aa, Artificial Sequence
  Description of Artificial Sequence Synthetic peptide
HPVAITASSNLNLLGFGINAENNE

SEQ ID NO: 241 - protein, 15 aa, Artificial Sequence
  Description of Artificial Sequence Synthetic peptide
LVDLVIPVNGPGKFE

SEQ ID NO: 242 - protein, 11 aa, Artificial Sequence
  Description of Artificial Sequence Synthetic peptide
LVIPVNGPGKF

SEQ ID NO: 243 - protein, 18 aa, Artificial Sequence
  Description of Artificial Sequence Synthetic peptide
TIKLPAGTTSYLVNQDDE

SEQ ID NO: 244 - protein, 18 aa, Artificial Sequence
  Description of Artificial Sequence Synthetic peptide
DLRLVDLVIPVNGPGKFE

SEQ ID NO: 245 - protein, 19 aa, Artificial Sequence
  Description of Artificial Sequence Synthetic peptide
EDLRLVDLVIPVNGPGKFE

SEQ ID NO: 246 - protein, 17 aa, Artificial Sequence
  Description of Artificial Sequence Synthetic peptide
HPVAITASSNLNLLGFG

SEQ ID NO: 247 - protein, 21 aa, Artificial Sequence
  Description of Artificial Sequence Synthetic peptide
LVDLVIPVNGPGKFEAFDLAK

SEQ ID NO: 248 - protein, 13 aa, Artificial Sequence
  Description of Artificial Sequence Synthetic peptide
DNVISQIENPVKE

SEQ ID NO: 249 - protein, 24 aa, Artificial Sequence
  Description of Artificial Sequence Synthetic peptide
VVIIPAGHPVAITASSNLNLLGFG

SEQ ID NO: 250 - protein, 17 aa, Artificial Sequence
  Description of Artificial Sequence Synthetic peptide
LVDLVIPVNGPGKFEAF

SEQ ID NO: 251 - protein, 9 aa, Artificial Sequence
  Description of Artificial Sequence Synthetic peptide
YPQLQDLDL

SEQ ID NO: 252 - protein, 10 aa, Artificial Sequence
  Description of Artificial Sequence Synthetic peptide
VIPVNGPGKF

SEQ ID NO: 253 - protein, 8 aa, Artificial Sequence
  Description of Artificial Sequence Synthetic peptide
SKKSLPSE

SEQ ID NO: 254 - protein, 17 aa, Artificial Sequence
  Description of Artificial Sequence Synthetic peptide
LPQHIDADLILVVLSGK

SEQ ID NO: 255 - protein, 19 aa, Artificial Sequence
  Description of Artificial Sequence Synthetic peptide
RGDTIKLPAGTTSYLVNQD

SEQ ID NO: 256 - protein, 9 aa, Artificial Sequence
  Description of Artificial Sequence Synthetic peptide
IPVNGPGKF

SEQ ID NO: 257 - protein, 9 aa, Artificial Sequence
  Description of Artificial Sequence Synthetic peptide
LPQHIDADL

SEQ ID NO: 258 - protein, 10 aa, Artificial Sequence
  Description of Artificial Sequence Synthetic peptide
LVIPVNGPGK

SEQ ID NO: 259 - protein, 10 aa, Artificial Sequence
  Description of Artificial Sequence Synthetic peptide
IFLPQHIDAD

SEQ ID NO: 260 - protein, 8 aa, Artificial Sequence
  Description of Artificial Sequence Synthetic peptide
LPQHIDAD

SEQ ID NO: 261 - protein, 9 aa, Artificial Sequence
  Description of Artificial Sequence Synthetic peptide
VIPVNGPGK

SEQ ID NO: 262 - protein, 9 aa, Artificial Sequence
  Description of Artificial Sequence Synthetic peptide
IFLPQHIDA

SEQ ID NO: 263 - protein, 19 aa, Artificial Sequence
  Description of Artificial Sequence Synthetic peptide
TIKLPAGTTSYLVNQDDEE

SEQ ID NO: 264 - protein, 21 aa, Artificial Sequence
  Description of Artificial Sequence Synthetic peptide
HGEWRPSYEKEEDEEEGQRER

SEQ ID NO: 265 - protein, 23 aa, Artificial Sequence
  Description of Artificial Sequence Synthetic peptide
EKRHGEWRPSYEKEEDEEEGQRE

SEQ ID NO: 266 - protein, 19 aa, Artificial Sequence
  Description of Artificial Sequence Synthetic peptide
LPAGTTSYLVNQDDEEDLR

SEQ ID NO: 267 - protein, 16 aa, Artificial Sequence
  Description of Artificial Sequence Synthetic peptide
PSYEKEEDEEEGQRER

SEQ ID NO: 268 - protein, 12 aa, Artificial Sequence
  Description of Artificial Sequence Synthetic peptide
EKRHGEWRPSYE

SEQ ID NO: 269 - protein, 20 aa, Artificial Sequence
  Description of Artificial Sequence Synthetic peptide
TIKLPAGTTSYLVNQDDEED

SEQ ID NO: 270 - protein, 17 aa, Artificial Sequence
  Description of Artificial Sequence Synthetic peptide
HGEWRPSYEKQEDEEEK

SEQ ID NO: 271 - protein, 14 aa, Artificial Sequence
  Description of Artificial Sequence Synthetic peptide
EWRPSYEKEEDEEE

SEQ ID NO: 272 - protein, 14 aa, Artificial Sequence
  Description of Artificial Sequence Synthetic peptide
PSYEKEEDEEEGQR

SEQ ID NO: 273 - protein, 11 aa, Artificial Sequence
  Description of Artificial Sequence Synthetic peptide
EKEEDEEEGQR

SEQ ID NO: 274 - protein, 18 aa, Artificial Sequence
  Description of Artificial Sequence Synthetic peptide
EWRPSYEKEEDEEEGQRE

SEQ ID NO: 275 - protein, 10 aa, Artificial Sequence
  Description of Artificial Sequence Synthetic peptide
KEEDEEEGQR

SEQ ID NO: 276 - protein, 19 aa, Artificial Sequence
  Description of Artificial Sequence Synthetic peptide
VQPGRERWEREEDEEQVDE

SEQ ID NO: 277 - protein, 12 aa, Artificial Sequence
  Description of Artificial Sequence Synthetic peptide
DVVIIPAGHPVA

SEQ ID NO: 278 - protein, 14 aa, Artificial Sequence
  Description of Artificial Sequence Synthetic peptide
HGEWRPSYEKQEDE

SEQ ID NO: 279 - protein, 9 aa, Artificial Sequence
  Description of Artificial Sequence Synthetic peptide
EEDEEEGQR

SEQ ID NO: 280 - protein, 19 aa, Artificial Sequence
  Description of Artificial Sequence Synthetic peptide
HGEWRPSYEKEEDEEEGQR

SEQ ID NO: 281 - protein, 14 aa, Artificial Sequence
  Description of Artificial Sequence Synthetic peptide
EEWRGSQRREDPEE

SEQ ID NO: 282 - protein, 23 aa, Artificial Sequence
  Description of Artificial Sequence Synthetic peptide
REEDEEQVDEEWRGSQRREDPEE

SEQ ID NO: 283 - protein, 9 aa, Artificial Sequence
  Description of Artificial Sequence Synthetic peptide
RHGEWRPSY

SEQ ID NO: 284 - protein, 15 aa, Artificial Sequence
  Description of Artificial Sequence Synthetic peptide
HGEWRPSYEKQEDEE

SEQ ID NO: 285 - protein, 11 aa, Artificial Sequence
  Description of Artificial Sequence Synthetic peptide
VVIIPAGHPVA

SEQ ID NO: 286 - protein, 9 aa, Artificial Sequence
  Description of Artificial Sequence Synthetic peptide
HGEWRPSYE

SEQ ID NO: 287 - protein, 12 aa, Artificial Sequence
  Description of Artificial Sequence Synthetic peptide
KEEDEEEGQRER

SEQ ID NO: 288 - protein, 13 aa, Artificial Sequence
  Description of Artificial Sequence Synthetic peptide
VVIIPAGHPVAIT

SEQ ID NO: 289 - protein, 17 aa, Artificial Sequence
  Description of Artificial Sequence Synthetic peptide
EKRHGEWRPSYEKEEDE

SEQ ID NO: 290 - protein, 17 aa, Artificial Sequence
  Description of Artificial Sequence Synthetic peptide
QVDEEWRGSQRREDPEE

SEQ ID NO: 291 - protein, 24 aa, Artificial Sequence
  Description of Artificial Sequence Synthetic peptide
GDTIKLPAGTTSYLVNQDDEEDLR

SEQ ID NO: 292 - protein, 11 aa, Artificial Sequence
  Description of Artificial Sequence Synthetic peptide
GSEPRVPAQRE

SEQ ID NO: 293 - protein, 15 aa, Artificial Sequence
  Description of Artificial Sequence Synthetic peptide
EEKRHGEWRPSYEKE

SEQ ID NO: 294 - protein, 13 aa, Artificial Sequence
  Description of Artificial Sequence Synthetic peptide
EWRPSYEKEEDEE

SEQ ID NO: 295 - protein, 15 aa, Artificial Sequence
  Description of Artificial Sequence Synthetic peptide
NYDEGSEPRVPAQRE

SEQ ID NO: 296 - protein, 12 aa, Artificial Sequence
  Description of Artificial Sequence Synthetic peptide
VIIPAGHPVAIT

SEQ ID NO: 297 - protein, 11 aa, Artificial Sequence
  Description of Artificial Sequence Synthetic peptide
RHGEWRPSYEK

SEQ ID NO: 298 - protein, 9 aa, Artificial Sequence
  Description of Artificial Sequence Synthetic peptide
NYDEGSEPR

SEQ ID NO: 299 - protein, 12 aa, Artificial Sequence
  Description of Artificial Sequence Synthetic peptide
WRPSYEKEEDEE

SEQ ID NO: 300 - protein, 13 aa, Artificial Sequence
  Description of Artificial Sequence Synthetic peptide
WRPSYEKQEDEEE

SEQ ID NO: 301 - protein, 19 aa, Artificial Sequence
  Description of Artificial Sequence Synthetic peptide
EKRHGEWRPSYEKQEDEEE

SEQ ID NO: 302 - protein, 14 aa, Artificial Sequence
  Description of Artificial Sequence Synthetic peptide
VVIIPAGHPVAITA

SEQ ID NO: 303 - protein, 11 aa, Artificial Sequence
  Description of Artificial Sequence Synthetic peptide
KRHGEWRPSYE

SEQ ID NO: 304 - protein, 16 aa, Artificial Sequence
  Description of Artificial Sequence Synthetic peptide
GSDDNVISQIENPVKE

SEQ ID NO: 305 - protein, 10 aa, Artificial Sequence
  Description of Artificial Sequence Synthetic peptide
VVIIPAGHPV

SEQ ID NO: 306 - protein, 8 aa, Artificial Sequence
  Description of Artificial Sequence Synthetic peptide
HGEWRPSY

SEQ ID NO: 307 - protein, 15 aa, Artificial Sequence
  Description of Artificial Sequence Synthetic peptide
RPSYEKEEDEEEGQR

SEQ ID NO: 308 - protein, 10 aa, Artificial Sequence
  Description of Artificial Sequence Synthetic peptide
HGEWRPSYEK

SEQ ID NO: 309 - protein, 14 aa, Artificial Sequence
  Description of Artificial Sequence Synthetic peptide
KRHGEWRPSYEKEE

SEQ ID NO: 310 - protein, 15 aa, Artificial Sequence
  Description of Artificial Sequence Synthetic peptide
VVIIPAGHPVAITAS

SEQ ID NO: 311 - protein, 23 aa, Artificial Sequence
  Description of Artificial Sequence Synthetic peptide
RGDTIKLPAGTTSYLVNQDDEED

SEQ ID NO: 312 - protein, 18 aa, Artificial Sequence
  Description of Artificial Sequence Synthetic peptide
KRHGEWRPSYEKQEDEEE

SEQ ID NO: 313 - protein, 20 aa, Artificial Sequence
  Description of Artificial Sequence Synthetic peptide
DEEQVDEEWRGSQRREDPEE

SEQ ID NO: 314 - protein, 10 aa, Artificial Sequence
  Description of Artificial Sequence Synthetic peptide
RHGEWRPSYE

SEQ ID NO: 315 - protein, 11 aa, Artificial Sequence
  Description of Artificial Sequence Synthetic peptide
HGEWRPSYEKE

SEQ ID NO: 316 - protein, 18 aa, Artificial Sequence
  Description of Artificial Sequence Synthetic peptide
KRHGEWRPSYEKEEDEEE

SEQ ID NO: 317 - protein, 19 aa, Artificial Sequence
  Description of Artificial Sequence Synthetic peptide
EKRHGEWRPSYEKEEDEEE

SEQ ID NO: 318 - protein, 25 aa, Artificial Sequence
  Description of Artificial Sequence Synthetic peptide
TIKLPAGTTSYLVNQDDEEDLRLVD

SEQ ID NO: 319 - protein, 17 aa, Artificial Sequence
  Description of Artificial Sequence Synthetic peptide
WRPSYEKEEDEEEGQRE

SEQ ID NO: 320 - protein, 17 aa, Artificial Sequence
  Description of Artificial Sequence Synthetic peptide
KRHGEWRPSYEKEEDEE

SEQ ID NO: 321 - protein, 12 aa, Artificial Sequence
  Description of Artificial Sequence Synthetic peptide
VVIIPAGHPVAI

SEQ ID NO: 322 - protein, 13 aa, Artificial Sequence
  Description of Artificial Sequence Synthetic peptide
EWRGSQRREDPEE

SEQ ID NO: 323 - protein, 19 aa, Artificial Sequence
  Description of Artificial Sequence Synthetic peptide
HGEWRPSYEKQEDEEEKQK

SEQ ID NO: 324 - protein, 17 aa, Artificial Sequence
  Description of Artificial Sequence Synthetic peptide
SGSDDNVISQIENPVKE

SEQ ID NO: 325 - protein, 17 aa, Artificial Sequence
  Description of Artificial Sequence Synthetic peptide
RPSYEKEEDEEEGQRER

SEQ ID NO: 326 - protein, 13 aa, Artificial Sequence
  Description of Artificial Sequence Synthetic peptide
EKEEDEEEGQRER

SEQ ID NO: 327 - protein, 11 aa, Artificial Sequence
  Description of Artificial Sequence Synthetic peptide
HGEWRPSYEKQ

SEQ ID NO: 328 - protein, 13 aa, Artificial Sequence
  Description of Artificial Sequence Synthetic peptide
WRPSYEKEEDEEE

SEQ ID NO: 329 - protein, 13 aa, Artificial Sequence
  Description of Artificial Sequence Synthetic peptide
LAKNKNQYLRGFS

SEQ ID NO: 330 - protein, 10 aa, Artificial Sequence
  Description of Artificial Sequence Synthetic peptide
NKNQYLRGFS

SEQ ID NO: 331 - protein, 10 aa, Artificial Sequence
  Description of Artificial Sequence Synthetic peptide
LRGFSKNILE

SEQ ID NO: 332 - protein, 15 aa, Artificial Sequence
  Description of Artificial Sequence Synthetic peptide
LAKNKNQYLRGFSKN

SEQ ID NO: 333 - protein, 11 aa, Artificial Sequence
  Description of Artificial Sequence Synthetic peptide
TVLSPNDRNSY

SEQ ID NO: 334 - protein, 12 aa, Artificial Sequence
  Description of Artificial Sequence Synthetic peptide
QYLRGFSKNILE

SEQ ID NO: 335 - protein, 19 aa, Artificial Sequence
  Description of Artificial Sequence Synthetic peptide
GKAILTVLSPNDRNSYNLE

SEQ ID NO: 336 - protein, 9 aa, Artificial Sequence
  Description of Artificial Sequence Synthetic peptide
RGFSKNILE

SEQ ID NO: 337 - protein, 15 aa, Artificial Sequence
  Description of Artificial Sequence Synthetic peptide
NKNQYLRGFSKNILE

SEQ ID NO: 338 - protein, 15 aa, Artificial Sequence
  Description of Artificial Sequence Synthetic peptide
ASSNLNLLGFGINAE

SEQ ID NO: 339 - protein, 10 aa, Artificial Sequence
  Description of Artificial Sequence Synthetic peptide
ASSNLNLLGF

SEQ ID NO: 340 - protein, 14 aa, Artificial Sequence
  Description of Artificial Sequence Synthetic peptide
LAKNKNQYLRGFSK

SEQ ID NO: 341 - protein, 22 aa, Artificial Sequence
  Description of Artificial Sequence Synthetic peptide
RGDTIKLPAGTTSYLVNQDDEE

SEQ ID NO: 342 - protein, 24 aa, Artificial Sequence
  Description of Artificial Sequence Synthetic peptide
ARLSPGDVVIIPAGHPVAITASSN

SEQ ID NO: 343 - protein, 12 aa, Artificial Sequence
  Description of Artificial Sequence Synthetic peptide
VQRYEARLSPGD

SEQ ID NO: 344 - protein, 20 aa, Artificial Sequence
  Description of Artificial Sequence Synthetic peptide
ARLSPGDVVIIPAGHPVAIT

SEQ ID NO: 345 - protein, 21 aa, Artificial Sequence
  Description of Artificial Sequence Synthetic peptide
RGDTIKLPAGTTSYLVNQDDE

SEQ ID NO: 346 - protein, 18 aa, Artificial Sequence
  Description of Artificial Sequence Synthetic peptide
ARLSPGDVVIIPAGHPVA

SEQ ID NO: 347 - protein, 20 aa, Artificial Sequence
  Description of Artificial Sequence Synthetic peptide
GALMLPHYNSRAIVVLLVNE

SEQ ID NO: 348 - protein, 23 aa, Artificial Sequence
  Description of Artificial Sequence Synthetic peptide
ARLSPGDVVIIPAGHPVAITASS

SEQ ID NO: 349 - protein, 37 aa, Artificial Sequence; identical to SEQ ID NO: 34
  Description of Artificial Sequence Synthetic polypeptide

SEQ ID NO: 350 - protein, 22 aa, Artificial Sequence
  Description of Artificial Sequence Synthetic peptide
ARLSPGDVVIIPAGHPVAITAS

SEQ ID NO: 351 - protein, 23 aa, Artificial Sequence
  Description of Artificial Sequence Synthetic peptide
LSPGDVVIIPAGHPVAITASSNL

SEQ ID NO: 352 - protein, 21 aa, Artificial Sequence
  Description of Artificial Sequence Synthetic peptide
ARLSPGDVVIIPAGHPVAITA

SEQ ID NO: 353 - protein, 16 aa, Artificial Sequence
  Description of Artificial Sequence Synthetic peptide
HGPVEMPYTLLYPSSK

SEQ ID NO: 354 - protein, 9 aa, Artificial Sequence
  Description of Artificial Sequence Synthetic peptide
LDALEPDNR

SEQ ID NO: 355 - protein, 8 aa, Artificial Sequence
  Description of Artificial Sequence Synthetic peptide
DALEPDNR

SEQ ID NO: 356 - protein, 24 aa, Artificial Sequence
  Description of Artificial Sequence Synthetic peptide
HGSLHKNAMFVPHYNLNANSIIYA

SEQ ID NO: 357 - protein, 20 aa, Artificial Sequence
  Description of Artificial Sequence Synthetic peptide
LAGTSSVINNLPLDVVAATF

SEQ ID NO: 358 - protein, 16 aa, Artificial Sequence
  Description of Artificial Sequence Synthetic peptide
FREGDIIAVPTGIVFW

SEQ ID NO: 359 - protein, 24 aa, Artificial Sequence
  Description of Artificial Sequence Synthetic peptide
GTSSVINNLPLDVVAATFNLQRNE

SEQ ID NO: 360 - protein, 18 aa, Artificial Sequence
  Description of Artificial Sequence Synthetic peptide
KGAIVKVKGGLSIISPPE

SEQ ID NO: 361 - protein, 15 aa, Artificial Sequence
  Description of Artificial Sequence Synthetic peptide
RLAGTSSVINNLPLD

SEQ ID NO: 362 - protein, 25 aa, Artificial Sequence
  Description of Artificial Sequence Synthetic peptide
AGTSSVINNLPLDVVAATFNLQRNE

SEQ ID NO: 363 - protein, 12 aa, Artificial Sequence
  Description of Artificial Sequence Synthetic peptide
AGTSSVINNLPL

SEQ ID NO: 364 - protein, 17 aa, Artificial Sequence
  Description of Artificial Sequence Synthetic peptide
LAGTSSVINNLPLDVVA

SEQ ID NO: 365 - protein, 14 aa, Artificial Sequence
  Description of Artificial Sequence Synthetic peptide
AGTSSVINNLPLDV

SEQ ID NO: 366 - protein, 17 aa, Artificial Sequence
  Description of Artificial Sequence Synthetic peptide
AGRIKTVTSLDLPVLRW

SEQ ID NO: 367 - protein, 16 aa, Artificial Sequence
  Description of Artificial Sequence Synthetic peptide
AGRIKTVTSLDLPVLR

SEQ ID NO: 368 - protein, 15 aa, Artificial Sequence
  Description of Artificial Sequence Synthetic peptide
FREGDIIAVPTGIVF

SEQ ID NO: 369 - protein, 13 aa, Artificial Sequence
  Description of Artificial Sequence Synthetic peptide
AGTSSVINNLPLD

SEQ ID NO: 370 - protein, 13 aa, Artificial Sequence
  Description of Artificial Sequence Synthetic peptide
LAGTSSVINNLPL

SEQ ID NO: 371 - protein, 16 aa, Artificial Sequence
  Description of Artificial Sequence Synthetic peptide
LAGTSSVINNLPLDVV

SEQ ID NO: 372 - protein, 13 aa, Artificial Sequence
  Description of Artificial Sequence Synthetic peptide
EGDIIAVPTGIVF

SEQ ID NO: 373 - protein, 15 aa, Artificial Sequence
  Description of Artificial Sequence Synthetic peptide
LAGTSSVINNLPLDV

SEQ ID NO: 374 - protein, 20 aa, Artificial Sequence
  Description of Artificial Sequence Synthetic peptide
AGRALTVPQNYAVAAKSLSD

SEQ ID NO: 375 - protein, 12 aa, Artificial Sequence
  Description of Artificial Sequence Synthetic peptide
AGRALTVPQNYA

SEQ ID NO: 376 - protein, 14 aa, Artificial Sequence
  Description of Artificial Sequence Synthetic peptide
LAGTSSVINNLPLD

SEQ ID NO: 377 - protein, 23 aa, Artificial Sequence
  Description of Artificial Sequence Synthetic peptide
RAGIARLAGTSSVINNLPLDVVA

SEQ ID NO: 378 - protein, 16 aa, Artificial Sequence
  Description of Artificial Sequence Synthetic peptide
RASSNLNLLGFGINAE

SEQ ID NO: 379 - protein, 12 aa, Artificial Sequence
  Description of Artificial Sequence Synthetic peptide
VTVNEGKGDFEL

SEQ ID NO: 380 - protein, 17 aa, Artificial Sequence
  Description of Artificial Sequence Synthetic peptide
VRASSNLNLLGFGINAE

SEQ ID NO: 381 - protein, 13 aa, Artificial Sequence
  Description of Artificial Sequence Synthetic peptide
VRASSNLNLLGFG

SEQ ID NO: 382 - protein, 17 aa, Artificial Sequence
  Description of Artificial Sequence Synthetic peptide
HPVAVRASSNLNLLGFG

SEQ ID NO: 383 - protein, 16 aa, Artificial Sequence
  Description of Artificial Sequence Synthetic peptide
TKNQVQSYKAKLTPGD

SEQ ID NO: 384 - protein, 15 aa, Artificial Sequence
  Description of Artificial Sequence Synthetic peptide
HPVAVRASSNLNLLG

SEQ ID NO: 385 - protein, 15 aa, Artificial Sequence
  Description of Artificial Sequence Synthetic peptide
KAKLTPGDVFVIPAG

SEQ ID NO: 386 - protein, 19 aa, Artificial Sequence
  Description of Artificial Sequence Synthetic peptide
DLTFPGSAQEVDRLLENQK

SEQ ID NO: 387 - protein, 9 aa, Artificial Sequence
  Description of Artificial Sequence Synthetic peptide
PAGHPVAVR

SEQ ID NO: 388 - protein, 18 aa, Artificial Sequence
  Description of Artificial Sequence Synthetic peptide
AKLTPGDVFVIPAGHPVA

SEQ ID NO: 389 - protein, 21 aa, Artificial Sequence
  Description of Artificial Sequence Synthetic peptide
SYKAKLTPGDVFVIPAGHPVA

SEQ ID NO: 390 - protein, 18 aa, Artificial Sequence
  Description of Artificial Sequence Synthetic peptide
LTPGDVFVIPAGHPVAVR

SEQ ID NO: 391 - protein, 19 aa, Artificial Sequence
  Description of Artificial Sequence Synthetic peptide
VQSYKAKLTPGDVFVIPAG

SEQ ID NO: 392 - protein, 20 aa, Artificial Sequence
  Description of Artificial Sequence Synthetic peptide
YKAKLTPGDVFVIPAGHPVA

SEQ ID NO: 393 - protein, 12 aa, Artificial Sequence
  Description of Artificial Sequence Synthetic peptide
FVIPAGHPVAVR

SEQ ID NO: 394 - protein, 16 aa, Artificial Sequence
  Description of Artificial Sequence Synthetic peptide
YKAKLTPGDVFVIPAG

SEQ ID NO: 395 - protein, 13 aa, Artificial Sequence
  Description of Artificial Sequence Synthetic peptide
DLTFPGSAQEVDR

SEQ ID NO: 396 - protein, 20 aa, Artificial Sequence
  Description of Artificial Sequence Synthetic peptide
AKLTPGDVFVIPAGHPVAVR

SEQ ID NO: 397 - protein, 12 aa, Artificial Sequence
  Description of Artificial Sequence Synthetic peptide
LTPGDVFVIPAG

SEQ ID NO: 398 - protein, 17 aa, Artificial Sequence
  Description of Artificial Sequence Synthetic peptide
SYKAKLTPGDVFVIPAG

SEQ ID NO: 399 - protein, 23 aa, Artificial Sequence
  Description of Artificial Sequence Synthetic peptide
SYKAKLTPGDVFVIPAGHPVAVR

SEQ ID NO: 400 - protein, 11 aa, Artificial Sequence
  Description of Artificial Sequence Synthetic peptide
VIPAGHPVAVR

SEQ ID NO: 401 - protein, 20 aa, Artificial Sequence
  Description of Artificial Sequence Synthetic peptide
QVQSYKAKLTPGDVFVIPAG

SEQ ID NO: 402 - protein, 14 aa, Artificial Sequence
  Description of Artificial Sequence Synthetic peptide
AKLTPGDVFVIPAG

SEQ ID NO: 403 - protein, 21 aa, Artificial Sequence
  Description of Artificial Sequence Synthetic peptide
HPVAVRASSNLNLLGFGINAE

SEQ ID NO: 404 - protein, 22 aa, Artificial Sequence
  Description of Artificial Sequence Synthetic peptide
YKAKLTPGDVFVIPAGHPVAVR

SEQ ID NO: 405 - protein, 23 aa, Artificial Sequence
  Description of Artificial Sequence Synthetic peptide
TKNQVQSYKAKLTPGDVFVIPAG

SEQ ID NO: 406 - protein, 12 aa, Artificial Sequence
  Description of Artificial Sequence Synthetic peptide
PFNLKSSDPIYS

SEQ ID NO: 407 - protein, 8 aa, Artificial Sequence
  Description of Artificial Sequence Synthetic peptide
IEKILLEE

SEQ ID NO: 408 - protein, 16 aa, Artificial Sequence
  Description of Artificial Sequence Synthetic peptide
SRSEPFNLKSSDPIYS

SEQ ID NO: 409 - protein, 13 aa, Artificial Sequence
  Description of Artificial Sequence Synthetic peptide
HPVAVRASSNLNL

SEQ ID NO: 410 - protein, 20 aa, Artificial Sequence
  Description of Artificial Sequence Synthetic peptide
TLFLPQYTDADFILVVLSGK

SEQ ID NO: 411 - protein, 28 aa, Artificial Sequence
  Description of Artificial Sequence Synthetic peptide
NWENVLLGLGVAGSAPGIEGDEIAPLAK

SEQ ID NO: 412 - protein, 17 aa, Artificial Sequence
  Description of Artificial Sequence Synthetic peptide
YDQYKDAWDTSVVAEIK

SEQ ID NO: 413 - protein, 16 aa, Artificial Sequence
  Description of Artificial Sequence Synthetic peptide
SSFDFIDGYDTPVEGR

SEQ ID NO: 414 - protein, 13 aa, Artificial Sequence
  Description of Artificial Sequence Synthetic peptide
GPDTGVDYKDNQM

SEQ ID NO: 415 - protein, 11 aa, Artificial Sequence
  Description of Artificial Sequence Synthetic peptide
ILNLNNNPYFK

SEQ ID NO: 416 - protein, 9 aa, Artificial Sequence
  Description of Artificial Sequence Synthetic peptide
VVGTPAYEE

SEQ ID NO: 417 - protein, 11 aa, Artificial Sequence
  Description of Artificial Sequence Synthetic peptide
IDGYDTPVEGR

SEQ ID NO: 418 - protein, 8 aa, Artificial Sequence
  Description of Artificial Sequence Synthetic peptide
VVGTPAYE

SEQ ID NO: 419 - protein, 11 aa, Artificial Sequence
  Description of Artificial Sequence Synthetic peptide
IYGPDTGVDYK

SEQ ID NO: 420 - protein, 12 aa, Artificial Sequence
  Description of Artificial Sequence Synthetic peptide
VAGSAPGIEGDE

SEQ ID NO: 421 - protein, 16 aa, Artificial Sequence
  Description of Artificial Sequence Synthetic peptide
IYGPDTGVDYKDNQMR

SEQ ID NO: 422 - protein, 12 aa, Artificial Sequence
  Description of Artificial Sequence Synthetic peptide
VVGTPAYEEMVR

SEQ ID NO: 423 - protein, 13 aa, Artificial Sequence
  Description of Artificial Sequence Synthetic peptide
DFIDGYDTPVEGR

SEQ ID NO: 424 - protein, 22 aa, Artificial Sequence
  Description of Artificial Sequence Synthetic peptide
LGLGVAGSAPGIEGDEIAPLAK

SEQ ID NO: 425 - protein, 21 aa, Artificial Sequence
  Description of Artificial Sequence Synthetic peptide
FNAPLAHLIMAGADVLAVPSR

SEQ ID NO: 426 - protein, 16 aa, Artificial Sequence
  Description of Artificial Sequence Synthetic peptide
LGLGVAGSAPGIEGDE

SEQ ID NO: 427 - protein, 20 aa, Artificial Sequence
  Description of Artificial Sequence Synthetic peptide
LGLGVAGSAPGIEGDEIAPL

SEQ ID NO: 428 - protein, 18 aa, Artificial Sequence
  Description of Artificial Sequence Synthetic peptide
VLTVSPYYAEELISGIAR

SEQ ID NO: 429 - protein, 13 aa, Artificial Sequence
  Description of Artificial Sequence Synthetic peptide
EALQAEAGLPVDR

SEQ ID NO: 430 - protein, 15 aa, Artificial Sequence
  Description of Artificial Sequence Synthetic peptide
LGLGVAGSAPGIEGD

SEQ ID NO: 431 - protein, 13 aa, Artificial Sequence
  Description of Artificial Sequence Synthetic peptide
IMAGADVLAVPSR

SEQ ID NO: 432 - protein, 15 aa, Artificial Sequence
  Description of Artificial Sequence Synthetic peptide
GLGVAGSAPGIEGDE

SEQ ID NO: 433 - protein, 14 aa, Artificial Sequence
  Description of Artificial Sequence Synthetic peptide
EALQAEAGLPVDRK

SEQ ID NO: 434 - protein, 21 aa, Artificial Sequence
  Description of Artificial Sequence Synthetic peptide
TGGLGDVLGGLPPAMAANGHR

SEQ ID NO: 435 - protein, 11 aa, Artificial Sequence
  Description of Artificial Sequence Synthetic peptide
LEEQKGPDVMA

SEQ ID NO: 436 - protein, 20 aa, Artificial Sequence
  Description of Artificial Sequence Synthetic peptide
LGVAGSAPGIEGDEIAPLAK

SEQ ID NO: 437 - protein, 21 aa, Artificial Sequence
  Description of Artificial Sequence Synthetic peptide
GLGVAGSAPGIEGDEIAPLAK

SEQ ID NO: 438 - protein, 15 aa, Artificial Sequence
  Description of Artificial Sequence Synthetic peptide
TGGLGDVLGGLPPAM

SEQ ID NO: 439 - protein, 19 aa, Artificial Sequence
  Description of Artificial Sequence Synthetic peptide
NVLLGLGVAGSAPGIEGDE

SEQ ID NO: 440 - protein, 12 aa, Artificial Sequence
  Description of Artificial Sequence Synthetic peptide
TVFDGVLRPGQL

SEQ ID NO: 441 - protein, 16 aa, Artificial Sequence
  Description of Artificial Sequence Synthetic peptide
RLQSQNDQRGEIIHVK

SEQ ID NO: 442 - protein, 15 aa, Artificial Sequence
  Description of Artificial Sequence Synthetic peptide
EGYYGEQQQQPGMTR

SEQ ID NO: 443 - protein, 14 aa, Artificial Sequence
  Description of Artificial Sequence Synthetic peptide
GYYGEQQQQPGMTR

SEQ ID NO: 444 - protein, 16 aa, Artificial Sequence
  Description of Artificial Sequence Synthetic peptide
EEGYYGEQQQQPGMTR

SEQ ID NO: 445 - protein, 20 aa, Artificial Sequence
  Description of Artificial Sequence Synthetic peptide
YYGGEGSSSEQGYYGEGSSE

SEQ ID NO: 446 - protein, 19 aa, Artificial Sequence
  Description of Artificial Sequence Synthetic peptide
YGGEGSSSEQGYYGEGSSE

SEQ ID NO: 447 - protein, 18 aa, Artificial Sequence
  Description of Artificial Sequence Synthetic peptide
SSEEGYYGEQQQQPGMTR

SEQ ID NO: 448 - protein, 17 aa, Artificial Sequence
  Description of Artificial Sequence Synthetic peptide
SEEGYYGEQQQQPGMTR

SEQ ID NO: 449 - protein, 11 aa, Artificial Sequence
  Description of Artificial Sequence Synthetic peptide
QGYYGEGSSEE

SEQ ID NO: 450 - protein, 22 aa, Artificial Sequence
  Description of Artificial Sequence Synthetic peptide
YGGEGSSSEQGYYGEGSSEEGY

SEQ ID NO: 451 - protein, 12 aa, Artificial Sequence
  Description of Artificial Sequence Synthetic peptide
YGEQQQQPGMTR

SEQ ID NO: 452 - protein, 13 aa, Artificial Sequence
  Description of Artificial Sequence Synthetic peptide
YYGEQQQQPGMTR

SEQ ID NO: 453 - protein, 20 aa, Artificial Sequence
  Description of Artificial Sequence Synthetic peptide
SYEESMPMPLEQGWSSSSSE

SEQ ID NO: 454 - protein, 23 aa, Artificial Sequence
  Description of Artificial Sequence Synthetic peptide
YYGEGSSEEGYYGEQQQQPGMTR

SEQ ID NO: 455 - protein, 10 aa, Artificial Sequence
  Description of Artificial Sequence Synthetic peptide
QQQQPGMTRV

SEQ ID NO: 456 - protein, 11 aa, Artificial Sequence
  Description of Artificial Sequence Synthetic peptide
GEQQQQPGMTR

SEQ ID NO: 457 - protein, 21 aa, Artificial Sequence
  Description of Artificial Sequence Synthetic peptide
SYEESMPMPLEQGWSSSSSEY

SEQ ID NO: 458 - protein, 23 aa, Artificial Sequence
  Description of Artificial Sequence Synthetic peptide
YYGGEGSSSEQGYYGEGSSEEGY

SEQ ID NO: 459 - protein, 14 aa, Artificial Sequence
  Description of Artificial Sequence Synthetic peptide
YGEQQQQPGMTRVR

SEQ ID NO: 460 - protein, 21 aa, Artificial Sequence
  Description of Artificial Sequence Synthetic peptide
GEGSSEEGYYGEQQQQPGMTR

SEQ ID NO: 461 - protein, 22 aa, Artificial Sequence
  Description of Artificial Sequence Synthetic peptide
YGEGSSEEGYYGEQQQQPGMTR

SEQ ID NO: 462 - protein, 11 aa, Artificial Sequence
  Description of Artificial Sequence Synthetic peptide
QQQQPGMTRVR

SEQ ID NO: 463 - protein, 25 aa, Artificial Sequence
  Description of Artificial Sequence Synthetic peptide
QYAAQLPSMCRVEPQQCSIFAAGQY

SEQ ID NO: 464 - protein, 12 aa, Artificial Sequence
  Description of Artificial Sequence Synthetic peptide
TVFNGVLRPGQL

SEQ ID NO: 465 - protein, 13 aa, Artificial Sequence
  Description of Artificial Sequence Synthetic peptide
TVFNGVLRPGQLL

SEQ ID NO: 466 - protein, 20 aa, Artificial Sequence
  Description of Artificial Sequence Synthetic peptide
SGFNNELLSEALGVNALVAK

SEQ ID NO: 467 - protein, 9 aa, Artificial Sequence
  Description of Artificial Sequence Synthetic peptide
NGVLRPGQL

SEQ ID NO: 468 - protein, 17 aa, Artificial Sequence
  Description of Artificial Sequence Synthetic peptide
ALVAKRLQGQNDQRGEI

SEQ ID NO: 469 - protein, 10 aa, Artificial Sequence
  Description of Artificial Sequence Synthetic peptide
VPRYSNTPGM

SEQ ID NO: 470 - protein, 10 aa, Artificial Sequence
  Description of Artificial Sequence Synthetic peptide
PRYSNTPGMV

SEQ ID NO: 471 - protein, 9 aa, Artificial Sequence
  Description of Artificial Sequence Synthetic peptide
YSNTPGMVY

SEQ ID NO: 472 - protein, 11 aa, Artificial Sequence
  Description of Artificial Sequence Synthetic peptide
LVPRYSNTPGM

SEQ ID NO: 473 - protein, 10 aa, Artificial Sequence
  Description of Artificial Sequence Synthetic peptide
FYNEGDAPVV

SEQ ID NO: 474 - protein, 12 aa, Artificial Sequence
  Description of Artificial Sequence Synthetic peptide
FYNEGDAPVVAL

SEQ ID NO: 475 - protein, 15 aa, Artificial Sequence
  Description of Artificial Sequence Synthetic peptide
FEPLRRVRSEAGVTE

SEQ ID NO: 476 - protein, 13 aa, Artificial Sequence
  Description of Artificial Sequence Synthetic peptide
FYNEGDAPVVALY

SEQ ID NO: 477 - protein, 11 aa, Artificial Sequence
  Description of Artificial Sequence Synthetic peptide
FYNEGDAPVVA

SEQ ID NO: 478 - protein, 10 aa, Artificial Sequence
  Description of Artificial Sequence Synthetic peptide
EVEERSQNIF

SEQ ID NO: 479 - protein, 12 aa, Artificial Sequence
  Description of Artificial Sequence Synthetic peptide
VEERSQNIFSGF

SEQ ID NO: 480 - protein, 12 aa, Artificial Sequence
  Description of Artificial Sequence Synthetic peptide
ASLQEQEQGQVQ

SEQ ID NO: 481 - protein, 11 aa, Artificial Sequence
  Description of Artificial Sequence Synthetic peptide
QEQEQGQVQSR

SEQ ID NO: 482 - protein, 14 aa, Artificial Sequence
  Description of Artificial Sequence Synthetic peptide
ASLQEQEQGQVQSR

SEQ ID NO: 483 - protein, 13 aa, Artificial Sequence
  Description of Artificial Sequence Synthetic peptide
EVEERSQNIFSGF

SEQ ID NO: 484 - protein, 17 aa, Artificial Sequence
  Description of Artificial Sequence Synthetic peptide
VTDLNNGANQLDPRQRD

SEQ ID NO: 485 - protein, 14 aa, Artificial Sequence
  Description of Artificial Sequence Synthetic peptide
VEHGLSLLQPYASL

SEQ ID NO: 486 - protein, 21 aa, Artificial Sequence
  Description of Artificial Sequence Synthetic peptide
IYVTDLNNGANQLDPRQRDFL

SEQ ID NO: 487 - protein, 19 aa, Artificial Sequence
  Description of Artificial Sequence Synthetic peptide
VTDLNNGANQLDPRQRDFL

SEQ ID NO: 488 - protein, 25 aa, Artificial Sequence
  Description of Artificial Sequence Synthetic peptide
VEHGLSLLQPYASLQEQEQGQVQSR

SEQ ID NO: 489 - protein, 14 aa, Artificial Sequence
  Description of Artificial Sequence Synthetic peptide
VTDLNNGANQLDPR

SEQ ID NO: 490 - protein, 19 aa, Artificial Sequence
  Description of Artificial Sequence Synthetic peptide
IYVTDLNNGANQLDPRQRD

SEQ ID NO: 491 - protein, 20 aa, Artificial Sequence
  Description of Artificial Sequence Synthetic peptide
YVTDLNNGANQLDPRQRDFL

SEQ ID NO: 492 - protein, 15 aa, Artificial Sequence
  Description of Artificial Sequence Synthetic peptide
YVTDLNNGANQLDPR

SEQ ID NO: 493 - protein, 17 aa, Artificial Sequence
  Description of Artificial Sequence Synthetic peptide
STELLSEALGVSSQVAR

SEQ ID NO: 494 - protein, 16 aa, Artificial Sequence
  Description of Artificial Sequence Synthetic peptide
HGLSLLQPYASLQEQE

SEQ ID NO: 495 - protein, 20 aa, Artificial Sequence
  Description of Artificial Sequence Synthetic peptide
SGFSTELLSEALGVSSQVAR

SEQ ID NO: 496 - protein, 13 aa, Artificial Sequence
  Description of Artificial Sequence Synthetic peptide
GAFTPLQYKSYQD

SEQ ID NO: 497 - protein, 15 aa, Artificial Sequence
  Description of Artificial Sequence Synthetic peptide
GLLLPHYTNGASLVY

SEQ ID NO: 498 - protein, 16 aa, Artificial Sequence
  Description of Artificial Sequence Synthetic peptide
FLLAGNKRNPQAYRRE

SEQ ID NO: 499 - protein, 12 aa, Artificial Sequence
  Description of Artificial Sequence Synthetic peptide
ALPTDVLANAYR

SEQ ID NO: 500 - protein, 8 aa, Artificial Sequence
  Description of Artificial Sequence Synthetic peptide
DFLLAGNK

SEQ ID NO: 501 - protein, 8 aa, Artificial Sequence
  Description of Artificial Sequence Synthetic peptide
DVLANAYR

SEQ ID NO: 502 - protein, 9 aa, Artificial Sequence
  Description of Artificial Sequence Synthetic peptide
GAFTPLQYK

SEQ ID NO: 503 - protein, 14 aa, Artificial Sequence
  Description of Artificial Sequence Synthetic peptide
QGDVIALPAGVAHW

SEQ ID NO: 504 - protein, 12 aa, Artificial Sequence
  Description of Artificial Sequence Synthetic peptide
FGAFTPLQYKSY

SEQ ID NO: 505 - protein, 14 aa, Artificial Sequence
  Description of Artificial Sequence Synthetic peptide
FLLAGNKRNPQAYR

SEQ ID NO: 506 - protein, 13 aa, Artificial Sequence
  Description of Artificial Sequence Synthetic peptide
FGAFTPLQYKSYQ

SEQ ID NO: 507 - protein, 15 aa, Artificial Sequence
  Description of Artificial Sequence Synthetic peptide
GLSLLQPYASLQEQE

SEQ ID NO: 508 - protein, 8 aa, Artificial Sequence
  Description of Artificial Sequence Synthetic peptide
AFTPLQYK

SEQ ID NO: 509 - protein, 14 aa, Artificial Sequence
  Description of Artificial Sequence Synthetic peptide
FGAFTPLQYKSYQD

SEQ ID NO: 510 - protein, 13 aa, Artificial Sequence
  Description of Artificial Sequence Synthetic peptide
GDEFGAFTPLQYK

SEQ ID NO: 511 - protein, 15 aa, Artificial Sequence
  Description of Artificial Sequence Synthetic peptide
FGAFTPLQYKSYQDV

SEQ ID NO: 512 - protein, 10 aa, Artificial Sequence
  Description of Artificial Sequence Synthetic peptide
FGAFTPLQYK

SEQ ID NO: 513 - protein, 11 aa, Artificial Sequence
  Description of Artificial Sequence Synthetic peptide
FGAFTPLQYKS

SEQ ID NO: 514 - protein, 14 aa, Artificial Sequence
  Description of Artificial Sequence Synthetic peptide
VYIIQGRGITGPTF

SEQ ID NO: 515 - protein, 13 aa, Artificial Sequence
  Description of Artificial Sequence Synthetic peptide
YIIQGRGITGPTF

SEQ ID NO: 516 - protein, 14 aa, Artificial Sequence
  Description of Artificial Sequence Synthetic peptide
KTNPNSMVSHIAGK

SEQ ID NO: 517 - protein, 13 aa, Artificial Sequence
  Description of Artificial Sequence Synthetic peptide
TNPNSMVSHIAGK

SEQ ID NO: 518 - protein, 12 aa, Artificial Sequence
  Description of Artificial Sequence Synthetic peptide
PNSMVSHIAGKS

SEQ ID NO: 519 - protein, 20 aa, Artificial Sequence
  Description of Artificial Sequence Synthetic peptide
NIDNPNRADTYNPRAGRVTN

SEQ ID NO: 520 - protein, 11 aa, Artificial Sequence
  Description of Artificial Sequence Synthetic peptide
QRDFLLAGNKR

SEQ ID NO: 521 - protein, 12 aa, Artificial Sequence
  Description of Artificial Sequence Synthetic peptide
LLQPYASLQEQE

SEQ ID NO: 522 - protein, 10 aa, Artificial Sequence
  Description of Artificial Sequence Synthetic peptide
QRDFLLAGNK

SEQ ID NO: 523 - protein, 11 aa, Artificial Sequence
  Description of Artificial Sequence Synthetic peptide
QEQEQGQMQSR

SEQ ID NO: 524 - protein, 13 aa, Artificial Sequence
  Description of Artificial Sequence Synthetic peptide
SLLQPYASLQEQE

SEQ ID NO: 525 - protein, 11 aa, Artificial Sequence
  Description of Artificial Sequence Synthetic peptide
ASLQEQEQGQM

SEQ ID NO: 526 - protein, 14 aa, Artificial Sequence
  Description of Artificial Sequence Synthetic peptide
ASLQEQEQGQMQSR

SEQ ID NO: 527 - protein, 9 aa, Artificial Sequence
  Description of Artificial Sequence Synthetic peptide
DFLLAGNKR

SEQ ID NO: 528 - protein, 18 aa, Artificial Sequence
  Description of Artificial Sequence Synthetic peptide
QAFEPIRSVRSQAGTTEF

SEQ ID NO: 529 - protein, 18 aa, Artificial Sequence
  Description of Artificial Sequence Synthetic peptide
KTNPNSMVSHIAGKSSIF

SEQ ID NO: 530 - protein, 21 aa, Artificial Sequence
  Description of Artificial Sequence Synthetic peptide
VRRVIEPRGLLLPHYTNGASL

SEQ ID NO: 531 - protein, 17 aa, Artificial Sequence
  Description of Artificial Sequence Synthetic peptide
FGAFTPLQYKSYQDVYN

SEQ ID NO: 532 - protein, 10 aa, Artificial Sequence
  Description of Artificial Sequence Synthetic peptide
IALPAGVAHW

SEQ ID NO: 533 - protein, 25 aa, Artificial Sequence
  Description of Artificial Sequence Synthetic peptide
RVRQNIDNPNRADTYNPRAGRVTNL

SEQ ID NO: 534 - protein, 21 aa, Artificial Sequence
  Description of Artificial Sequence Synthetic peptide
NIDNPNRADTYNPRAGRVTNL

SEQ ID NO: 535 - protein, 16 aa, Artificial Sequence
  Description of Artificial Sequence Synthetic peptide
GAFTPLQYKSYQDVYN

SEQ ID NO: 536 - protein, 16 aa, Artificial Sequence
  Description of Artificial Sequence Synthetic peptide
PNSMVSHIAGKSSIFR

SEQ ID NO: 537 - protein, 19 aa, Artificial Sequence
  Description of Artificial Sequence Synthetic peptide
RLQAFEPIRSVRSQAGTTE

SEQ ID NO: 538 - protein, 18 aa, Artificial Sequence
  Description of Artificial Sequence Synthetic peptide
TNPNSMVSHIAGKSSIFR

SEQ ID NO: 539 - protein, 11 aa, Artificial Sequence
  Description of Artificial Sequence Synthetic peptide
ELGAPDVGHPM

SEQ ID NO: 540 - protein, 10 aa, Artificial Sequence
  Description of Artificial Sequence Synthetic peptide
LGAPDVGHPM

SEQ ID NO: 541 - protein, 15 aa, Artificial Sequence
  Description of Artificial Sequence Synthetic peptide
ELGAPDVGHPMSEVF

SEQ ID NO: 542 - protein, 12 aa, Artificial Sequence
  Description of Artificial Sequence Synthetic peptide
ELGAPDVGHPMS

SEQ ID NO: 543 - protein, 16 aa, Artificial Sequence
  Description of Artificial Sequence Synthetic peptide
ELGAPDVGHPMSEVFR

SEQ ID NO: 544 - protein, 14 aa, Artificial Sequence
  Description of Artificial Sequence Synthetic peptide
ELGAPDVGHPMSEV

SEQ ID NO: 545 - protein, 13 aa, Artificial Sequence
  Description of Artificial Sequence Synthetic peptide
ELGAPDVGHPMSE

SEQ ID NO: 546 - protein, 12 aa, Artificial Sequence
  Description of Artificial Sequence Synthetic peptide
LGAPDVGHPMSE

SEQ ID NO: 547 - protein, 15 aa, Artificial Sequence
  Description of Artificial Sequence Synthetic peptide
YRELGAPDVGHPMSE

SEQ ID NO: 548 - protein, 13 aa, Artificial Sequence
  Description of Artificial Sequence Synthetic peptide
LGAPDVGHPMSEV

SEQ ID NO: 549 - protein, 14 aa, Artificial Sequence
  Description of Artificial Sequence Synthetic peptide
RELGAPDVGHPMSE

SEQ ID NO: 550 - protein, 16 aa, Artificial Sequence
  Description of Artificial Sequence Synthetic peptide
APTGTFIASGVVVGKD

SEQ ID NO: 551 - protein, 17 aa, Artificial Sequence
  Description of Artificial Sequence Synthetic peptide
LAIVKFSPNEQNKHIGE

SEQ ID NO: 552 - protein, 21 aa, Artificial Sequence
  Description of Artificial Sequence Synthetic peptide
RHQITDTTNGHYAPVTYIQVE

SEQ ID NO: 553 - protein, 19 aa, Artificial Sequence
  Description of Artificial Sequence Synthetic peptide
GDLAIVKFSPNEQNKHIGE

SEQ ID NO: 554 - protein, 18 aa, Artificial Sequence
  Description of Artificial Sequence Synthetic peptide
NPDNPDNPNNPDNPNNPD

SEQ ID NO: 555 - protein, 14 aa, Artificial Sequence
  Description of Artificial Sequence Synthetic peptide
VLDLAIPVNRPGQL

SEQ ID NO: 556 - protein, 17 aa, Artificial Sequence
  Description of Artificial Sequence Synthetic peptide
VLDLAIPVNRPGQLQSF

SEQ ID NO: 557 - protein, 15 aa, Artificial Sequence
  Description of Artificial Sequence Synthetic peptide
SFLLSGNQNQQNYLS

SEQ ID NO: 558 - protein, 10 aa, Artificial Sequence
  Description of Artificial Sequence Synthetic peptide
VLDLAIPVNR

SEQ ID NO: 559 - protein, 18 aa, Artificial Sequence
  Description of Artificial Sequence Synthetic peptide
SFLLSGNQNQQNYLSGFS

SEQ ID NO: 560 - protein, 18 aa, Artificial Sequence
  Description of Artificial Sequence Synthetic peptide
LAIPVNRPGQLQSFLLSG

SEQ ID NO: 561 - protein, 16 aa, Artificial Sequence
  Description of Artificial Sequence Synthetic peptide
SFLLSGNQNQQNYLSG

SEQ ID NO: 562 - protein, 13 aa, Artificial Sequence
  Description of Artificial Sequence Synthetic peptide
LDLAIPVNRPGQL

SEQ ID NO: 563 - protein, 15 aa, Artificial Sequence
  Description of Artificial Sequence Synthetic peptide
VLDLAIPVNRPGQLQ

SEQ ID NO: 564 - protein, 22 aa, Artificial Sequence
  Description of Artificial Sequence Synthetic peptide
LAIPVNRPGQLQSFLLSGNQNQ

SEQ ID NO: 565 - protein, 23 aa, Artificial Sequence
  Description of Artificial Sequence Synthetic peptide
SFLLSGNQNQQNYLSGFSKNILE

SEQ ID NO: 566 - protein, 9 aa, Artificial Sequence
  Description of Artificial Sequence Synthetic peptide
GSLLLPHYN

SEQ ID NO: 567 - protein, 10 aa, Artificial Sequence
  Description of Artificial Sequence Synthetic peptide
GSLLLPHYNS

SEQ ID NO: 568 - protein, 10 aa, Artificial Sequence
  Description of Artificial Sequence Synthetic peptide
SSNLDLLGFG

SEQ ID NO: 569 - protein, 16 aa, Artificial Sequence
  Description of Artificial Sequence Synthetic peptide
AFDLAKNKNQYLRGFS

SEQ ID NO: 570 - protein, 12 aa, Artificial Sequence
  Description of Artificial Sequence Synthetic peptide
QYLRGFSKNILE

SEQ ID NO: 571 - protein, 10 aa, Artificial Sequence
  Description of Artificial Sequence Synthetic peptide
NLLGFGINAE

SEQ ID NO: 572 - protein, 9 aa, Artificial Sequence
  Description of Artificial Sequence Synthetic peptide
SNLNLLGFG

SEQ ID NO: 573 - protein, 15 aa, Artificial Sequence
  Description of Artificial Sequence Synthetic peptide
LAKNKNQYLRGFSKN

SEQ ID NO: 574 - protein, 14 aa, Artificial Sequence
  Description of Artificial Sequence Synthetic peptide
LAKNKNQYLRGFSK

SEQ ID NO: 575 - protein, 10 aa, Artificial Sequence
  Description of Artificial Sequence Synthetic peptide
LRGFSKNILE

SEQ ID NO: 576 - protein, 10 aa, Artificial Sequence
  Description of Artificial Sequence Synthetic peptide
YSNKFGKLFE

SEQ ID NO: 577 - protein, 15 aa, Artificial Sequence
  Description of Artificial Sequence Synthetic peptide
AFDLAKNKNQYLRGF

SEQ ID NO: 578 - protein, 17 aa, Artificial Sequence
  Description of Artificial Sequence Synthetic peptide
AFDLAKNKNQYLRGFSK

SEQ ID NO: 579 - protein, 10 aa, Artificial Sequence
  Description of Artificial Sequence Synthetic peptide
NKNQYLRGFS

SEQ ID NO: 580 - protein, 15 aa, Artificial Sequence
  Description of Artificial Sequence Synthetic peptide
NKNQYLRGFSKNILE

SEQ ID NO: 581 - protein, 10 aa, Artificial Sequence
  Description of Artificial Sequence Synthetic peptide
SSNLNLLGFG

SEQ ID NO: 582 - protein, 11 aa, Artificial Sequence
  Description of Artificial Sequence Synthetic peptide
EYSNKFGKLFE

SEQ ID NO: 583 - protein, 9 aa, Artificial Sequence
  Description of Artificial Sequence Synthetic peptide
ASSNLNLLG

SEQ ID NO: 584 - protein, 8 aa, Artificial Sequence
  Description of Artificial Sequence Synthetic peptide
LNLLGFGI

SEQ ID NO: 585 - protein, 8 aa, Artificial Sequence
  Description of Artificial Sequence Synthetic peptide
NKFGKLFE

SEQ ID NO: 586 - protein, 19 aa, Artificial Sequence
  Description of Artificial Sequence Synthetic peptide
VQPGRERWEREEDEEQVDE

SEQ ID NO: 587 - protein, 15 aa, Artificial Sequence
  Description of Artificial Sequence Synthetic peptide
RERWEREEDEEQVDE

SEQ ID NO: 588 - protein, 10 aa, Artificial Sequence
  Description of Artificial Sequence Synthetic peptide
ASSNLNLLGF

SEQ ID NO: 589 - protein, 13 aa, Artificial Sequence
  Description of Artificial Sequence Synthetic peptide
LAKNKNQYLRGFS

SEQ ID NO: 590 - protein, 8 aa, Artificial Sequence
  Description of Artificial Sequence Synthetic peptide
ELLGLKNE

SEQ ID NO: 591 - protein, 8 aa, Artificial Sequence
  Description of Artificial Sequence Synthetic peptide
ASSNLNLL

SEQ ID NO: 592 - protein, 9 aa, Artificial Sequence
  Description of Artificial Sequence Synthetic peptide
YPQLQDLDL

SEQ ID NO: 593 - protein, 10 aa, Artificial Sequence
  Description of Artificial Sequence Synthetic peptide
LLGLKNEQQE

SEQ ID NO: 594 - protein, 10 aa, Artificial Sequence
  Description of Artificial Sequence Synthetic peptide
LVVLSGKAIL

SEQ ID NO: 595 - protein, 10 aa, Artificial Sequence
  Description of Artificial Sequence Synthetic peptide
YDATTAIEAK

SEQ ID NO: 596 - protein, 10 aa, Artificial Sequence
  Description of Artificial Sequence Synthetic peptide
NGLQLLKPTL

SEQ ID NO: 597 - protein, 9 aa, Artificial Sequence
  Description of Artificial Sequence Synthetic peptide
GLQLLKPTL

SEQ ID NO: 598 - protein, 9 aa, Artificial Sequence
  Description of Artificial Sequence Synthetic peptide
GVLRPGQLL

SEQ ID NO: 599 - protein, 10 aa, Artificial Sequence
  Description of Artificial Sequence Synthetic peptide
DGVLRPGQLL

SEQ ID NO: 600 - protein, 13 aa, Artificial Sequence
  Description of Artificial Sequence Synthetic peptide
LQLLKPTLTQQQE

SEQ ID NO: 601 - protein, 9 aa, Artificial Sequence
  Description of Artificial Sequence Synthetic peptide
FLLAGNNNR

SEQ ID NO: 602 - protein, 10 aa, Artificial Sequence
  Description of Artificial Sequence Synthetic peptide
EFLLAGNNNR

SEQ ID NO: 603 - protein, 21 aa, Artificial Sequence
  Description of Artificial Sequence Synthetic peptide
FLLAGNNNRAQQQQVYGSSIE

SEQ ID NO: 604 - protein, 14 aa, Artificial Sequence
  Description of Artificial Sequence Synthetic peptide
FLLAGNNNRAQQQQ

SEQ ID NO: 605 - protein, 17 aa, Artificial Sequence
  Description of Artificial Sequence Synthetic peptide
FLLAGNNNRAQQQQVYG

SEQ ID NO: 606 - protein, 16 aa, Artificial Sequence
  Description of Artificial Sequence Synthetic peptide
FLLAGNNNRAQQQQVY

SEQ ID NO: 607 - protein, 19 aa, Artificial Sequence
  Description of Artificial Sequence Synthetic peptide
FQQQYYPGLSNESESETSE

SEQ ID NO: 608 - protein, 10 aa, Artificial Sequence
  Description of Artificial Sequence Synthetic peptide
LSEALGVNAL

SEQ ID NO: 609 - protein, 18 aa, Artificial Sequence
  Description of Artificial Sequence Synthetic peptide
LRPAFAQQQEQAQQQEQA

SEQ ID NO: 610 - protein, 10 aa, Artificial Sequence
  Description of Artificial Sequence Synthetic peptide
LRPAFAQQQE

SEQ ID NO: 611 - protein, 16 aa, Artificial Sequence
  Description of Artificial Sequence Synthetic peptide
LRPAFAQQQEQAQQQE

SEQ ID NO: 612 - protein, 10 aa, Artificial Sequence
  Description of Artificial Sequence Synthetic peptide
HGLSLLQPYA

SEQ ID NO: 613 - protein, 12 aa, Artificial Sequence
  Description of Artificial Sequence Synthetic peptide
HGLSLLQPYASL

SEQ ID NO: 614 - protein, 9 aa, Artificial Sequence
  Description of Artificial Sequence Synthetic peptide
HGLSLLQPY

SEQ ID NO: 615 - protein, 9 aa, Artificial Sequence
  Description of Artificial Sequence Synthetic peptide
GSLLLPHYN

SEQ ID NO: 616 - protein, 10 aa, Artificial Sequence
  Description of Artificial Sequence Synthetic peptide
GSLLLPHYNS

SEQ ID NO: 617 - protein, 10 aa, Artificial Sequence
  Description of Artificial Sequence Synthetic peptide
SSNLDLLGFG

SEQ ID NO: 618 - protein, 8 aa, Artificial Sequence
  Description of Artificial Sequence Synthetic peptide
ELLGLKNE

SEQ ID NO: 619 - protein, 9 aa, Artificial Sequence
  Description of Artificial Sequence Synthetic peptide
SNLNLLGFG

SEQ ID NO: 620 - protein, 8 aa, Artificial Sequence
  Description of Artificial Sequence Synthetic peptide
ASSNLNLL

SEQ ID NO: 621 - protein, 9 aa, Artificial Sequence
  Description of Artificial Sequence Synthetic peptide
YPQLQDLDL

SEQ ID NO: 622 - protein, 10 aa, Artificial Sequence
  Description of Artificial Sequence Synthetic peptide
SSNLNLLGFG

SEQ ID NO: 623 - protein, 9 aa, Artificial Sequence
  Description of Artificial Sequence Synthetic peptide
ASSNLNLLG

SEQ ID NO: 624 - protein, 10 aa, Artificial Sequence
  Description of Artificial Sequence Synthetic peptide
LLGLKNEQQE

SEQ ID NO: 625 - protein, 10 aa, Artificial Sequence
  Description of Artificial Sequence Synthetic peptide
LVVLSGKAIL

SEQ ID NO: 626 - protein, 8 aa, Artificial Sequence
  Description of Artificial Sequence Synthetic peptide
LNLLGFGI

SEQ ID NO: 627 - protein, 10 aa, Artificial Sequence
  Description of Artificial Sequence Synthetic peptide
ASSNLNLLGF

SEQ ID NO: 628 - protein, 8 aa, Artificial Sequence
  Description of Artificial Sequence Synthetic peptide
LQNYRLLE

SEQ ID NO: 629 - protein, 12 aa, Artificial Sequence
  Description of Artificial Sequence Synthetic peptide
LLSGTQNQPSLL

SEQ ID NO: 630 - protein, 9 aa, Artificial Sequence
  Description of Artificial Sequence Synthetic peptide
LLLPNYNSR

SEQ ID NO: 631 - protein, 9 aa, Artificial Sequence
  Description of Artificial Sequence Synthetic peptide
NLQNYRLLE

SEQ ID NO: 632 - protein, 10 aa, Artificial Sequence
  Description of Artificial Sequence Synthetic peptide
GSLLLPNYNS

SEQ ID NO: 633 - protein, 8 aa, Artificial Sequence
  Description of Artificial Sequence Synthetic peptide
NLQNYRLL

SEQ ID NO: 634 - protein, 10 aa, Artificial Sequence
  Description of Artificial Sequence Synthetic peptide
NGLQLLKPTL

SEQ ID NO: 635 - protein, 9 aa, Artificial Sequence
  Description of Artificial Sequence Synthetic peptide
GLQLLKPTL

SEQ ID NO: 636 - protein, 9 aa, Artificial Sequence
  Description of Artificial Sequence Synthetic peptide
GVLRPGQLL

SEQ ID NO: 637 - protein, 10 aa, Artificial Sequence
  Description of Artificial Sequence Synthetic peptide
DGVLRPGQLL

SEQ ID NO: 638 - protein, 13 aa, Artificial Sequence
  Description of Artificial Sequence Synthetic peptide
LQLLKPTLTQQQE

SEQ ID NO: 639 - protein, 10 aa, Artificial Sequence
  Description of Artificial Sequence Synthetic peptide
LSEALGVNAL

SEQ ID NO: 640 - protein, 18 aa, Artificial Sequence
  Description of Artificial Sequence Synthetic peptide
LNSQKFPILNLVQLSATR

SEQ ID NO: 641 - protein, 10 aa, Artificial Sequence
  Description of Artificial Sequence Synthetic peptide
HGLSLLQPYA

SEQ ID NO: 642 - protein, 12 aa, Artificial Sequence
  Description of Artificial Sequence Synthetic peptide
HGLSLLQPYASL

SEQ ID NO: 643 - protein, 9 aa, Artificial Sequence
  Description of Artificial Sequence Synthetic peptide
HGLSLLQPY

SEQ ID NO: 644 - protein, 8 aa, Artificial Sequence
  Description of Artificial Sequence Synthetic peptide
DKIILGPK

SEQ ID NO: 645 - protein, 16 aa, Artificial Sequence
  Description of Artificial Sequence Synthetic peptide
DRKRRQQGEETDAIVK

SEQ ID NO: 646 - protein, 15 aa, Artificial Sequence
  Description of Artificial Sequence Synthetic peptide
IGINGFGRIGRLVAR

SEQ ID NO: 647 - protein, 14 aa, Artificial Sequence
  Description of Artificial Sequence Synthetic peptide
ILNRGHKIKGTVVL

SEQ ID NO: 648 - protein, 13 aa, Artificial Sequence
  Description of Artificial Sequence Synthetic peptide
INGFGRIGRLVAR

SEQ ID NO: 649 - protein, 9 aa, Artificial Sequence
  Description of Artificial Sequence Synthetic peptide
KKNEPWWPK

SEQ ID NO: 650 - protein, 13 aa, Artificial Sequence
  Description of Artificial Sequence Synthetic peptide
LLLLGIIFLASVV

SEQ ID NO: 651 - protein, 13 aa, Artificial Sequence
  Description of Artificial Sequence Synthetic peptide
RLLQKFDQRSKIF

SEQ ID NO: 652 - protein, 13 aa, Artificial Sequence
  Description of Artificial Sequence Synthetic peptide
TIKSRFPLLLLLG

SEQ ID NO: 653 - protein, 12 aa, Artificial Sequence
  Description of Artificial Sequence Synthetic peptide
TKAVKNTVGRAL

SEQ ID NO: 654 - protein, 7 aa, Artificial Sequence
  Description of Artificial Sequence Synthetic peptide
VIVKLSR

SEQ ID NO: 655 - protein, 4 aa, Artificial Sequence
  Description of Artificial Sequence Synthetic peptide
HSPR

SEQ ID NO: 656 - protein, 3 aa, Artificial Sequence
  Description of Artificial Sequence Synthetic peptide
HWF

SEQ ID NO: 657 - protein, 11 aa, Artificial Sequence
  Description of Artificial Sequence Synthetic peptide
IFEDAITIPGR

SEQ ID NO: 658 - protein, 10 aa, Artificial Sequence
  Description of Artificial Sequence Synthetic peptide
ELTFPGSVQE

SEQ ID NO: 659 - protein, 9 aa, Artificial Sequence
  Description of Artificial Sequence Synthetic peptide
ELTFPGSVQ

SEQ ID NO: 660 - protein, 11 aa, Artificial Sequence
  Description of Artificial Sequence Synthetic peptide
DSINALEPDHR

SEQ ID NO: 661 - protein, 13 aa, Artificial Sequence
  Description of Artificial Sequence Synthetic peptide
LDALEPDNRIESE

SEQ ID NO: 662 - protein, 12 aa, Artificial Sequence
  Description of Artificial Sequence Synthetic peptide
EEGIQLVAEAIR

SEQ ID NO: 663 - protein, 9 aa, Artificial Sequence
  Description of Artificial Sequence Synthetic peptide
TFAEETWGK

SEQ ID NO: 664 - protein, 12 aa, Artificial Sequence
  Description of Artificial Sequence Synthetic peptide
LVSHPIAAHEGR

SEQ ID NO: 665 - protein, 11 aa, Artificial Sequence
  Description of Artificial Sequence Synthetic peptide
NLAQAPAQALL

SEQ ID NO: 666 - protein, 11 aa, Artificial Sequence
  Description of Artificial Sequence Synthetic peptide
ILVDGSHDIER

SEQ ID NO: 667 - protein, 10 aa, Artificial Sequence
  Description of Artificial Sequence Synthetic peptide
LDVTPLSLGL

SEQ ID NO: 668 - protein, 11 aa, Artificial Sequence
  Description of Artificial Sequence Synthetic peptide
WTIVQGLPIDE

SEQ ID NO: 669 - protein, 10 aa, Artificial Sequence
  Description of Artificial Sequence Synthetic peptide
KTLDYWPSLR

SEQ ID NO: 670 - protein, 9 aa, Artificial Sequence
  Description of Artificial Sequence Synthetic peptide
RHGEWGPSY

SEQ ID NO: 671 - protein, 4 aa, Artificial Sequence
  Description of Artificial Sequence Synthetic peptide
HMPP

SEQ ID NO: 672 - protein, 4 aa, Artificial Sequence
  Description of Artificial Sequence Synthetic peptide
HMPS

SEQ ID NO: 673 - protein, 5 aa, Artificial Sequence
  Description of Artificial Sequence Synthetic peptide
MPPSS

SEQ ID NO: 674 - protein, 4 aa, Artificial Sequence
  Description of Artificial Sequence Synthetic peptide
PRRF

SEQ ID NO: 675 - protein, 4 aa, Artificial Sequence
  Description of Artificial Sequence Synthetic peptide
FHMP

SEQ ID NO: 676 - protein, 4 aa, Artificial Sequence
  Description of Artificial Sequence Synthetic peptide
NNPF

SEQ ID NO: 677 - protein, 3 aa, Artificial Sequence
  Description of Artificial Sequence Synthetic peptide
FWM

SEQ ID NO: 678 - protein, 4 aa, Artificial Sequence
  Description of Artificial Sequence Synthetic peptide
PHMP

SEQ ID NO: 679 - protein, 5 aa, Artificial Sequence
  Description of Artificial Sequence Synthetic peptide
FHMPP

SEQ ID NO: 680 - protein, 5 aa, Artificial Sequence
  Description of Artificial Sequence Synthetic peptide
HMPSS

SEQ ID NO: 681 - protein, 4 aa, Artificial Sequence
  Description of Artificial Sequence Synthetic peptide
HRRS

SEQ ID NO: 682 - protein, 4 aa, Artificial Sequence
  Description of Artificial Sequence Synthetic peptide
MPPS

SEQ ID NO: 683 - protein, 4 aa, Artificial Sequence
  Description of Artificial Sequence Synthetic peptide
MPRR

SEQ ID NO: 684 - protein, 5 aa, Artificial Sequence
  Description of Artificial Sequence Synthetic peptide
PHMPS

SEQ ID NO: 685 - protein, 9 aa, Artificial Sequence
  Description of Artificial Sequence Synthetic peptide
HGGEGGRPY

SEQ ID NO: 686 - protein, 9 aa, Artificial Sequence
  Description of Artificial Sequence Synthetic peptide
GYPMYPLPR

SEQ ID NO: 687 - protein, 10 aa, Artificial Sequence
  Description of Artificial Sequence Synthetic peptide
LQQAPPPPQR

SEQ ID NO: 688 - protein, 11 aa, Artificial Sequence
  Description of Artificial Sequence Synthetic peptide
VGWGEQPWSPY

SEQ ID NO: 689 - protein, 11 aa, Artificial Sequence
  Description of Artificial Sequence Synthetic peptide
HPRPPKPDAPR

SEQ ID NO: 690 - protein, 3 aa, Artificial Sequence
  Description of Artificial Sequence Synthetic peptide
FWN

SEQ ID NO: 691 - protein, 4 aa, Artificial Sequence
  Description of Artificial Sequence Synthetic peptide
MRFR

SEQ ID NO: 692 - protein, 3 aa, Artificial Sequence
  Description of Artificial Sequence Synthetic peptide
WHT

SEQ ID NO: 693 - protein, 4 aa, Artificial Sequence
  Description of Artificial Sequence Synthetic peptide
FRRP

SEQ ID NO: 694 - protein, 4 aa, Artificial Sequence
  Description of Artificial Sequence Synthetic peptide
HRFR

SEQ ID NO: 695 - protein, 4 aa, Artificial Sequence
  Description of Artificial Sequence Synthetic peptide
MFRR

SEQ ID NO: 696 - protein, 5 aa, Artificial Sequence
  Description of Artificial Sequence Synthetic peptide
MFRRP

SEQ ID NO: 697 - protein, 4 aa, Artificial Sequence
  Description of Artificial Sequence Synthetic peptide
NMPS

SEQ ID NO: 698 - protein, 3 aa, Artificial Sequence
  Description of Artificial Sequence Synthetic peptide
WMK

SEQ ID NO: 699 - protein, 9 aa, Artificial Sequence
  Description of Artificial Sequence Synthetic peptide
YSLKPLVPR

SEQ ID NO: 700 - protein, 11 aa, Artificial Sequence
  Description of Artificial Sequence Synthetic peptide
PVEMPTLLYPS

SEQ ID NO: 701 - protein, 9 aa, Artificial Sequence
  Description of Artificial Sequence Synthetic peptide
QSFLLSGNQ

SEQ ID NO: 702 - protein, 14 aa, Artificial Sequence
  Description of Artificial Sequence Synthetic peptide
SLTLEDVPNHGTIR

SEQ ID NO: 703 - protein, 7 aa, Artificial Sequence
  Description of Artificial Sequence Synthetic peptide
LSLTDLK

SEQ ID NO: 704 - protein, 10 aa, Artificial Sequence
  Description of Artificial Sequence Synthetic peptide
THPMNFLNER

SEQ ID NO: 705 - protein, 10 aa, Artificial Sequence
  Description of Artificial Sequence Synthetic peptide
LGLSPQDALK

SEQ ID NO: 706 - protein, 12 aa, Artificial Sequence
  Description of Artificial Sequence Synthetic peptide
TRPPVPSTIPTK

SEQ ID NO: 707 - protein, 13 aa, Artificial Sequence
  Description of Artificial Sequence Synthetic peptide
RGPQQYAEWQINE

SEQ ID NO: 708 - protein, 13 aa, Artificial Sequence
  Description of Artificial Sequence Synthetic peptide
GIARLAGTSSVIN

SEQ ID NO: 709 - protein, 6 aa, Artificial Sequence
  Description of Artificial Sequence Synthetic peptide
YLRGFS

SEQ ID NO: 710 - protein, 6 aa, Artificial Sequence
  Description of Artificial Sequence Synthetic peptide
LRGFSK

SEQ ID NO: 711 - protein, 11 aa, Artificial Sequence
  Description of Artificial Sequence Synthetic peptide
GALMLPHYNSR

SEQ ID NO: 712 - protein, 9 aa, Artificial Sequence
  Description of Artificial Sequence Synthetic peptide
GALMLPHYN

SEQ ID NO: 713 - protein, 6 aa, Artificial Sequence
  Description of Artificial Sequence Synthetic peptide
RSQNIF

SEQ ID NO: 714 - protein, 4 aa, Artificial Sequence
  Description of Artificial Sequence Synthetic peptide
GHPM

SEQ ID NO: 715 - protein, 3 aa, Artificial Sequence
  Description of Artificial Sequence Synthetic peptide
WDP

SEQ ID NO: 716 - protein, 3 aa, Artificial Sequence
  Description of Artificial Sequence Synthetic peptide
WHN

SEQ ID NO: 717 - protein, 4 aa, Artificial Sequence
  Description of Artificial Sequence Synthetic peptide
PMPL

SEQ ID NO: 718 - protein, 4 aa, Artificial Sequence
  Description of Artificial Sequence Synthetic peptide
HNPR

SEQ ID NO: 719 - protein, 4 aa, Artificial Sequence
  Description of Artificial Sequence Synthetic peptide
HPSF

SEQ ID NO: 720 - protein, 4 aa, Artificial Sequence
  Description of Artificial Sequence Synthetic peptide
PNSM

SEQ ID NO: 721 - protein, 4 aa, Artificial Sequence
  Description of Artificial Sequence Synthetic peptide
HPMS

SEQ ID NO: 722 - protein, 4 aa, Artificial Sequence
  Description of Artificial Sequence Synthetic peptide
MPMP

SEQ ID NO: 723 - protein, 9 aa, Artificial Sequence
  Description of Artificial Sequence Synthetic peptide
VFDGVLRPG

SEQ ID NO: 724 - protein, 10 aa, Artificial Sequence
  Description of Artificial Sequence Synthetic peptide
RLQSQNDQRG

SEQ ID NO: 725 - protein, 6 aa, Artificial Sequence
  Description of Artificial Sequence Synthetic peptide
LQSQND

SEQ ID NO: 726 - protein, 6 aa, Artificial Sequence
  Description of Artificial Sequence Synthetic peptide
LEPDNR

SEQ ID NO: 727 - protein, 14 aa, Artificial Sequence
  Description of Artificial Sequence Synthetic peptide
QSQNDQRGEIIHVK

SEQ ID NO: 728 - protein, 8 aa, Artificial Sequence
  Description of Artificial Sequence Synthetic peptide
RGEIIHVK

SEQ ID NO: 729 - protein, 8 aa, Artificial Sequence
  Description of Artificial Sequence Synthetic peptide
RLQSQNDQ

SEQ ID NO: 730 - protein, 14 aa, Artificial Sequence
  Description of Artificial Sequence Synthetic peptide
RLQSQNDQRGEIIH

SEQ ID NO: 731 - protein, 11 aa, Artificial Sequence
  Description of Artificial Sequence Synthetic peptide
LQSQNDQRGEI

SEQ ID NO: 732 - protein, 7 aa, Artificial Sequence
  Description of Artificial Sequence Synthetic peptide
FLPQHTD

SEQ ID NO: 733 - protein, 8 aa, Artificial Sequence
  Description of Artificial Sequence Synthetic peptide
PQQYAEWQ

SEQ ID NO: 734 - protein, 12 aa, Artificial Sequence
  Description of Artificial Sequence Synthetic peptide
QSFLLSGNQNQQ

SEQ ID NO: 735 - protein, 9 aa, Artificial Sequence
  Description of Artificial Sequence Synthetic peptide
QSFLLSGNQ

SEQ ID NO: 736 - protein, 12 aa, Artificial Sequence
  Description of Artificial Sequence Synthetic peptide
PGQLQSFLLSGN

SEQ ID NO: 737 - protein, 22 aa, Artificial Sequence
  Description of Artificial Sequence Synthetic peptide
PGQLQSFLLSGNQNQQNYLSGF

SEQ ID NO: 738 - protein, 22 aa, Artificial Sequence
  Description of Artificial Sequence Synthetic peptide
QLQSFLLSGNQNQQNYLSGFSK

SEQ ID NO: 739 - protein, 12 aa, Artificial Sequence
  Description of Artificial Sequence Synthetic peptide
QNQQNYLSGFSK

SEQ ID NO: 740 - protein, 13 aa, Artificial Sequence
  Description of Artificial Sequence Synthetic peptide
GPQQYAEWQINEK

SEQ ID NO: 741 - protein, 7 aa, Artificial Sequence
  Description of Artificial Sequence Synthetic peptide
RGPQQYA

SEQ ID NO: 742 - protein, 7 aa, Artificial Sequence
  Description of Artificial Sequence Synthetic peptide
EWQINEK

SEQ ID NO: 743 - protein, 18 aa, Artificial Sequence
  Description of Artificial Sequence Synthetic peptide
GKIKIGINGFGRIGRLVA

SEQ ID NO: 744 - protein, 4 aa, Artificial Sequence
  Description of Artificial Sequence Synthetic peptide
MMAP

SEQ ID NO: 745 - protein, 4 aa, Artificial Sequence
  Description of Artificial Sequence Synthetic peptide
MAPH

SEQ ID NO: 746 - protein, 6 aa, Artificial Sequence
  Description of Artificial Sequence Synthetic peptide
ERGVLY

SEQ ID NO: 747 - protein, 6 aa, Artificial Sequence
  Description of Artificial Sequence Synthetic peptide
RVLNGL

SEQ ID NO: 748 - protein, 6 aa, Artificial Sequence
  Description of Artificial Sequence Synthetic peptide
SLLSGE

SEQ ID NO: 749 - protein, 6 aa, Artificial Sequence
  Description of Artificial Sequence Synthetic peptide
LLSGED

SEQ ID NO: 750 - protein, 6 aa, Artificial Sequence
  Description of Artificial Sequence Synthetic peptide
LSGEDA

SEQ ID NO: 751 - protein, 4 aa, Artificial Sequence
  Description of Artificial Sequence Synthetic peptide
HRHA

SEQ ID NO: 752 - protein, 11 aa, Artificial Sequence
  Description of Artificial Sequence Synthetic peptide
SRAIVIVTVNE

SEQ ID NO: 753 - protein, 8 aa, Artificial Sequence
  Description of Artificial Sequence Synthetic peptide
AKLTPGDV

SEQ ID NO: 754 - protein, 10 aa, Artificial Sequence
  Description of Artificial Sequence Synthetic peptide
IVIVTVNEGK

SEQ ID NO: 755 - protein, 16 aa, Artificial Sequence
  Description of Artificial Sequence Synthetic peptide
LDALEPDNRIESEGGL

SEQ ID NO: 756 - protein, 10 aa, Artificial Sequence
  Description of Artificial Sequence Synthetic peptide
RPYYSNAPQE

SEQ ID NO: 757 - protein, 25 aa, Artificial Sequence
  Description of Artificial Sequence Synthetic peptide
LDALEPDNRIESEGGLIETWNPNNK

SEQ ID NO: 758 - protein, 11 aa, Artificial Sequence
  Description of Artificial Sequence Synthetic peptide
AIVIVTVNEGK

SEQ ID NO: 759 - protein, 16 aa, Artificial Sequence
  Description of Artificial Sequence Synthetic peptide
LQVVNCNGNTVFDGEL

SEQ ID NO: 760 - protein, 15 aa, Artificial Sequence
  Description of Artificial Sequence Synthetic peptide
QVVNCNGNTVFDGEL

SEQ ID NO: 761 - protein, 10 aa, Artificial Sequence
  Description of Artificial Sequence Synthetic peptide
IIAVPTGIVF

SEQ ID NO: 762 - protein, 16 aa, Artificial Sequence
  Description of Artificial Sequence Synthetic peptide
GRRYRDRHQKVNRFRE

SEQ ID NO: 763 - protein, 11 aa, Artificial Sequence
  Description of Artificial Sequence Synthetic peptide
RPYYSNAPQEI

SEQ ID NO: 764 - protein, 12 aa, Artificial Sequence
  Description of Artificial Sequence Synthetic peptide
RLDALEPDNRIE

SEQ ID NO: 765 - protein, 14 aa, Artificial Sequence
  Description of Artificial Sequence Synthetic peptide
RLDALEPDNRIESE

SEQ ID NO: 766 - protein, 20 aa, Artificial Sequence
  Description of Artificial Sequence Synthetic peptide
LDALEPDNRIESEGGLIETW

SEQ ID NO: 767 - protein, 11 aa, Artificial Sequence
  Description of Artificial Sequence Synthetic peptide
LDALEPDNRIE

SEQ ID NO: 768 - protein, 18 aa, Artificial Sequence
  Description of Artificial Sequence Synthetic peptide
LDALEPDNRIESEGGLIE

SEQ ID NO: 769 - protein, 16 aa, Artificial Sequence
  Description of Artificial Sequence Synthetic peptide
LDALEPDNRIESEGGL

SEQ ID NO: 770 - protein, 10 aa, Artificial Sequence
  Description of Artificial Sequence Synthetic peptide
RPYYSNAPQE

SEQ ID NO: 771 - protein, 25 aa, Artificial Sequence
  Description of Artificial Sequence Synthetic peptide
LDALEPDNRIESEGGLIETWNPNNK

SEQ ID NO: 772 - protein, 27 aa, Artificial Sequence
  Description of Artificial Sequence Synthetic peptide
VEHGLSLLQPYASLQEQEQGQVQSRER

SEQ ID NO: 773 - protein, 9 aa, Artificial Sequence
  Description of Artificial Sequence Synthetic peptide
RSQNIFSGF

SEQ ID NO: 774 - protein, 14 aa, Artificial Sequence
  Description of Artificial Sequence Synthetic peptide
GITGPTFPGCPESY

SEQ ID NO: 775 - protein, 4 aa, Artificial Sequence
  Description of Artificial Sequence Synthetic peptide
CNGS

SEQ ID NO: 776 - protein, 5 aa, Artificial Sequence
  Description of Artificial Sequence Synthetic peptide
SPREC

SEQ ID NO: 777 - protein, 4 aa, Artificial Sequence
  Description of Artificial Sequence Synthetic peptide
PREC

SEQ ID NO: 778 - protein, 5 aa, Artificial Sequence
  Description of Artificial Sequence Synthetic peptide
PRECR

SEQ ID NO: 779 - protein, 4 aa, Artificial Sequence
  Description of Artificial Sequence Synthetic peptide
CPES

SEQ ID NO: 780 - protein, 4 aa, Artificial Sequence
  Description of Artificial Sequence Synthetic peptide
SGCS

SEQ ID NO: 781 - protein, 4 aa, Artificial Sequence
  Description of Artificial Sequence Synthetic peptide
CSNG

SEQ ID NO: 782 - protein, 12 aa, Artificial Sequence
  Description of Artificial Sequence Synthetic peptide
RSQNIFSGFSTE

SEQ ID NO: 783 - protein, 14 aa, Artificial Sequence
  Description of Artificial Sequence Synthetic peptide
VEEWSQNIFSGFST

SEQ ID NO: 784 - protein, 13 aa, Artificial Sequence
  Description of Artificial Sequence Synthetic peptide
WSQNIFSGFSTEL

SEQ ID NO: 785 - protein, 12 aa, Artificial Sequence
  Description of Artificial Sequence Synthetic peptide
WSQNIFSGFSTE

SEQ ID NO: 786 - protein, 10 aa, Artificial Sequence
  Description of Artificial Sequence Synthetic peptide
STSQWQSSRR

SEQ ID NO: 787 - protein, 4 aa, Artificial Sequence
  Description of Artificial Sequence Synthetic peptide
NRPI

SEQ ID NO: 788 - protein, 4 aa, Artificial Sequence
  Description of Artificial Sequence Synthetic peptide
CDGS

SEQ ID NO: 789 - protein, 4 aa, Artificial Sequence
  Description of Artificial Sequence Synthetic peptide
PRGC

SEQ ID NO: 790 - protein, 5 aa, Artificial Sequence
  Description of Artificial Sequence Synthetic peptide
PRGCR

SEQ ID NO: 791 - protein, 4 aa, Artificial Sequence
  Description of Artificial Sequence Synthetic peptide
RGCR

SEQ ID NO: 792 - protein, 4 aa, Artificial Sequence
  Description of Artificial Sequence Synthetic peptide
GCRF

SEQ ID NO: 793 - protein, 4 aa, Artificial Sequence
  Description of Artificial Sequence Synthetic peptide
PTFP

SEQ ID NO: 794 - protein, 5 aa, Artificial Sequence
  Description of Artificial Sequence Synthetic peptide
PGCPE

SEQ ID NO: 795 - protein, 4 aa, Artificial Sequence
  Description of Artificial Sequence Synthetic peptide
GCPE

SEQ ID NO: 796 - protein, 4 aa, Artificial Sequence
  Description of Artificial Sequence Synthetic peptide
CPET

SEQ ID NO: 797 - protein, 4 aa, Artificial Sequence
  Description of Artificial Sequence Synthetic peptide
AHWC

SEQ ID NO: 798 - protein, 4 aa, Artificial Sequence
  Description of Artificial Sequence Synthetic peptide
HWCY

SEQ ID NO: 799 - protein, 4 aa, Artificial Sequence
  Description of Artificial Sequence Synthetic peptide
SGCP

SEQ ID NO: 800 - protein, 5 aa, Artificial Sequence
  Description of Artificial Sequence Synthetic peptide
SGCPN

SEQ ID NO: 801 - protein, 4 aa, Artificial Sequence
  Description of Artificial Sequence Synthetic peptide
GCPN

SEQ ID NO: 802 - protein, 4 aa, Artificial Sequence
  Description of Artificial Sequence Synthetic peptide
CPNG

SEQ ID NO: 803 - protein, 5 aa, Artificial Sequence
  Description of Artificial Sequence Synthetic peptide
TFCTM

SEQ ID NO: 804 - protein, 4 aa, Artificial Sequence
  Description of Artificial Sequence Synthetic peptide
FCTM

SEQ ID NO: 805 - protein, 5 aa, Artificial Sequence
  Description of Artificial Sequence Synthetic peptide
FCTMR

SEQ ID NO: 806 - protein, 4 aa, Artificial Sequence
  Description of Artificial Sequence Synthetic peptide
CTMR

SEQ ID NO: 807 - protein, 4 aa, Artificial Sequence
  Description of Artificial Sequence Synthetic peptide
EGCA

SEQ ID NO: 808 - protein, 13 aa, Artificial Sequence
  Description of Artificial Sequence Synthetic peptide
SQNIFSGFSTELL

SEQ ID NO: 809 - protein, 11 aa, Artificial Sequence
  Description of Artificial Sequence Synthetic peptide
SQNIFSGFSTE

SEQ ID NO: 810 - protein, 10 aa, Artificial Sequence
  Description of Artificial Sequence Synthetic peptide
QNDQRGEIVR

SEQ ID NO: 811 - protein, 12 aa, Artificial Sequence
  Description of Artificial Sequence Synthetic peptide
SQNIFSGFSTEL

SEQ ID NO: 812 - protein, 12 aa, Artificial Sequence
  Description of Artificial Sequence Synthetic peptide
QLQCQNDQRGEI

SEQ ID NO: 813 - protein, 12 aa, Artificial Sequence
  Description of Artificial Sequence Synthetic peptide
LGQSTSQWQSSR

SEQ ID NO: 814 - protein, 15 aa, Artificial Sequence
  Description of Artificial Sequence Synthetic peptide
QQLLGQSTSQWQSSR

SEQ ID NO: 815 - protein, 13 aa, Artificial Sequence
  Description of Artificial Sequence Synthetic peptide
LLGQSTSQWQSSR

SEQ ID NO: 816 - protein, 9 aa, Artificial Sequence
  Description of Artificial Sequence Synthetic peptide
NDQRGEIVR

SEQ ID NO: 817 - protein, 11 aa, Artificial Sequence
  Description of Artificial Sequence Synthetic peptide
GQSTSQWQSSR

SEQ ID NO: 818 - protein, 9 aa, Artificial Sequence
  Description of Artificial Sequence Synthetic peptide
STSQWQSSR

SEQ ID NO: 819 - protein, 13 aa, Artificial Sequence
  Description of Artificial Sequence Synthetic peptide
GITGPTFPGCPET

SEQ ID NO: 820 - protein, 14 aa, Artificial Sequence
  Description of Artificial Sequence Synthetic peptide
GITGPTFPGCPETY

SEQ ID NO: 821 - protein, 12 aa, Artificial Sequence
  Description of Artificial Sequence Synthetic peptide
SQNIFSGFSTEL

SEQ ID NO: 822 - protein, 12 aa, Artificial Sequence
  Description of Artificial Sequence Synthetic peptide
QLQCQNDQRGEI

SEQ ID NO: 823 - protein, 12 aa, Artificial Sequence
  Description of Artificial Sequence Synthetic peptide
LGQSTSQWQSSR

SEQ ID NO: 824 - protein, 15 aa, Artificial Sequence
  Description of Artificial Sequence Synthetic peptide
QQLLGQSTSQWQSSR

SEQ ID NO: 825 - protein, 13 aa, Artificial Sequence
  Description of Artificial Sequence Synthetic peptide
LLGQSTSQWQSSR

SEQ ID NO: 826 - protein, 9 aa, Artificial Sequence
  Description of Artificial Sequence Synthetic peptide
IFFANQTYL

SEQ ID NO: 827 - protein, 14 aa, Artificial Sequence
  Description of Artificial Sequence Synthetic peptide
EHLEPNLEGLTVEE

SEQ ID NO: 828 - protein, 36 aa, Artificial Sequence
  Description of Artificial Sequence Synthetic polypeptide
IFFANQTYLPSETPAPLVHYREEELNNLRGDGTGER

SEQ ID NO: 829 - protein, 18 aa, Artificial Sequence
  Description of Artificial Sequence Synthetic peptide
IHFEWDDDMGIPGAFYIK

SEQ ID NO: 830 - protein, 29 aa, Artificial Sequence
  Description of Artificial Sequence Synthetic peptide
IFFANQTYLPSETPAPLVHYREEELNNLR

SEQ ID NO: 831 - protein, 11 aa, Artificial Sequence
  Description of Artificial Sequence Synthetic peptide
TEQALPADLIK

SEQ ID NO: 832 - protein, 20 aa, Artificial Sequence
  Description of Artificial Sequence Synthetic peptide
EHLEPNLEGLTVEEAIQNKK

SEQ ID NO: 833 - protein, 23 aa, Artificial Sequence
  Description of Artificial Sequence Synthetic peptide
ISKEHLEPNLEGLTVEEAIQNKK

SEQ ID NO: 834 - protein, 16 aa, Artificial Sequence
  Description of Artificial Sequence Synthetic peptide
LSLPHPQGDEHGAVSY

SEQ ID NO: 835 - protein, 22 aa, Artificial Sequence
  Description of Artificial Sequence Synthetic peptide
ISKEHLEPNLEGLTVEEAIQNK

SEQ ID NO: 836 - protein, 19 aa, Artificial Sequence
  Description of Artificial Sequence Synthetic peptide
EHLEPNLEGLTVEEAIQNK

SEQ ID NO: 837 - protein, 18 aa, Artificial Sequence
  Description of Artificial Sequence Synthetic peptide
LSTTGGNSGSPVFNEKNE

SEQ ID NO: 838 - protein, 17 aa, Artificial Sequence
  Description of Artificial Sequence Synthetic peptide
QSFLLSGNQNQQNYLSG

SEQ ID NO: 839 - protein, 16 aa, Artificial Sequence
  Description of Artificial Sequence Synthetic peptide
VLDLAIPVNRPGQLQS

SEQ ID NO: 840 - protein, 18 aa, Artificial Sequence
  Description of Artificial Sequence Synthetic peptide
VLDLAIPVNRPGQLQSFL

SEQ ID NO: 841 - protein, 15 aa, Artificial Sequence
  Description of Artificial Sequence Synthetic peptide
FLLSGNQNQQNYLSG

SEQ ID NO: 842 - protein, 18 aa, Artificial Sequence
  Description of Artificial Sequence Synthetic peptide
FLLSGNQNQQNYLSGFSK

SEQ ID NO: 843 - protein, 18 aa, Artificial Sequence
  Description of Artificial Sequence Synthetic peptide
DPQNPFIFKSNKFQTLFE

SEQ ID NO: 844 - protein, 19 aa, Artificial Sequence
  Description of Artificial Sequence Synthetic peptide
ELAFPGSAQEVDRILENQK

SEQ ID NO: 845 - protein, 11 aa, Artificial Sequence
  Description of Artificial Sequence Synthetic peptide
AIVIVTVNEGK

SEQ ID NO: 846 - protein, 18 aa, Artificial Sequence
  Description of Artificial Sequence Synthetic peptide
INAVAAKRLQSQNDQRGE

SEQ ID NO: 847 - protein, 14 aa, Artificial Sequence
  Description of Artificial Sequence Synthetic peptide
NRAQQQQVYGSSIE

SEQ ID NO: 848 - protein, 10 aa, Artificial Sequence
  Description of Artificial Sequence Synthetic peptide
PSTNPWHSPR

SEQ ID NO: 849 - protein, 4 aa, Artificial Sequence
  Description of Artificial Sequence Synthetic peptide
CHGS

SEQ ID NO: 850 - protein, 5 aa, Artificial Sequence
  Description of Artificial Sequence Synthetic peptide
CHGSM

SEQ ID NO: 851 - protein, 4 aa, Artificial Sequence
  Description of Artificial Sequence Synthetic peptide
PWHS

SEQ ID NO: 852 - protein, 4 aa, Artificial Sequence
  Description of Artificial Sequence Synthetic peptide
FREC

SEQ ID NO: 853 - protein, 4 aa, Artificial Sequence
  Description of Artificial Sequence Synthetic peptide
RECR

SEQ ID NO: 854 - protein, 4 aa, Artificial Sequence
  Description of Artificial Sequence Synthetic peptide
ECRF

SEQ ID NO: 855 - protein, 4 aa, Artificial Sequence
  Description of Artificial Sequence Synthetic peptide
CRFD

SEQ ID NO: 856 - protein, 5 aa, Artificial Sequence
  Description of Artificial Sequence Synthetic peptide
CRFDR

SEQ ID NO: 857 - protein, 4 aa, Artificial Sequence
  Description of Artificial Sequence Synthetic peptide
CTGT

SEQ ID NO: 858 - protein, 4 aa, Artificial Sequence
  Description of Artificial Sequence Synthetic peptide
FPGC

SEQ ID NO: 859 - protein, 5 aa, Artificial Sequence
  Description of Artificial Sequence Synthetic peptide
FPGCP

SEQ ID NO: 860 - protein, 4 aa, Artificial Sequence
  Description of Artificial Sequence Synthetic peptide
PGCP

SEQ ID NO: 861 - protein, 5 aa, Artificial Sequence
  Description of Artificial Sequence Synthetic peptide
PGCPA

SEQ ID NO: 862 - protein, 6 aa, Artificial Sequence
  Description of Artificial Sequence Synthetic peptide
PGCPAT

SEQ ID NO: 863 - protein, 4 aa, Artificial Sequence
  Description of Artificial Sequence Synthetic peptide
GCPA

SEQ ID NO: 864 - protein, 4 aa, Artificial Sequence
  Description of Artificial Sequence Synthetic peptide
CPAT

SEQ ID NO: 865 - protein, 4 aa, Artificial Sequence
  Description of Artificial Sequence Synthetic peptide
ENFC

SEQ ID NO: 866 - protein, 4 aa, Artificial Sequence
  Description of Artificial Sequence Synthetic peptide
NFCT

SEQ ID NO: 867 - protein, 5 aa, Artificial Sequence
  Description of Artificial Sequence Synthetic peptide
NFCTI

SEQ ID NO: 868 - protein, 4 aa, Artificial Sequence
  Description of Artificial Sequence Synthetic peptide
FCTI

SEQ ID NO: 869 - protein, 14 aa, Artificial Sequence
  Description of Artificial Sequence Synthetic peptide
AQQQQVYGSSIEQH

SEQ ID NO: 870 - protein, 23 aa, Artificial Sequence
  Description of Artificial Sequence Synthetic peptide
AQQQQVYGSSIEQHSGQNIFSGF

SEQ ID NO: 871 - protein, 14 aa, Artificial Sequence
  Description of Artificial Sequence Synthetic peptide
AAKRLQSQNDQRGE

SEQ ID NO: 872 - protein, 12 aa, Artificial Sequence
  Description of Artificial Sequence Synthetic peptide
QARSLKNNRGEE

SEQ ID NO: 873 - protein, 15 aa, Artificial Sequence
  Description of Artificial Sequence Synthetic peptide
FNPSTNPWHSPRQGS

SEQ ID NO: 874 - protein, 12 aa, Artificial Sequence
  Description of Artificial Sequence Synthetic peptide
QARSLKNNRGEE

SEQ ID NO: 875 - protein, 26 aa, Artificial Sequence
  Description of Artificial Sequence Synthetic peptide
AAASLPAFCNVDIPNGGGGVCYWLAR

SEQ ID NO: 876 - protein, 18 aa, Artificial Sequence
  Description of Artificial Sequence Synthetic peptide
VAGSAPGIEGDEIAPLAK

SEQ ID NO: 877 - protein, 22 aa, Artificial Sequence
  Description of Artificial Sequence Synthetic peptide
LGVAGSAPGIEGDEIAPLAKEN

SEQ ID NO: 878 - protein, 17 aa, Artificial Sequence
  Description of Artificial Sequence Synthetic peptide
GSAPGIEGDEIAPLAKE

SEQ ID NO: 879 - protein, 15 aa, Artificial Sequence
  Description of Artificial Sequence Synthetic peptide
VAGSAPGIEGDEIAP

SEQ ID NO: 880 - protein, 19 aa, Artificial Sequence
  Description of Artificial Sequence Synthetic peptide
GVAGSAPGIEGDEIAPLAK

SEQ ID NO: 881 - protein, 21 aa, Artificial Sequence
  Description of Artificial Sequence Synthetic peptide
GVAGSAPGIEGDEIAPLAKEN

SEQ ID NO: 882 - protein, 20 aa, Artificial Sequence
  Description of Artificial Sequence Synthetic peptide
VAGSAPGIEGDEIAPLAKEN

SEQ ID NO: 883 - protein, 15 aa, Artificial Sequence
  Description of Artificial Sequence Synthetic peptide
SAPGIEGDEIAPLAK

SEQ ID NO: 884 - protein, 16 aa, Artificial Sequence
  Description of Artificial Sequence Synthetic peptide
GSAPGIEGDEIAPLAK

SEQ ID NO: 885 - protein, 17 aa, Artificial Sequence
  Description of Artificial Sequence Synthetic peptide
SAPGIEGDEIAPLAKEN

SEQ ID NO: 886 - protein, 15 aa, Artificial Sequence
  Description of Artificial Sequence Synthetic peptide
FNPSTNPWHSPRQGS

SEQ ID NO: 887 - protein, 12 aa, Artificial Sequence
  Description of Artificial Sequence Synthetic peptide
VDLVIPVNGPGK

SEQ ID NO: 888 - protein, 13 aa, Artificial Sequence
  Description of Artificial Sequence Synthetic peptide
LVDLVIPVNGPGK

SEQ ID NO: 889 - protein, 10 aa, Artificial Sequence
  Description of Artificial Sequence Synthetic peptide
IKLPAGTTSY

SEQ ID NO: 890 - protein, 11 aa, Artificial Sequence
  Description of Artificial Sequence Synthetic peptide
IKLPAGTTSYL

SEQ ID NO: 891 - protein, 12 aa, Artificial Sequence
  Description of Artificial Sequence Synthetic peptide
RRNPFLFKSNKF

SEQ ID NO: 892 - protein, 19 aa, Artificial Sequence
  Description of Artificial Sequence Synthetic peptide
IENPVKELTFPGSVQEINR

SEQ ID NO: 893 - protein, 14 aa, Artificial Sequence
  Description of Artificial Sequence Synthetic peptide
RRNPFLFKSNKFLT

SEQ ID NO: 894 - protein, 14 aa, Artificial Sequence
  Description of Artificial Sequence Synthetic peptide
AKPHTIFLPQHIDA

SEQ ID NO: 895 - protein, 15 aa, Artificial Sequence
  Description of Artificial Sequence Synthetic peptide
AKPHTIFLPQHIDAD

SEQ ID NO: 896 - protein, 9 aa, Artificial Sequence
  Description of Artificial Sequence Synthetic peptide
KQKYRYQRE

SEQ ID NO: 897 - protein, 9 aa, Artificial Sequence
  Description of Artificial Sequence Synthetic peptide
KQKYQYQRE

SEQ ID NO: 898 - protein, 4 aa, Artificial Sequence
  Description of Artificial Sequence Synthetic peptide
MLPH

SEQ ID NO: 899 - protein, 19 aa, Artificial Sequence
  Description of Artificial Sequence Synthetic peptide
RRNPFLFKSNKFLTLFENE

SEQ ID NO: 900 - protein, 14 aa, Artificial Sequence
  Description of Artificial Sequence Synthetic peptide
PFLFKSNKFLTLFE

SEQ ID NO: 901 - protein, 20 aa, Artificial Sequence
  Description of Artificial Sequence Synthetic peptide
SQERRNPFLFKSNKFLTLFE

SEQ ID NO: 902 - protein, 17 aa, Artificial Sequence
  Description of Artificial Sequence Synthetic peptide
RRNPFLFKSNKFLTLFE

SEQ ID NO: 903 - protein, 22 aa, Artificial Sequence
  Description of Artificial Sequence Synthetic peptide
SQERRNPFLFKSNKFLTLFENE

SEQ ID NO: 904 - protein, 9 aa, Artificial Sequence
  Description of Artificial Sequence Synthetic peptide
LTFPGSVQE

SEQ ID NO: 905 - protein, 13 aa, Artificial Sequence
  Description of Artificial Sequence Synthetic peptide
ELTFPGSVQEINR

SEQ ID NO: 906 - protein, 10 aa, Artificial Sequence
  Description of Artificial Sequence Synthetic peptide
KNPQLQDLDI

SEQ ID NO: 907 - protein, 11 aa, Artificial Sequence
  Description of Artificial Sequence Synthetic peptide
GQSTSQWQSSR

SEQ ID NO: 908 - protein, 10 aa, Artificial Sequence
  Description of Artificial Sequence Synthetic peptide
QSTSQWQSSR

SEQ ID NO: 909 - protein, 10 aa, Artificial Sequence
  Description of Artificial Sequence Synthetic peptide
QSTSQWQSSR

SEQ ID NO: 910 - protein, 12 aa, Artificial Sequence
  Description of Artificial Sequence Synthetic peptide
EEEEQGEEEINK

SEQ ID NO: 911 - protein, 10 aa, Artificial Sequence
  Description of Artificial Sequence Synthetic peptide
PSTNPWHSPR

SEQ ID NO: 912 - protein, 14 aa, Artificial Sequence
  Description of Artificial Sequence Synthetic peptide
AQAQDQYQQVQYSE

SEQ ID NO: 913 - protein, 26 aa, Artificial Sequence
  Description of Artificial Sequence Synthetic peptide
SEAGVTEYFDEKNELFQCTGTFVIRR

SEQ ID NO: 914 - protein, 15 aa, Artificial Sequence
  Description of Artificial Sequence Synthetic peptide
QAQAQDQYQQVQYSE

SEQ ID NO: 915 - protein, 13 aa, Artificial Sequence
  Description of Artificial Sequence Synthetic peptide
GSMGLTFPGCPAT

SEQ ID NO: 916 - protein, 14 aa, Artificial Sequence
  Description of Artificial Sequence Synthetic peptide
GSMGLTFPGCPATY

SEQ ID NO: 917 - protein, 8 aa, Artificial Sequence
  Description of Artificial Sequence Synthetic peptide
LGAFTPRY

SEQ ID NO: 918 - protein, 10 aa, Artificial Sequence
  Description of Artificial Sequence Synthetic peptide
LGAFTPRYQQ

SEQ ID NO: 919 - protein, 16 aa, Artificial Sequence
  Description of Artificial Sequence Synthetic peptide
ALGVNALVAKRLQGQN

SEQ ID NO: 920 - protein, 9 aa, Artificial Sequence
  Description of Artificial Sequence Synthetic peptide
LGAFTPRYQ

SEQ ID NO: 921 - protein, 13 aa, Artificial Sequence
  Description of Artificial Sequence Synthetic peptide
GSMGLTFPGCPAT

SEQ ID NO: 922 - protein, 14 aa, Artificial Sequence
  Description of Artificial Sequence Synthetic peptide
GSMGLTFPGCPATY

SEQ ID NO: 923 - protein, 14 aa, Artificial Sequence
  Description of Artificial Sequence Synthetic peptide
SNNPFKFLVPARQS

SEQ ID NO: 924 - protein, 9 aa, Artificial Sequence
  Description of Artificial Sequence Synthetic peptide
CAGVFVIRR

SEQ ID NO: 925 - protein, 10 aa, Artificial Sequence
  Description of Artificial Sequence Synthetic peptide
GSPLQSPRGF

SEQ ID NO: 926 - protein, 9 aa, Artificial Sequence
  Description of Artificial Sequence Synthetic peptide
RSSWQQQSY

SEQ ID NO: 927 - protein, 11 aa, Artificial Sequence
  Description of Artificial Sequence Synthetic peptide
SFGGSPLQSPR

SEQ ID NO: 928 - protein, 11 aa, Artificial Sequence
  Description of Artificial Sequence Synthetic peptide
YLPTKQLQPTW

SEQ ID NO: 929 - protein, 10 aa, Artificial Sequence
  Description of Artificial Sequence Synthetic peptide
GKPRSSWQQQ

SEQ ID NO: 930 - protein, 11 aa, Artificial Sequence
  Description of Artificial Sequence Synthetic peptide
FGGSPLQSPRG

SEQ ID NO: 931 - protein, 14 aa, Artificial Sequence
  Description of Artificial Sequence Synthetic peptide
LNLLGFGINAENNE

SEQ ID NO: 932 - protein, 22 aa, Artificial Sequence
  Description of Artificial Sequence Synthetic peptide
SGPFNLRSRNPIYSNKFGKFFE

SEQ ID NO: 933 - protein, 23 aa, Artificial Sequence
  Description of Artificial Sequence Synthetic peptide
TSKQVQLYKAKLSPGDVFVIPAG

SEQ ID NO: 934 - protein, 17 aa, Artificial Sequence
  Description of Artificial Sequence Synthetic peptide
GHIRLLQKFDKRSKIFE

SEQ ID NO: 935 - protein, 25 aa, Artificial Sequence
  Description of Artificial Sequence Synthetic peptide
TSKQVQLYKAKLSPGDVFVIPAGHP

SEQ ID NO: 936 - protein, 26 aa, Artificial Sequence
  Description of Artificial Sequence Synthetic peptide
TSKQVQLYKAKLSPGDVFVIPAGHPV

SEQ ID NO: 937 - protein, 27 aa, Artificial Sequence
  Description of Artificial Sequence Synthetic peptide
TSKQVQLYKAKLSPGDVFVIPAGHPVA

SEQ ID NO: 938 - protein, 28 aa, Artificial Sequence
  Description of Artificial Sequence Synthetic peptide
TSKQVQLYKAKLSPGDVFVIPAGHPVAI

SEQ ID NO: 939 - protein, 29 aa, Artificial Sequence
  Description of Artificial Sequence Synthetic peptide
TSKQVQLYKAKLSPGDVFVIPAGHPVAIN

SEQ ID NO: 940 - protein, 27 aa, Artificial Sequence
  Description of Artificial Sequence Synthetic peptide
KFKDEHQKIHRFRQGDVIALPAGVAHW

SEQ ID NO: 941 - protein, 28 aa, Artificial Sequence
  Description of Artificial Sequence Synthetic peptide
KFKDEHQKIHRFRQGDVIALPAGVAHWC

SEQ ID NO: 942 - protein, 29 aa, Artificial Sequence
  Description of Artificial Sequence Synthetic peptide
KFKDEHQKIHRFRQGDVIALPAGVAHWCY

SEQ ID NO: 943 - protein, 29 aa, Artificial Sequence
  Description of Artificial Sequence Synthetic peptide
SPFWNINAHSVVYITQGRARVQVVNNNGK

SEQ ID NO: 944 - protein, 21 aa, Artificial Sequence
  Description of Artificial Sequence Synthetic peptide
QKIHRFRQGDVIALPAGVAHW

SEQ ID NO: 945 - protein, 19 aa, Artificial Sequence
  Description of Artificial Sequence Synthetic peptide
VVRRVIEPRGLLLPHYTNG

SEQ ID NO: 946 - protein, 18 aa, Artificial Sequence
  Description of Artificial Sequence Synthetic peptide
VRRVIEPRGLLLPHYTNG

SEQ ID NO: 947 - protein, 15 aa, Artificial Sequence
  Description of Artificial Sequence Synthetic peptide
IRLLQKFDQRSKIFE

SEQ ID NO: 948 - protein, 25 aa, Artificial Sequence
  Description of Artificial Sequence Synthetic peptide
AVAAKRLQSQNDQRGEIIHVKNGLQ

SEQ ID NO: 949 - protein, 24 aa, Artificial Sequence
  Description of Artificial Sequence Synthetic peptide
VAAKRLQSQNDQRGEIIHVKNGLQ

SEQ ID NO: 950 - protein, 24 aa, Artificial Sequence
  Description of Artificial Sequence Synthetic peptide
TVFDGVLRPGQLLIIPQHYAVLKK

SEQ ID NO: 951 - protein, 10 aa, Artificial Sequence
  Description of Artificial Sequence Synthetic peptide
PSTNPWHSPR

SEQ ID NO: 952 - protein, 16 aa, Artificial Sequence
  Description of Artificial Sequence Synthetic peptide
NPSTNPWHSPRQGSFR

SEQ ID NO: 953 - protein, 19 aa, Artificial Sequence
  Description of Artificial Sequence Synthetic peptide
QLFNPSTNPWHSPRQGSFR

SEQ ID NO: 954 - protein, 22 aa, Artificial Sequence
  Description of Artificial Sequence Synthetic peptide
SMAQLFNPSTNPWHSPRQGSFR

SEQ ID NO: 955 - protein, 23 aa, Artificial Sequence
  Description of Artificial Sequence Synthetic peptide
QLFNPSTNPWHSPRQGSFRECRF

SEQ ID NO: 956 - protein, 25 aa, Artificial Sequence
  Description of Artificial Sequence Synthetic peptide
CHGSMAQLFNPSTNPWHSPRQGSFR

SEQ ID NO: 957 - protein, 29 aa, Artificial Sequence
  Description of Artificial Sequence Synthetic peptide
CHGSMAQLFNPSTNPWHSPRQGSFRECRF

SEQ ID NO: 958 - protein, 28 aa, Artificial Sequence
  Description of Artificial Sequence Synthetic peptide
LLLCHGSMAQLFNPSTNPWHSPRQGSFR

SEQ ID NO: 959 - protein, 16 aa, Artificial Sequence
  Description of Artificial Sequence Synthetic peptide
PSTNPWHSPRQGSFRE

SEQ ID NO: 960 - protein, 12 aa, Artificial Sequence
  Description of Artificial Sequence Synthetic peptide
PWHSPRQGSFRE

SEQ ID NO: 961 - protein, 18 aa, Artificial Sequence
  Description of Artificial Sequence Synthetic peptide
QLFNPSTNPWHSPRQGSF

SEQ ID NO: 962 - protein, 12 aa, Artificial Sequence
  Description of Artificial Sequence Synthetic peptide
RVIQPQGLLVPR

SEQ ID NO: 963 - protein, 16 aa, Artificial Sequence
  Description of Artificial Sequence Synthetic peptide
FNPSTNPWHSPRQGSF

SEQ ID NO: 964 - protein, 15 aa, Artificial Sequence
  Description of Artificial Sequence Synthetic peptide
FNPSTNPWHSPRQGS

SEQ ID NO: 965 - protein, 19 aa, Artificial Sequence
  Description of Artificial Sequence Synthetic peptide
GPNVNPWHNPRQGGFRECR

SEQ ID NO: 966 - protein, 19 aa, Artificial Sequence
  Description of Artificial Sequence Synthetic peptide
QLFGPNVNPWHNPRQGGFR

SEQ ID NO: 967 - protein, 22 aa, Artificial Sequence
  Description of Artificial Sequence Synthetic peptide
QLFGPNVNPWHNPRQGGFRECR

SEQ ID NO: 968 - protein, 22 aa, Artificial Sequence
  Description of Artificial Sequence Synthetic peptide
SMAQLFGPNVNPWHNPRQGGFR

SEQ ID NO: 969 - protein, 25 aa, Artificial Sequence
  Description of Artificial Sequence Synthetic peptide
CHGSMAQLFGPNVNPWHNPRQGGFR

SEQ ID NO: 970 - protein, 25 aa, Artificial Sequence
  Description of Artificial Sequence Synthetic peptide
SMAQLFGPNVNPWHNPRQGGFRECR

SEQ ID NO: 971 - protein, 28 aa, Artificial Sequence
  Description of Artificial Sequence Synthetic peptide
LLLCHGSMAQLFGPNVNPWHNPRQGGFR

SEQ ID NO: 972 - protein, 9 aa, Artificial Sequence
  Description of Artificial Sequence Synthetic peptide
WRPSYEKEE

SEQ ID NO: 973 - protein, 35 aa, Artificial Sequence
  Description of Artificial Sequence Synthetic polypeptide
AKPHTIFLPQHIDADLILVVLSGKAILTVLSPNDR

SEQ ID NO: 974 - protein, 26 aa, Artificial Sequence
  Description of Artificial Sequence Synthetic peptide
PRVPAQRERGRQEGEKEEKRHGEWRP

SEQ ID NO: 975 - protein, 27 aa, Artificial Sequence
  Description of Artificial Sequence Synthetic peptide
PRVPAQRERGRQEGEKEEKRHGEWRPS

SEQ ID NO: 976 - protein, 28 aa, Artificial Sequence
  Description of Artificial Sequence Synthetic peptide
PRVPAQRERGRQEGEKEEKRHGEWRPSY

SEQ ID NO: 977 - protein, 25 aa, Artificial Sequence
  Description of Artificial Sequence Synthetic peptide
PRVPAQRERGRQEGEKEEKRHGEWR

SEQ ID NO: 978 - protein, 29 aa, Artificial Sequence
  Description of Artificial Sequence Synthetic peptide
GSEPRVPAQRERGRQEGEKEEKRHGEWRP

SEQ ID NO: 979 - protein, 12 aa, Artificial Sequence
  Description of Artificial Sequence Synthetic peptide
KRHGEWRPSYEK

SEQ ID NO: 980 - protein, 14 aa, Artificial Sequence
  Description of Artificial Sequence Synthetic peptide
KKEQKEVQPGRERW

SEQ ID NO: 981 - protein, 16 aa, Artificial Sequence
  Description of Artificial Sequence Synthetic peptide
KKEQKEVQPGRERWER

SEQ ID NO: 982 - protein, 16 aa, Artificial Sequence
  Description of Artificial Sequence Synthetic peptide
REKKEQKEVQPGRERW

SEQ ID NO: 983 - protein, 17 aa, Artificial Sequence
  Description of Artificial Sequence Synthetic peptide
QREKKEQKEVQPGRERW

SEQ ID NO: 984 - protein, 18 aa, Artificial Sequence
  Description of Artificial Sequence Synthetic peptide
REKKEQKEVQPGRERWER

SEQ ID NO: 985 - protein, 19 aa, Artificial Sequence
  Description of Artificial Sequence Synthetic peptide
QREKKEQKEVQPGRERWER

SEQ ID NO: 986 - protein, 19 aa, Artificial Sequence
  Description of Artificial Sequence Synthetic peptide
QYQREKKEQKEVQPGRERW

SEQ ID NO: 987 - protein, 20 aa, Artificial Sequence
  Description of Artificial Sequence Synthetic peptide
YQYQREKKEQKEVQPGRERW

SEQ ID NO: 988 - protein, 21 aa, Artificial Sequence
  Description of Artificial Sequence Synthetic peptide
QYQREKKEQKEVQPGRERWER

SEQ ID NO: 989 - protein, 22 aa, Artificial Sequence
  Description of Artificial Sequence Synthetic peptide
QKYQYQREKKEQKEVQPGRERW

SEQ ID NO: 990 - protein, 22 aa, Artificial Sequence
  Description of Artificial Sequence Synthetic peptide
YQYQREKKEQKEVQPGRERWER

SEQ ID NO: 991 - protein, 23 aa, Artificial Sequence
  Description of Artificial Sequence Synthetic peptide
KQKYQYQREKKEQKEVQPGRERW

SEQ ID NO: 992 - protein, 23 aa, Artificial Sequence
  Description of Artificial Sequence Synthetic peptide
QKYQYQREKKEQKEVQPGRERWE

SEQ ID NO: 993 - protein, 24 aa, Artificial Sequence
  Description of Artificial Sequence Synthetic peptide
KQKYQYQREKKEQKEVQPGRERWE

SEQ ID NO: 994 - protein, 24 aa, Artificial Sequence
  Description of Artificial Sequence Synthetic peptide
QKYQYQREKKEQKEVQPGRERWER

SEQ ID NO: 995 - protein, 25 aa, Artificial Sequence
  Description of Artificial Sequence Synthetic peptide
EEKQKYQYQREKKEQKEVQPGRERW

SEQ ID NO: 996 - protein, 25 aa, Artificial Sequence
  Description of Artificial Sequence Synthetic peptide
KQKYQYQREKKEQKEVQPGRERWER

SEQ ID NO: 997 - protein, 25 aa, Artificial Sequence
  Description of Artificial Sequence Synthetic peptide
QKYQYQREKKEQKEVQPGRERWERE

SEQ ID NO: 998 - protein, 26 aa, Artificial Sequence
  Description of Artificial Sequence Synthetic peptide
KQKYQYQREKKEQKEVQPGRERWERE

SEQ ID NO: 999 - protein, 27 aa, Artificial Sequence
  Description of Artificial Sequence Synthetic peptide
EEKQKYQYQREKKEQKEVQPGRERWER

SEQ ID NO: 1000 - protein, 27 aa, Artificial Sequence
  Description of Artificial Sequence Synthetic peptide
KQKYQYQREKKEQKEVQPGRERWEREE

SEQ ID NO: 1001 - protein, 28 aa, Artificial Sequence
  Description of Artificial Sequence Synthetic peptide
PINLRSHKPEYSNKFGKLFEITPEKKYP

SEQ ID NO: 1002 - protein, 10 aa, Artificial Sequence
  Description of Artificial Sequence Synthetic peptide
KRHGEWRPSY

SEQ ID NO: 1003 - protein, 23 aa, Artificial Sequence
  Description of Artificial Sequence Synthetic peptide
PAQRERGRQEGEKEEKRHGEWRP

SEQ ID NO: 1004 - protein, 24 aa, Artificial Sequence
  Description of Artificial Sequence Synthetic peptide
PAQRERGRQEGEKEEKRHGEWRPS

SEQ ID NO: 1005 - protein, 24 aa, Artificial Sequence
  Description of Artificial Sequence Synthetic peptide
PRVPAQRERGRQEGEKEEKRHGEW

SEQ ID NO: 1006 - protein, 25 aa, Artificial Sequence
  Description of Artificial Sequence Synthetic peptide
EWRGSQRREDPEERARLRHREERTK

SEQ ID NO: 1007 - protein, 25 aa, Artificial Sequence
  Description of Artificial Sequence Synthetic peptide
PAQRERGRQEGEKEEKRHGEWRPSY

SEQ ID NO: 1008 - protein, 26 aa, Artificial Sequence
  Description of Artificial Sequence Synthetic peptide
EWRGSQRREDPEERARLRHREERTKR

SEQ ID NO: 1009 - protein, 26 aa, Artificial Sequence
  Description of Artificial Sequence Synthetic peptide
PAQRERGRQEGEKEEKRHGEWRPSYE

SEQ ID NO: 1010 - protein, 27 aa, Artificial Sequence
  Description of Artificial Sequence Synthetic peptide
EEWRGSQRREDPEERARLRHREERTKR

SEQ ID NO: 1011 - protein, 27 aa, Artificial Sequence
  Description of Artificial Sequence Synthetic peptide
EWRGSQRREDPEERARLRHREERTKRD

SEQ ID NO: 1012 - protein, 27 aa, Artificial Sequence
  Description of Artificial Sequence Synthetic peptide
GSEPRVPAQRERGRQEGEKEEKRHGEW

SEQ ID NO: 1013 - protein, 27 aa, Artificial Sequence
  Description of Artificial Sequence Synthetic peptide
PAQRERGRQEGEKEEKRHGEWRPSYEK

SEQ ID NO: 1014 - protein, 28 aa, Artificial Sequence
  Description of Artificial Sequence Synthetic peptide
EWRGSQRREDPEERARLRHREERTKRDR

SEQ ID NO: 1015 - protein, 28 aa, Artificial Sequence
  Description of Artificial Sequence Synthetic peptide
GSEPRVPAQRERGRQEGEKEEKRHGEWR

SEQ ID NO: 1016 - protein, 28 aa, Artificial Sequence
  Description of Artificial Sequence Synthetic peptide
PAQRERGRQEGEKEEKRHGEWRPSYEKE

SEQ ID NO: 1017 - protein, 29 aa, Artificial Sequence
  Description of Artificial Sequence Synthetic peptide
EEWRGSQRREDPEERARLRHREERTKRDR

SEQ ID NO: 1018 - protein, 29 aa, Artificial Sequence
  Description of Artificial Sequence Synthetic peptide
EWRGSQRREDPEERARLRHREERTKRDRR

SEQ ID NO: 1019 - protein, 11 aa, Artificial Sequence
  Description of Artificial Sequence Synthetic peptide
KEEKRHGEWRP

SEQ ID NO: 1020 - protein, 12 aa, Artificial Sequence
  Description of Artificial Sequence Synthetic peptide
KEEKRHGEWRPS

SEQ ID NO: 1021 - protein, 13 aa, Artificial Sequence
  Description of Artificial Sequence Synthetic peptide
GEKEEKRHGEWRP

SEQ ID NO: 1022 - protein, 13 aa, Artificial Sequence
  Description of Artificial Sequence Synthetic peptide
KEEKRHGEWRPSY

SEQ ID NO: 1023 - protein, 13 aa, Artificial Sequence
  Description of Artificial Sequence Synthetic peptide
KEQKEVQPGRERW

SEQ ID NO: 1024 - protein, 13 aa, Artificial Sequence
  Description of Artificial Sequence Synthetic peptide
KRHGEWRPSYEKE

SEQ ID NO: 1025 - protein, 14 aa, Artificial Sequence
  Description of Artificial Sequence Synthetic peptide
EEKRHGEWRPSYEK

SEQ ID NO: 1026 - protein, 14 aa, Artificial Sequence
  Description of Artificial Sequence Synthetic peptide
GEKEEKRHGEWRPS

SEQ ID NO: 1027 - protein, 14 aa, Artificial Sequence
  Description of Artificial Sequence Synthetic peptide
KEEKRHGEWRPSYE

SEQ ID NO: 1028 - protein, 14 aa, Artificial Sequence
  Description of Artificial Sequence Synthetic peptide
KEQKEVQPGRERWE

SEQ ID NO: 1029 - protein, 15 aa, Artificial Sequence
  Description of Artificial Sequence Synthetic peptide
EEKRHGEWRPSYEKQ

SEQ ID NO: 1030 - protein, 15 aa, Artificial Sequence
  Description of Artificial Sequence Synthetic peptide
GEKEEKRHGEWRPSY

SEQ ID NO: 1031 - protein, 15 aa, Artificial Sequence
  Description of Artificial Sequence Synthetic peptide
KEEKRHGEWRPSYEK

SEQ ID NO: 1032 - protein, 15 aa, Artificial Sequence
  Description of Artificial Sequence Synthetic peptide
KEQKEVQPGRERWER

SEQ ID NO: 1033 - protein, 15 aa, Artificial Sequence
  Description of Artificial Sequence Synthetic peptide
KKEQKEVQPGRERWE

SEQ ID NO: 1034 - protein, 16 aa, Artificial Sequence
  Description of Artificial Sequence Synthetic peptide
KEEKRHGEWRPSYEKE

SEQ ID NO: 1035 - protein, 16 aa, Artificial Sequence
  Description of Artificial Sequence Synthetic peptide
KEQKEVQPGRERWERE

SEQ ID NO: 1036 - protein, 16 aa, Artificial Sequence
  Description of Artificial Sequence Synthetic peptide
RQEGEKEEKRHGEWRP

SEQ ID NO: 1037 - protein, 17 aa, Artificial Sequence
  Description of Artificial Sequence Synthetic peptide
GEKEEKRHGEWRPSYEK

SEQ ID NO: 1038 - protein, 17 aa, Artificial Sequence
  Description of Artificial Sequence Synthetic peptide
GRQEGEKEEKRHGEWRP

SEQ ID NO: 1039 - protein, 17 aa, Artificial Sequence
  Description of Artificial Sequence Synthetic peptide
KKEQKEVQPGRERWERE

SEQ ID NO: 1040 - protein, 17 aa, Artificial Sequence
  Description of Artificial Sequence Synthetic peptide
REKKEQKEVQPGRERWE

SEQ ID NO: 1041 - protein, 17 aa, Artificial Sequence
  Description of Artificial Sequence Synthetic peptide
RQEGEKEEKRHGEWRPS

SEQ ID NO: 1042 - protein, 18 aa, Artificial Sequence
  Description of Artificial Sequence Synthetic peptide
GEKEEKRHGEWRPSYEKQ

SEQ ID NO: 1043 - protein, 18 aa, Artificial Sequence
  Description of Artificial Sequence Synthetic peptide
GRQEGEKEEKRHGEWRPS

SEQ ID NO: 1044 - protein, 18 aa, Artificial Sequence
  Description of Artificial Sequence Synthetic peptide
KKEQKEVQPGRERWEREE

SEQ ID NO: 1045 - protein, 18 aa, Artificial Sequence
  Description of Artificial Sequence Synthetic peptide
QREKKEQKEVQPGRERWE

SEQ ID NO: 1046 - protein, 18 aa, Artificial Sequence
  Description of Artificial Sequence Synthetic peptide
RQEGEKEEKRHGEWRPSY

SEQ ID NO: 1047 - protein, 19 aa, Artificial Sequence
  Description of Artificial Sequence Synthetic peptide
ERGRQEGEKEEKRHGEWRP

SEQ ID NO: 1048 - protein, 19 aa, Artificial Sequence
  Description of Artificial Sequence Synthetic peptide
GRQEGEKEEKRHGEWRPSY

SEQ ID NO: 1049 - protein, 19 aa, Artificial Sequence
  Description of Artificial Sequence Synthetic peptide
REKKEQKEVQPGRERWERE

SEQ ID NO: 1050 - protein, 20 aa, Artificial Sequence
  Description of Artificial Sequence Synthetic peptide
ERGRQEGEKEEKRHGEWRPS

SEQ ID NO: 1051 - protein, 20 aa, Artificial Sequence
  Description of Artificial Sequence Synthetic peptide
QREKKEQKEVQPGRERWERE

SEQ ID NO: 1052 - protein, 20 aa, Artificial Sequence
  Description of Artificial Sequence Synthetic peptide
QYQREKKEQKEVQPGRERWE

SEQ ID NO: 1053 - protein, 20 aa, Artificial Sequence
  Description of Artificial Sequence Synthetic peptide
REKKEQKEVQPGRERWEREE

SEQ ID NO: 1054 - protein, 20 aa, Artificial Sequence
  Description of Artificial Sequence Synthetic peptide
RERGRQEGEKEEKRHGEWRP

SEQ ID NO: 1055 - protein, 20 aa, Artificial Sequence
  Description of Artificial Sequence Synthetic peptide
RQEGEKEEKRHGEWRPSYEK

SEQ ID NO: 1056 - protein, 21 aa, Artificial Sequence
  Description of Artificial Sequence Synthetic peptide
ERGRQEGEKEEKRHGEWRPSY

SEQ ID NO: 1057 - protein, 21 aa, Artificial Sequence
  Description of Artificial Sequence Synthetic peptide
GRQEGEKEEKRHGEWRPSYEK

SEQ ID NO: 1058 - protein, 21 aa, Artificial Sequence
  Description of Artificial Sequence Synthetic peptide
PAQRERGRQEGEKEEKRHGEW

SEQ ID NO: 1059 - protein, 21 aa, Artificial Sequence
  Description of Artificial Sequence Synthetic peptide
QREKKEQKEVQPGRERWEREE

SEQ ID NO: 1060 - protein, 21 aa, Artificial Sequence
  Description of Artificial Sequence Synthetic peptide
RERGRQEGEKEEKRHGEWRPS

SEQ ID NO: 1061 - protein, 21 aa, Artificial Sequence
  Description of Artificial Sequence Synthetic peptide
RQEGEKEEKRHGEWRPSYEKQ

SEQ ID NO: 1062 - protein, 21 aa, Artificial Sequence
  Description of Artificial Sequence Synthetic peptide
YQYQREKKEQKEVQPGRERWE

SEQ ID NO: 1063 - protein, 22 aa, Artificial Sequence
  Description of Artificial Sequence Synthetic peptide
GQRERGRQEGEKEEKRHGEWRP

SEQ ID NO: 1064 - protein, 22 aa, Artificial Sequence
  Description of Artificial Sequence Synthetic peptide
PAQRERGRQEGEKEEKRHGEWR

SEQ ID NO: 1065 - protein, 22 aa, Artificial Sequence
  Description of Artificial Sequence Synthetic peptide
QYQREKKEQKEVQPGRERWERE

SEQ ID NO: 1066 - protein, 22 aa, Artificial Sequence
  Description of Artificial Sequence Synthetic peptide
RERGRQEGEKEEKRHGEWRPSY

SEQ ID NO: 1067 - protein, 23 aa, Artificial Sequence
  Description of Artificial Sequence Synthetic peptide
ERGRQEGEKEEKRHGEWRPSYEK

SEQ ID NO: 1068 - protein, 23 aa, Artificial Sequence
  Description of Artificial Sequence Synthetic peptide
GQRERGRQEGEKEEKRHGEWRPS

SEQ ID NO: 1069 - protein, 23 aa, Artificial Sequence
  Description of Artificial Sequence Synthetic peptide
QYQREKKEQKEVQPGRERWEREE

SEQ ID NO: 1070 - protein, 23 aa, Artificial Sequence
  Description of Artificial Sequence Synthetic peptide
RERGRQEGEKEEKRHGEWRPSYE

SEQ ID NO: 1071 - protein, 23 aa, Artificial Sequence
  Description of Artificial Sequence Synthetic peptide
YQYQREKKEQKEVQPGRERWERE

SEQ ID NO: 1072 - protein, 24 aa, Artificial Sequence
  Description of Artificial Sequence Synthetic peptide
ERGRQEGEKEEKRHGEWRPSYEKQ

SEQ ID NO: 1073 - protein, 24 aa, Artificial Sequence
  Description of Artificial Sequence Synthetic peptide
GQRERGRQEGEKEEKRHGEWRPSY

SEQ ID NO: 1074 - protein, 24 aa, Artificial Sequence
  Description of Artificial Sequence Synthetic peptide
RERGRQEGEKEEKRHGEWRPSYEK

SEQ ID NO: 1075 - protein, 24 aa, Artificial Sequence
  Description of Artificial Sequence Synthetic peptide
YQYQREKKEQKEVQPGRERWEREE

SEQ ID NO: 1076 - protein, 25 aa, Artificial Sequence
  Description of Artificial Sequence Synthetic peptide
GQRERGRQEGEKEEKRHGEWRPSYE

SEQ ID NO: 1077 - protein, 25 aa, Artificial Sequence
  Description of Artificial Sequence Synthetic peptide
RERGRQEGEKEEKRHGEWRPSYEKE

SEQ ID NO: 1078 - protein, 25 aa, Artificial Sequence
  Description of Artificial Sequence Synthetic peptide
RHGEWRPSYEKQEDEEEKQKYRYQR

SEQ ID NO: 1079 - protein, 26 aa, Artificial Sequence
  Description of Artificial Sequence Synthetic peptide
EEEKQKYQYQREKKEQKEVQPGRERW

SEQ ID NO: 1080 - protein, 26 aa, Artificial Sequence
  Description of Artificial Sequence Synthetic peptide
GQRERGRQEGEKEEKRHGEWRPSYEK

SEQ ID NO: 1081 - protein, 26 aa, Artificial Sequence
  Description of Artificial Sequence Synthetic peptide
QKYQYQREKKEQKEVQPGRERWEREE

SEQ ID NO: 1082 - protein, 27 aa, Artificial Sequence
  Description of Artificial Sequence Synthetic peptide
EEEKQKYQYQREKKEQKEVQPGRERWE

SEQ ID NO: 1083 - protein, 27 aa, Artificial Sequence
  Description of Artificial Sequence Synthetic peptide
GQRERGRQEGEKEEKRHGEWRPSYEKQ

SEQ ID NO: 1084 - protein, 27 aa, Artificial Sequence
  Description of Artificial Sequence Synthetic peptide
QKYQYQREKKEQKEVQPGRERWEREED

SEQ ID NO: 1085 - protein, 27 aa, Artificial Sequence
  Description of Artificial Sequence Synthetic peptide
RHGEWRPSYEKQEDEEEKQKYRYQREK

SEQ ID NO: 1086 - protein, 28 aa, Artificial Sequence
  Description of Artificial Sequence Synthetic peptide
EEEKQKYQYQREKKEQKEVQPGRERWER

SEQ ID NO: 1087 - protein, 28 aa, Artificial Sequence
  Description of Artificial Sequence Synthetic peptide
EEKQKYQYQREKKEQKEVQPGRERWERE

SEQ ID NO: 1088 - protein, 28 aa, Artificial Sequence
  Description of Artificial Sequence Synthetic peptide
GQRERGRQEGEKEEKRHGEWRPSYEKQE

SEQ ID NO: 1089 - protein, 28 aa, Artificial Sequence
  Description of Artificial Sequence Synthetic peptide
KQKYQYQREKKEQKEVQPGRERWEREED

SEQ ID NO: 1090 - protein, 28 aa, Artificial Sequence
  Description of Artificial Sequence Synthetic peptide
PSEFEPINLRSHKPEYSNKFGKLFEITP

SEQ ID NO: 1091 - protein, 29 aa, Artificial Sequence
  Description of Artificial Sequence Synthetic peptide
EEEKQKYQYQREKKEQKEVQPGRERWERE

SEQ ID NO: 1092 - protein, 29 aa, Artificial Sequence
  Description of Artificial Sequence Synthetic peptide
KEDEEEKQKYQYQREKKEQKEVQPGRERW

SEQ ID NO: 1093 - protein, 29 aa, Artificial Sequence
  Description of Artificial Sequence Synthetic peptide
KQKYQYQREKKEQKEVQPGRERWEREEDE

SEQ ID NO: 1094 - protein, 29 aa, Artificial Sequence
  Description of Artificial Sequence Synthetic peptide
LPSEFEPINLRSHKPEYSNKFGKLFEITP

SEQ ID NO: 1095 - protein, 10 aa, Artificial Sequence
  Description of Artificial Sequence Synthetic peptide
EEKRHGEWRP

SEQ ID NO: 1096 - protein, 10 aa, Artificial Sequence
  Description of Artificial Sequence Synthetic peptide
KEVQPGRERW

SEQ ID NO: 1097 - protein, 11 aa, Artificial Sequence
  Description of Artificial Sequence Synthetic peptide
QKEVQPGRERW

SEQ ID NO: 1098 - protein, 12 aa, Artificial Sequence
  Description of Artificial Sequence Synthetic peptide
EEKRHGEWRPSY

SEQ ID NO: 1099 - protein, 12 aa, Artificial Sequence
  Description of Artificial Sequence Synthetic peptide
RHGEWRPSYEKQ

SEQ ID NO: 1100 - protein, 13 aa, Artificial Sequence
  Description of Artificial Sequence Synthetic peptide
KEVQPGRERWERE

SEQ ID NO: 1101 - protein, 13 aa, Artificial Sequence
  Description of Artificial Sequence Synthetic peptide
RHGEWRPSYEKQE

SEQ ID NO: 1102 - protein, 14 aa, Artificial Sequence
  Description of Artificial Sequence Synthetic peptide
QKEVQPGRERWERE

SEQ ID NO: 1103 - protein, 18 aa, Artificial Sequence
  Description of Artificial Sequence Synthetic peptide
KKSLPSEFEPINLRSHKP

SEQ ID NO: 1104 - protein, 20 aa, Artificial Sequence
  Description of Artificial Sequence Synthetic peptide
EWRGSQRREDPEERARLRHR

SEQ ID NO: 1105 - protein, 20 aa, Artificial Sequence
  Description of Artificial Sequence Synthetic peptide
SSKKSLPSEFEPINLRSHKP

SEQ ID NO: 1106 - protein, 21 aa, Artificial Sequence
  Description of Artificial Sequence Synthetic peptide
KPEYSNKFGKLFEITPEKKYP

SEQ ID NO: 1107 - protein, 21 aa, Artificial Sequence
  Description of Artificial Sequence Synthetic peptide
SSSKKSLPSEFEPINLRSHKP

SEQ ID NO: 1108 - protein, 22 aa, Artificial Sequence
  Description of Artificial Sequence Synthetic peptide
HKPEYSNKFGKLFEITPEKKYP

SEQ ID NO: 1109 - protein, 23 aa, Artificial Sequence
  Description of Artificial Sequence Synthetic peptide
AKSSSKKSLPSEFEPINLRSHKP

SEQ ID NO: 1110 - protein, 27 aa, Artificial Sequence
  Description of Artificial Sequence Synthetic peptide
INLRSHKPEYSNKFGKLFEITPEKKYP

SEQ ID NO: 1111 - protein, 29 aa, Artificial Sequence
  Description of Artificial Sequence Synthetic peptide
AKSSSKKSLPSEFEPINLRSHKPEYSNKF

SEQ ID NO: 1112 - protein, 17 aa, Artificial Sequence
  Description of Artificial Sequence Synthetic peptide
RRNPFLFKSNKFLTLFE

SEQ ID NO: 1113 - protein, 29 aa, Artificial Sequence
  Description of Artificial Sequence Synthetic peptide
PINLRSHKPEYSNKFGKLFEITPEKKYPQ

SEQ ID NO: 1114 - protein, 24 aa, Artificial Sequence
  Description of Artificial Sequence Synthetic peptide
KPEYSNKFGKLFEITPEKKYPQLQ

SEQ ID NO: 1115 - protein, 25 aa, Artificial Sequence
  Description of Artificial Sequence Synthetic peptide
HKPEYSNKFGKLFEITPEKKYPQLQ

SEQ ID NO: 1116 - protein, 26 aa, Artificial Sequence
  Description of Artificial Sequence Synthetic peptide
SHKPEYSNKFGKLFEITPEKKYPQLQ

SEQ ID NO: 1117 - protein, 28 aa, Artificial Sequence
  Description of Artificial Sequence Synthetic peptide
FEPINLRSHKPEYSNKFGKLFEITPEKK

SEQ ID NO: 1118 - protein, 24 aa, Artificial Sequence
  Description of Artificial Sequence Synthetic peptide
MASINRPIVFFTVCLFLLCNGSLA

SEQ ID NO: 1119 - protein, 11 aa, Artificial Sequence
  Description of Artificial Sequence Synthetic peptide
FLLAGNKRNPQ

SEQ ID NO: 1120 - protein, 9 aa, Artificial Sequence
  Description of Artificial Sequence Synthetic peptide
FLLAGNKRN

SEQ ID NO: 1121 - protein, 34 aa, Artificial Sequence
  Description of Artificial Sequence Synthetic polypeptide
VLDLAIPVNRPGQLQSFLLSGNQNQQNYLSGFSK

SEQ ID NO: 1122 - protein, 17 aa, Artificial Sequence
  Description of Artificial Sequence Synthetic peptide
QSFLLSGNQNQQNYLSG

SEQ ID NO: 1123 - protein, 5 aa, Artificial Sequence
  Description of Artificial Sequence Synthetic peptide
RLSSV

SEQ ID NO: 1124 - protein, 12 aa, Artificial Sequence
  Description of Artificial Sequence Synthetic peptide
QKEFLLAGNNNR

SEQ ID NO: 1125 - protein, 19 aa, Artificial Sequence
  Description of Artificial Sequence Synthetic peptide
LLRPAFAQQQEQAQQQEQA

SEQ ID NO: 1126 - protein, 12 aa, Artificial Sequence
  Description of Artificial Sequence Synthetic peptide
VKNGLKLLRPAF

SEQ ID NO: 1127 - protein, 10 aa, Artificial Sequence
  Description of Artificial Sequence Synthetic peptide
FLLAGNNNRE

SEQ ID NO: 1128 - protein, 14 aa, Artificial Sequence
  Description of Artificial Sequence Synthetic peptide
GLKLLRPAFAQQQE

SEQ ID NO: 1129 - protein, 13 aa, Artificial Sequence
  Description of Artificial Sequence Synthetic peptide
LKLLRPAFAQQQE

SEQ ID NO: 1130 - protein, 11 aa, Artificial Sequence
  Description of Artificial Sequence Synthetic peptide
LLRPAFAQQQE

SEQ ID NO: 1131 - protein, 12 aa, Artificial Sequence
  Description of Artificial Sequence Synthetic peptide
QKEFLLAGNNNR

SEQ ID NO: 1132 - protein, 7 aa, Artificial Sequence
  Description of Artificial Sequence Synthetic peptide
LRGFSKN

SEQ ID NO: 1133 - protein, 14 aa, Artificial Sequence
  Description of Artificial Sequence Synthetic peptide
ILDLAIPVNRPGQL

SEQ ID NO: 1134 - protein, 14 aa, Artificial Sequence
  Description of Artificial Sequence Synthetic peptide
VLELAIPVNRPGQL

SEQ ID NO: 1135 - protein, 14 aa, Artificial Sequence
  Description of Artificial Sequence Synthetic peptide
VLDLAVPVNRPGQL

SEQ ID NO: 1136 - protein, 14 aa, Artificial Sequence
  Description of Artificial Sequence Synthetic peptide
VLDLAIPINRPGQL

SEQ ID NO: 1137 - protein, 14 aa, Artificial Sequence
  Description of Artificial Sequence Synthetic peptide
VLDLAIPVNKPGQL

SEQ ID NO: 1138 - protein, 14 aa, Artificial Sequence
  Description of Artificial Sequence Synthetic peptide
VLDLAIPVEKPGQL

SEQ ID NO: 1139 - protein, 14 aa, Artificial Sequence
  Description of Artificial Sequence Synthetic peptide
VLDLAIPVNKPGEL

SEQ ID NO: 1140 - protein, 14 aa, Artificial Sequence
  Description of Artificial Sequence Synthetic peptide
ILELAIPVNRPGQL

SEQ ID NO: 1141 - protein, 14 aa, Artificial Sequence
  Description of Artificial Sequence Synthetic peptide
ILDLAVPVNRPGQL

SEQ ID NO: 1142 - protein, 14 aa, Artificial Sequence
  Description of Artificial Sequence Synthetic peptide
VLELAVPVNRPGQL

SEQ ID NO: 1143 - protein, 14 aa, Artificial Sequence
  Description of Artificial Sequence Synthetic peptide
VLELAIPVNKPGQL

SEQ ID NO: 1144 - protein, 14 aa, Artificial Sequence
  Description of Artificial Sequence Synthetic peptide
ILDLAIPVNKPGQL

SEQ ID NO: 1145 - protein, 14 aa, Artificial Sequence
  Description of Artificial Sequence Synthetic peptide
VLDLAVPVNKPGQL

SEQ ID NO: 1146 - protein, 14 aa, Artificial Sequence
  Description of Artificial Sequence Synthetic peptide
VLDLAIPVEKPGEL

SEQ ID NO: 1147 - protein, 14 aa, Artificial Sequence
  Description of Artificial Sequence Synthetic peptide
ILDLAIPVNKPGEL

SEQ ID NO: 1148 - protein, 14 aa, Artificial Sequence
  Description of Artificial Sequence Synthetic peptide
VLELAIPVEKPGQL

SEQ ID NO: 1149 - protein, 14 aa, Artificial Sequence
  Description of Artificial Sequence Synthetic peptide
ILELAVPVNRPGQL

SEQ ID NO: 1150 - protein, 14 aa, Artificial Sequence
  Description of Artificial Sequence Synthetic peptide
ILELAIPVNKPGQL

SEQ ID NO: 1151 - protein, 14 aa, Artificial Sequence
  Description of Artificial Sequence Synthetic peptide
VLELAVPVNKPGQL

SEQ ID NO: 1152 - protein, 14 aa, Artificial Sequence
  Description of Artificial Sequence Synthetic peptide
ILELAIPVNRPGEL

SEQ ID NO: 1153 - protein, 14 aa, Artificial Sequence
  Description of Artificial Sequence Synthetic peptide
ILDLAIPVNKPGEL

SEQ ID NO: 1154 - protein, 14 aa, Artificial Sequence
  Description of Artificial Sequence Synthetic peptide
VLDLAVPVEKPGQL

SEQ ID NO: 1155 - protein, 14 aa, Artificial Sequence
  Description of Artificial Sequence Synthetic peptide
VLDLAVPVERPGEL

SEQ ID NO: 1156 - protein, 14 aa, Artificial Sequence
  Description of Artificial Sequence Synthetic peptide
VLELAIPVERPGEL

SEQ ID NO: 1157 - protein, 14 aa, Artificial Sequence
  Description of Artificial Sequence Synthetic peptide
KLDLAIIVNRPGQL

SEQ ID NO: 1158 - protein, 14 aa, Artificial Sequence
  Description of Artificial Sequence Synthetic peptide
VLDLAIPVNRPGQK

SEQ ID NO: 1159 - protein, 14 aa, Artificial Sequence
  Description of Artificial Sequence Synthetic peptide
VLDLAIPVNRPCQL

SEQ ID NO: 1160 - protein, 14 aa, Artificial Sequence
  Description of Artificial Sequence Synthetic peptide
VLDLWIPVNRPGQL

SEQ ID NO: 1161 - protein, 14 aa, Artificial Sequence
  Description of Artificial Sequence Synthetic peptide
VLDLAIPVNRPGQL

SEQ ID NO: 1162 - protein, 14 aa, Artificial Sequence
  Description of Artificial Sequence Synthetic peptide
VLYLAIPVNRPGQL

SEQ ID NO: 1163 - protein, 14 aa, Artificial Sequence
  Description of Artificial Sequence Synthetic peptide
VLDLYIPVGRPGQL

SEQ ID NO: 1164 - protein, 14 aa, Artificial Sequence
  Description of Artificial Sequence Synthetic peptide
VKDLAIPWNRPGQL

SEQ ID NO: 1165 - protein, 14 aa, Artificial Sequence
  Description of Artificial Sequence Synthetic peptide
VLDLAIPVNRPCCL

SEQ ID NO: 1166 - protein, 14 aa, Artificial Sequence
  Description of Artificial Sequence Synthetic peptide
VLDLAGGVNRPGQL

SEQ ID NO: 1167 - protein, 14 aa, Artificial Sequence
  Description of Artificial Sequence Synthetic peptide
VLDLAIPKNEPGQL

SEQ ID NO: 1168 - protein, 14 aa, Artificial Sequence
  Description of Artificial Sequence Synthetic peptide
PLDLAIPVNDPGQL

SEQ ID NO: 1169 - protein, 14 aa, Artificial Sequence
  Description of Artificial Sequence Synthetic peptide
VLDLAIPVNRPIQL

SEQ ID NO: 1170 - protein, 14 aa, Artificial Sequence
  Description of Artificial Sequence Synthetic peptide
VLDHAIPVNRPGQL

SEQ ID NO: 1171 - protein, 14 aa, Artificial Sequence
  Description of Artificial Sequence Synthetic peptide
VLDLAIPVNRPGGG

SEQ ID NO: 1172 - protein, 14 aa, Artificial Sequence
  Description of Artificial Sequence Synthetic peptide
VLDLHIPGNEPGQL

SEQ ID NO: 1173 - protein, 14 aa, Artificial Sequence
  Description of Artificial Sequence Synthetic peptide
VYKLAIPVNEPGQL

SEQ ID NO: 1174 - protein, 14 aa, Artificial Sequence
  Description of Artificial Sequence Synthetic peptide
VLDLAIPVNRPYPG

SEQ ID NO: 1175 - protein, 14 aa, Artificial Sequence
  Description of Artificial Sequence Synthetic peptide
VLDYAIPKNDPGQL

SEQ ID NO: 1176 - protein, 14 aa, Artificial Sequence
  Description of Artificial Sequence Synthetic peptide
RRRLAIPVNRPGQL

SEQ ID NO: 1177 - protein, 14 aa, Artificial Sequence
  Description of Artificial Sequence Synthetic peptide
VLDLAIGVNRGPQL

SEQ ID NO: 1178 - protein, 15 aa, Artificial Sequence
  Description of Artificial Sequence Synthetic peptide
VLDLAIPVNRPGFQL

SEQ ID NO: 1179 - protein, 15 aa, Artificial Sequence
  Description of Artificial Sequence Synthetic peptide
VLDLADIPVNRPGQL

SEQ ID NO: 1180 - protein, 15 aa, Artificial Sequence
  Description of Artificial Sequence Synthetic peptide
VLDLAIPVGNRPGQL

SEQ ID NO: 1181 - protein, 16 aa, Artificial Sequence
  Description of Artificial Sequence Synthetic peptide
VLQQDLAIPVNRPGQL

SEQ ID NO: 1182 - protein, 16 aa, Artificial Sequence
  Description of Artificial Sequence Synthetic peptide
VLDLAIPVNRGPGQKL

SEQ ID NO: 1183 - protein, 17 aa, Artificial Sequence
  Description of Artificial Sequence Synthetic peptide
VLDGLPLAIPVNRPGQL

SEQ ID NO: 1184 - protein, 16 aa, Artificial Sequence
  Description of Artificial Sequence Synthetic peptide
VLDLAIPVNRPGQLLL

SEQ ID NO: 1185 - protein, 17 aa, Artificial Sequence
  Description of Artificial Sequence Synthetic peptide
VLDLFLGAIPVNRPGQL

SEQ ID NO: 1186 - protein, 13 aa, Artificial Sequence
  Description of Artificial Sequence Synthetic peptide
VLDLAIPVNRGQL

SEQ ID NO: 1187 - protein, 13 aa, Artificial Sequence
  Description of Artificial Sequence Synthetic peptide
VLDLAPVNRPGQL

SEQ ID NO: 1188 - protein, 13 aa, Artificial Sequence
  Description of Artificial Sequence Synthetic peptide
LDLAIPVNRPGQL

SEQ ID NO: 1189 - protein, 13 aa, Artificial Sequence
  Description of Artificial Sequence Synthetic peptide
VLDLAIPVNRPGQ

SEQ ID NO: 1190 - protein, 12 aa, Artificial Sequence
  Description of Artificial Sequence Synthetic peptide
DLAIPVNRPGQL

SEQ ID NO: 1191 - protein, 12 aa, Artificial Sequence
  Description of Artificial Sequence Synthetic peptide
VLDLAIPVNRPG

SEQ ID NO: 1192 - protein, 12 aa, Artificial Sequence
  Description of Artificial Sequence Synthetic peptide
VLDLAINRPGQL

SEQ ID NO: 1193 - protein, 11 aa, Artificial Sequence
  Description of Artificial Sequence Synthetic peptide
VLDAIVNPGQL

SEQ ID NO: 1194 - protein, 14 aa, Artificial Sequence
  Description of Artificial Sequence Synthetic peptide
RGPQQYAEWQINER

SEQ ID NO: 1195 - protein, 14 aa, Artificial Sequence
  Description of Artificial Sequence Synthetic peptide
RGPQQYAEWQINDK

SEQ ID NO: 1196 - protein, 14 aa, Artificial Sequence
  Description of Artificial Sequence Synthetic peptide
RGPQQFAEWQINEK

SEQ ID NO: 1197 - protein, 14 aa, Artificial Sequence
  Description of Artificial Sequence Synthetic peptide
KGPQQYAEWQINEK

SEQ ID NO: 1198 - protein, 14 aa, Artificial Sequence
  Description of Artificial Sequence Synthetic peptide
RGPEQYAEWQINEK

SEQ ID NO: 1199 - protein, 14 aa, Artificial Sequence
  Description of Artificial Sequence Synthetic peptide
RGPQEYAEWQINEK

SEQ ID NO: 1200 - protein, 14 aa, Artificial Sequence
  Description of Artificial Sequence Synthetic peptide
RGPQQYADWQINEK

SEQ ID NO: 1201 - protein, 14 aa, Artificial Sequence
  Description of Artificial Sequence Synthetic peptide
RGPQQYAEYQINEK

SEQ ID NO: 1202 - protein, 14 aa, Artificial Sequence
  Description of Artificial Sequence Synthetic peptide
KGPEQYAEWQINEK

SEQ ID NO: 1203 - protein, 14 aa, Artificial Sequence
  Description of Artificial Sequence Synthetic peptide
KGPQEYAEWQINEK

SEQ ID NO: 1204 - protein, 14 aa, Artificial Sequence
  Description of Artificial Sequence Synthetic peptide
KGPQQFAEWQINEK

SEQ ID NO: 1205 - protein, 14 aa, Artificial Sequence
  Description of Artificial Sequence Synthetic peptide
RGPEQFAEWQINEK

SEQ ID NO: 1206 - protein, 14 aa, Artificial Sequence
  Description of Artificial Sequence Synthetic peptide
KGPQQYAEWQINER

SEQ ID NO: 1207 - protein, 14 aa, Artificial Sequence
  Description of Artificial Sequence Synthetic peptide
RGPQQYAEWQINDR

SEQ ID NO: 1208 - protein, 14 aa, Artificial Sequence
  Description of Artificial Sequence Synthetic peptide
RGPQQYADWQINDK

SEQ ID NO: 1209 - protein, 14 aa, Artificial Sequence
  Description of Artificial Sequence Synthetic peptide
RGPQQFAEWQINER

SEQ ID NO: 1210 - protein, 14 aa, Artificial Sequence
  Description of Artificial Sequence Synthetic peptide
RGPQQYAEWQVNEK

SEQ ID NO: 1211 - protein, 14 aa, Artificial Sequence
  Description of Artificial Sequence Synthetic peptide
RGPQQFAEWQINEK

SEQ ID NO: 1212 - protein, 14 aa, Artificial Sequence
  Description of Artificial Sequence Synthetic peptide
KGPQQFAEWQINER

SEQ ID NO: 1213 - protein, 14 aa, Artificial Sequence
  Description of Artificial Sequence Synthetic peptide
KGPQQFAEWQVNEK

SEQ ID NO: 1214 - protein, 14 aa, Artificial Sequence
  Description of Artificial Sequence Synthetic peptide
RGPQQFAEWQVNDK

SEQ ID NO: 1215 - protein, 14 aa, Artificial Sequence
  Description of Artificial Sequence Synthetic peptide
RGPQQYADWQINDR

SEQ ID NO: 1216 - protein, 14 aa, Artificial Sequence
  Description of Artificial Sequence Synthetic peptide
KGPQQYADWQINDK

SEQ ID NO: 1217 - protein, 14 aa, Artificial Sequence
  Description of Artificial Sequence Synthetic peptide
RGPQQFADYQINEK

SEQ ID NO: 1218 - protein, 14 aa, Artificial Sequence
  Description of Artificial Sequence Synthetic peptide
RGPQQYARWQINEK

SEQ ID NO: 1219 - protein, 14 aa, Artificial Sequence
  Description of Artificial Sequence Synthetic peptide
RGPQQYAEWQINEE

SEQ ID NO: 1220 - protein, 14 aa, Artificial Sequence
  Description of Artificial Sequence Synthetic peptide
HGPQQYAEWQINEK

SEQ ID NO: 1221 - protein, 14 aa, Artificial Sequence
  Description of Artificial Sequence Synthetic peptide
RGPYQYAEWQINEK

SEQ ID NO: 1222 - protein, 14 aa, Artificial Sequence
  Description of Artificial Sequence Synthetic peptide
RGPQQYMEWQINEK

SEQ ID NO: 1223 - protein, 14 aa, Artificial Sequence
  Description of Artificial Sequence Synthetic peptide
RGPQQYAEWQINEK

SEQ ID NO: 1224 - protein, 14 aa, Artificial Sequence
  Description of Artificial Sequence Synthetic peptide
RGPQQYAEWCINEK

SEQ ID NO: 1225 - protein, 14 aa, Artificial Sequence
  Description of Artificial Sequence Synthetic peptide
RGPQPYAEWQINEK

SEQ ID NO: 1226 - protein, 14 aa, Artificial Sequence
  Description of Artificial Sequence Synthetic peptide
RGGQQYAEWQINED

SEQ ID NO: 1227 - protein, 14 aa, Artificial Sequence
  Description of Artificial Sequence Synthetic peptide
RGPQQYARWKINEK

SEQ ID NO: 1228 - protein, 14 aa, Artificial Sequence
  Description of Artificial Sequence Synthetic peptide
RGGQQYAETQINEK

SEQ ID NO: 1229 - protein, 14 aa, Artificial Sequence
  Description of Artificial Sequence Synthetic peptide
RGPLQYAEWQNNEK

SEQ ID NO: 1230 - protein, 14 aa, Artificial Sequence
  Description of Artificial Sequence Synthetic peptide
EGPQQYAEWQINED

SEQ ID NO: 1231 - protein, 14 aa, Artificial Sequence
  Description of Artificial Sequence Synthetic peptide
RGPQQYAEWQINLL

SEQ ID NO: 1232 - protein, 14 aa, Artificial Sequence
  Description of Artificial Sequence Synthetic peptide
RGPQQGGEWQINEK

SEQ ID NO: 1233 - protein, 14 aa, Artificial Sequence
  Description of Artificial Sequence Synthetic peptide
RGPQQYAEWQIGGG

SEQ ID NO: 1234 - protein, 14 aa, Artificial Sequence
  Description of Artificial Sequence Synthetic peptide
RGPQQKYEWQINEK

SEQ ID NO: 1235 - protein, 14 aa, Artificial Sequence
  Description of Artificial Sequence Synthetic peptide
RGPQAQYEWQINEK

SEQ ID NO: 1236 - protein, 14 aa, Artificial Sequence
  Description of Artificial Sequence Synthetic peptide
RPHQQYAEWQINEK

SEQ ID NO: 1237 - protein, 14 aa, Artificial Sequence
  Description of Artificial Sequence Synthetic peptide
RGPQHHHEWQINEK

SEQ ID NO: 1238 - protein, 14 aa, Artificial Sequence
  Description of Artificial Sequence Synthetic peptide
RGPPQYAPPQINEK

SEQ ID NO: 1239 - protein, 14 aa, Artificial Sequence
  Description of Artificial Sequence Synthetic peptide
RGPQCYYEWCINEK

SEQ ID NO: 1240 - protein, 14 aa, Artificial Sequence
  Description of Artificial Sequence Synthetic peptide
RGPTQYAEGQINEG

SEQ ID NO: 1241 - protein, 15 aa, Artificial Sequence
  Description of Artificial Sequence Synthetic peptide
RGPQQYAEWQINEKG

SEQ ID NO: 1242 - protein, 15 aa, Artificial Sequence
  Description of Artificial Sequence Synthetic peptide
RGPQQYAEWQINEKY

SEQ ID NO: 1243 - protein, 16 aa, Artificial Sequence
  Description of Artificial Sequence Synthetic peptide
RGPQQYAFTEWQINEK

SEQ ID NO: 1244 - protein, 17 aa, Artificial Sequence
  Description of Artificial Sequence Synthetic peptide
RGPQSQYAEWQINEKPM

SEQ ID NO: 1245 - protein, 16 aa, Artificial Sequence
  Description of Artificial Sequence Synthetic peptide
RGPQQYAEWQINEKKK

SEQ ID NO: 1246 - protein, 17 aa, Artificial Sequence
  Description of Artificial Sequence Synthetic peptide
RRRRGPQQYAEWQINEK

SEQ ID NO: 1247 - protein, 13 aa, Artificial Sequence
  Description of Artificial Sequence Synthetic peptide
RGPQQYAEWQINE

SEQ ID NO: 1248 - protein, 12 aa, Artificial Sequence
  Description of Artificial Sequence Synthetic peptide
RGPQQYAEWQIN

SEQ ID NO: 1249 - protein, 11 aa, Artificial Sequence
  Description of Artificial Sequence Synthetic peptide
RGPQQYAEWQI

SEQ ID NO: 1250 - protein, 13 aa, Artificial Sequence
  Description of Artificial Sequence Synthetic peptide
GPQQYAEWQINEK

SEQ ID NO: 1251 - protein, 12 aa, Artificial Sequence
  Description of Artificial Sequence Synthetic peptide
PQQYAEWQINEK

SEQ ID NO: 1252 - protein, 11 aa, Artificial Sequence
  Description of Artificial Sequence Synthetic peptide
QQYAEWQINEK

SEQ ID NO: 1253 - protein, 8 aa, Artificial Sequence
  Description of Artificial Sequence Synthetic peptide
QQYAEWQI

SEQ ID NO: 1254 - protein, 11 aa, Artificial Sequence
  Description of Artificial Sequence Synthetic peptide
PQQYAEWQINE

SEQ ID NO: 1255 - protein, 10 aa, Artificial Sequence
  Description of Artificial Sequence Synthetic peptide
PQQYAEWQIN

SEQ ID NO: 1256 - protein, 7 aa, Artificial Sequence
  Description of Artificial Sequence Synthetic peptide
RGPQQYA

SEQ ID NO: 1257 - protein, 7 aa, Artificial Sequence
  Description of Artificial Sequence Synthetic peptide
EWQINEK

SEQ ID NO: 1258 - protein, 9 aa, Artificial Sequence
  Description of Artificial Sequence Synthetic peptide
QSFILSGNE

SEQ ID NO: 1259 - protein, 9 aa, Artificial Sequence
  Description of Artificial Sequence Synthetic peptide
ESFLLSGNQ

SEQ ID NO: 1260 - protein, 9 aa, Artificial Sequence
  Description of Artificial Sequence Synthetic peptide
QSYLLSGNQ

SEQ ID NO: 1261 - protein, 9 aa, Artificial Sequence
  Description of Artificial Sequence Synthetic peptide
QSFLLSGDQ

SEQ ID NO: 1262 - protein, 9 aa, Artificial Sequence
  Description of Artificial Sequence Synthetic peptide
QSYLLSGNE

SEQ ID NO: 1263 - protein, 9 aa, Artificial Sequence
  Description of Artificial Sequence Synthetic peptide
ESFLLSGNE

SEQ ID NO: 1264 - protein, 9 aa, Artificial Sequence
  Description of Artificial Sequence Synthetic peptide
ESYLLSGNQ

SEQ ID NO: 1265 - protein, 9 aa, Artificial Sequence
  Description of Artificial Sequence Synthetic peptide
QSFLLSGDE

SEQ ID NO: 1266 - protein, 9 aa, Artificial Sequence
  Description of Artificial Sequence Synthetic peptide
QSYLLSGDQ

SEQ ID NO: 1267 - protein, 9 aa, Artificial Sequence
  Description of Artificial Sequence Synthetic peptide
QSFRLSGNQ

SEQ ID NO: 1268 - protein, 9 aa, Artificial Sequence
  Description of Artificial Sequence Synthetic peptide
QSFLLSYNQ

SEQ ID NO: 1269 - protein, 9 aa, Artificial Sequence
  Description of Artificial Sequence Synthetic peptide
QFFLLSGNQ

SEQ ID NO: 1270 - protein, 9 aa, Artificial Sequence
  Description of Artificial Sequence Synthetic peptide
QSFLLSGAQ

SEQ ID NO: 1271 - protein, 9 aa, Artificial Sequence
  Description of Artificial Sequence Synthetic peptide
QSFLLSGNP

SEQ ID NO: 1272 - protein, 9 aa, Artificial Sequence
  Description of Artificial Sequence Synthetic peptide
QSFRRSGNQ

SEQ ID NO: 1273 - protein, 9 aa, Artificial Sequence
  Description of Artificial Sequence Synthetic peptide
QSFLLSYIQ

SEQ ID NO: 1274 - protein, 9 aa, Artificial Sequence
  Description of Artificial Sequence Synthetic peptide
QFFLLSGNL

SEQ ID NO: 1275 - protein, 9 aa, Artificial Sequence
  Description of Artificial Sequence Synthetic peptide
QSFLLSGAQ

SEQ ID NO: 1276 - protein, 9 aa, Artificial Sequence
  Description of Artificial Sequence Synthetic peptide
QSFLLSGNP

SEQ ID NO: 1277 - protein, 10 aa, Artificial Sequence
  Description of Artificial Sequence Synthetic peptide
QSFLLSGNQQ

SEQ ID NO: 1278 - protein, 10 aa, Artificial Sequence
  Description of Artificial Sequence Synthetic peptide
QSFLLLSGNQ

SEQ ID NO: 1279 - protein, 11 aa, Artificial Sequence
  Description of Artificial Sequence Synthetic peptide
AQSFGLLSGNQ

SEQ ID NO: 1280 - protein, 11 aa, Artificial Sequence
  Description of Artificial Sequence Synthetic peptide
RQSFLLISGNQ

SEQ ID NO: 1281 - protein, 10 aa, Artificial Sequence
  Description of Artificial Sequence Synthetic peptide
QSFLLSGNQK

SEQ ID NO: 1282 - protein, 8 aa, Artificial Sequence
  Description of Artificial Sequence Synthetic peptide
QFLLSGNQ

SEQ ID NO: 1283 - protein, 8 aa, Artificial Sequence
  Description of Artificial Sequence Synthetic peptide
SFLLSGNQ

SEQ ID NO: 1284 - protein, 8 aa, Artificial Sequence
  Description of Artificial Sequence Synthetic peptide
QSFLLSGN

SEQ ID NO: 1285 - protein, 8 aa, Artificial Sequence
  Description of Artificial Sequence Synthetic peptide
QSFLLGNQ

SEQ ID NO: 1286 - protein, 8 aa, Artificial Sequence
  Description of Artificial Sequence Synthetic peptide
QSFLSGNQ

SEQ ID NO: 1287 - protein, 8 aa, Artificial Sequence
  Description of Artificial Sequence Synthetic peptide
QSLLSGNQ

SEQ ID NO: 1288 - protein, 13 aa, Artificial Sequence
  Description of Artificial Sequence Synthetic peptide
VLDLAIPVNRPGQ

SEQ ID NO: 1289 - protein, 12 aa, Artificial Sequence
  Description of Artificial Sequence Synthetic peptide
VLDLAIPVNRPG

SEQ ID NO: 1290 - protein, 11 aa, Artificial Sequence
  Description of Artificial Sequence Synthetic peptide
VLDLAIPVNRP

SEQ ID NO: 1291 - protein, 13 aa, Artificial Sequence
  Description of Artificial Sequence Synthetic peptide
LDLAIPVNRPGQL

SEQ ID NO: 1292 - protein, 12 aa, Artificial Sequence
  Description of Artificial Sequence Synthetic peptide
DLAIPVNRPGQL

SEQ ID NO: 1293 - protein, 11 aa, Artificial Sequence
  Description of Artificial Sequence Synthetic peptide
LAIPVNRPGQL

SEQ ID NO: 1294 - protein, 12 aa, Artificial Sequence
  Description of Artificial Sequence Synthetic peptide
LDLAIPVNRPGQ

SEQ ID NO: 1295 - protein, 10 aa, Artificial Sequence
  Description of Artificial Sequence Synthetic peptide
DLAIPVNRPG

SEQ ID NO: 1296 - protein, 8 aa, Artificial Sequence
  Description of Artificial Sequence Synthetic peptide
LAIPVNRP

SEQ ID NO: 1297 - protein, 9 aa, Artificial Sequence
  Description of Artificial Sequence Synthetic peptide
VLDLAIPVN

SEQ ID NO: 1298 - protein, 10 aa, Artificial Sequence
  Description of Artificial Sequence Synthetic peptide
AIPVNRPGQL

SEQ ID NO: 1299 - protein, 7 aa, Artificial Sequence
  Description of Artificial Sequence Synthetic peptide
VNRPGQL

SEQ ID NO: 1300 - protein, 8 aa, Artificial Sequence
  Description of Artificial Sequence Synthetic peptide
VLDLAIPV

SEQ ID NO: 1301 - protein, 10 aa, Artificial Sequence
  Description of Artificial Sequence Synthetic peptide
VLDLAIPVNR

SEQ ID NO: 1302 - protein, 8 aa, Artificial Sequence
  Description of Artificial Sequence Synthetic peptide
QSFLLSGN

SEQ ID NO: 1303 - protein, 7 aa, Artificial Sequence
  Description of Artificial Sequence Synthetic peptide
QSFLLSG

SEQ ID NO: 1304 - protein, 6 aa, Artificial Sequence
  Description of Artificial Sequence Synthetic peptide
QSFLLS

SEQ ID NO: 1305 - protein, 8 aa, Artificial Sequence
  Description of Artificial Sequence Synthetic peptide
SFLLSGNQ

SEQ ID NO: 1306 - protein, 7 aa, Artificial Sequence
  Description of Artificial Sequence Synthetic peptide
FLLSGNQ

SEQ ID NO: 1307 - protein, 6 aa, Artificial Sequence
  Description of Artificial Sequence Synthetic peptide
LLSGNQ

SEQ ID NO: 1308 - protein, 7 aa, Artificial Sequence
  Description of Artificial Sequence Synthetic peptide
SFLLSGN

SEQ ID NO: 1309 - protein, 13 aa, Artificial Sequence
  Description of Artificial Sequence Synthetic peptide
DTFYNAAWDPSNR

SEQ ID NO: 1310 - protein, 11 aa, Artificial Sequence
  Description of Artificial Sequence Synthetic peptide
ALDWAIANLLR

SEQ ID NO: 1311 - protein, 12 aa, Artificial Sequence
  Description of Artificial Sequence Synthetic peptide
YDYENVDAGAAK

SEQ ID NO: 1312 - protein, 11 aa, Artificial Sequence
  Description of Artificial Sequence Synthetic peptide
EVQDSPLDACR

SEQ ID NO: 1313 - protein, 21 aa, Artificial Sequence
  Description of Artificial Sequence Synthetic peptide
ITSVNSQKFPILNLIQMSATR

SEQ ID NO: 1314 - protein, 11 aa, Artificial Sequence
  Description of Artificial Sequence Synthetic peptide
SRVQVVSNFGK

SEQ ID NO: 1315 - protein, 34 aa, Artificial Sequence
  Description of Artificial Sequence Synthetic polypeptide
VLDLAIPVNKPGQLQSFLLSGTQNQPSLLSGFSK

SEQ ID NO: 1316 - protein, 22 aa, Artificial Sequence
  Description of Artificial Sequence Synthetic peptide
NAMFVPHYNLNANSIIYALKGR

SEQ ID NO: 1317 - protein, 21 aa, Artificial Sequence
  Description of Artificial Sequence Synthetic peptide
LRPGVMFVVPAGHPFVNIASK

SEQ ID NO: 1318 - protein, 12 aa, Artificial Sequence
  Description of Artificial Sequence Synthetic peptide
GLFDLGHPLVNR

SEQ ID NO: 1319 - protein, 12 aa, Artificial Sequence
  Description of Artificial Sequence Synthetic peptide
RPYYSNAPQEIF

SEQ ID NO: 1320 - protein, 12 aa, Artificial Sequence
  Description of Artificial Sequence Synthetic peptide
VLLEQQEQEPQH

SEQ ID NO: 1321 - protein, 15 aa, Artificial Sequence
  Description of Artificial Sequence Synthetic peptide
QQYGIAASPFLQSAA